SEQUENCE LISTING (114 sequences)

SEQ ID NO: 1 - protein, 5 aa, Artificial Sequence
  Synthetic
XNXXX

SEQ ID NO: 2 - protein, 7 aa, Artificial Sequence
  Synthetic
XDXNXXX

SEQ ID NO: 3 - protein, 1424 aa, Artificial Sequence
  Factor VIII amino acid sequence wherein the B-domain (amino acid residues 741-1648) is removed
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 1438 aa, Artificial Sequence
  amino acid sequence for a B-domain deleted Factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 1465 aa, Artificial Sequence
  amino acid sequence for a B-domain deleted Factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRRRAQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 1445 aa, Artificial Sequence
  amino acid sequence for a B-domain deleted Factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - protein, 165 aa, Artificial Sequence
  EPO amino acid sequence
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

SEQ ID NO: 8 - protein, 2351 aa, Artificial Sequence [sequence omitted]

SEQ ID NO: 9 - protein, 2332 aa, Artificial Sequence
  amino acid sequence for Factor VIII
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 10 - protein, 140 aa, Artificial Sequence
  biologically active portion of the full-lenghth BMP-7 sequence (sequence S.1)
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 11 - protein, 140 aa, Artificial Sequence
  BMP-7 variant
MNLTSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 12 - protein, 140 aa, Artificial Sequence
  BMP-7 variant
MSNLTKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 13 - protein, 140 aa, Artificial Sequence
  BMP-7 variant
MSTNLTQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 14 - protein, 140 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACNLT

SEQ ID NO: 15 - protein, 141 aa, Artificial Sequence
  BMP-7 variant
MNLTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 16 - protein, 141 aa, Artificial Sequence
  BMP-7 variant
MSNLTSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 17 - protein, 141 aa, Artificial Sequence
  BMP-7 variant
MSTNLTKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 18 - protein, 141 aa, Artificial Sequence
  BMP-7 variant
MSTGNLTQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 19 - protein, 141 aa, Artificial Sequence
  BMP-7 variant
MSTGSNLTRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 20 - protein, 141 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGNLT

SEQ ID NO: 21 - protein, 142 aa, Artificial Sequence
  BMP-7 variant
MNLTTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 22 - protein, 142 aa, Artificial Sequence
  BMP-7 variant
MSNLTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 23 - protein, 142 aa, Artificial Sequence
  BMP-7 variant
MSTNLTSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 24 - protein, 142 aa, Artificial Sequence
  BMP-7 variant
MSTGNLTKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 25 - protein, 142 aa, Artificial Sequence
  BMP-7 variant
MSTGSNLTQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 26 - protein, 142 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCNLT

SEQ ID NO: 27 - protein, 143 aa, Artificial Sequence
  BMP-7 variant
MNLTSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 28 - protein, 143 aa, Artificial Sequence
  BMP-7 variant
MSNLTTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 29 - protein, 143 aa, Artificial Sequence
  BMP-7 variant
MSTNLTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 30 - protein, 143 aa, Artificial Sequence
  BMP-7 variant
MSTGNLTSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 31 - protein, 143 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHNLT

SEQ ID NO: 32 - protein, 5 aa, Artificial Sequence
  N-linked glycosylation sequence
DRNLT

SEQ ID NO: 33 - protein, 140 aa, Artificial Sequence
  BMP-7 variant
MDRNLTQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 34 - protein, 140 aa, Artificial Sequence
  BMP-7 variant
MSDRNLTRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 35 - protein, 140 aa, Artificial Sequence
  BMP-7 variant
MSTDRNLTSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 36 - protein, 140 aa, Artificial Sequence
  BMP-7 variant
MSTGDRNLTQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 37 - protein, 140 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRDRNLT

SEQ ID NO: 38 - protein, 145 aa, Artificial Sequence
  BMP-7 variant
MDRNLTSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 39 - protein, 144 aa, Artificial Sequence
  BMP-7 variant
MDRNLTTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 40 - protein, 143 aa, Artificial Sequence
  BMP-7 variant
MDRNLTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 41 - protein, 142 aa, Artificial Sequence
  BMP-7 variant
MDRNLTSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 42 - protein, 141 aa, Artificial Sequence
  BMP-7 variant
MDRNLTKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 43 - protein, 145 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHDRNLT

SEQ ID NO: 44 - protein, 144 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCDRNLT

SEQ ID NO: 45 - protein, 143 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGDRNLT

SEQ ID NO: 46 - protein, 142 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACDRNLT

SEQ ID NO: 47 - protein, 141 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRADRNLT

SEQ ID NO: 48 - protein, 5 aa, Artificial Sequence
  N-linked glycosylation sequence
DFNVS

SEQ ID NO: 49 - protein, 141 aa, Artificial Sequence
  BMP-7 variant
MDFNVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 50 - protein, 141 aa, Artificial Sequence
  BMP-7 variant
MSDFNVSQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 51 - protein, 141 aa, Artificial Sequence
  BMP-7 variant
MSTDFNVSRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 52 - protein, 141 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRADFNVS

SEQ ID NO: 53 - protein, 142 aa, Artificial Sequence
  BMP-7 variant
MDFNVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 54 - protein, 142 aa, Artificial Sequence
  BMP-7 variant
MSDFNVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 55 - protein, 142 aa, Artificial Sequence
  BMP-7 variant
MSTDFNVSQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 56 - protein, 142 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACDFNVS

SEQ ID NO: 57 - protein, 143 aa, Artificial Sequence
  BMP-7 variant
MDFNVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 58 - protein, 143 aa, Artificial Sequence
  BMP-7 variant
MSDFNVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 59 - protein, 143 aa, Artificial Sequence
  BMP-7 variant
MSTDFNVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 60 - protein, 143 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGDFNVS

SEQ ID NO: 61 - protein, 144 aa, Artificial Sequence
  BMP-7 variant
MDFNVSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 62 - protein, 144 aa, Artificial Sequence
  BMP-7 variant
MSDFNVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 63 - protein, 144 aa, Artificial Sequence
  BMP-7 variant
MSTDFNVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 64 - protein, 144 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCDFNVS

SEQ ID NO: 65 - protein, 145 aa, Artificial Sequence
  BMP-7 variant
MDFNVSSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 66 - protein, 145 aa, Artificial Sequence
  BMP-7 variant
MSDFNVSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 67 - protein, 145 aa, Artificial Sequence
  BMP-7 variant
MSTDFNVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 68 - protein, 145 aa, Artificial Sequence
  BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHDFNVS

SEQ ID NO: 69 - protein, 31 aa, Artificial Sequence
  selected polypeptide region of BMP-7 from A73 through P103
AFPLNSYMNATNHAIVQTLVHFINPETVPKP

SEQ ID NO: 70 - protein, 31 aa, Artificial Sequence
  selected polypeptide region of BMP-7 variant including NLT between A73 and A82
NLTLNSYMNATNHAIVQTLVHFINPETVPKP

SEQ ID NO: 71 - protein, 31 aa, Artificial Sequence
  selected polypeptide region of BMP-7 variant including NLT between A73 and A82
ANLTNSYMNATNHAIVQTLVHFINPETVPKP

SEQ ID NO: 72 - protein, 31 aa, Artificial Sequence
  selected polypeptide region of BMP-7 variant including NLT between A73 and A82
AFNLTSYMNATNHAIVQTLVHFINPETVPKP

SEQ ID NO: 73 - protein, 31 aa, Artificial Sequence
  selected polypeptide region of BMP-7 variant including NLT between A73 and A82
AFPNLTYMNATNHAIVQTLVHFINPETVPKP

SEQ ID NO: 74 - protein, 31 aa, Artificial Sequence
  selected polypeptide region of BMP-7 variant including NLT between A73 and A82
AFPLNLTMNATNHAIVQTLVHFINPETVPKP

SEQ ID NO: 75 - protein, 31 aa, Artificial Sequence
  selected polypeptide region of BMP-7 variant including NLT between A73 and A82
AFPLNNLTNATNHAIVQTLVHFINPETVPKP

SEQ ID NO: 76 - protein, 31 aa, Artificial Sequence
  selected polypeptide region of BMP-7 variant including NLT between A73 and A82
AFPLNSNLTATNHAIVQTLVHFINPETVPKP

SEQ ID NO: 77 - protein, 31 aa, Artificial Sequence
  selected polypeptide region of BMP-7 variant including NLT between A73 and A82
AFPLNSYNLTTNHAIVQTLVHFINPETVPKP

SEQ ID NO: 78 - protein, 31 aa, Artificial Sequence
  selected polypeptide region of BMP-7 variant including NLT between I95 and P103
AFPLNSYMNATNHAIVQTLVHFNLTETVPKP

SEQ ID NO: 79 - protein, 31 aa, Artificial Sequence
  selected polypeptide region of BMP-7 variant including NLT between A73 and A82
AFPLNSYMNATNHAIVQTLVHFINLTTVPKP

SEQ ID NO: 80 - protein, 31 aa, Artificial Sequence
  selected polypeptide region of BMP-7 variant including NLT between A73 and A82
AFPLNSYMNATNHAIVQTLVHFINNLTVPKP

SEQ ID NO: 81 - protein, 31 aa, Artificial Sequence
  selected polypeptide region of BMP-7 variant including NLT between A73 and A82
AFPLNSYMNATNHAIVQTLVHFINPNLTPKP

SEQ ID NO: 82 - protein, 31 aa, Artificial Sequence
  selected polypeptide region of BMP-7 variant including NLT between A73 and A82
AFPLNSYMNATNHAIVQTLVHFINPENLTKP

SEQ ID NO: 83 - protein, 31 aa, Artificial Sequence
  selected polypeptide region of BMP-7 variant including NLT between A73 and A82
AFPLNSYMNATNHAIVQTLVHFINPETNLTP

SEQ ID NO: 84 - protein, 31 aa, Artificial Sequence
  selected polypeptide region of BMP-7 variant including NLT between A73 and A82
AFPLNSYMNATNHAIVQTLVHFINPETVNLT

SEQ ID NO: 85 - protein, 32 aa, Artificial Sequence
  selected polypeptide region of BMP-7 variant
NLTPLNSYMNATNHAIVQTLVHFINPETVPKP

SEQ ID NO: 86 - protein, 32 aa, Artificial Sequence
  selected polypeptide region of BMP-7 variant
ANLTLNSYMNATNHAIVQTLVHFINPETVPKP

SEQ ID NO: 87 - protein, 32 aa, Artificial Sequence
  selected polypeptide region of BMP-7 variant
AFNLTNSYMNATNHAIVQTLVHFINPETVPKP

SEQ ID NO: 88 - protein, 32 aa, Artificial Sequence
  selected polypeptide region of BMP-7 variant
AFPNLTSYMNATNHAIVQTLVHFINPETVPKP

SEQ ID NO: 89 - protein, 32 aa, Artificial Sequence
  selected polypeptide region of BMP-7 variant
AFPLNLTYMNATNHAIVQTLVHFINPETVPKP

SEQ ID NO: 90 - protein, 32 aa, Artificial Sequence
  selected polypeptide region of BMP-7 variant
AFPLNNLTMNATNHAIVQTLVHFINPETVPKP

SEQ ID NO: 91 - protein, 32 aa, Artificial Sequence
  selected polypeptide region of BMP-7 variant
AFPLNSNLTNATNHAIVQTLVHFINPETVPKP

SEQ ID NO: 92 - protein, 32 aa, Artificial Sequence
  selected polypeptide region of BMP-7 variant
AFPLNSYNLTATNHAIVQTLVHFINPETVPKP

SEQ ID NO: 93 - protein, 32 aa, Artificial Sequence
  selected polypeptide region of BMP-7 variant
AFPLNSYMNLTTNHAIVQTLVHFINPETVPKP

SEQ ID NO: 94 - protein, 32 aa, Artificial Sequence
  selected polypeptide region of BMP-7 variant
AFPLNSYMNATNHAIVQTLVHFNLTPETVPKP

SEQ ID NO: 95 - protein, 32 aa, Artificial Sequence
  selected polypeptide region of BMP-7 variant
AFPLNSYMNATNHAIVQTLVHFINLTETVPKP

SEQ ID NO: 96 - protein, 32 aa, Artificial Sequence
  selected polypeptide region of BMP-7 variant
AFPLNSYMNATNHAIVQTLVHFINNLTTVPKP

SEQ ID NO: 97 - protein, 32 aa, Artificial Sequence
  selected polypeptide region of BMP-7 variant
AFPLNSYMNATNHAIVQTLVHFINPNLTVPKP

SEQ ID NO: 98 - protein, 32 aa, Artificial Sequence
  selected polypeptide region of BMP-7 variant
AFPLNSYMNATNHAIVQTLVHFINPENLTPKP

SEQ ID NO: 99 - protein, 32 aa, Artificial Sequence
  selected polypeptide region of BMP-7 variant
AFPLNSYMNATNHAIVQTLVHFINPETNLTKP

SEQ ID NO: 100 - protein, 32 aa, Artificial Sequence
  selected polypeptide region of BMP-7 variant
AFPLNSYMNATNHAIVQTLVHFINPETVNLTP

SEQ ID NO: 101 - protein, 32 aa, Artificial Sequence
  selected polypeptide region of BMP-7 variant
AFPLNSYMNATNHAIVQTLVHFINPETVPNLT

SEQ ID NO: 102 - protein, 713 aa, Campylobacter jejuni
MLKKEYLKNPYLVLFAMIVLAYVFSVFCRFYWVWWASEFNEYFFNNQLMIISNDGYAFAEGARDMIAGFHQPNDLSYYGSSLSTLTYWLYKITPFSFESIILYMSTFLSSLVVIPIILLANEYKRPLMGFVAALLASVANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDFSEFMRRISGSEIVFLFSLFGFVWLLRKHKSMIMALPILVLGFLALKGGLRFTIYSVPVMALGFGFLLSEFKAILVKKYSQLTSNVCIVFATILTLAPVFIHIYNYKAPTVFSQNEASLLNQLKNIANREDYVVTWWDYGYPVRYYSDVKTLVDGGKHLGKDNFFPSFSLSKDEQAAANMARLSVEYTEKSFYAPQNDILKSDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 103 - protein, 413 aa, Neisseria gonorrhoeae
MSKAVKRLFDIIASASGLIVLSPVFLVLIYLIRKNLGSPVFFIRERPGKDGKPFKMVKFRSMRDALDSDGIPLPDSERLTDFGKKLRATSLDELPELWNVLKGEMSLVGPRPLLMQYLPLYNKFQNRRHEMKPGITGWAQVNGRNALSWDEKFSCDVWYTDNFSFWLDMKILFLTVKKVLIKEGISAQGEATMPPFAGNRKLAVIGAGGHGKVVAELAAALGTYGEIVFLDDRTQGSVNGFPVIGTTLLLENSLSPEQFDITVAVGNNRIRRQITENAAALGFKLPVLIHPDATVSPSAIIGQGSVVMAKAVVQAGSVLKDGVIVNTAATVDHDCLLDAFVHISPGAHLSGNTRIGEESRIGTGACSRQQTTVGSGVTAGAGAVIVCDIPDGMTVAGNPAKPLTGKNPKTGTA

SEQ ID NO: 104 - protein, 332 aa, Saccharomyces cerevisiae
MKWCSTYIIIWLAIIFHKFQKSTATASHNIDDILQLKGDTGVITVTADNYPLLSRGVPGYFNILYITMRGTNSNGMSCQLCHDFEKTYQAVADVIRSQAPQSLNLFFTVDVNEVPQLVKDLKLQNVPHLVVYPPAESNKQSQFEWKTSPFYQYSLVPENAENTLQFGDFLAKILNISITVPQAFNVQEFVYYFVACMVVFIFIKKVILPKVTNKWKLFSMILSLGILLPSITGYKFVEMNAIPFIARDAKNRIMYFSGGSGWQFGIEIFSVSLMYIVMSALSVLLIYVPKISCVSEKMRGLLSSFLACVLFYFFSYFISCYLIKNPGYPIVF

SEQ ID NO: 105 - protein, 456 aa, Homo sapiens
MGYFRCAGAGSFGRRRKMEPSTAARAWALFWLLLPLLGAVCASGPRTLVLLDNLNVRETHSLFFRSLKDRGFELTFKTADDPSLSLIKYGEFLYDNLIIFSPSVEDFGGNINVETISAFIDGGGSVLVAASSDIGDPLRELGSECGIEFDEEKTAVIDHHNYDISDLGQHTLIVADTENLLKAPTIVGKSSLNPILFRGVGMVADPDNPLVLDILTGSSTSYSFFPDKPITQYPHAVGKNTLLIAGLQARNNARVIFSGSLDFFSDSFFNSAVQKAAPGSQRYSQTGNYELAVALSRWVFKEEGVLRVGPVSHHRVGETAPPNAYTVTDLVEYSIVIQQLSNAKWVPFDGDDIQLEFVRIDPFVRTFLKKKGGKYSVQFKLPDVYGVFQFKVDYNRLGYTHLYSSTQVSVRPLQHTQYERFIPSAYPYYASAFPMMLGLFIFSIVFLHMKEKEKSD

SEQ ID NO: 106 - protein, 441 aa, Mus musculus
MKMDPRLAVRAWPLCGLLLAVLGCVCASGPRTLVLLDNLNVRDTHSLFFRSLKDRGFELTFKTADDPSLSLIKYGEFLYDNLIIFSPSVEDFGGNINVETISAFIDGGGSVLVAASSDIGDPLRELGSECGIEFDEEKTAVIDHHNYDVSDLGQHTLIVADTENLLKAPTIVGKSSLNPILFRGVGMVADPDNPLVLDILTGSSTSYSFFPDKPITQYPHAVGRNTLLIAGLQARNNARVIFSGSLDFFSDAFFNSAVQKATPGAQRYSQTGNYELAVALSRWVFKEEGVLRVGPVSHHRVGEMAPPNAYTVTDLVEYSIVIEQLSNGKWVPFDGDDIQLEFVRIDPFVRTFLKRKGGKYSVQFKLPDVYGVFQFKVDYNRLGYTHLYSSTQVSVRPLQHTQYERFIPSAYPYYASAFSMMAGLFIFSIVFLHMKEKEKSD

SEQ ID NO: 107 - protein, 171 aa, Candida albicans
MAKSASNKKSIPTTSSSSTTTSAASSSVVLKEVKSTLTTTINNYFDTISAQPRLKLIDLFLIFLVLLGILQFIYVLIIGNFPFNSFLGGFISCVGQFVLLVSLRLQINDSTTTTTNKESDDQLELDEDKIENGTTGGGNGRLFKEITPERSFGDFIFASLILHFIVIHFIN

SEQ ID NO: 108 - protein, 301 aa, Homo sapiens
MIFLGFADDVLNLRWRHKLLLPTAASLPLLMVYFTNFGNTTIVVPKPFRPILGLHLDLGILYYVYMGLLAVFCTNAINILAGINGLEAGQSLVISASIIVFNLVELEGDCRDDHVFSLYFMIPFFFTTLGLLYHNWYPSRVFVGDTFCYFAGMTFAVVGILGHFSKTMLLFFMPQVFNFLYSLPQLLHIIPCPRHRIPRLNIKTGKLEMSYSKFKTKSLSFLGTFILKVAESLQLVTVHQSETEDGEFTECNNMTLINLLLKVLGPIHERNLTLLLLLLQILGSAITFSIRYQLVRLFYDV

SEQ ID NO: 109 - protein, 408 aa, Homo sapiens
MWAFSELPMPLLINLIVSLLGFVATVTLIPAFRGHFIAARLCGQDLNKTSRQQIPESQGVISGAVFLIILFCFIPFPFLNCFVKEQCKAFPHHEFVALIGALLAICCMIFLGFADDVLNLRWRHKLLLPTAASLPLLMVYFTNFGNTTIVVPKPFRPILGLHLDLGILYYVYMGLLAVFCTNAINILAGINGLEAGQSLVISASIIVFNLVELEGDCRDDHVFSLYFMIPFFFTTLGLLYHNWYPSRVFVGDTFCYFAGMTFAVVGILGHFSKTMLLFFMPQVFNFLYSLPQLLHIIPCPRHRIPRLNIKTGKLEMSYSKFKTKSLSFLGTFILKVAESLQLVTVHQSETEDGEFTECNNMTLINLLLKVLGPIHERNLTLLLLLLQILGSAITFSIRYQLVRLFYDV

SEQ ID NO: 110 - protein, 410 aa, Mus musculus
MWAFPELPLPLPLLVNLIGSLLGFVATVTLIPAFRSHFIAARLCGQDLNKLSQQQIPESQGVISGAVFLIILFCFIPFPFLNCFVEEQCKAFPHHEFVALIGALLAICCMIFLGFADDVLNLRWRHKLLLPTAASLPLLMVYFTNFGNTTIVVPKPFRWILGLHLDLGILYYVYMGLLAVFCTNAINILAGINGLEAGQSLVISASIIVFNLVELEGDYRDDHIFSLYFMIPFFFTTLGLLYHNWYPSRVFVGDTFCYFAGMTFAVVGILGHFSKTMLLFFMPQVFNFLYSLPQLFHIIPCPRHRMPRLNAKTGKLEMSYSKFKTKNLSFLGTFILKVAENLRLVTVHQGESEDGAFTECNNMTLINLLLKVFGPIHERNLTLLLLLLQVLSSAATFSIRYQLVRLFYDV

SEQ ID NO: 111 - protein, 448 aa, Saccharomyces cerevisiae
MLRLFSLALITCLIYYSKNQGPSALVAAVGFGIAGYLATDMLIPRVGKSFIKIGLFGKDLSKPGRPVLPETIGAIPAAVYLFVMFIYIPFIFYKYMVITTSGGGHRDVSVVEDNGMNSNIFPHDKLSEYLSAILCLESTVLLGIADDLFDLRWRHKFFLPAIAAIPLLMVYYVDFGVTHVLIPGFMERWLKKTSVDLGLWYYVYMASMAIFCPNSINILAGVNGLEVGQCIVLAILALLNDLLYFSMGPLATRDSHRFSAVLIIPFLGVSLALWKWNRWPATVFVGDTYCYFAGMVFAVVGILGHFSKTMLLLFIPQIVNFIYSCPQLFKLVPCPRHRLPKFNEKDGLMYPSRANLKEEPPKSIFKPILKLLYCLHLIDLEFDENNEIISTSNMTLINLTLVWFGPMREDKLCNTILKLQFCIGILALLGRHAIGAIIFGHDNLWTVR

SEQ ID NO: 112 - protein, 200 aa, Campylobacter jejuni
MYEKVFKRIFDFILALVLLVLFSPVILITALLLKITQGSVIFTQNRPGLDEKIFKIYKFKTMSDERDEKGELLSDELRLKAFGKIVRSLSLDELLQLFNVLKGDMSFVGPRPLLVEYLSLYNEEQKLRHKVRPGITGWAQVNGRNAISWQKKFELDVYYVKNISFLLDLKIMFLTALKVLKRSGVSKEGHVTTEKFNGKN

SEQ ID NO: 113 - protein, 391 aa, Neisseria gonorrhoea
MLNTALSPWPSFTREEADAVSKVLLSNKVNYWTGSECREFEKEFAAFAGTRYAVALSNGTLALDAALKAIGIGAGDDVIVTSRTFLASASCIVNAGANPVFADVDLNSQNISAETVKAVLTPNTKAVIVVHLAGMPAEMDGIMALAKEHDLWVIEDCAQAHGATYKGKSVGSIGHVGAWSFCQDKIITTGGEGGMVTTNDKTLWEKMWAYKDHGKSYDAVYHREHAPGFRWLHESFGTNWRMMEMQAVIGRIQLKHLPEWTARRQENAAKLAESLRKFKSIRLIEVAGYIGHAQYKFYAFVKPEHLKDDWTRDRIVSELNARNVPCYQGGCSEVYLEKAFDNTPWRPKERLKNAVELGGTALTFLVHPTLTDDEIAFCKKHIEAVLTEAAR

SEQ ID NO: 114 - protein, 308 aa, Methanobrevibacter smithii
MKTAVLIPCYNEELTIKKVILDFKKALPKADIYVYDNNSTDNSYEIAKDTGAIVKREYRQGKGNVVRSMFRDIDADCYILVDGDDTYPAEASKEIEELILSKKADMVIGDRLSSTYFEENKRRFHNSGNKLVRKLINTIFNSDISDIMTGMRGFSYEFVKSFPISSKEFEIETEMTIFALNHNFLIKELPIEYRDRMDGSESKLNTFSDGYKVISLLFGLFRDIRPLFFFSLVTLVLLIIAGLYFFPILIDFYRTGFVEKVPTLITVGVVAIVAVIIFFTGVVLHVIRKQHDENFEHHLNLIAQNKKR

(19) United States
(12) Patent Application Publication
(10) Pub. No.: US 2010/0286067 A1
(43) Pub. Date: Nov. 11, 2010

(54) GLYCOCONJUGATION OF POLYPEPTIDES USING OLIGOSACCHARYLTRANSFERASES

(75) Inventor: Shawn DeFrees, North Wales, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: BIOGENERIX AG, Mannheim (DE)

(21) Appl. No.: 12/811,963

(22) PCT Filed: Jan. 8, 2009
(86) PCT No.: PCT/US09/30503

§ 371 Date: Jul. 20, 2010

Related U.S. Application Data
(60) Provisional application No. 61/019,805, filed on Jan. 8, 2008.

(51) Int. Cl. A61K 38/14; C07K 7/06; C12N 9/96; C12N 5/00; C07H 21/00; C12N 15/63; C40B 40/10; C12P 21/06

(52) U.S. Cl. 514/20.9; 530/322; 435/188; 435/325; 536/17.1; 536/23.1; 435/320.1; 506/18; 435/68.1

(57) ABSTRACT

The current invention provides polypeptides and polypeptide conjugates that include an exogenous N-linked glycosylation sequence. The N-linked glycosylation sequence is preferably a substrate for an oligosaccharyltransferase (e.g., bacterial PgIB), which can catalyze the transfer of a glycosyl moiety from a lipid-bound glycosyl donor molecule (e.g., a lipid-pyrophosphate-linked glycosyl moiety) to an asparagine (N) residue of the glycosylation sequence. In one example, the asparagine residue is part of an exogenous N-linked glycosylation sequence of the invention. The invention further provides methods of making the polypeptide conjugates that include contacting a polypeptide having an N-linked glycosylation sequence of the invention and a lipid-pyrophosphate-linked glycosyl moiety (or phospholipid-linked glycosyl moiety) in the presence of an oligosaccharyltransferase under conditions sufficient for the enzyme to transfer the glycosyl moiety to an asparagine residue of the N-linked glycosylation sequence. Exemplary glycosyl moieties that can be conjugated to the glycosylation sequence include GlcNAc, GlcNH, bacillosamine, 6-hydroybacillosamine, GalNAc, GaINH, GlcNAc-GlcNAc, GlcNAc-GlcNH, GlcNAc-Gal, GlcNAc-GlcNAc-Gal-Sia, GlcNAc-Gal-Sia, GlcNAc-GlcNAc-Man, and GlcNAc-GlcNAc-Man(Man)2. The transferred glycosyl moiety is optionally modified with a modifying group, such as a polymer (e.g., PEG). In one example, the modified glycosyl moiety is a GIcNAc or a sialic acid moiety.

### CROSS-REFERENCES TO RELATED APPLICATIONS

This application claims the benefit of U.S. Provisional Patent Application No. 61/019,805 filed on Jan. 8, 2008, the contents of which is incorporated herein by reference in its entirety for all purposes.

### FIELD OF THE INVENTION

The invention pertains to the field of polypeptide modification by glycosylation. In particular, the invention relates to a method of preparing glycosylated polypeptides using short enzyme-recognized N-linked glycosylation sequences.

### BACKGROUND OF THE INVENTION

The administration of glycosylated and non-glycosylated polypeptides for engendering a particular physiological response is well known in the medicinal arts. For example, both purified and recombinant human growth hormone (hGH) are used for treating conditions and diseases associated with hGH deficiency, e.g., dwarfism in children. Other examples involve interferon, which has known antiviral activity as well as granulocyte colony stimulating factor (G-CSF), which stimulates the production of white blood cells.

The lack of expression systems that can be used to manufacture polypeptides with wild-type glycosylation patterns has limited the use of such polypeptides as therapeutic agents. It is known in the art that improperly or incompletely glycosylated polypeptides can be immunogenic, leading to rapid neutralization of the peptide and/or the development of an allergic response. Other deficiencies of recombinantly produced glycopeptides include suboptimal potency and rapid clearance from the bloodstream.

One approach to solving the problems inherent in the production of glycosylated polypeptide therapeutics has been to modify the polypeptides in vitro after their expression. Post-expression in vitro modification of polypeptides has been used for both the modification of existing glycan structures and the attachment of glycosyl moieties to non-glycosylated amino acid residues. A comprehensive selection of recombinant eukaryotic glycosyltransferases has become available, making in vitro enzymatic synthesis of mammalian glycoconjugates with custom designed glycosylation patterns and glycosyl structures possible. See, for example, U.S. Pat. Nos. 5,876,980; 6,030,815; 5,728,554; 5,922,577; as well as WO/9831826; US2003180835; and WO 03/031464.

In addition, glycopeptides have been derivatized with one or more non-saccharide modifying groups, such as water soluble polymers. An exemplary polymer that has been conjugated to peptides is poly(ethylene glycol) (“PEG”). PEG-conjugation, which increases the molecular size of the polypeptide, has been used to reduce immunogenicity and to prolong blood clearance time of PEG-conjugated polypeptides. For example, U.S. Pat. No. 4,179,337 to Davis et al. discloses non-immunogenic polypeptides such as enzymes and polypeptide-hormones coupled to polyethylene glycol (PEG) or polypropylene glycol (PPG).

The principal method for the attachment of PEG and its derivatives to polypeptides involves non-specific bonding through an amino acid residue (see e.g., U.S. Pat. No. 4,088,538 U.S. Pat. No. 4,496,689, U.S. Pat. No. 4,414,147, U.S. Pat. No. 4,055,635, and PCT WO 87/00056). Another method of PEG-conjugation involves the non-specific oxidation of glycosyl residues of a glycopeptide (see e.g., WO 94/05332).

In these non-specific methods, PEG is added in a random, non-specific manner to reactive residues on a polypeptide backbone. This approach has significant drawbacks, including a lack of homogeneity of the final product, and the possibility of reduced biological or enzymatic activity of the modified polypeptide. Therefore, a derivatization method for therapeutic polypeptides that results in the formation of a specifically labeled, readily characterizable and essentially homogeneous product is highly desirable.

Specifically modified, homogeneous polypeptide therapeutics can be produced in vitro through the use of enzymes. Unlike non-specific methods for attaching a modifying group, such as a synthetic polymer, to a polypeptide, enzyme-based syntheses have the advantages of regioselectivity and stereoselectivity. Two principal classes of enzymes for use in the synthesis of labeled polypeptides are glycosyltransferases (e.g., sialyltransferases, oligosaccharyltransferases, N-acetylglucosaminyltransferases), and glycosidases. These enzymes can be used for the specific attachment of sugars which can subsequently be altered to comprise a modifying group. Alternatively, glycosyltransferases and modified glycosidases can be used to directly transfer modified sugars to a polypeptide backbone (see e.g., U.S. Pat. No. 6,399,336, and U.S. Patent Application Publications 20030040037, 20040132640, 20040137557, 20040126838, and 20040142856, each of which are incorporated by reference herein). Methods combining both chemical and enzymatic approaches are also known (see e.g., Yamamoto et al., Carbohydr. Res. 305: 415-422 (1998) and U.S. Patent Application Publication 20040137557, which is incorporated herein by reference).

Carbohydrates are attached to glycopeptides in several ways of which N-linked to asparagine and O-linked to serine and threonine are the most relevant for recombinant glycoprotein therapeutics.

Not all polypeptides comprise a glycosylation sequence as part of their amino acid sequence. In addition, existing glycosylation sequences may not be suitable for the attachment of a modifying group. Such modification may, for example, cause an undesirable decrease in biological activity of the modified polypeptide. Thus, there is a need in the art for precise and reproducible glycosylation and glycomodification methods. The current invention addresses these and other needs.

### SUMMARY OF THE INVENTION

The present invention includes the discovery that enzymatic glycoconjugation or glycoPEGylation reactions can be specifically targeted to certain N-linked glycosylation sequences within a polypeptide. In one example, the targeted glycosylation sequence is introduced into a parent polypeptide (e.g., wild-type polypeptide) by mutation creating a mutant polypeptide that includes an N-linked glycosylation sequence, wherein the N-linked glycosylation sequence is not present, or not present at the same position, in the corresponding parent polypeptide (exogenous N-linked glycosylation sequence). Such mutant polypeptides are termed “sequon polypeptides”.

In one aspect, the present invention provides polypeptides that include at least one exogenous N-linked glycosylation sequence and methods of making such polypeptides. The invention also provides libraries of sequon polypeptides. In a representative embodiment, the library includes a plurality of different members, wherein each member of the library corresponds to a common parent polypeptide and wherein each member of the library includes an exogenous N-linked glycosylation sequence of the invention. Also provided are methods of making and using such libraries.

In one embodiment, each N-linked glycosylation sequence is a substrate for an enzyme, such as an oligosaccharyltransferase, such as those described herein (e.g., PglB or Stt3), which can transfer a modified or non-modified glycosyl moiety from a glycosyl donor species onto an asparagine residue of the N-linked glycosylation sequence. Hence, in another aspect, the invention provides a covalent conjugate between a glycosylated polypeptide and a modifying group (e.g., a polymeric modifying group), wherein the polypeptide includes an exogenous N-linked glycosylation sequence. The polymeric modifying group is covalently conjugated to the polypeptide at an asparagine residue within the N-linked glycosylation sequence via a glycosyl linking group interposed between and covalently linked to both the polypeptide and the polymeric modifying group, wherein the glycosyl linking group is a member selected from monosaccharides and oligosaccharides. The invention further provides pharmaceutical compositions including a polypeptide conjugate of the invention.

Exemplary N-linked glycosylation sequences of use in polypeptides of the invention are selected from SEQ ID NO: 1 and SEQ ID NO: 2:

| | |
|---|---|
| X¹ N X² X³ X⁴; | (SEQ ID NO: 1) |
| and | |
| X¹ D X^{2′} N X² X³ X⁴, | (SEQ ID NO: 2) |

wherein N is asparagine; D is aspartic acid; X³ is a member selected from threonine (T) and serine (S); X¹ is either present or absent and when present is an amino acid; X⁴ is either present or absent and when present is an amino acid; and X² and X^{2′} are independently selected amino acids. In one embodiment, X² and X^{2′} are not proline (P).

The invention further provides methods of making and using the polypeptide conjugates. In one example, the polypeptide conjugate is formed between a polypeptide and a modifying group (e.g., a polymeric modifying group) using a cell-free in vitro method. The polypeptide includes a N-linked glycosylation sequence of the invention including an asparagine residue. The modifying group is covalently linked to the polypeptide at the asparagine residue via a glycosyl linking group that is interposed between and covalently linked to both the polypeptide and the modifying group. The method includes contacting the polypeptide and a glycosyl donor species of the invention in the presence of an oligosaccharyltransferase under conditions sufficient for the oligosaccharyltransferase to transfer a glycosyl moiety from the glycosyl donor species onto the asparagine residue of the N-linked glycosylation sequence.

Another exemplary method of forming a covalent conjugate between a polypeptide and a modifying group (e.g., a polymeric modifying group) involves intracellular glycosylation within a host cell, in which the polypeptide is expressed. The method takes advantage of endogenous and/or co-expressed oligosaccharyl transferases. The method includes contacting the polypeptide, which includes an N-linked glycosylation sequence (e.g. a polypeptide of the invention, and a glycosyl donor species in the presence of an intracellular enzyme (e.g., an oligosaccharyltransferase) under conditions sufficient for the enzyme to transfer a glycosyl moiety from the glycosyl donor species onto an asparagine residue of the N-linked glycosylation sequence. In one example, the glycosyl donor species is added to the cell culture medium, internalized by the host cell and used as a substrate by an intracellular (endogenous or co-expressed) oligosaccharyltransferase.

In another aspect, the invention provides glycosyl donor species useful in the methods of the invention. Exemplary glycosyl donor species have a structure according to Formula (X):

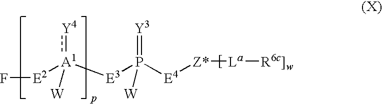

wherein w is an integer selected from 1 to 20. In one example, w is selected from 1-8. The integer p is selected from 0 and 1. F is a lipid moiety; Z* is a glycosyl moiety selected from monosaccharides and oligosaccharides; each L^{a} is a linker moiety independently selected from a single bond, a functional group, substituted or unsubstituted alkyl, substituted or unsubstituted heteroalkyl, substituted or unsubstituted aryl, substituted or unsubstituted heteroaryl and substituted or unsubstituted heterocycloalkyl; each R^{6c} is an independently selected modifying group, such as a linear or branched polymeric modifying group described herein (e.g., PEG); A¹ is a member selected from P (phosphorus) and C (carbon); Y³ is a member selected from oxygen (O) and sulfur (S); Y⁴ is a member selected from O, S, SR¹, OR¹, OQ, CR¹R² and NR³R⁴; E², E³ and E⁴ are members independently selected from CR¹R², O, S and NR³; and each W is a member independently selected from SR¹, OR¹, OQ, NR³R⁴, substituted or unsubstituted alkyl, substituted or unsubstituted heteroalkyl, substituted or unsubstituted aryl, substituted or unsubstituted heteroaryl and substituted or unsubstituted heterocycloalkyl, wherein each Q is a member independently selected from H, a single negative charge and a cation (e.g., Na⁺ or K⁺). Each R¹, each R², each R³ and each R⁴ is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

Additional aspects, advantages and objects of the present invention will be apparent from the detailed description that follows.

### BRIEF DESCRIPTION OF THE DRAWINGS

FIG. 1A and FIG. 1B (SEQ ID NO: 8 and SEQ ID NO: 9, respectively) each show an exemplary amino acid sequence for Factor VIII.

FIG. 2 is an exemplary Factor VIII amino acid sequence, wherein the B-domain (amino acid residues 741-1648) is removed (SEQ ID NO: 3). Exemplary polypeptides of the invention include those in which the deleted B-domain is replaced with at least one amino acid residue (B-domain replacement sequence). In one embodiment, the B-domain replacement sequence between Arg⁷⁴⁰ and Glu¹⁶⁴⁹ includes at least one O-linked or N-linked glycosylation sequence.

FIG. 3 is an exemplary amino acid sequence for B-domain deleted Factor VIII (SEQ ID NO: 4).

FIG. 4 is an exemplary amino acid sequence for B-domain deleted Factor VIII (SEQ ID NO: 5).

FIG. 5 is an exemplary amino acid sequence for B-domain deleted Factor VIII (SEQ ID NO: 6).

FIG. 6 is a Table outlining exemplary embodiments of the invention, in which a particular polypeptide of the invention is used in conjunction with a particular N-linked glycosylation sequence of the invention. Each row in FIG. 6 represents an exemplary embodiment of the invention, in which the N-linked glycosylation sequence is introduced into the polypeptide at the indicated position within the amino acid sequence of the polypeptide.

### DETAILED DESCRIPTION OF THE INVENTION

### I. Abbreviations

PEG, poly(ethyleneglycol); m-PEG, methoxy-poly(ethylene glycol); PPG, poly(propyleneglycol); m-PPG, methoxy-poly(propylene glycol); Fuc, fucose or fucosyl; Gal, galactose or galactosyl; GalNAc, N-acetylgalactosamine or N-acetylgalactosaminyl; Glc, glucose or glucosyl; GlcNAc, N-acetylglucosamine or N-acetylglucosaminyl; Man, mannose or mannosyl; ManAc, mannosamine acetate or mannosaminyl acetate; Sia, sialic acid or sialyl; and NeuAc, N-acetylneuramine or N-acetylneuraminyl.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures of analytical and synthetic organic chemistry described below are those well known and commonly employed in the art. Standard techniques, or modifications thereof, are used for chemical syntheses and chemical analyses.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, for example, Essentials of Glycobiology Varki et al. eds. CSHL Press (1999). Oligosaccharides may include a glycosyl mimetic moiety as one of the sugar components. Oligosaccharides are considered to have a reducing end and a non-reducing end, whether or not the saccharide at the reducing end is in fact a reducing sugar.

The term “glycosyl moiety” means any radical derived from a sugar residue. “Glycosyl moiety” includes mono- and oligosaccharides and encompasses “glycosyl-mimetic moiety.”

The term “glycosyl-mimetic moiety,” as used herein refers to a moiety, which structurally resembles a glycosyl moiety (e.g., a hexose or a pentose). Examples of “glycosyl-mimetic moiety” include those moieties, wherein the glycosidic oxygen or the ring oxygen of a glycosyl moiety, or both, has been replaced with a bond or another atom (e.g., sulfur), or another moiety, such as a carbon- (e.g., CH₂), or nitrogen-containing group (e.g., NH). Examples include substituted or unsubstituted cyclohexyl derivatives, cyclic thioethers, cyclic secondary amines, moieties including a thioglycosidic bond, and the like. In one example, the “glycosyl-mimetic moiety” is transferred in an enzymatically catalyzed reaction onto an amino acid residue of a polypeptide or a glycosyl moiety of a glycopeptide. This can, for instance, be accomplished by activating the “glycosyl-mimetic moiety” with a leaving group, such as a halogen.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

The term “uncharged amino acid” refers to amino acids, that do not include an acidic (e.g., —COOH) or basic (e.g., —NH₂) functional group. Basic amino acids include lysine (K) and arginine (R). Acidic amino acids include aspartic acid (D) and glutamic acid (E). “Uncharged amino acids include, e.g., glycine (G), valine (V), leucine (L), isoleucine (I), phenylalanine (F), but also those amino acids that include —OH, —SH or —SCH₃ groups (e.g., threonine (T), serine (S), tyrosine (Y), cysteine (C) and methionine (M).

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Peptide” refers to a polymer including monomers derived from amino acids joined together through amide bonds. Peptides of the present invention can vary in size, e.g., from two amino acids to hundreds or thousands of amino acids. A larger peptide (e.g., at least 10, at least 20, at least 30 or at least 50 amino acid residues) is alternatively referred to as a “polypeptide” or “protein”. Additionally, unnatural amino acids, for example, β-alanine, phenylglycine, homoarginine and homophenylalanine are also included. Amino acids that are not gene-encoded may also be used in the present invention. Furthermore, amino acids that have been modified to include reactive groups, glycosylation sequences, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” or “polypeptide” refers to both glycosylated and non-glycosylated peptides or “polypeptides”. Also included are polypetides that are incompletely glycosylated by a system that expresses the polypeptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983). The term “polypeptide” also includes all possible forms of that polypeptide, such as mutated forms (one or more mutations), truncated forms, elongated forms, fusion proteins including the polyeptide, tagged polypetides, variants, in which a particular domain is removed or partially removed, and the like. The term “polypeptide” includes monomers, oligomers and polymers of that polypeptide. For example, the term “von Willebrand Factor” (vWF) includes monomeric, dimeric and oligomeric forms of vWF.

In the present application, amino acid residues are numbered (typically in the superscript) according to their relative positions from the N-terminal amino acid (e.g., N-terminal methionine) of the polypeptide, which is numbered “1”. The N-terminal amino acid may be a methionine (M), numbered “1”. The numbers associated with each amino acid residue can be readily adjusted to reflect the absence of N-terminal methionine if the N-terminus of the polypeptide starts without a methionine. It is understood that the N-terminus of an exemplary polypeptide can start with or without a methionine.

The term “parent polypeptide” refers to any polypeptide, which has an amino acid sequence, which does not include an “exogenous” N-linked glycosylation sequence of the invention. However, a “parent polypeptide” may include one or more naturally occurring (endogenous) N-linked glycosylation sequence. For example, a wild-type polypeptide may include the N-linked glycosylation sequence “NLT”. The term “parent polypeptide” refers to any polypeptide including wild-type polypeptides, fusion polypeptides, synthetic polypeptides, recombinant polypeptides (e.g., therapeutic polypeptides) as well as any variants thereof (e.g., previously modified through one or more replacement of amino acids, insertions of amino acids, deletions of amino acids and the like) as long as such modification does not amount to forming an N-linked glycosylation sequence of the invention. In one embodiment, the amino acid sequence of the parent polypeptide, or the nucleic acid sequence encoding the parent polypeptide, is defined and accessible to the public in any way. For example, the parent polypeptide is a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank and the like). In another example, the parent polypeptide is not a wild-type polypeptide but is used as a therapeutic polypeptide (i.e., authorized drug) and the sequence of such polypeptide is publicly available in a scientific publication or patent. In yet another example, the amino acid sequence of the parent polypeptide or the nucleic acid sequence encoding the parent polypeptide was accessible to the public in any way at the time of the invention. In one embodiment, the parent polypeptide is part of a larger structure. For example, the parent polypeptide corresponds to the constant region (F_{c}) region or C_{H}2 domain of an antibody, wherein these domains may be part of an entire antibody. In one embodiment, the parent polypeptide is not an antibody of unknown sequence.

The term “mutant polypeptide” or “polypeptide variant” refers to a form of a polypeptide, wherein its amino acid sequence differs from the amino acid sequence of its corresponding wild-type form, naturally existing form or any other parent form. A mutant polypeptide can contain one or more mutations, e.g., replacement, insertion, deletion, etc. which result in the mutant polypeptide.

The term “sequon polypeptide” refers to a polypeptide variant that includes in its amino acid sequence an “exogenous N-linked glycosylation sequence.” A “sequon polypeptide” contains at least one exogenous N-linked glycosylation sequence, but may also include one or more endogenous (e.g., naturally occurring) N-linked glycosylation sequence.

The term “exogenous N-linked glycosylation sequence” refers to an N-linked glycosylation sequence that is introduced into the amino acid sequence of a parent polypeptide (e.g., wild-type polypeptide), wherein the parent polypeptide either does not include an N-linked glycosylation sequence or includes an N-linked glycosylation sequence at a different position. In one example, an N-linked glycosylation sequence is introduced into a wild-type polypeptide that does not have an N-linked glycosylation sequence. In another example, a wild-type polypeptide naturally includes a first N-linked glycosylation sequence at a first position. A second N-linked glycosylation is introduced into this wild-type polypeptide at a second position. This modification results in a polypeptide having an “exogenous N-linked glycosylation sequence” at the second position. The exogenous N-linked glycosylation sequence may be introduced into the parent polypeptide by mutation. Alternatively, a polypeptide with an exogenous N-linked glycosylation sequence can be made by chemical synthesis.

The term “corresponding to a parent polypeptide” (or grammatical variations of this term) is used to describe a sequon polypeptide of the invention, wherein the amino acid sequence of the sequon polypeptide differs from the amino acid sequence of the corresponding parent polypeptide only by the presence of at least one exogenous N-linked glycosylation sequence of the invention. Typically, the amino acid sequences of the sequon polypeptide and the parent polypeptide exhibit a high percentage of identity. In one example, “corresponding to a parent polypetide” means that the amino acid sequence of the sequon polypeptide has at least about 50% identity, at least about 60%, at least about 70%, at least about 80%, at least about 90%, at least about 95% or at least about 98% identity to the amino acid sequence of the parent polypeptide. In another example, the nucleic acid sequence that encodes the sequon polypeptide has at least about 50% identity, at least about 60%, at least about 70%, at least about 80%, at least about 90%, at least about 95% or at least about 98% identity to the nucleic acid sequence encoding the parent polypeptide.

The term “introducing (or adding, etc.) a glycosylation sequence (e.g., an N-linked glycosylation sequence) into a parent polypeptide” (or grammatical variations thereof), or “modifying a parent polypeptide” to include a glycosylation sequence (or grammatical variations thereof) does not necessarily mean that the parent polypeptide is a physical starting material for such conversion, but rather that the parent polypeptide provides the guiding amino acid sequence for the making of another polypeptide. In one example, “introducing a glycosylation sequence into a parent polypeptide” means that the gene for the parent polypeptide is modified through appropriate mutations to create a nucleotide sequence that encodes a sequon polypeptide. In another example, “introducing a glycosylation sequence into a parent polypeptide” means that the resulting polypeptide is theoretically designed using the parent polypeptide sequence as a guide. The designed polypeptide may then be generated by chemical or other means.

The term “lead polypeptide” refers to a sequon polypeptide of the invention that can be effectively glycosylated and/or glycoconjugated (e.g., glycoPEGylated), e.g. by a method of the invention. For a sequon polypeptide of the invention to qualify as a lead polypeptide, such polypeptide, when subjected to suitable reaction conditions, is preferably glycosylated or glycoconjugated (e.g., glycoPEGylated) with a reaction yield of at least about 50%, preferably at least about 60%, more preferably at least about 70% and even more preferably about 80%, about 85%, about 90% or about 95%. Most preferred are those lead polypeptides of the invention, which can be glycosylated or glycoconjugated (e.g., glycoPEGylated) with a reaction yield of greater than 80%, greater than 85%, greater than 90%, or greater than 95%. In one preferred embodiment, the lead polypeptide is glycosylated or glycoPEGylated in such a fashion that only one amino acid residue of each N-linked glycosylation sequence is glycosylated or glycoconjugated (e.g., glycoPEGylated) (mono-glycosylation). In various embodiments, the single amino acid residue glycosylated or glycoconjugated is located within the exogenous N-linked glycosylation sequence.

The term “library” refers to a collection of different polypeptides, each member of the library corresponding to a common parent polypeptide. Each polypeptide species in the library is referred to as a “member” of the library. Preferably, the library of the present invention is a collection of polypeptides of sufficient number and diversity to afford a population from which to identify a lead polypeptide. A library includes at least two different polypeptides. In one embodiment, the library includes from about 2 to about 10 members. In another embodiment, the library includes from about 10 to about 20 members. In yet another embodiment, the library includes from about 20 to about 30 members. In a further embodiment, the library includes from about 30 to about 50 members. In another embodiment, the library includes from about 50 to about 100 members. In yet another embodiment, the library includes more than 100 members. The members of the library may be part of a mixture or may be isolated from each other. In one example, the members of the library are part of a mixture that optionally includes other components. For example, at least two sequon polypeptides are present in a volume of cell-culture broth. In another example, the members of the library are each expressed separately and are optionally isolated. The isolated sequon polypeptides may optionally be contained in a multi-well container, in which each well contains a different type of sequon polypeptide.

The term “C_{H}2” domain of the present invention is meant to describe an immunoglobulin heavy chain constant C_{H}2 domain. In defining an immunoglobulin C_{H}2 domain reference is made to immunoglobulins in general and in particular to the domain structure of immunoglobulins as applied to human IgG1 by Kabat E. A. (1978) Adv. Protein Chem. 32:1-75.

The term “polypeptide comprising a C_{H}2 domain” or “polypeptide comprising at least one C_{H}2 domain” is intended to include whole antibody molecules, antibody fragments (e.g., Fc domain), or fusion proteins that include a region equivalent to the C_{H}2 region of an immunoglobulin.

The term “polypeptide conjugate,” refers to species of the invention in which a polypeptide is glycoconjugated with a sugar moiety (e.g., modified sugar) as set forth herein. In a representative example, the polypeptide is a sequon polypeptide having an exogenous O-linked glycosylation sequence.

“Proximate a proline residue” or “in proximity to a proline residue” as used herein refers to an amino acid that is less than about 10 amino acids removed from a proline residue, preferably, less than about 9, 8, 7, 6 or 5 amino acids removed from a proline residue, more preferably, less than about 4, 3 or 2 residues removed from a proline residue. The amino acid “proximate a proline residue” may be on the C- or N-terminal side of the proline residue.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

As used herein, the term “modified sugar,” refers to a naturally- or non-naturally-occurring carbohydrate. In one embodiment, the “modified sugar” is enzymatically added onto an amino acid or a glycosyl residue of a polypeptide using a method of the invention. The modified sugar is selected from a number of enzyme substrates including, but not limited to sugar nucleotides (mono-, di-, and tri-phosphates), activated sugars (e.g., glycosyl halides, glycosyl mesylates) and sugars that are neither activated nor nucleotides. The “modified sugar” is covalently functionalized with a “modifying group.” Useful modifying groups include, but are not limited to, polymeric modifying groups (e.g., water-soluble polymers), therapeutic moieties, diagnostic moieties, biomolecules and the like. In one embodiment, the modifying group is not a naturally occurring glycosyl moiety (e.g., naturally occurring polysaccharide). The modifying group is preferably non-naturally occurring. In one example, the “non-naturally occurring modifying group” is a polymeric modifying group, in which at least one polymeric moiety is non-naturally occurring. In another example, the non-naturally occurring modifying group is a modified carbohydrate. The locus of functionalization with the modifying group is selected such that it does not prevent the “modified sugar” from being added enzymatically to a polypeptide. “Modified sugar” also refers to any glycosyl mimetic moiety that is functionalized with a modifying group and which is a substrate for a natural or modified enzyme, such as a glycosyltransferase.

As used herein, the term “polymeric modifying group” is a modifying group that includes at least one polymeric moiety (polymer). In one example, the polymeric modifying group when added to a polypeptide can alter at least one biological property of such polypeptide, for example, its bioavailability, biological activity, its in vivo half-life or immunogenicity. Exemplary polymers include water soluble and water insoluble polymers. A polymeric modifying group can be linear or branched and can include one or more independently selected polymeric moieties, such as poly(alkylene glycol) and derivatives thereof. In one example, the polymer is non-naturally occurring. In an exemplary embodiment, the polymeric modifying group includes a water-soluble polymer, e.g., poly(ethylene glycol) and derivatived thereof (PEG, m-PEG), poly(propylene glycol) and derivatives thereof (PPG, m-PPG) and the like. In a preferred embodiment, the poly(ethylene glycol) or poly(propylene glycol) has a molecular weight that is essentially homodisperse. In one embodiment the polymeric modifying group is a naturally occurring or non-naturally occurring polysaccharide (e.g., polysialic acid).

The term “water-soluble” refers to moieties that have some detectable degree of solubility in water. Methods to detect and/or quantify water solubility are well known in the art. Exemplary water-soluble polymers include peptides, oligo- and polysaccharides, poly(ethers), poly(amines), poly(carboxylic acids) and the like. Peptides can have mixed sequences or be composed of a single amino acid [poly(amino acid), e.g., poly(lysine)]. An exemplary polysaccharide is poly(sialic acid). An exemplary poly(ether) is poly(ethylene glycol), e.g., m-PEG. Poly(ethylene imine) is an exemplary polyamine, and poly(acrylic) acid is a representative poly(carboxylic acid).

The polymer backbone of the water-soluble polymer can be poly(ethylene glycol) (i.e. PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, difunctional PEG, multiarmed PEG, forked PEG, branched PEG, pendent PEG (i.e. PEG or related polymers having one or more functional groups pendent to the polymer backbone), or PEG with degradable linkages therein. Likewise, the term poly(alkylene oxide) is meant to include all forms of such material and includes materials incorporating more than one type of poly(alkylene oxide), such as combinations of PEG and PPG.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine or cysteine. In one example, the branched poly(ethylene glycol) can be represented in general form as R(-PEG-OH)ₘ in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as the polymer backbone.

Many other polymers are also suitable for the invention. Polymer backbones that are non-peptidic and water-soluble, are particularly useful in the invention. Examples of suitable polymers include, but are not limited to, other poly(alkylene glycols), such as poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, as well as copolymers, terpolymers, and mixtures thereof. Although the molecular weight of each chain of the polymer backbone can vary, it is typically in the range of from about 100 Da to about 100,000 Da, often from about 5,000 Da to about 80,000 Da.

The term “glycoconjugation,” as used herein, refers to the enzymatically mediated conjugation of a modified sugar species to an amino acid or glycosyl residue of a polypeptide, e.g., a mutant human growth hormone of the present invention. In one example, the modified sugar is covalently attached to one or more modifying groups. A subgenus of “glycoconjugation” is “glycol-PEGylation” or “glyco-PEGylation”, in which the modifying group of the modified sugar is poly(ethylene glycol) or a derivative thereof, such as an alkyl derivative (e.g., m-PEG) or a derivative with a reactive functional group (e.g., H₂N-PEG, HOOC-PEG).

The terms “large-scale” and “industrial-scale” are used interchangeably and refer to a reaction cycle that produces at least about 250 mg, preferably at least about 500 mg, and more preferably at least about 1 gram of glycoconjugate at the completion of a single reaction cycle.

The term “N-linked glycosylation sequence” or “sequon” refers to any amino acid sequence (e.g., containing from about 3 to about 9 amino acids, preferably about 3 to about 6 amino acids) that includes at least one asparagine (N) residue. In one embodiment, the N-linked glycosylation sequence is a substrate for an enzyme, such as an oligosaccharyltransferase, preferably when part of an amino acid sequence of a polypeptide. In a typical embodiment, the enzyme transfers a glycosyl moiety onto the N-linked glycosylation sequence by modifying the amino group of the above described asparagine residue, which is referred to as the “site of glycosylation”. The invention distinguishes between an N-linked glycosylation sequence that is naturally occurring in a wild-type polypeptide or any other parent form thereof (endogenous N-linked glycosylation sequence) and an “exogenous N-linked glycosylation sequence”. A polypeptide that includes an exogenous N-linked glycosylation sequence is termed “sequon polypeptide”. The amino acid sequence of a parent polypeptide may be modified to include an exogenous N-linked glycosylation sequence through recombinant technology, chemical syntheses or other means.

The term, “glycosyl linking group,” as used herein refers to a glycosyl residue to which a modifying group (e.g., PEG moiety, therapeutic moiety, biomolecule) is covalently attached; the glycosyl linking group joins the modifying group to the remainder of the conjugate. In the methods of the invention, the “glycosyl linking group” becomes covalently attached to a glycosylated or unglycosylated polypeptide, thereby linking the modifying group to an amino acid and/or glycosyl residue of the polypeptide. A “glycosyl linking group” is generally derived from a “modified sugar” by the enzymatic attachment of the “modified sugar” to an amino acid and/or glycosyl residue of the polypeptide. The glycosyl linking group can be a saccharide-derived structure that is degraded during formation of modifying group-modified sugar cassette (e.g., oxidation→Schiff base formation→reduction), or the glycosyl linking group may be an “intact glycosyl linking group”. A “glycosyl linking group” may include a glycosyl-mimetic moiety. For example, the glycosyl transferase (e.g., sialyl transferase), which is used to add the modified sugar to a glycosylated polypeptide, exhibits tolerance for a glycosyl-mimetic substrate (e.g., a modified sugar in which the sugar moiety is a glycosyl-mimetic moiety—e.g., sialyl-mimetic moiety). The transfer of the modified glycosyl-mimetic sugar results in a conjugate having a glycosyl linking group that is a glycosyl-mimetic moiety.

The term “intact glycosyl linking group” refers to a glycosyl linking group that is derived from a glycosyl moiety, in which the saccharide monomer that links the modifying group to the remainder of the conjugate is not degraded, e.g., chemically oxidized using an. For example, the ring structure is opened by oxidation e.g., by sodium metaperiodate or wherein. An exemplary “intact glycosyl linking groups” of the invention is a sialic acid moiety, in which the C-6 side chain is intact (CHOH—CHOH—CH₂OH).

The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO and the like.

The term “linking group” is any chemical group that links two moities. In one example, the linking group includes at least one heteroatom. Exemplar linking groups include ether, thioether, amine, carboxamide, sulfonamide, hydrazine, carbonyl, carbamate, urea, thiourea, ester and carbonate.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is bound to a carrier, e.g, multivalent agents. Therapeutic moiety also includes proteins and constructs that include proteins. Exemplary proteins include, but are not limited to, Erythropoietin (EPO), Granulocyte Colony Stimulating Factor (GCSF), Granulocyte Macrophage Colony Stimulating Factor (GMCSF), Interferon (e.g., Interferon-α, -β, -γ), Interleukin (e.g., Interleukin II), serum proteins (e.g., Factors VII, VIIa, VIII, IX, and X), Human Chorionic Gonadotropin (HCG), Follicle Stimulating Hormone (FSH) and Lutenizing Hormone (LH) and antibody fusion proteins (e.g. Tumor Necrosis Factor Receptor ((TNFR)/Fc domain fusion protein)).

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of polypeptides with anti-tumor activity, e.g. TNF-α. Conjugates include, but are not limited to those formed between a therapeutic protein and a glycoprotein of the invention. A representative conjugate is that formed between PSGL-1 and TNF-α.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diptheria toxin, and snake venom (e.g., cobra venom).

As used herein, “a radioactive agent” includes any radioisotope that is effective in diagnosing or destroying a tumor. Examples include, but are not limited to, indium-111, cobalt-60. Additionally, naturally occurring radioactive elements such as uranium, radium, and thorium, which typically represent mixtures of radioisotopes, are suitable examples of a radioactive agent. The metal ions are typically chelated with an organic chelating moiety.

Many useful chelating groups, crown ethers, cryptands and the like are known in the art and can be incorporated into the compounds of the invention (e.g., EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

As used herein, “pharmaceutically acceptable carrier” includes any material, which when combined with the conjugate retains the conjugates' activity and is non-reactive with the subject's immune systems. “Pharmaceutically acceptable carrier” includes solids and liquids, such as vehicles, diluents and solvents. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

The term “therapy” refers to “treating” or “treatment” of a disease or condition including preventing the disease or condition from occurring in a subject (e.g., human) that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any gramatically equivalent term means the amount that, when administered to an animal or human for treating a disease, is sufficient to effect treatment for that disease.

The term “isolated” refers to a material that is substantially or essentially free from components, which are used to produce the material. For polypeptide conjugates of the invention, the term “isolated” refers to material that is substantially or essentially free from components, which normally accompany the material in the mixture used to prepare the polypeptide conjugate. “Isolated” and “pure” are used interchangeably. Typically, isolated polypeptide conjugates of the invention have a level of purity preferably expressed as a range. The lower end of the range of purity for the polypeptide conjugates is about 60%, about 70% or about 80% and the upper end of the range of purity is about 70%, about 80%, about 90% or more than about 90%.

When the polypeptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, mass-spectroscopy, or a similar means).

“Essentially each member of the population,” as used herein, describes a characteristic of a population of polypeptide conjugates of the invention in which a selected percentage of the modified sugars added to a polypeptide are added to multiple, identical acceptor sites on the polypeptide. “Essentially each member of the population” speaks to the “homogeneity” of the sites on the polypeptide conjugated to a modified sugar and refers to conjugates of the invention, which are at least about 80%, preferably at least about 90% and more preferably at least about 95% homogenous.

“Homogeneity,” refers to the structural consistency across a population of acceptor moieties to which the modified sugars are conjugated. Thus, in a polypeptide conjugate of the invention in which each modified sugar moiety is conjugated to an acceptor site having the same structure as the acceptor site to which every other modified sugar is conjugated, the polypeptide conjugate is said to be about 100% homogeneous. Homogeneity is typically expressed as a range. The lower end of the range of homogeneity for the polypeptide conjugates is about 50%, about 60%, about 70% or about 80% and the upper end of the range of purity is about 70%, about 80%, about 90% or more than about 90%.

When the polypeptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The purity of the polypeptide conjugates is typically determined by one or more methods known to those of skill in the art, e.g., liquid chromatography-mass spectrometry (LC-MS), matrix assisted laser desorption mass time of flight spectrometry (MALDITOF), capillary electrophoresis, and the like.

“Substantially uniform glycoform” or a “substantially uniform glycosylation pattern,” when referring to a glycopeptide species, refers to the percentage of acceptor moieties that are glycosylated by the glycosyltransferase of interest (e.g., GalNAc transferase). For example, in the case of a α1,2 fucosyltransferase, a substantially uniform fucosylation pattern exists if substantially all (as defined below) of the Galβ1,4-GlcNAc-R and sialylated analogues thereof are fucosylated in a peptide conjugate of the invention. It will be understood by one of skill in the art, that the starting material may contain glycosylated acceptor moieties (e.g., fucosylated Galβ1,4-GlcNAc-R moieties). Thus, the calculated percent glycosylation will include acceptor moieties that are glycosylated by the methods of the invention, as well as those acceptor moieties already glycosylated in the starting material.

The term “substantially” in the above definitions of “substantially uniform” generally means at least about 40%, at least about 70%, at least about 80%, or more preferably at least about 90%, and still more preferably at least about 95% of the acceptor moieties for a particular glycosyltransferase are glycosylated.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl” by itself or as part of another substituent, means, unless otherwise stated, a straight or branched chain, or cyclic (i.e., cycloalkyl)hydrocarbon radical, or combination thereof, which may be fully saturated, mono- or polyunsaturated and can include di- (e.g., alkylene) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″)═NR″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R″′ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R″′)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “acyl” describes a substituent containing a carbonyl residue, C(O)R. Exemplary species for R include H, halogen, alkoxy, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si), boron (B) and phosphorus (P).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

The term “pharmaceutically acceptable salts” includes salts, which are prepared with relatively nontoxic acids or bases, depending on the particular substituents found on the compounds described herein. When compounds of the present invention contain relatively acidic functionalities, base addition salts can be obtained by contacting such compounds (e.g., their neutral form) with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting such compounds (e.g., their neutral form) with a sufficient amount of the desired acid, either neat or in a suitable inert solvent. Examples of pharmaceutically acceptable acid addition salts include those derived from inorganic acids like hydrochloric, sulfonic, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The graphic representations of racemic, ambiscalemic and scalemic or enantiomerically pure compounds used herein are taken from Maehr, J. Chem. Ed., 62: 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

The terms “enantiomeric excess” and diastereomeric excess” are used interchangeably herein. Compounds with a single stereocenter are referred to as being present in “enantiomeric excess,” those with at least two stereocenters are referred to as being present in “diastereomeric excess.”

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds. ORGANIC FUNCTIONAL GROUP PREPARATIONS, Academic Press, San Diego, 1989).

“Non-covalent protein binding groups” are moieties that interact with an intact or denatured polypeptide in an associative manner. The interaction may be either reversible or irreversible in a biological milieu. The incorporation of a “non-covalent protein binding group” into a chelating agent or complex of the invention provides the agent or complex with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, thiophosphate, phosphonate, carboxylate, boronate, sulfate, sulfone, sulfonate, thiosulfate, and thiosulfonate.

“enzyme truncation” or “truncated enzyme” or grammatical variants, as well as “domain-deleted enzyme” or grammatical variants, refer to an enzyme that has fewer amino acid residues than the corresponding naturally occurring enzyme, but that retains certain enzymatic activity. Any number of amino acid residues can be deleted so long as the enzyme retains activity. In some embodiments, domains or portions of domains can be deleted, e.g., a membrane-anchor domain can be deleted leaving a soluble enzyme. Some GalNAcT enzymes, such as GalNAc-T2, have a C-terminal lectin domain that can be deleted without diminishing enzymatic activity.

“Refolding expression system” refers to a bacteria or other microorganism with an oxidative intracellular environment, which has the ability to refold disulfide-containing protein in their proper/active form when expressed in this microorganism. Exemplars include systems based on E. coli (e.g., Origami™ (modified E. coli trxB-/gor-), Origami 2™ and the like), Pseudomonas (e.g., fluorescens). For exemplary references on Origami™ technology see, e.g., Lobel et al. (2001) Endocrine 14(2), 205-212; and Lobel et al. (2002) Protein Express. Purif. 25(1), 124-133.

### III. Introduction

The present invention provides polypeptides that include at least one exogenous N-linked glycosylation sequence (sequon polypeptide). Each polypeptide corresponds to a parent polypeptide. The parent polypeptide can be any polypeptide including wild-type polypeptides and other polypeptides for which amino acid sequences or nucleotide sequences are known (e.g., pharmaceutical drugs). In one embodiment, the parent polypeptide does not include an N-linked glycosylation sequence. In another embodiment, the parent polypeptide (e.g., wild-type polypeptide) naturally includes an N-linked glycosylation sequence. The sequon polypeptide that corresponds to such parent polypeptide includes an additional N-linked glycosylation sequence at a different position. In one embodiment, the parent polypeptide is a therapeutic polypeptide, such as human growth hormone (hGH), erythropoietin (EPO), a therapeutic antibody, a bone morphogenetic protein (e.g., BMP-7) or a blood factor (e.g., Factor VI, Factor VIII or Factor IX). Accordingly, the present invention provides therapeutic polypeptide variants that include within their amino acid sequence one or more exogenous N-linked glycosylation sequence.

In one embodiment, the N-linked glycosylation sequence is a substrate for an enzyme (e.g., an oligosaccharyltransferase, such as PglB). The enzyme catalyses the transfer of a glycosyl moiety from a glycosyl donor species (e.g., a lipid-pyrophosphate-linked glycosyl moiety) to an asparagine (N) residue, which is part of the N-linked glycosylation sequence. Exemplary glycosyl moieties that can be conjugated to the glycosylation sequence include GlcNAc, GlcNH, bacillosamine, 6-hydroybacillosamine, GalNAc, GalNH, GlcNAc-GlcNAc, GlcNAc-GlcNH, GlcNAc-Gal, GlcNAc-GlcNAc-Gal-Sia, GlcNAc-Gal-Sia, GlcNAc-GlcNAc-Man, and GlcNAc-GlcNAc-Man(Man)₂. Exemplary glycosyl donor species are described herein.

Accordingly, the invention provides polypeptide conjugates, in which a modified or non-modified sugar moiety is attached to an N-linked glycosylation sequence of the invention. The invention further provides methods of making such polypeptide conjugates. In a representative embodiment, the method is a cell-free in vitro method, wherein the polypetide is contacted (e.g., in a reaction vessel) with a glycosyl donor species (e.g, a lipid-pyrophosphate-linked glycosyl moiety, such as a undecaprenyl-pyrophosphate-linked glycosyl moiety) in the presence of an oligosaccharyl transferase for which the glycosyl donor species is a substrate. The glycosyl moiety in this glycosyl donor species is optionally derivatized with a modifying group, such as a water-soluble polymeric modifying group. The enzyme transfers the modified or non-modified glycosyl moiety onto the polypeptide thereby creating the polypeptide conjugate. When the modifying group includes at least one poly(ethylene glycol) moiety, then such glycosylation reaction is referred to as glycoPEGylation.

In another representative method, the above described enzymatic reaction occurs within a host-cell, in which the polypeptide is expressed. The oligosaccharyl transferase may be endogenously present in the host-cell or may be over-expressed in the host cell. Intracellular glycosylation according to this method offers a variety of advantages over cell-free in vitro glycosylation. For example, there is no need for purification of the polypeptide from cell-culture before glycosylation. In addition, advantage may be taken of other endogenous or co-expressed enzymes, which can be utilized for further modification of the initially formed glycosylated polypeptide.

The glycomodification (e.g., glycoPEGylation) methods of the invention can be practiced on any polypeptide incorporating an N-linked glycosylation sequence. In one embodiment, the methods of the invention provide polypeptide conjugates with increased therapeutic half-life due to, for example, reduced clearance rate, or reduced rate of uptake by the immune or reticuloendothelial system (RES). In another embodiment, the methods of the invention provide a means for masking antigenic determinants on polypeptides, thus reducing or eliminating a host immune response against the polypeptide. Selective attachment of targeting agents to a polypeptide using an appropriate modified sugar can be used to target a polypeptide to a particular tissue or cell surface receptor that is specific for the particular targeting agent. Also provided are polypeptides that display enhanced resistance to degradation by proteolysis, a result that is achieved by altering certain sites on the polypeptide that are cleaved by or recognized by proteolytic enzymes. In one embodiment, such sites are replaced or partially replaced with an N-linked glycosylation sequence of the invention.

In addition, the methods of the invention can be used to modulate the “biological activity profile” of a parent polypeptide. The inventors have recognized that the covalent attachment of a modifying group, such as a water soluble polymer (e.g., mPEG) to a parent polypeptide using the methods of the invention can alter not only bioavailability, pharmacodynamic properties, immunogenicity, metabolic stability, biodistribution and water solubility of the resulting polypeptide species, but can also lead to the reduction of undesired therapeutic activities or to the augmentation of desired therapeutic activities. For example, the former has been observed for the hematopoietic agent erythropoietin (EPO). For example, certain chemically PEgylated EPO variants showed reduced erythropoietic activity while the tissue-protective activity of the wild-type polypeptide was maintained. Such results are described e.g., in U.S. Pat. No. 6,531,121; WO2004/096148, WO2006/014466, WO2006/014349, WO2005/025606 and WO2002/053580. Exemplary cell-lines, which are useful for the evaluation of differential biological activities of selected polypeptides are summarized in Table 1, below:

TABLE 1
Cell-lines used for biological evaluation of various polypeptides

| Polypeptide | Cell-line | Biological Activity |
|---|---|---|
| EPO | UT7 | erythropoiesis |
| | SY5Y | neuroprotection |
| BMP-7 | MG-63 | osteoinduction |
| | HK-2 | nephrotoxicity |
| NT-3 | Neuro2 | neuroprotection (TrkC binding) |
| | NIH3T3 | neuroprotection (p75 binding) |

In one embodiment, a polypeptide conjugate of the invention shows reduced or enhanced binding affinity to a biological target protein (e.g., a receptor), a natural ligand or a non-natural ligand, such as an inhibitor. For instance, abrogating binding affinity to a class of specific receptors may reduce or eliminate associated cellular signaling and downstream biological events (e.g., immune response). Hence, the methods of the invention can be used to create polypeptide conjugates, which have identical, similar or different therapeutic profiles than the parent polypeptide to which the conjugates correspond. The methods of the invention can be used to identify glycoPEGylated therapeutics with specific (e.g., improved) biological functions and to “fine-tune” the therapeutic profile of any therapeutic polypeptide or other biologically active polypeptide. GlycoPEGylation™ is a Trademark of Neose Technologies and refers to technologies disclosed in commonly owned patents and patent applications, e.g., (WO2007/053731; WO2007/022512; WO2006/127896; WO2005/055946; WO2006/121569; and WO2005/070138).

### IV. Compositions

### Polypeptides

In one aspect, the invention provides a polypeptide that has an amino acid sequence, which includes at least one exogenous N-linked glycosylation sequence of the invention (sequon polypeptide). N-linked glycosylation sequences are described herein, below. In one embodiment, the amino acid sequence of the polypeptide includes an exogenous N-linked glycosylation sequence, which is a substrate for one or more wild-type, mutant or truncated oligosaccharyltransferase. Exemplary oligosaccharyltransferases are described herein, below and include full-length or truncated versions of those enzymes described herein (e.g., SEQ ID NOs: 102 to 114).

In an exemplary embodiment, the polypeptide of the invention is generated through recombinant technology by altering the amino acid sequence of a corresponding parent polypeptide (e.g., wild-type polypeptide). Methods for the preparation of recombinant polypeptides are known to those of skill in the art. Exemplary methods are described herein below. The amino acid sequence of the polypeptide may contain a combination of naturally occurring and exogenous (i.e., non-naturally occurring) N-linked glycosylation sequences.

The polypeptide or parent polypeptide of the invention can be any polypeptide. In various embodiments, the polypeptide is a therapeutic polypeptide. In one example, the polypeptide is a recombinant polypeptide. The polypeptide can be a glycopeptide and can have any number of amino acids. In one embodiment, the polypeptide of the invention has a molecular weight of about 5 kDa to about 500 kDa. In another embodiment, the polypeptide has a molecular weight of about 10 kDa to about 400 kDa, about 10 kDa to about 350 kDa, about 10 kDa to about 300 kDa, about 10 kDa to about 250 kDa, about 10 kDa to about 200 kDa, or about 10 kDa to about 150 kDa. In another embodiment, the polypeptide has a molecular weight of about 10 kDa to about 100 kDa. In yet another embodiment, the polypeptide has a molecular weight of about 10 kDa to about 50 kDa. In a further embodiment, the polypeptide has a molecular weight of about 10 kDa to about 25 kDa.

Exemplary polypeptides include wild-type polypeptides and fragments thereof as well as polypeptides, which are modified from their naturally occurring counterpart (e.g., by mutation or truncation). A polypeptide may also be a fusion protein. Exemplary fusion proteins include those in which the polypeptide is fused to a fluorescent protein (e.g., GFP), a therapeutic polypeptide, an antibody, a receptor ligand, a proteinaceous toxin, MBP, a His-tag, and the like.

In one example, the polypeptide of the invention includes an N-linked glycosylation sequence of the invention and in addition includes an O-linked glycosylation sequence. Exemplary O-linked glycosylation sequences and exemplary enzymes useful to glycosylate an O-linked glycosylation sequence, are described in U.S. patent application Ser. No. 11/781,885 filed Jul. 23, 2007, which is incorporated herein by reference in its entirety. O-linked glycosylation techniques using GlcNAc transferases are described in U.S. Provisional Patent Application 60/941,926 and PCT/US2008/065825 filed Jun. 4, 2008, the disclosures of which are also incorporated herein in their entirety.

In one embodiment, the polypeptide is a therapeutic polypeptide, such as those currently used as pharmaceutical agents (i.e., authorized drugs). A non-limiting selection of polypeptides is shown in FIG. 28 of U.S. patent application Ser. No. 10/552,896 filed Jun. 8, 2006, which is incorporated herein by reference.

Exemplary polypeptides include growth factors, such as hepatocyte growth factor (HGF), nerve growth factors (NGF), epidermal growth factors (EGF), fibroblast growth factors (e.g., FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22 and FGF-23), keratinocyte growth factor (KGF), megakaryocyte growth and development factor (MGDF), platelet-derived growth factor (PDGF), transforming growth factors (e.g., TGF-alpha, TGF-beta, TGF-beta2, TGF-beta3), vascular endothelial growth factors (VEGF; e.g., VEGF-2), VEGF inhibitors, such as VEGF-TRAP (Aflibercept), bone growth factor (BGF), glial growth factor, heparin-binding neurite-promoting factor (HBNF), C1 esterase inhibitor, hormones, such as human growth hormone (hGH), follicle stimulating hormone (FSH), thyroid stimulating hormone (TSH) and parathyroid hormone, follitropins (e.g., follitropin-alpha, follitropin-beta), follistatin, luteinizing hormone (LH), as well as cytokines, such as interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18), interferons (e.g., INF-alpha, INF-beta, INF-gamma, INF-omega, INF-tau) and insulin.

Other exemplary polypeptides include enzymes, such as glucocerebrosidase, alpha-galactosidase (e.g., Fabrazyme™), acid-alpha-glucosidase (acid maltase), iduronidases, such as alpha-L-iduronidase (e.g., Aldurazyme™), thyroid peroxidase (TPO), beta-glucosidase (see e.g., enzymes described in U.S. patent application Ser. No. 10/411,044), arylsulfatase, asparaginase, alpha-glucoceramidase (e.g., imiglucerase), sphingomyelinase, butyrylcholinesterase, urokinase and alpha-galactosidase A (see e.g., enzymes described in U.S. Pat. No. 7,125,843).

Other exemplary parent polypeptides include bone morphogenetic proteins (e.g., BMP-1, BMP-2, BMP-3, BMP-4, BMP-5, BMP-6, BMP-7, BMP-8, BMP-9, BMP-10, BMP-11, BMP-12, BMP-13, BMP-14, BMP-15), neurotrophins (e.g., NT-3, NT-4, NT-5), erythropoietins (EPO), novel erythropoiesis stimulating protein (NESP; e.g., Aranesp), growth differentiation factors (e.g., GDF-5), glial cell line-derived neurotrophic factor (GDNF), brain derived neurotrophic factor (BDNF), myostatin, nerve growth factor (NGF), granulocyte colony stimulating factor (G-CSF; e.g., Neupogen®, Neulasta®), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), osteopontin, osteoprotegrin, protein C, somatomedin-1, somatotropin, somatropin, chimeric diphtheria toxin-IL-2, glucagon-like peptides (e.g., GLP-1 and GLP-2), thrombin, thrombopoietin, thrombospondin-2, anti-thrombin III (AT-III), prokinetisin, CD4, αCD20, tumor necrosis factors (e.g., TNF-alpha), TNF-alpha inhibitor, TNF receptor (TNF-R), P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, extendin-4, BDNF, beta-2-microglobulin, ciliary neurotrophic factor (CNTF), lymphotoxin-beta receptor (LT-beta receptor), fibrinogen, GDF (e.g., GDF-1, GDF-2, GDF-3, GDF-4, GDF-5, GDF-6, GDF-7, GDF-8, GDF-9, GDF-10, GDF-11, GDF-12, GDF-13, GDF-14, GDF-15), GLP-1, insulin-like growth factors (e.g., IGF1), insulin-like growth factor binding proteins (e.g., IGB-5), IGF/IBP-2, IGF/IBP-3, IGF/IBP-4, IGF/IBP-5, IGF/IBP-6, IGF/IBP-7, IGF/IBP-8, IGF/IBP-9, IGF/IBP-10, IGF/IBP-11, IGF/IBP-12 and IGF/IBP-13. Exemplary amino acid sequences for some of the above listed polypeptides are described in U.S. Pat. No. 7,214,660, all of which are incorporated herein by reference.

In one example, the polypeptide is von Willebrand factor (vWF) or a portion of vWF. Recombinant vWF has been described (see, e.g., Fischer B. E. et al., Cell. Mol. Life. Sci. 1997, 53:943-950, which is incorporated herein by reference. In another example, the polypeptide is vWF-cleaving protease (vWF-protease, vWF-degrading protease).

In one example, the polypeptide of the invention is a blood coagulation factor (blood factor). Exemplary blood factors include Factor V, Factor VII, Factor VIII (e.g., Factor VIII-2, Factor VIII-3), Factor IX, Factor X and Factor XIII. In another example, the p[olypeptide is a blood factor inhibitor (e.g., Factor Xa inhibitor).

In a particular example, the polypeptide is Factor VIII. Factor VIII and Factor VIII variants are know in the art. For example, U.S. Pat. No. 5,668,108 describes Factor VIII variants, in which the aspartic acid at position 1241 is replaced by a glutamic acid. U.S. Pat. No. 5,149,637 describes Factor VIII variants comprising the C-terminal fraction, either glycosylated or unglycosylated, and U.S. Pat. No. 5,661,008 describes Factor VIII variants comprising amino acids 1-740 linked to amino acids 1649 to 2332 by at least 3 amino acid residues. Therefore, variants, derivatives, modifications and complexes of Factor VIII are well known in the art, and are encompassed in the present invention. Expression systems for the production of Factor VIII are also well known in the art, and include prokaryotic and eukaryotic cells, as exemplified in U.S. Pat. Nos. 5,633,150, 5,804,420, and 5,422,250. Any of the above discussed Factor VIII sequences may be modified to include an exogenous O-linked, S-linked or N-linked glycosylation sequence.

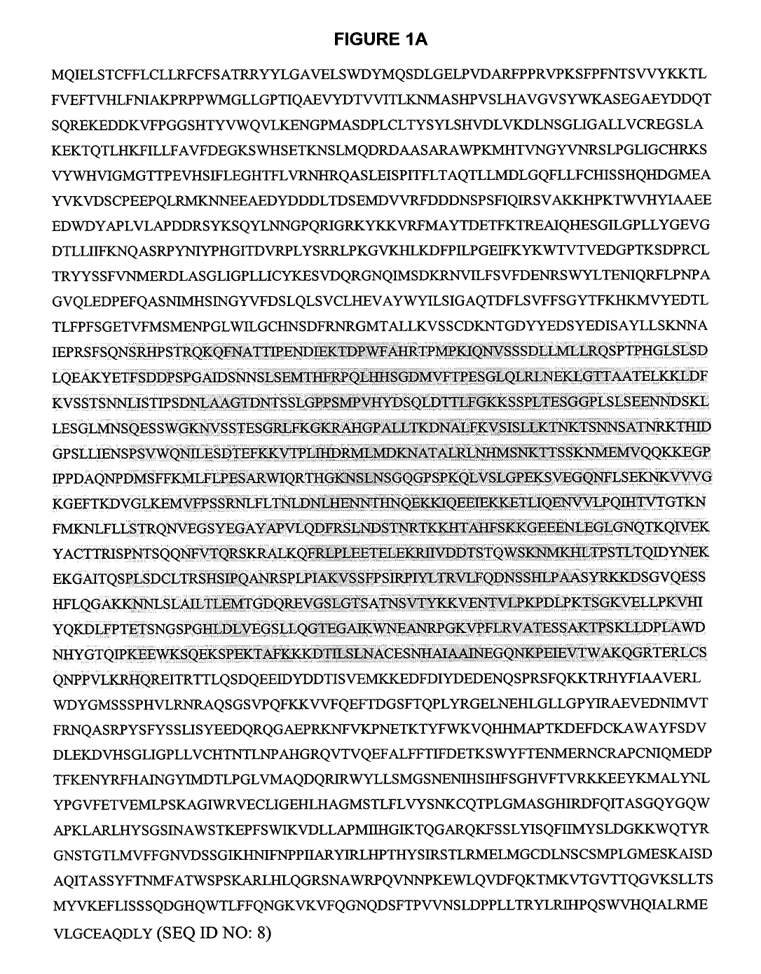

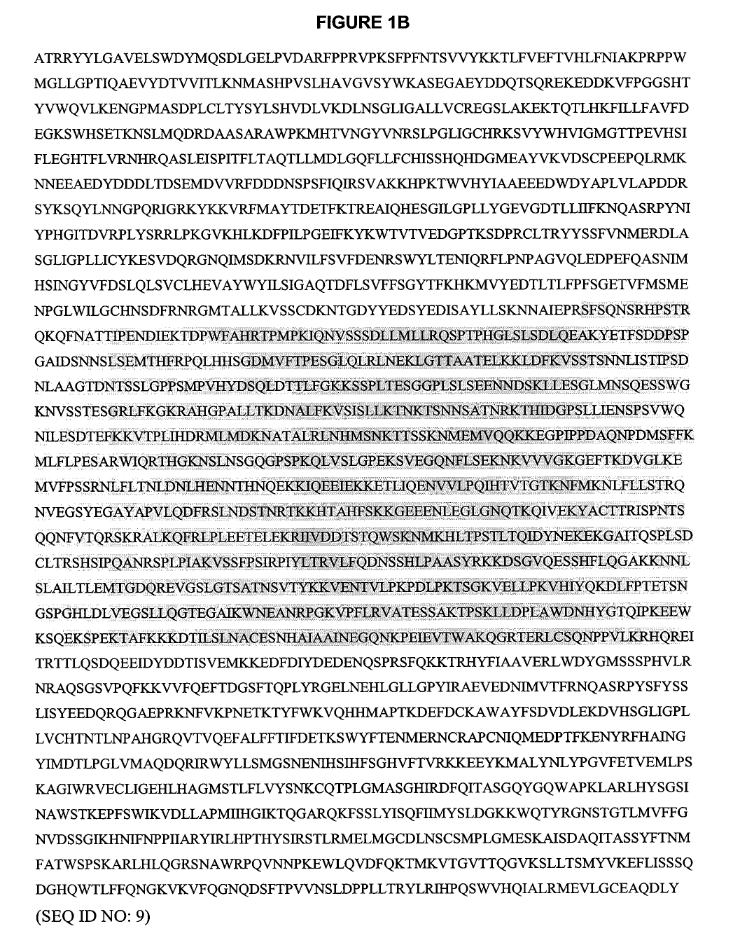

In one example, the Factor VIII is a full-length or wild-type Factor VIII polypeptide. An exemplary amino acid sequence for full-length Factor VIII polypeptides are shown in FIGS. 1A and 1B (SEQ ID NO: 8, SEQ ID NO: 9). In yet another example, the polypeptide is a Factor VIII polypeptide, in which the B-domain includes less amino acid residues than the B-domain of wild-type or full-length Factor VIII. Those Factor VIII polypeptides are referred to as B-domain deleted or partial B-domain deleted Factor VIII. A person of skill in the art will be able to identify the B-domain within a given Factor VIII polypeptide. In one example, the B-domain includes amino acid residues between the two flanking sequences IEPR (on the N-terminal side) and EITR (on the C-terminal side). However, a person of skill in the art will appreciate that these two flanking sequences may not be present or may be modified, e.g., by mutation. A typical location of the B-domain within the Factor VIII polypeptide is illustrated in the following diagram:

B-domain within exemplary Factor VIII polypeptide:

### . . . IEPR-B-Domain-EITR . . .

In one example, the B-domain is found between amino acid residues Arg⁷⁴⁰ and Glu¹⁶⁴⁹ of the full length Factor VIII sequence (e.g., sequence shown in FIG. 1B):

### . . . IEPR⁷⁴⁰-B-domain-E¹⁶⁴⁹ITR . . .

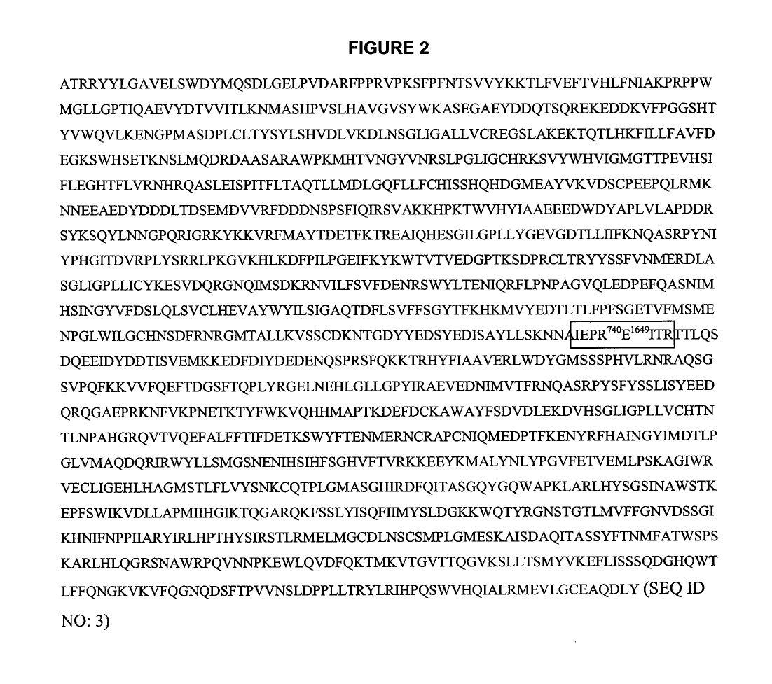

In one embodiment, the Factor VIII polypeptide of the current invention does not include any amino acid residues normally associated with the B-domain (complete B-domain deletion). An exemplary amino acid sequence according to this embodiment is shown in FIG. 2, wherein all amino acid residues between Arg⁷⁴⁰ and Glu¹⁶⁴⁹ of the full length Factor VIII sequence (FIG. 1B) are removed. In another embodiment, the original B-domain is replaced with another sequence (B-domain replacement sequence). In on example, the B-domain replacement sequence of the Factor VIII polypeptide includes at least two amino acids. For example, at least two, at least 3, at least 4, at least 5, at least 6, at least 7, at least 8, at least 9 or at least 10 amino acid residues are found between Arg⁷⁴⁰ and Glu¹⁶⁴⁹ in FIG. 2. The replacement sequence can include any number of amino acid residues and can have any amino acid sequence.

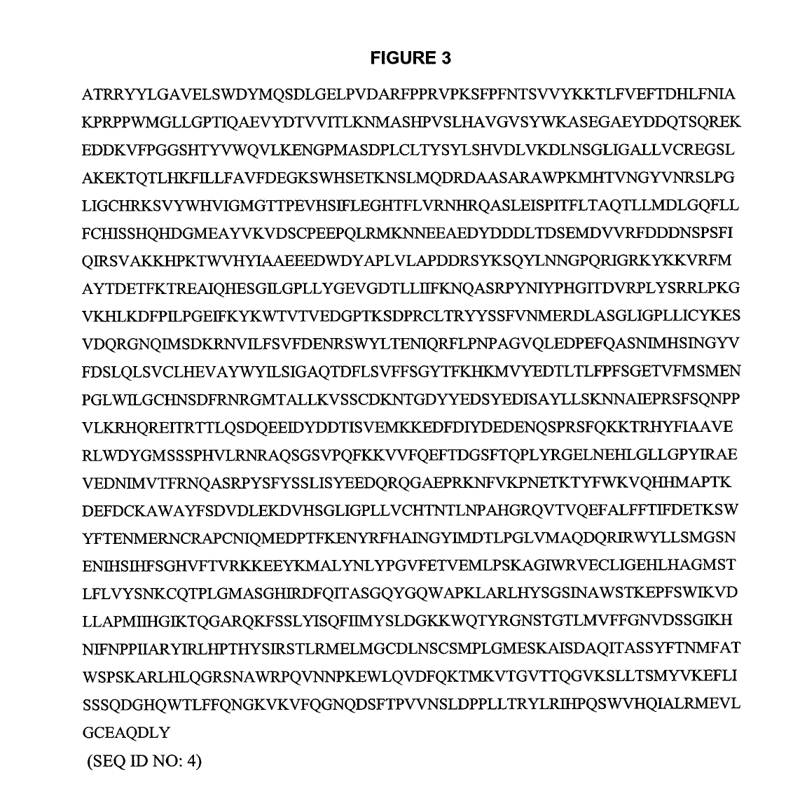

In one example, the sequence replacing the B-domain includes a partial B-domain sequence. For example, the sequence replacing the B-domain includes about 2, about 4, about 6, about 8, about 10, about 12, about 14 or more than 14 of the N-terminal amino acids of the B-domain (e.g., between Arg⁷⁴⁰ and Glu¹⁶⁴⁹ in FIG. 2). For example, the replacement sequence may include a partial N-terminal B-domain sequence selected from SFSQN, SFSQNS, SFSQNSR and SFSQNSRH. In another example, the sequence replacing the B-domain includes about 2, about 4, about 6, about 8, about 10, about 12, about 14 or more than 14 of the C-terminal amino acids of the original B-domain (e.g., between Arg⁷⁴⁰ and Glu¹⁶⁴⁹ in FIG. 2). For example, the replacement sequence may include a partial C-terminal B-domain sequence selected from QR, HQR, RHQR, KRHQR, LKRHQR, VLKRHQR, and PPVLKRHQR. In yet another example, the amino acid sequence replacing the B-domain includes a combination of more than one partial sequence. For example, the replacement sequence includes a partial N-terminal sequence linked to a partial C-terminal sequence of the original B-domain, wherein the N-terminal and C-terminal B-domain sequences are optionally linked via additional amino acid residues, e.g., one or more arginine residues. Exemplary amino acid sequences for B-domain deleted Factor VIII polypeptides include those sequences shown in FIGS. 3-5 (SEQ ID NOs: 4-6).

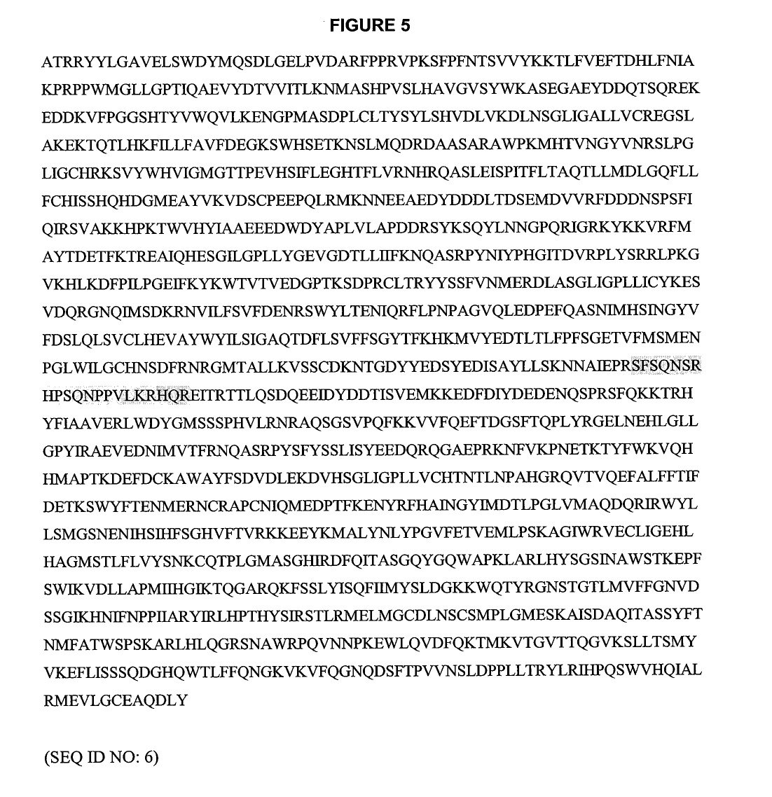

In one embodiment, the B-domain replacement sequence includes a naturally or non-naturally occurring (e.g., exogenous) N-linked or O-linked glycosylation sequence. In one example, the original B-domain is truncated in such a way as to leave at least one of the O-linked or N-linked glycosylation sequences intact, which are naturally present in the original B-domain. In another example, the combination of partial B-domain sequences as described above, results in the formation of a glycosylation sequence. An example can be observed in FIG. 5: P⁷⁴⁹SQNP.

In yet another example, the B-domain replacement sequence includes an amino acid sequence, which is not present in the naturally occurring B-domain, wherein this non-naturally occurring sequence includes an exogenous O-linked or N-linked glycosylation sequence (e.g., an O-linked glycosylation sequence of the invention). In one example, the B-domain replacement sequence includes an exogenous O-linked glycosylation sequence of the invention, such as PTP, PTEI, PTEIP, PTQA, PTQAP, PTINT, PTINTP, PTTVS, PTTVL, PTQGAM, PTQGAMP, TETP, PTVL, PTVLP, PTLSP, PTDAP, PTENP, PTQDP, PTASP, PTTVSP, PTQGA, PTSAV, PTTLYV, PTTLYVP, PSSGP or PSDGP. In another example, the B-domain replacement sequence includes an exogenous N-linked glycosylation sequence of the invention, such as NLT.

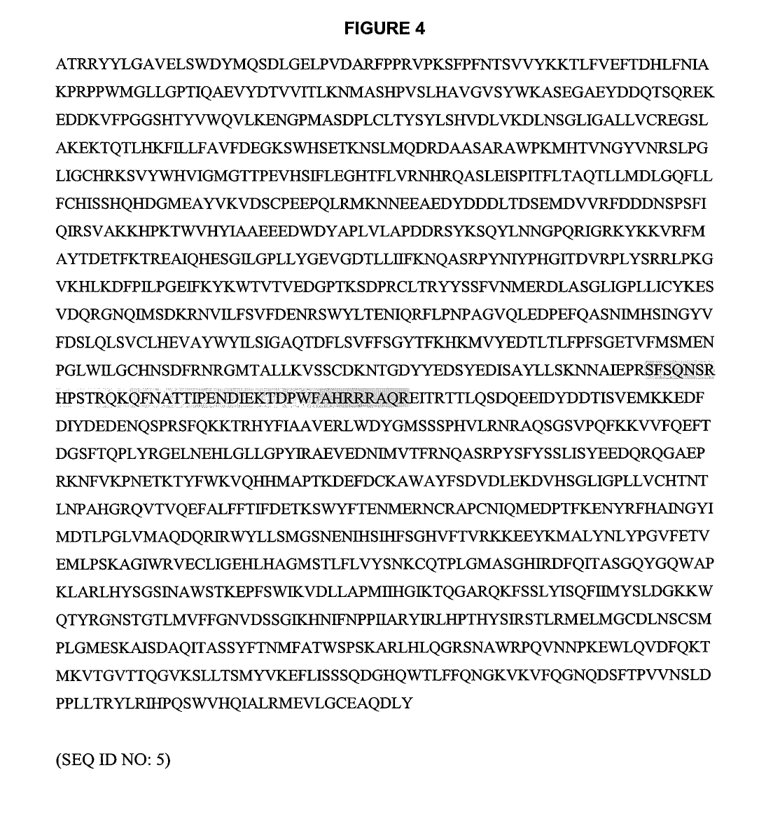

In one embodiment, the invention provides a Factor VIII polypeptide including an amino acid sequence according to FIG. 1A, FIG. 1B, FIG. 2, FIG. 3, FIG. 4 or FIG. 5, and further including an exogenous N-linked glycosylation sequence introduced into said amino acid sequence at the N-terminus or at an amino acid position selected from 1 to 740 (heavy chain). In another exemplary embodiment, the invention provides a Factor VIII polypeptide comprising an amino acid sequence according to FIG. 4 and further comprising an exogenous N-linked glycosylation sequence introduced into said amino acid sequence at an amino acid position selected from 782 to 1,465 (light chain). In another exemplary embodiment, the invention provides a Factor VIII polypeptide comprising an amino acid sequence according to FIG. 1A, FIG. 1B, FIG. 2, FIG. 3, FIG. 4 or FIG. 5, and further comprising an exogenous N-linked glycosylation sequence introduced into said amino acid sequence at an amino acid position within the light chain of said Factor VIII polypeptide. In another exemplary embodiment, the invention provides a Factor VIII polypeptide comprising an amino acid sequence according to FIG. 4 and further comprising an exogenous N-linked glycosylation sequence introduced into said amino acid sequence at an amino acid position selected from 741 to 781 (B-domain fragment). In another exemplary embodiment, the invention provides a Factor VIII polypeptide comprising an amino acid sequence according to FIG. 1A, FIG. 1B, FIG. 2, FIG. 3, FIG. 4 or FIG. 5, and further comprising an exogenous N-linked glycosylation sequence introduced into said amino acid sequence within B-domain or B-domain fragment of said Factor VIII polypeptide. In one example, the Factor VIII polypeptide of the invention is produced in CHO cells. In another example, the Factor VIII polypeptide is produced using a trxB gor mutant E. coli expression system (Origami) known in the art.

In another example, the polypeptide is a fusion protein between two or more polypeptides. In another example, the polypeptide is a complex between two or more polypeptides. In an exemplary embodiment, the complex includes a blood factor. In another exemplary embodiment, the complex includes Factor VIII. The Factor VIII polypeptide in this complex may be full-length, B-domain deleted, or partial B-domain deleted Factor VIII. In one example, the complex is between Factor VIII and von Willebrandt Factor (vWF).

Also within the scope of the invention are polypeptides that are antibodies. The term antibody is meant to include immunoglobulins, antibody fragments (e.g., Fc domains), single chain antibodies, Lama antibodies, nano-bodies and the like. Also included in the term are antibody-fusion proteins, such as Ig chimeras. Preferred antibodies include humanized, monoclonal antibodies or fragments thereof. All known isotypes of such antibodies are within the scope of the invention. Exemplary antibodies include those to growth factors, such as endothelial growth factor (EGF), vascular endothelial growth factors (e.g., monoclonal antibody to VEGF-A, such as ranibizumab (Lucentis™)) and fibroblast growth factors, such as FGF-7, FGF-21 and FGF-23) and antibodies to their respective receptors. Other exemplary antibodies include anti-TNF antibodies, such as anti-TNF-alpha monoclonal antibodies (see e.g., U.S. patent application Ser. No. 10/411,043), TNF receptor-IgG Fc region fusion protein (e.g., Enbrel™), anti-HER2 monoclonal antibodies (e.g., Herceptin™), monoclonal antibodies to protein F of respiratory syncytial virus (e.g., Synagis™), monoclonal antibodies to TNF-α (e.g., Remicade™), monoclonal antibodies to glycoproteins, such as IIb/IIIa (e.g., Reopro™), monoclonal antibodies to CD20 (e.g., Rituxan™), CD4, alpha-CD3, CD40L and CD154 (e.g., Ruplizumab), monoclonal antibodies to PSGL-1 and CEA. Any modified (e.g., mutated) version of any of the above listed polypeptides is also within the scope of the invention.

In an exemplary embodiment, the parent polypeptide is EPO comprising the amino acid sequence of (SEQ ID NO: 7), which is shown below:

Ala Pro Pro Arg Leu Ile Cys Asp Ser Arg Val Leu Glu Arg Tyr Leu Leu Glu Ala Lys Glu
Ala Glu Asn²⁴ Ile Thr Thr Gly Cys Ala Glu His Cys Ser Leu Asn Glu Asn³⁸ Ile Thr Val Pro
Asp Thr Lys Val Asn Phe Tyr Ala Trp Lys Arg Met Glu Val Gly Gln Gln Ala Val Glu Val
Trp Gln Gly Leu Ala Leu Leu Ser Glu Ala Val Leu Arg Gly Gln Ala Leu Leu Val Asn⁸³ Ser
Ser Gln Pro Trp Glu Pro Leu Gln Leu His Val Asp Lys Ala Val Ser Gly Leu Arg Ser Leu Thr
Thr Leu Leu Arg Ala Leu Gly Ala Gln Lys Glu Ala Ile Ser Pro Pro Asp Ala Ala Ser¹²⁶ Ala
Ala Pro Leu Arg Thr Ile Thr Ala Asp Thr Phe Arg Lys Leu Phe Arg Val Tyr Ser Asn Phe
Leu Arg Gly Lys Leu Lys Leu Tyr Thr Gly Glu Ala Cys Arg Thr Gly Asp

In an exemplary embodiment, the parent polypeptide includes an amino acid sequence having at least one mutation replacing a basic amino acid residue, such as arginine or lysine, with an uncharged amino acid, such as glycine or alanine. In another embodiment, the EPO polypeptide includes an amino acid sequence having at least one mutation, selected from Arg¹³⁹ to Ala¹³⁹, Arg¹⁴³ to Ala¹⁴³ and Lys¹⁵⁴ to Ala¹⁵⁴.

### N-Linked Glycosylation Sequence

The N-linked glycosylation sequence of the invention can be any short amino acid sequence. In one embodiment, the N-linked glycosylation sequence includes from about 3 to about 20, preferably about 3 to about 10, more preferably about 3 to about 9 and most preferably about 3 to about 7 amino acid residues. The N-linked glycosylation sequence of the invention includes at least one amino acid residue having an amino group. In one embodiment, the N-linked glycosylation sequence of the invention includes at least one asparagine (N) residue. In another embodiment, the amino group of the asparagine residue is glycosylated when the sequon polypeptide is subjected to an enzymatic glycosylation or glycoconjugation reaction. During this reaction, a hydrogen atom of the amino group is replaced with a glycosyl moiety. The amino acid residue receiving the glycosyl moiety is referred to as the “site of glycosylation” or “glycosylation site.”

In one embodiment, the N-linked glycosylation sequence of the invention is naturally present in a wild-type polypeptide. Polypeptide conjugates of such wild-type polypeptides are within the scope of the invention. In another embodiment, the N-linked glycosylation sequence is not present or not present at the same position, in the corresponding parent polpeptide (exogenous N-linked glycosylation sequence). Introduction of an exogenous N-linked glycosylation sequence into a parent polypeptide generates a sequon polypeptide of the invention. The N-linked glycosylation sequence may be introduced into the parent polypeptide by mutation. In another example, the N-linked glycosylation sequence is introduced into the amino acid sequence of a parent polypeptide by chemical synthesis of the sequon polypeptide.

In one embodiment, the N-linked glycosylation sequence of the invention includes an amino acid sequence according to Formula (I) (SEQ ID NO: 1). In another embodiment, the N-linked glycosylation sequence includes an amino acid sequence according to Formula (II) (SEQ ID NO: 2). In yet another embodiment, the N-linked glycosylation sequence consists of an amino acid sequence according to Formula (I). In a further embodiment, the N-linked glycosylation sequence consists of an amino acid sequence according to Formula (II):

| | |
|---|---|
| X¹ N X² X³ X⁴ | (I) (SEQ ID NO: 1) |
| X¹ D X^{2′} N X² X³ X⁴. | (II) (SEQ ID NO: 2) |

In Formula (I) and Formula (II), N is asparagine and D is aspartic acid. In one embodiment, X³ is threonine (T). In another embodiment, X³ is serine (S). X¹ is either present or absent. When present, X¹ can be any amino acid. In one embodiment, X¹ is a member selected from glycine (G), alanine (A), valine (V), leucine (L), isoleucine (I), phenylalanine (F), methionine (M), asparagine (N), glutamic acid (E), glutamine (Q), histidine (H), lysine (K), arginine (R), serine (S), threonine (T), tyrosine (Y), tryptophan (W), cysteine (C) and proline (P). X⁴ is either present or absent. When present, X⁴ can be any amino acid. In one embodiment, X⁴ is a member selected from glycine (G), alanine (A), valine (V), leucine (L), isoleucine (I), phenylalanine (F), methionine (M), asparagine (N), glutamic acid (E), glutamine (Q), histidine (H), lysine (K), arginine (R), serine (S), threonine (T), tyrosine (Y), tryptophan (W), cysteine (C), proline (P).

In Formula (I) and Formula (II), X² can be any amino acid. In a preferred embodiment, X² is not proline (P). X^{2′} can be any amino acid. In one embodiment, X^{2′} is not proline. In one embodiment, X² and X^{2′} are members independently selected from glycine (G), alanine (A), valine (V), leucine (L), isoleucine (I), phenylalanine (F), methionine (M), asparagine (N), glutamic acid (E), glutamine (Q), histidine (H), lysine (K), arginine (R), serine (S), threonine (T), tyrosine (Y), tryptophan (W) and cysteine (C). The N-linked glycosylation sequence may include additional C- or N-terminal amino acid residues. In one embodiment, the additional amino acids are useful to modulate the tertiary structure of the polypetide in proximity to the glycosylation site.

In one embodiment, X² in Formula (I) is an uncharged amino acid. In an exemplary embodiment, the N-linked-glycosylation sequence is a member selected from X¹NGSX⁴, X¹NGTX⁴, X¹NASX⁴, X¹NATX⁴, X¹NVSX⁴, X¹NVTX⁴, X¹NLSX⁴, X¹NLTX⁴, X¹NISX⁴, X¹NITX⁴, X¹NFSX⁴, X¹NFTX⁴, X¹NSSX⁴, X¹NSTX⁴, X¹NTSX⁴, X¹NTTX⁴, X¹NCSX⁴, X¹NCTX⁴, X¹NYSX⁴ and X¹NYTX⁴ wherein X¹ and X⁴ are defined as above. In one example according to this embodiment, X¹ is not present. In another example, X⁴ is not present. In yet another embodiment, both X¹ and X⁴ are not present.

Accordingly, in another example, the N-linked glycosylation sequence is a member selected from NGS, NGT, NAS, NAT, NVS, NVT, NLS, NLT, NIS, NIT, NFS, NFT, NSS, NST, NTS, NTT, NCS, NCT, NYS and NYT.

In one embodiment, the N-linked glycosylation sequence is an extended glycosylation sequence according to Formula (II). In another embodiment, the extended glycosylation sequence is used when the oligosaccharyl transferase is an enzyme of bacterial origin (e.g., PglB). In another embodiment, X² in Formula (II) is an uncharged amino acid. In one example according to this embodiment, the N-glycosylation sequence is a member selected from X¹D X^{2′}NGSX⁴, X¹DX^{2′}NGTX⁴, X¹DX^{2′}NASX⁴, X¹DX^{2′}NATX⁴, X¹DX^{2′}NVSX⁴, X¹DX^{2′}NVTX⁴, X¹DX^{2′}NLSX⁴, X¹DX^{2′}NLTX⁴, X¹DX^{2′}NISX⁴, X¹DX^{2′}NITX⁴, X¹DX^{2′}NFSX⁴ and X¹DX^{2′}NFTX⁴, wherein X¹, X^{2′} and X⁴ are defined as above.

In another example, the N-glycosylation sequence is a member selected from D X^{2′}NGS, DX^{2′}NGT, DX^{2′}NAS, DX^{2′}NAT, DX^{2′}NVS, DX^{2′}NVT, DX^{2′}NLS, DX^{2′}NLT, DX^{2′}NIS, DX^{2′}NIT, DX^{2′}NFS and DX^{2′}NFT, wherein X^{2′} is defined as above. In another example, X^{2′} in any of the above embodiments is selected from uncharged amino acids. In one example, X^{2′} is G. In another example, X^{2′} is A. In yet another example, X^{2′} is V. In a further example, X^{2′} is L. In a further embodiment, X^{2′} is I. In another example, X^{2′} is F.

### Positioning of N-Linked Glycosylation Sequences

In one embodiment, the N-linked glycosylation sequence, when part of a polypeptide (e.g., a sequon polypeptide of the invention), is a substrate for an oligosaccharyl transferase (e.g., Stt3p or PglB). In another example, the glycosylation sequence is a substrate for a modified enzyme, such as an enzyme having a deleted or truncated membrane-anchoring domain. The efficiency, with which each N-linked glycosylation sequence of the invention is glycosylated during an appropriate glycosylation reaction, may depend on the type and nature of the enzyme, and may also depend on the context of the glycosylation sequence, especially the three-dimensional structure of the polypeptide around the glycosylation site.

Generally, an N-linked glycosylation sequence can be introduced at any position within the amino acid sequence of the polypeptide. In a preferred embodiment, the N-linked glycosylation sequence (under the reaction conditions used) is accessible to an oligosaccharyl transferase. In one example, the glycosylation sequence is introduced at the N-terminus of the parent polypeptide (i.e., preceding the first amino acid or immediately following the first amino acid) (amino-terminal mutants). In another example, the N-linked glycosylation sequence is introduced near the amino-terminus (e.g., within 10 amino acid residues of the N-terminus) of the parent polypeptide. In another example, the N-linked glycosylation sequence is located at the C-terminus of the parent polypeptide immediately following the last amino acid of the parent polypeptide (carboxy-terminal mutants). In yet another example, the N-linked glycosylation sequence is introduced near the C-terminus (e.g., within 10 amino acid residues of the C-terminus) of the parent polypeptide. In yet another example, the N-linked glycosylation sequence is located anywhere between the N-terminus and the C-terminus of the parent polypeptide (internal mutants). It is generally preferred that the modified polypeptide is biologically active, even if that biological activity is altered from the biological activity of the corresponding parent polypeptide.

An important factor influencing glycosylation efficiencies of sequon polypeptides is the accessibility of the glycosylation site (e.g., asparagine side chain) for the glycosyl/saccharyl transferase and other reaction partners, including solvent molecules. If the glycosylation sequence is positioned within an internal domain of the polypeptide, glycosylation will likely be inefficient. Hence, in one embodiment, the glycosylation sequence is introduced at a region of the polypeptide, which corresponds to the polypeptide's solvent exposed surface. An exemplary polypeptide conformation is one, in which the target amino group of the glycosylation sequence is not oriented inwardly, forming hydrogen bonds with other regions of the polypeptide. Another exemplary conformation is one, in which the amino group is unlikely to form hydrogen bonds with neighboring proteins.

In one example, the N-linked glycosylation sequence is created within a pre-selected, specific region of the parent protein. In nature, glycosylation of the polypeptide backbone usually occurs within loop regions of the polypeptide and typically not within helical or beta-sheet structures. Therefore, in one embodiment, the sequon polypeptide of the invention is generated by introducing an N-linked glycosylation sequence into an area of the parent polypeptide, which corresponds to a loop domain.

For example, the crystal structure of the protein BMP-7 contains two extended loop regions between Ala⁷² and Ala⁸⁶ as well as Ile⁹⁶ and Pro¹⁰³. Generating BMP-7 mutants, in which the N-linked glycosylation sequence is placed within those regions of the polypeptide sequence, may result in polypeptides, wherein the mutation causes little or no disruption of the original tertiary structure of the polypeptide.

However, introduction of an N-linked glycosylation sequence at an amino acid position that falls within a beta-sheet or alpha-helical conformation may also lead to sequon polypeptides, which are efficiently glycosylated at the newly introduced N-linked glycosylation sequence. Introduction of an N-linked glycosylation sequence into a beta-sheet or alpha-helical domain may cause structural changes to the polypeptide, which, in turn, enable efficient glycosylation.

The crystal structure of a protein can be used to identify domains of a wild-type or parent polypeptide that are most suitable for introduction of an N-linked glycosylation sequence and may allow for the pre-selection of promising modification sites.

When a crystal structures is not available, the amino acid sequence of the polypeptide can be used to pre-select promising modification sites (e.g., prediction of loop domains versus alpha-helical domains). However, even if the three-dimensional structure of the polypeptide is known, structural dynamics and enzyme/receptor interactions are variable in solution. Hence, the identification of suitable mutation sites as well as the selection of suitable glycosylation sequences, may involve the creation of several sequon polypeptides (e.g., libraries of sequon polypeptides of the invention) and testing those variants for desirable characteristics using appropriate screening protocols, e.g., those described herein.

In one embodiment, in which the parent polypeptide is an antibody or antibody fragment, the constant region (e.g., C_{H}2 domain) of an antibody or antibody fragment is modified with an N-linked glycosylation sequence of the invention. In one example, the N-linked glycosylation sequence is introduced in such a way that a naturally occurring glycosylation sequence is replaced or functionally impaired. Amino acid and nucleic acid sequences for the constant region of antibodies are known to those of skill in the art.

In one embodiment sequon scanning is performed through a selected area of the C_{H}2 domain creating a library of antibodies, each including an exogenous N-linked glycosylation sequence of the invention. In yet another embodiment, resulting polypeptide variants are subjected to an enzymatic glycosylation reaction aimed at adding a glycosyl moiety to the glycosylation sequence. Those variants that are sufficiently glycosylated can be analyzed for their ability to bind a suitable receptor (e.g., F_{c} receptor, such as F_{c}γRIIIa). In one embodiment, such glycosylated antibody or antibody fragments exhibits increased binding affinity to the F_{c} receptor when compared with the parent antibody or a naturally glycosylated version thereof. This aspect of the invention is further described in U.S. Provisional Patent Application 60/881,130 filed Jan. 18, 2007, the disclosure of which is incorporated herein in its entirety. The described modification can change the effector function of the antibody. In one embodiment, the glycosylated antibody variant exhibits reduced effector function, e.g., reduced binding affinity to a receptor found on the surface of a natural killer cell or on the surface of a killer T-cell. In another example, glycoconjugation of the antibody is useful to modify the pharmacokinetic and/or pharmacodynamic properties of the modified antibody when compared to the non-modified antibody. For example, the glycoconjugated antibody has a longer in vivo half-life than the non-modified antibody.

### Peptide Linker Fragment Including an N-Linked Glycosylation Sequence

In another embodiment, the N-linked glycosylation sequence is not introduced within the parent polypeptide sequence, but rather the sequence of the parent polypeptide is extended though addition of a peptide linker fragment to either the N- or C-terminus of the parent polypeptide, wherein the peptide linker fragment includes an N-linked glycosylation sequence of the invention, such as “NLT” or “DFNVS”. The peptide linker fragment can have any number of amino acids. In one embodiment the peptide linker fragment includes at least about 5, at least about 10, at least about 15, at least about 20, at least about 30, at least about 50 or more than 50 amino acid residues. The peptide linker fragment optionally includes an internal or terminal amino acid residue that has a reactive functional group, such as an amino group (e.g., lysine) or a sufhydryl group (e.g., cysteine). Such reactive functional group may be used to link the polypeptide to another moiety, such as another polypeptide, a cytotoxin, a small-molecule drug or another modifying group of the invention. This aspect of the invention is further described in U.S. Provisional Patent Application 60/881,130 filed Jan. 18, 2007, the disclosure of which is incorporated herein in its entirety.

In one embodiment, the parent polypeptide that is modified with a peptide linker fragment of the invention is an antibody or antibody fragment. In one example according to this embodiment, the parent polypeptide is scFv. Methods described herein can be used to prepare scFvs of the present invention in which the scFv or the linker is modified with a glycosyl moiety or a modifying group attached to the peptide through a glycosyl linking group. Exemplary methods of glycosylation and glycoconjugation are set forth in, e.g., PCT/US02/32263 and U.S. patent application Ser. No. 10/411,012, each of which is incorporated by reference herein in its entirety.

In one embodiment, certain amino acid residues are included into the N-linked glycosylation sequence to modulate expressability of the mutated polypeptide in a particular organism, such as E. coli, proteolytic stability, structural characteristics and/or other properties of the polypeptide.

### Exemplary Polypeptides

The N-linked glycosylation sequences of the invention can be introduced into any parent polypeptide, creating a sequon polypeptide of the invention. The sequon polypeptides of the invention can be generated using methods known in the art and described herein below (e.g., through recombinant technology or chemical synthesis). In one embodiment, the parent sequence is modified in such a way that the N-linked-glycosylation sequence is inserted into the parent sequence adding the entire length and respective number of amino acids to the amino acid sequence of the parent polypeptide. In another embodiment, the N-linked glycosylation sequence replaces one or more amino acids of the parent polypeptide. In another embodiment, the N-linked glycosylation sequence is introduced into the parent polypeptide using one or more of the pre-existing amino acids to be part of the glycosylation sequence. For instance, an asparagine residue in the parent peptide is maintained and those amino acids immediately following the proline are mutated to create an N-linked-glycosylation sequence of the invention. In yet another embodiment, the N-linked glycosylation sequence is created employing a combination of amino acid insertion and replacement of existing amino acids.

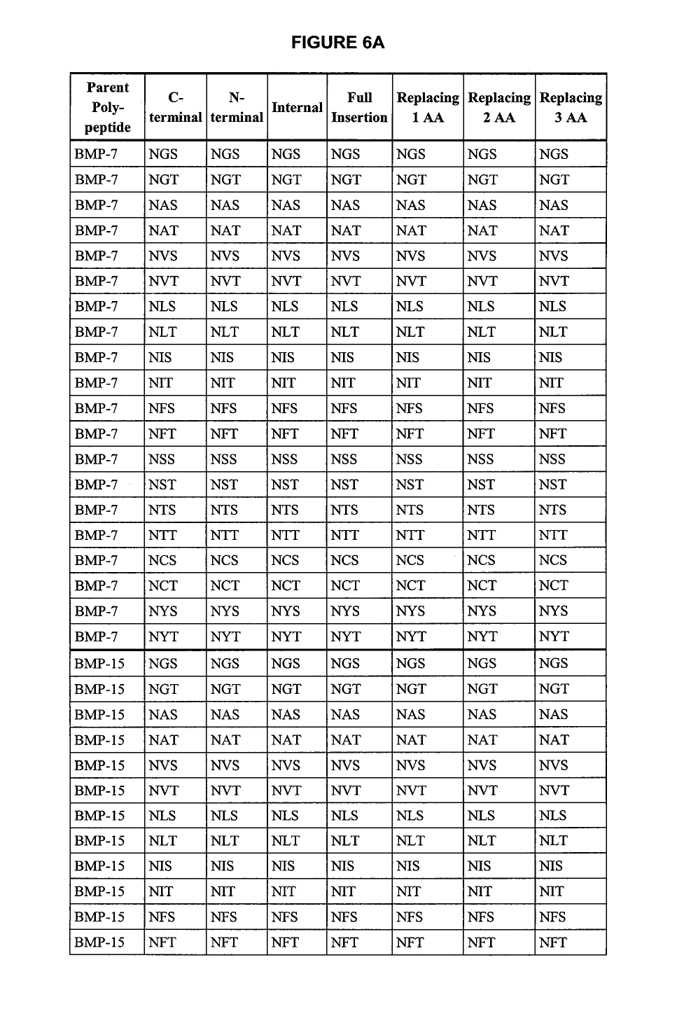

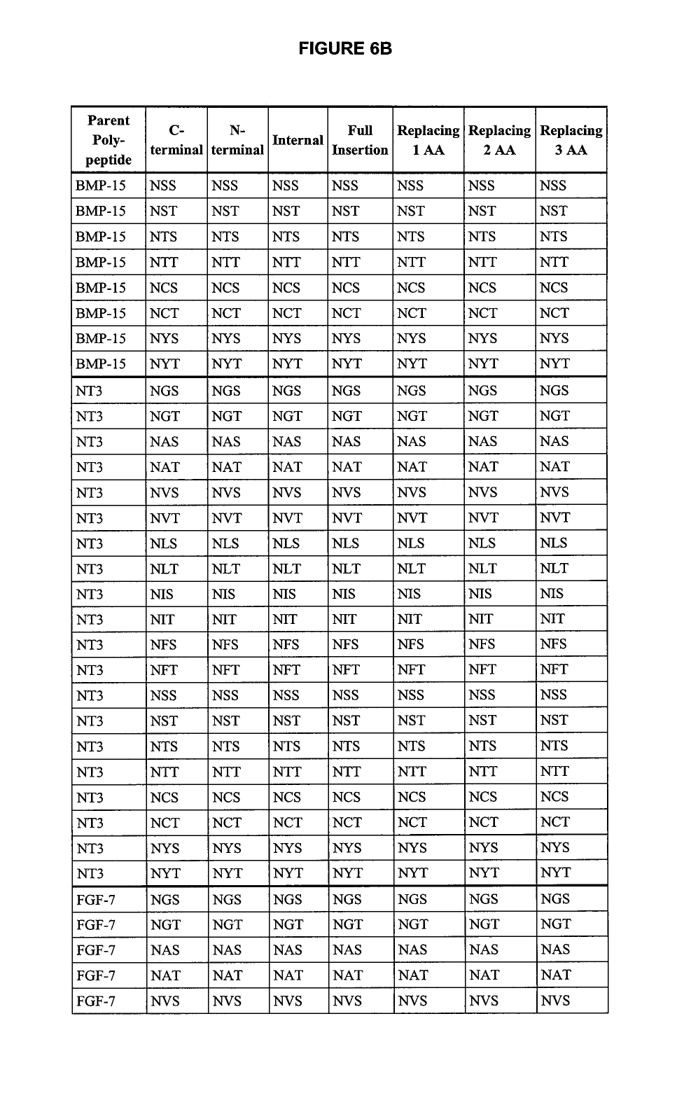

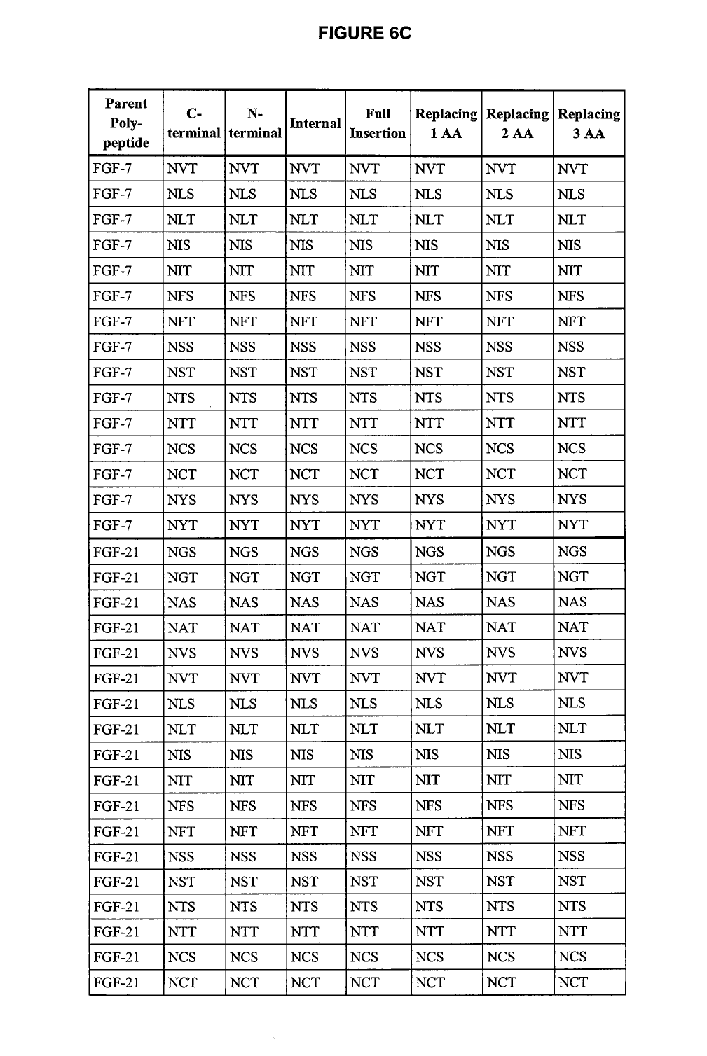

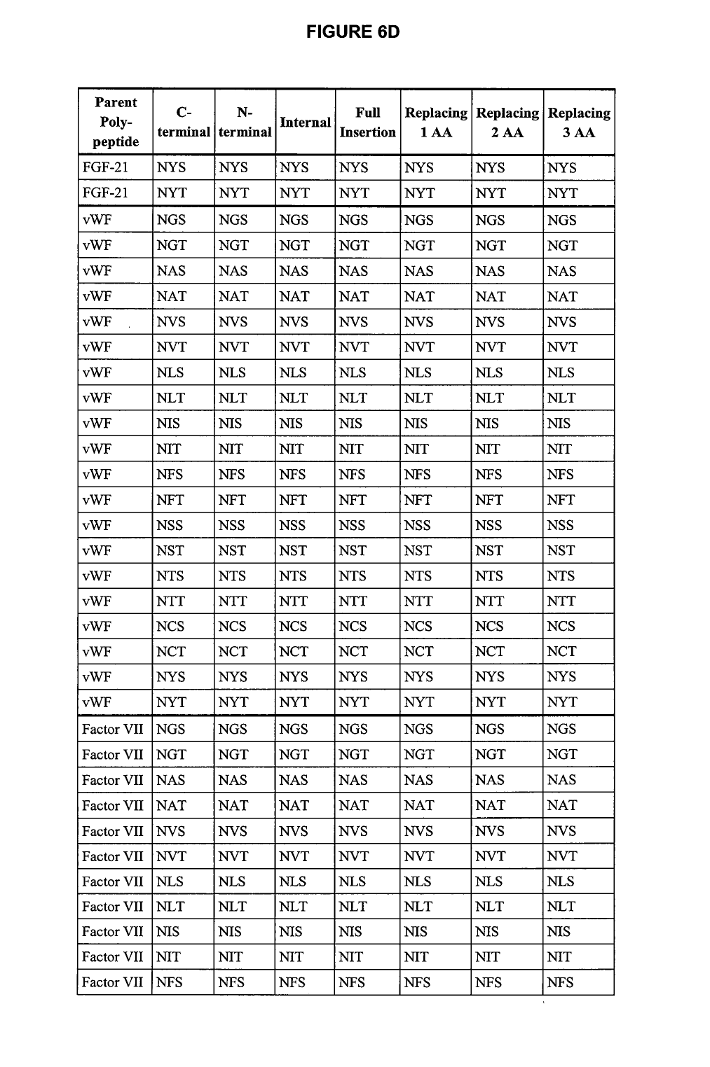

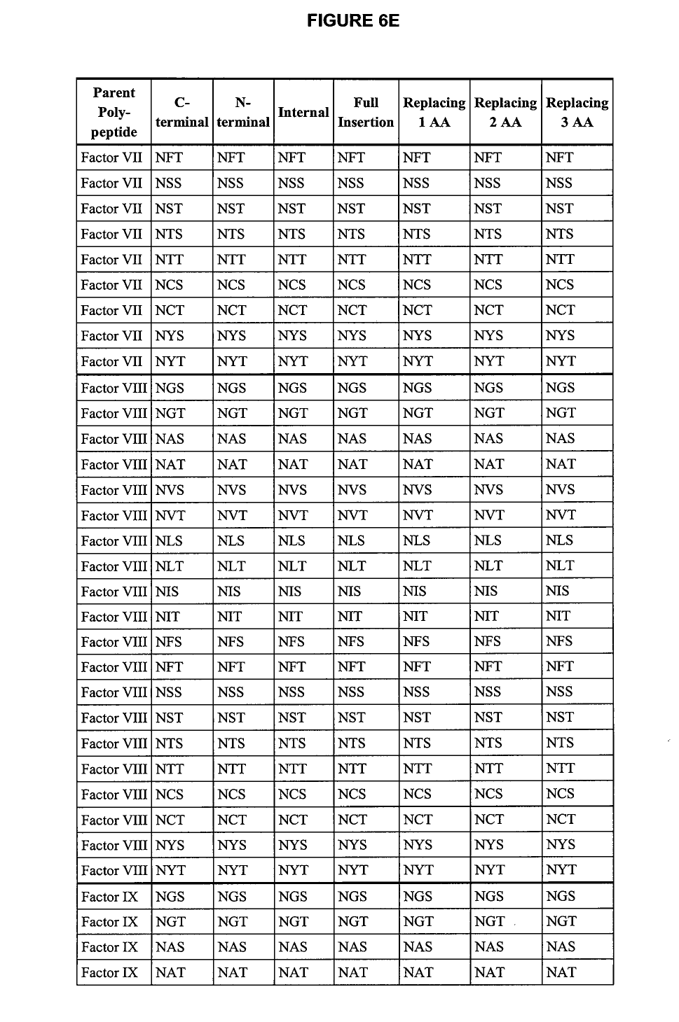

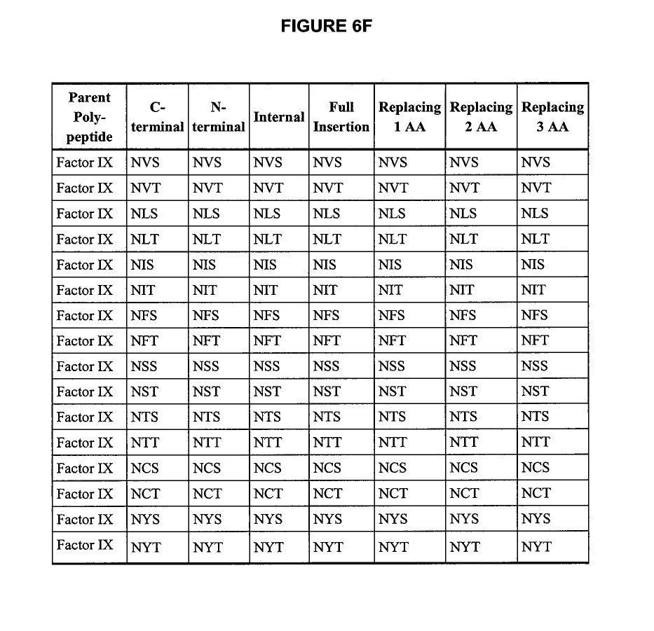

In certain embodiments, a particular parent polypeptide of the invention is used in conjunction with a particular N-linked glycosylation sequence of the invention. Exemplary parent polypeptide/N-linked glycosylation sequence combinations are summarized in FIG. 6. Each row in FIG. 6 represents an exemplary embodiment of the invention. The combinations shown may be used in all aspects of the invention including single sequon polypeptides, libraries of polypeptides, polypeptide conjugates and methods of the invention. One of skill in the art will appreciate that the embodiments set forth in FIG. 6 for the indicated parent polypeptides can equally apply to other parent polypeptides set forth herein. One of skill in the art will also appreciate that the listed polypeptides can be used in the illustrated manner with any glycosylation sequence set forth herein.

### Libraries of Polypeptides

One strategy for the identification of polypeptides, which are glycosylated or glycoconjugated (e.g., glycoPEGylated) efficiently (e.g., with a satisfactory yield) when subjected to a glycosylation or glycoconjugation (e.g., glycoPEGylation) reaction, is to insert an N-linked glycosylation sequence of the invention at a variety of different positions within the amino acid sequence of a parent polypeptide, e.g., including beta-sheet domains and alpha-helical domains, and then to test a number of the resulting sequon polypeptides for their ability to function as an efficient substrate for an oligosaccharyltransferase.

Hence, in another aspect, the invention provides a library of sequon polypeptides including a plurality of different members, wherein each member of the library corresponds to a common parent polypeptide and includes at least one independently selected exogenous N-linked glycosylation sequence of the invention. In one embodiment, each member of the library includes the same N-linked glycosylation sequence, each at a different amino acid position within the parent polypeptide. In another embodiment, each member of the library includes a different N-linked glycosylation sequence, however at the same amino acid position within the parent polypeptide. N-linked glycosylation sequences, which are useful in conjunction with the libaries of the invention are described herein. In one embodiment, the N-linked glycosylation sequence used in a library of the invention has an amino acid sequence according to Formula (I) (SEQ ID NO: 1). In another embodiment, the N-linked glycosylation sequence used in a library of the invention has an amino acid sequence according to Formula (II) (SEQ ID NO: 2). Formula (I) and Formula (II) are described herein, above.

In a preferred embodiment, the N-linked glycosylation sequence used in conjunction with the libraries of the invention has an amino acid sequence, which is selected from: X¹NGSX⁴, X¹NGTX⁴, X¹NASX⁴, X¹NATX⁴, X¹NVSX⁴, X¹NVTX⁴, X¹NLSX⁴, X¹NLTX⁴, X¹NISX⁴, X¹NITX⁴, X¹NFSX⁴ and X¹NFTX⁴, X¹D X^{2′}NGSX⁴, X¹DX^{2′}NGTX⁴, X¹DX^{2′}NASX⁴, X¹DX^{2′}NATX⁴, X¹DX^{2′}NVSX⁴, X¹DX^{2′}NVTX⁴, X¹DX^{2′}NLSX⁴, X¹DX^{2′}NLTX⁴, X¹DX^{2′}NISX⁴, X¹DX^{2′}NITX⁴, X¹DX^{2′}NFSX⁴ and X¹DX^{2′}NFTX⁴, wherein X¹, X^{2′} and X⁴ are defined as above.

In one embodiment, in which each member of the library has a common N-linked glycosylation sequence, the parent polypeptide has an amino acid sequence that includes “m” amino acids. In one example, the library of sequon polypeptides includes (a) a first sequon polypeptide having the N-linked glycosylation sequence at a first amino acid position (AA)ₙ within the parent polypeptide, wherein n is a member selected from 1 to m; and (b) at least one additional sequon polypeptide, wherein in each additional sequon polypeptide the N-linked glycosylation sequence is introduced at an additional amino acid position, each additional amino acid position selected from (AA)ₙ₊ₓ and (AA)ₙ₋ₓ, wherein x is a member selected from 1 to (m-n). For example, a first sequon polypeptide is generated through introduction of a selected N-linked glycosylation sequence at the first amino acid position. Subsequent sequon polypeptides may then be generated by introducing the same N-linked glycosylation sequence at an amino acid position, which is located further towards the N- or C-terminus of the parent polypeptide.

In this context, when n-x is 0 (AA₀) then the glycosylation sequence is introduced immediately preceding the N-terminal amino acid of the parent polypeptide. An exemplary sequon polypeptide may have the partial sequence: “NLTM¹ . . . .”

The first amino acid position (AA)ₙ can be anywhere within the amino acid sequence of the parent polypeptide. In one embodiment, the first amino acid position is selected (e.g., at the beginning of a loop domain).

Each additional amino acid position can be anywhere within the parent polypeptide. In one example, the library of sequon polypeptides includes a second sequon polypeptide having the N-linked glycosylation sequence at an amino acid position selected from (AA)ₙ₊ₚ and (AA)ₙ₋ₚ, wherein p is selected from 1 to about 10, preferably from 1 to about 8, more preferably from 1 to about 6, even more preferably from 1 to about 4 and most preferably from 1 to about 2. In one embodiment, the library of sequon polypeptides includes a first sequon polypeptide having an N-linked glycosylation sequence at amino acid position (AA)ₙ and a second sequon polypeptide having an N-linked glycosylation sequence at amino acid position (AA)ₙ₊₁ or (AA)ₙ₋₁.

In another example, each of the additional amino acid position is immediately adjacent to a previously selected amino acid position. In yet another example, each additional amino acid position is exactly 1, 2, 3, 4, 5, 6, 7, 8, 9 or 10 amino acid(s) removed from a previously selected amino acid position.

Introduction of an N-linked glycosylation sequence “at a given amino acid position” of the parent polypeptide means that the mutation is introduced starting immediately next to the given amino acid position (towards the C-terminus). Introduction can occur through full insertion (not replacing any existing amino acids), or by replacing any number of existing amino acids.

In an exemplary embodiment, the library of sequon polypeptides is generated by introducing the N-linked glycosylation sequence at consecutive amino acid positions of the parent polypeptide, each located immediately adjacent to the previously selected amino acid position, thereby “scanning” the glycosylation sequence through the amino acid chain, until a desired, final amino acid position is reached. Immediately adjacent means exactly one amino acid position further towards the N- or C-terminus of the parent polypeptide. For instance, the first mutant is created by introduction of the glycosylation sequence at amino acid position AAₙ. The second member of the library is generated through introduction of the glycosylation site at amino acid position AAₙ₊₁, the third mutant at AAₙ₊₂, and so forth. This procedure has been termed “sequon scanning”. One of skill in the art will appreciate that sequon scanning can involve designing the library so that the first member has the glycosylation sequence at amino acid position (AA)ₙ, the second member at amino acid position (AA)ₙ₊₂, the third at (AA)ₙ₊₄ etc. Likewise, the members of the library may be characterized by other strategic placements of the glycosylation sequence. For example:

A) member 1: (AA)ₙ; member 2: (AA)ₙ₊₃; member 3: (AA)ₙ₊₆; member 4: (AA)ₙ₊₉ etc.
B) member 1: (AA)ₙ; member 2: (AA)ₙ₊₄; member 3: (AA)ₙ₊₈; member 4: (AA)ₙ₊₁₂ etc.
C) member 1: (AA)ₙ; member 2: (AA)ₙ₊₅; member 3: (AA)ₙ₊₁₀; member 4: (AA)ₙ₊₁₅ etc.

In one embodiment, a first library of sequon polypeptides is generated by scanning a selected N-linked glycosylation sequence of the invention through a particular region of the parent polypeptide (e.g., from the beginning of a particular loop region to the end of that loop region). A second library is then generated by scanning the same glycosylation sequence through another region of the polypeptide, “skipping” those amino acid positions, which are located between the first region and the second region. The part of the polypeptide chain that is left out may, for instance, correspond to a binding domain important for biological activity or another region of the polypeptide sequence known to be unsuitable for glycosylation. Any number of additional libraries can be generated by performing “sequon scanning” for additional stretches of the polypeptide. In an exemplary embodiment, a library is generated by scanning the N-linked glycosylation sequence through the entire polypeptide introducing the mutation at each amino acid position within the parent polypeptide.

In one embodiment, the members of the library are part of a mixture of polypeptides. For example, a cell culture is infected with a plurality of expression vectors, wherein each vector includes the nucleic acid sequence for a different sequon polypeptide of the invention. Upon expression, the culture broth may contain a plurality of different sequon polypeptides, and thus includes a library of sequon polypeptides. This technique may be useful to determine, which sequon polypeptide of a library is expressed most efficiently in a given expression system.

In another embodiment, the members of the library exist isolated from each other. For example, at least two of the sequon polypeptides of the above mixture may be isolated. Together, the isolated polypeptides represent a library. Alternatively, each sequon polypeptide of the library is expressed separately and the sequon polypeptides are optionally isolated. In another example, each member of the library is synthesized by chemical means and optionally purified.

### Exemplary Polypeptides and Polypeptide Libraries

An exemplary parent polypeptide is recombinant human BMP-7. The selection of BMP-7 as an exemplary parent polypeptide is for illustrative purposes and is not meant to limit the scope of the invention. A person of skill in the art will appreciate that any parent polypeptide (e.g., those set forth herein) are equally suitable for the following exemplary modifications. Any polypeptide variant thus obtained falls within the scope of the invention. Biologically active BMP-7 variants of the present invention include any BMP-7 polypeptide, in part or in whole, that includes at least one modification that does not result in substantial or entire loss of its biological activity as measured by any suitable functional assay known to one skilled in the art. The following sequence (140 amino acids) represents a biologically active portion of the full-length BMP-7 sequence (sequence S.1):

(SEQ ID NO: 10)

M¹STGSKQRSQNRSKTPKNQEALRMANVAENSSSDQRQACK
KHELYVSFRDLGWQDWIIAPEGYAAYYCEGECAFPLNSYMNATNHAI
VQTLVHFINPETVPKPCCAPTQLNAISVLYFDDSSNVILKKYRNMVVR
ACGCH

Exemplary BMP-7 variant polypeptides, which are based on the above parent polypeptide sequence, are listed in Tables 3-11, below.

In one exemplary embodiment, mutations are introduced into the wild-type BMP-7 amino acid sequence S.1 (SEQ ID NO: 10) replacing the corresponding number of amino acids in the parent sequence, resulting in a sequon polypeptide that contains the same number of amino acid residues as the parent polypeptide. For instance, directly substituting three amino acids normally in BMP-7 with the N-linked glycosylation sequence “asparagine-leucine-threonine” (NLT) and then sequentially moving the NLT sequence towards the C-terminus of the polypeptide provides 137 BMP-7 variants each including NLT. Exemplary sequences according to this embodiment are listed in Table 3, below.

TABLE 3

Exemplary library of BMP-7 variants including 140 amino acids wherein
three existing amino acids are replaced with the N-linked glycosylation
sequence “NLT”

Introduction at position 1, replacing 3 existing amino acids:
M¹NLTSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 11)
Introduction at position 2, replacing 3 existing amino acids:
M¹SNLTKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 12)
Introduction at position 3, replacing 3 existing amino acids:
M¹STNLTQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 13)
Additional BMP-7 variants can be generated by “scanning” the glycosylation sequence
through the entire sequence in the above fashion. All variant BMP-7 sequences thus obtained
are within the scope of the invention. The final sequon polypeptide so generated has the
following sequence:
Introduction at position 137, replacing 3 existing amino acids:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACNLT (SEQ ID NO: 14)

In another exemplary embodiment, the N-linked glycosylation sequence is introduced into the wild-type BMP-7 amino acid sequence S.1 (SEQ ID NO: 10) by adding one or more amino acids to the parent sequence. For instance, the N-linked glycosylation sequence NLT is added to the parent BMP-7 sequence replacing either 2, 1 or none of the amino acids in the parent sequence. In this example, the maximum number of added amino acid residues corresponds to the length of the inserted glycosylation sequence. In an exemplary embodiment, the parent sequence is extended by exactly one amino acid. For example, the N-linked glycosylation sequence NLT is added to the parent BMP-7 peptide replacing 2 amino acids normally present in BMP-7. Exemplary sequences according to this embodiment are listed in Table 4, below.

TABLE 4

Exemplary library of mutant BMP-7 polypeptides including 141 amino acids,
wherein two existing amino acids are replaced with the N-linked glycosylation
sequence “NLT”

Introduction at position 1, replacing 2 amino acids (ST)
M¹NLTGSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 15)
Introduction at position 2, replacing 2 amino acids (TG)
M¹SNLTSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 16)
Introduction at position 3, replacing 2 amino acids (GS)
M¹STNLTKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 17)
Introduction at position 4, replacing 2 amino acids (SK)
M¹STGNLTQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 18)
Introduction at position 5, replacing 2 amino acids (KQ)
M¹STGSNLTRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 19)
Additional BMP-7 variants can be generated by “scanning” the glycosylation sequence
through the entire sequence in the above fashion until the following sequence is reached:
Introduction at position 138, replacing 2 existing amino acids (CH):
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGNLT (SEQ ID NO: 20)
All BMP-7 variants thus obtained are within the scope of the invention.

Another example involves the addition of an N-linked glycosylation sequence (e.g., NLT) to the parent polypeptide (e.g., BMP-7) replacing 1 amino acid normally present in the parent polypeptide (double amino acid insertion). Exemplary sequences according to this embodiment are listed in Table 5, below.

TABLE 5

Exemplary library of BMP-7 mutants including NLT; replacement of one
existing amino acid (142 amino acids)

Introduction at position 1, replacing 1 amino acid (S)
M¹NLTTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 21)
Introduction at position 2, replacing 1 amino acid (T)
M¹SNLTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 22)
Introduction at position 3, replacing 1 amino acid (G)
M¹STNLTSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 23)
Introduction at position 4, replacing 1 amino acid (S)
M¹STGNLTKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 24)
Introduction at position 5, replacing 1 amino acid (K)
M¹STGSNLTQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 25)
Additional BMP-7 variants can be generated by “scanning” the glycosylation sequence
through the entire sequence in the above fashion until the following sequence is reached:
Introduction at position 139, replacing 1 existing amino acid (H):
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCNLT (SEQ ID NO: 26)
All BMP-7 variants thus obtained are within the scope of the invention.

Yet another example involves the creation of an N-linked glycosylation sequence within the parent polypeptide (e.g., BMP-7) replacing none of the amino acids normally present in the parent polypeptide and adding the entire length of the glycosylation sequence (e.g., triple amino acid insertion for NLT). Exemplary sequences according to this embodiment are listed in Table 6, below.

TABLE 6

Exemplary library of BMP-7 variants including NLT; addition of 3 amino
acids (143 amino acids)

Introduction at position 1, adding 3 amino acids
M¹NLTSTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 27)
Introduction at position 2, adding 3 amino acids
M¹SNLTTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 28)
Introduction at position 3, adding 3 amino acids
M¹STNLTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 29)
Introduction at position 4, adding 3 amino acids
M¹STGNLTSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 30)
Additional BMP-7 mutants can be generated by “scanning” the glycosylation sequence
through the entire sequence in the above fashion until a final sequence is reached:
Introduction at position 140, adding 3 amino acids:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCHNLT (SEQ ID NO: 31)
All BMP-7 variants thus obtained are within the scope of the invention.

BMP-7 variants analogous to those examples in Tables 3-6 can be generated using any of the N-linked glycosylation sequences of the invention. All resulting BMP-7 variants are within the scope of the invention. For instance, instead of NLT the sequence DRNLT (SEQ ID NO: 32) can be used. In an exemplary embodiment DRNLT is introduced into the parent polypeptide replacing 5 amino acids normally present in BMP-7. Exemplary sequences according to this embodiment are listed in Table 7, below.

TABLE 7

Exemplary library of BMP-7 variants including DRNLT; replacement of 5
amino acids (140 amino acids)

M¹DRNLTQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 33)
M¹SDRNLTRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 34)
M¹STDRNLTSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 35)
M¹STGDRNLTQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 36)
Additional BMP-7 mutants can be generated by “scanning” the glycosylation sequence
through the entire sequence in the above fashion until a final sequence is reached:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRDRNLT (SEQ ID NO: 37)
All mutant BMP-7 sequences thus obtained are within the scope of the invention.

In another example the N-linked glycosylation sequence DRNLT is added to the parent polypeptide (e.g., BMP-7) at or close to either the N- or C-terminal of the parent sequence, adding 1 to 5 amino acids to the parent polypeptide. Exemplary sequences according to this embodiment are listed in Table 8, below.

TABLE 8

Exemplary libraries of BMP-7 variants including DRNLT
(141-145 amino acids)

Amino-terminal mutants:
Introduction at position 1, adding 5 amino acids
M¹DRNLTSTGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 38)
Introduction at position 1, adding 4 amino acids, replacing 1 amino acid (S)
M¹DRNLTTGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 39)
Introduction at position 1, adding 3 amino acids, replacing 2 amino acids (ST)
M¹DRNLTGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 40)
Introduction at position 1, adding 2 amino acids, replacing 3 amino acids (STG)
M¹DRNLTSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 41)
Introduction at position 1, adding 1 amino acids, replacing 4 amino acids (STGS)
M¹DRNLTKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 42)
Carboxy-terminal mutants
Introduction at position 140, adding 5 amino acids
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCHDRNLT (SEQ ID NO: 43)
Introduction at position 139, adding 4 amino acids, replacing 1 amino acid (H)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCDRNLT (SEQ ID NO: 44)
Introduction at position 138, adding 3 amino acids, replacing 2 amino acid (CH)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGDRNLT (SEQ ID NO: 45)
Introduction at position 137, adding 2 amino acids, replacing 3 amino acid (GCH)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACDRNLT (SEQ ID NO: 46)
Introduction at position 136, adding 1 amino acids, replacing 4 amino acid (CGCH)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRADRNLT (SEQ ID NO: 47)

Yet another example involves insertion of the N-linked glycosylation sequence DFNVS (SEQ ID NO: 48) into the parent polypeptide (e.g., BMP-7), adding 1 to 5 amino acids to the parent sequence. Exemplary sequences according to this embodiment are listed in Table 9, below.

TABLE 9
Exemplary library of BMP-7 variants including DFNVS

Insertion of one amino acid
M¹DFNVSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 49)
M¹SDFNVSQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 50)
M¹STDFNVSRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 51)
Additional BMP-7 mutants can be generated by “scanning” the glycosylation sequence
through the entire sequence in the above fashion until a final sequence is reached:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRADFNVS (SEQ ID NO: 52)
All BMP-7 variants thus obtained are within the scope of the invention.
Insertion of two amino acids
M¹DFNVSSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 53)
M¹SDFNVSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 54)
M¹STDFNVSQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 55)
Additional BMP-7 variants can be generated by “scanning” the glycosylation sequence
through the entire sequence in the above fashion until a final sequence is reached:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACDFNVS (SEQ ID NO: 56)
All BMP-7 variants thus obtained are within the scope of the invention.
Insertion of three amino acids
M¹DFNVSGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 57)
M¹SDFNVSSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 58)
M¹STDFNVSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 59)
Additional BMP-7 variants can be generated by “scanning” the glycosylation sequence
through the entire sequence in the above fashion until a final sequence is reached:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGDFNVS (SEQ ID NO: 60)
All BMP-7 variants thus obtained are within the scope of the invention.
Insertion of four amino acids
M¹DFNVSTGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 61)
M¹SDFNVSGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 62)
M¹STDFNVSSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 63)
Additional BMP-7 variants can be generated by “scanning” the glycosylation sequence
through the entire sequence in the above fashion until a final sequence is reached:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCDFNVS (SEQ ID NO: 64)
All BMP-7 variants thus obtained are within the scope of the invention.
Insertion of five amino acids
M¹DFNVSSTGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 65)
M¹SDFNVSTGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 66)
M¹STDFNVSGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 67)
Additional BMP-7 variants can be generated by “scanning” the glycosylation sequence
through the entire sequence in the above fashion until a final sequence is reached:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCHDFNVS (SEQ ID NO: 68)
All BMP-7 variants thus obtained are within the scope of the invention.

In one example, the N-linked glycosylation sequence (e.g., NLT or NVS) is placed at all possible amino acid positions within selected polypeptide regions either by substitution of existing amino acids and/or by insertion. Exemplary sequences according to this embodiment are listed in Table 10 and Table 11, below.

TABLE 10
Exemplary library of BMP-7 variants including NLT between A⁷³ and A⁸²

Substitution of existing amino acids
---A⁷³FPLNSYMNA⁸²TNHAIVQTLVHFI⁹⁵NPETVPKP¹⁰³---(SEQ ID NO: 69) (parent)
---N⁷³LTLNSYMNA⁸²TNHAIVQTLVHFI⁹⁵NPETVPKP¹⁰³---(SEQ ID NO: 70)
---A⁷³NLTNSYMNA⁸²TNHAIVQTLVHFI⁹⁵NPETVPKP¹⁰³---(SEQ ID NO: 71)
---A⁷³FNLTSYMNA⁸²TNHAIVQTLVHFI⁹⁵NPETVPKP¹⁰³---(SEQ ID NO: 72)
---A⁷³FPNLTYMNA⁸²TNHAIVQTLVHFI⁹⁵NPETVPKP¹⁰³---(SEQ ID NO: 73)
---A⁷³FPLNLTMNA⁸²TNHAIVQTLVHFI⁹⁵NPETVPKP¹⁰³---(SEQ ID NO: 74)
---A⁷³FPLNNLTNA⁸²TNHAIVQTLVHFI⁹⁵NPETVPKP¹⁰³---(SEQ ID NO: 75)
---A⁷³FPLNSNLTA⁸²TNHAIVQTLVHFI⁹⁵NPETVPKP¹⁰³---(SEQ ID NO: 76)
---A⁷³FPLNSYNLT⁸²TNHAIVQTLVHFI⁹⁵NPETVPKP¹⁰³---(SEQ ID NO: 77)

TABLE 11

Exemplary library of BMP-7 variants including
NLT between I⁹⁵ and P¹⁰³

Substitution of existing amino acids
---A⁷³FPLNSYMNA⁸²TNHAIVQTLVHFN⁹⁵LTETVPKP¹⁰³---(SEQ ID NO: 78)
---A⁷³FPLNSYMNA⁸²TNHAIVQTLVHFI⁹⁵NLTTVPKP¹⁰³---(SEQ ID NO: 79)
---A⁷³FPLNSYMNA⁸²TNHAIVQTLVHFI⁹⁵NNLTVPKP¹⁰³---(SEQ ID NO: 80)
---A⁷³FPLNSYMNA⁸²TNHAIVQTLVHFI⁹⁵NPNLTPKP¹⁰³---(SEQ ID NO: 81)
---A⁷³FPLNSYMNA⁸²TNHAIVQTLVHFI⁹⁵NPENLTKP¹⁰³---(SEQ ID NO: 82)
---A⁷³FPLNSYMNA⁸²TNHAIVQTLVHFI⁹⁵NPETNLTP¹⁰³---(SEQ ID NO: 83)
---A⁷³FPLNSYMNA⁸²TNHAIVQTLVHFI⁹⁵NPETVNLT¹⁰³---(SEQ ID NO: 84)
Insertion (with one amino acid added) between existing amino acids
---N⁷³LTPLNSYMNA⁸³TNHAIVQTLVHFI⁹⁶NPETVPKP¹⁰⁴---(SEQ ID NO: 85)
---A⁷³NLTLNSYMNA⁸³TNHAIVQTLVHFI⁹⁶NPETVPKP¹⁰⁴---(SEQ ID NO: 86)
---A⁷³FNLTNSYMNA⁸³TNHAIVQTLVHFI⁹⁶NPETVPKP¹⁰⁴---(SEQ ID NO: 87)
---A⁷³FPNLTSYMNA⁸³TNHAIVQTLVHFI⁹⁶NPETVPKP¹⁰⁴---(SEQ ID NO: 88)
---A⁷³FPLNLTYMNA⁸³TNHAIVQTLVHFI⁹⁶NPETVPKP¹⁰⁴---(SEQ ID NO: 89)
---A⁷³FPLNNLTMNA⁸³TNHAIVQTLVHFI⁹⁶NPETVPKP¹⁰⁴---(SEQ ID NO: 90)
---A⁷³FPLNSNLTNA⁸³TNHAIVQTLVHFI⁹⁶NPETVPKP¹⁰⁴---(SEQ ID NO: 91)
---A⁷³FPLNSYNLTA⁸³TNHAIVQTLVHFI⁹⁶NPETVPKP¹⁰⁴---(SEQ ID NO: 92)
---A⁷³FPLNSYMNLT⁸³TNHAIVQTLVHFI⁹⁶NPETVPKP¹⁰⁴---(SEQ ID NO: 93)
Insertion (with one amino acid added) between existing amino acids
---A⁷³FPLNSYMNA⁸²TNHAIVQTLVHFN⁹⁵LTPETVPKP¹⁰⁴---(SEQ ID NO: 94)
---A⁷³FPLNSYMNA⁸²TNHAIVQTLVHFI⁹⁵NLTETVPKP¹⁰⁴---(SEQ ID NO: 95)
---A⁷³FPLNSYMNA⁸²TNHAIVQTLVHFI⁹⁵NNLTTVPKP¹⁰⁴---(SEQ ID NO: 96)
---A⁷³FPLNSYMNA⁸²TNHAIVQTLVHFI⁹⁵NPNLTVPKP¹⁰⁴---(SEQ ID NO: 97)
---A⁷³FPLNSYMNA⁸²TNHAIVQTLVHFI⁹⁵NPENLTPKP¹⁰⁴---(SEQ ID NO: 98)
---A⁷³FPLNSYMNA⁸²TNHAIVQTLVHFI⁹⁵NPETNLTKP¹⁰⁴---(SEQ ID NO: 99)
---A⁷³FPLNSYMNA⁸²TNHAIVQTLVHFI⁹⁵NPETVNLTP¹⁰⁴---(SEQ ID NO: 100)
---A⁷³FPLNSYMNA⁸²TNHAIVQTLVHFI⁹⁵NPETVPNLT¹⁰⁴---(SEQ ID NO: 101)

The above substitutions and insertions can be made using any N-linked glycosylation sequences of the invention, e.g. NLT and SEQ ID NOs: 32 and 48. All BMP-7 variants thus obtained are within the scope of the invention.

In another exemplary embodiment, one or more N-linked glycosylation sequence, such as those set forth above is inserted into a blood coagulation Factor, e.g., Factor VII, Factor VIII or Factor IX polypeptide. As set forth in the context of BMP-7, the N-linked glycosylation sequence can be inserted in any of the various motifs exemplified with BMP-7. For example, the N-linked glycosylation sequence can be inserted into the wild type sequence without replacing any amino acid(s) native to the wild type sequence. In an exemplary embodiment, the N-linked glycosylation sequence is inserted at or near the N- or C-terminus of the polypeptide. In another exemplary embodiment, one or more amino acid residue native to the wild type polypeptide sequence is removed prior to insertion of the N-linked glycosylation sequence. In yet another exemplary embodiment, one or more amino acid residue native to the wild type sequence is a component of the N-linked glycosylation sequence (e.g., a asparagine) and the N-linked glycosylation sequence encompasses the wild type amino acid(s). The wild type amino acid(s) can be at either terminus of the N-linked glycosylation sequence or internal to the N-linked glycosylation sequence.

Furthermore, any preexisting N-linked or O-linked glycosylation sequence can be replaced with an N-linked glycosylation sequence of the invention. In addition, an N-linked glycosylation sequence can be inserted adjacent to one or more O-linked glycosylation sequences. In one embodiment, the presence of the N-linked glycosylation sequence prevents the glycosylation of the O-linked glycosylation sequence.

In a representative example, the parent polypeptide is Factor VIII. In this embodiment, the N-linked glycosylation sequence can be inserted into the A-, B-, or C-domain according to any of the motifs set forth above. More than one N-linked glycosylation sequence can be inserted into a single domain or more than one domain; again, according to any of the motifs above. For example, an N-linked glycosylation sequence can be inserted into each of the A, B and C domains, the A and C domains, the A and B domains or the B and C domains. Alternatively, an N-linked glycosylation sequence can flank the A and B domain or the B and C domain. An exemplary amino acid sequence for Factor VIII is provided in FIG. 2.

In another exemplary embodiment, the Factor VIII polypeptide is a B-domain deleted (BDD) Factor VIII polypeptide. In this embodiment, the N-linked glycosylation sequence can be inserted into the peptide linker joining the 80 Kd and 90 Kd subunits of the Factor VIII heterodimer. Alternatively, the N-linked glycosylation sequence can flank the A domain and the linker or the C domain and linker. As set forth above in the context of BMP-7, the N-linked glycosylation sequence can be inserted without replacement of existing amino acids, or may be inserted replacing one or more amino acids of the parent polypeptide. An exemplary sequence for B-domain deleted (BDD) Factor VIII is provided in FIG. 3.

Other B-domain deleted Factor VIII polypeptides are also suitable for use with the invention, including, for example, the B-domain deleted Factor VIII polypeptide disclosed in Sandberg et al., Seminars in Hematology 38(2):4-12 (2000), the disclosure of which is incorporated herein by reference.

As will be apparent to one of skill in the art, that polypeptides including more than one mutant N-linked glycosylation sequence of the invention are also within the scope of the present invention. Additional mutations may be introduced to allow for the modulation of polypeptide properties, such e.g., biological activity, metabolic stability (e.g., reduced proteolysis), pharmacokinetics and the like.

Once a variety of variants are prepared, they can be evaluated for their ability to function as a substrate for N-linked glycosylation or glycoPEGylation. Succesfull glycosylation and/or glycoPEGylation may be detected and quantified using methods known in the art, such as mass spectroscopy (e.g., MALDI-TOF or Q-TOF), gel electrophoresis (e.g., in combination with densitometry) or chromatographic analyses (e.g., HPLC). Biological assays, such as enzyme inhibition assays, receptor-binding assays and/or cell-based assays can be used to analyze biological activities of a given polypeptide or polypeptide conjugate. Evaluation strategies are described in more detail herein, below (see e.g., “Identification of Lead polypeptides”). It will be within the abilities of a person skilled in the art to select and/or develop an appropriate assay system useful for the chemical and biological evaluation of each polypeptide.

### Polypeptide Conjugates

In another aspect, the present invention provides a covalent conjugate between a polypeptide (e.g., a sequon polypeptide) and a selected modifying group (e.g., a polymeric modifying group), in which the modifying group is conjugated to the polypeptide via a glycosyl linking group (e.g., an intact glycosyl linking group). The glycosyl linking group is interposed between and covalently linked to both the polypeptide and the modifying group. Exemplary methods useful in the preparation of the current polypeptide conjugates are set forth herein. Other useful methods are set forth in U.S. Pat. Nos. 5,876,980; 6,030,815; 5,728,554; and 5,922,577, as well as WO 98/31826; WO2003/031464; WO2005/070138; WO2004/99231; WO2004/10327; WO2006/074279; and U.S. Patent Application Publication 2003180835, all of which are incorporated herein by reference for all purposes.

The conjugates of the invention will typically correspond to the general structure:

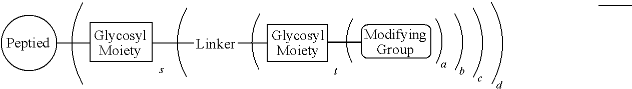

in which the symbols a, b, c, d and s represent a positive, non-zero integer; and t is either 0 or a positive integer. The “modifying group” can be a therapeutic agent, a bioactive agent (e.g., a toxin), a detectable label, a polymer (e.g., water-soluble polymer) or the like. The linker can be any of a wide array of linking groups, infra. Alternatively, the linker may be a single bond. The identity of the polypeptide is without limitation.

Exemplary polypeptide conjugates include an N-linked GlcNAc or GlcNH residue that is bound to the N-linked glycosylation sequence through the action of an oligosaccharyl transferase. In one embodiment, GlcNAc or GlcNH itself is derivatized with a modifying group and represents the glycosyl linking group. In another embodiment, additional glycosyl residues are bound to the GlcNAc moiety. For example, another GlcNAc, a Gal or Gal-Sia moiety, each of which can be modified with a modifying group, is bound to the GlcNAc moiety. In representative embodiments, the N-linked saccharyl residue is GlcNAc-X*, GlcNH-X*, GlcNAc-GlcNAc-X*, GlcNAc-GlcNH-X*, GlcNAc-Gal-X*, GlcNAc-Gal-Sia-X*, GlcNAc-GlcNAc-Gal-Sia-X*, in which X* is a modifying group (e.g., water-soluble polymeric modifying group).

In one embodiment, the present invention provides polypeptide conjugates that are highly homogenous in their substitution patterns. Using the methods of the invention, it is possible to form polypeptide conjugates in which essentially all of the modified sugar moieties across a population of conjugates of the invention are attached to a structurally identical amino acid or glycosyl residue. Thus, in an exemplary embodiment, the invention provides polypeptide conjugates including at least one modifying group (e.g., water-soluble polymeric modifying group) covalently bound to an amino acid residue (e.g., asparagine) within an N-linked glycosylation sequence through a glycosyl linking group. In one example, each amino acid residue having a glycosyl linking group attached thereto has the same structure. In another exemplary embodiment, essentially each member of the population of modifying groups (e.g., water-soluble polymeric moieties) is bound via a glycosyl linking group to a glycosyl residue of the polypeptide, and each glycosyl residue of the polypeptide to which the glycosyl linking group is attached has the same structure.

In one aspect the invention provides a covalent conjugate between a polypeptide and a modifying group (e.g., a polymeric modifying group), wherein the polypeptide comprises an exogenous N-linked glycosylation sequence of the invention. Typically, the N-linked glycosylation sequence includes an asparagine (N) residue. The polymeric modifying group is covalently conjugated to the polypeptide at the asparagine residue of the N-linked glycosylation sequence via a glycosyl linking group interposed between and covalently linked to both the polypeptide and the polymeric modifying group. The glycosyl linking group can be a monosaccharide or an oligosaccharide. Exemplary N-linked glycosylation sequences are described herein and may have a structure according to SEQ ID NO: 1 or SEQ ID NO: 2. Exemplary polymeric modifying groups, such as water soluble polymeric modifying groups (e.g., PEG or m-PEG) are also described herein.

In one aspect, the invention provides a covalent conjugate comprising a sequon polypeptide having an N-linked glycosylation sequence (e.g., an exogenous N-linked glycosylation sequence). In one embodiment, the polypeptide conjugate includes a moiety according to Formula (III):

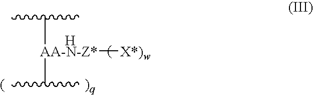

In Formula (III), w is an integer selected from 0 to 20. In one embodiment, w is selected from 0 to 8. In another embodiment, w is selected from 0 to 4. In yet another embodiment, w is selected from 0 to 1. In one particular example, w is 1. When w is 0, then (X*)_{w} is replaced with H. X* is a modifying group (e.g., a linear or branched polymeric modifying group). In one example, X* includes a linker moiety that links the modifying group to Z*. In another example, X* is -L^{a}-R^{6c} or -L^{a}-R^{6b}. AA-NH— is a moiety derived from an amino acid within the N-linked glycosylation sequence having a side chain including an amino group (e.g., asparagine). In one embodiment, the integer q is 0 and the amino acid is an N-terminal or C-terminal amino acid. In another embodiment, q is 1 and the amino acid is an internal amino acid.

In Formula (III), Z* is a glycosyl moiety, which is selected from mono- and oligosaccharides. Z* may be a glycosyl-mimetic moiety. When w is 1 or greater, then Z* is a glycosyl linking group. In one embodiment, Z* is a naturally occurring N-linked glycan, such as a trimannosyl core moiety [GlcNAc-GlcNAc-Man(Man)₂], which is optionally substituted with a fucose residue. In one embodiment Z* is a mono-antennary glycan. In another embodiment, Z* is a di-antennary glycan. In yet another embodiment, Z* is a tri-antennary glycan. In a further embodiment, Z* is a tetra-antennary glycan. Each antenna of the Z* glycan may be covalently linked to an independently selected modifying group. For example, each terminal sugar moiety of Z* may be covalently linked to a modifying group.

In one embodiment, the moiety —Z*—(X*)_{w} is represented by the following formula, which includes mono-, di-, tri- and tetra-antennary glycans:

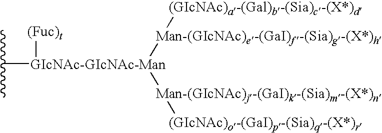

wherein the integers t, a′, b′, c′, d′, e′, f′, g′, h′, j′, k′, l′, m′, n′, o′, p′, q′ and r′ are integers independently selected from 0 and 1. In a preferred embodiment, t is 0.

Exemplary N-linked glycans that are optionally bound to a modifying group are summarized below:

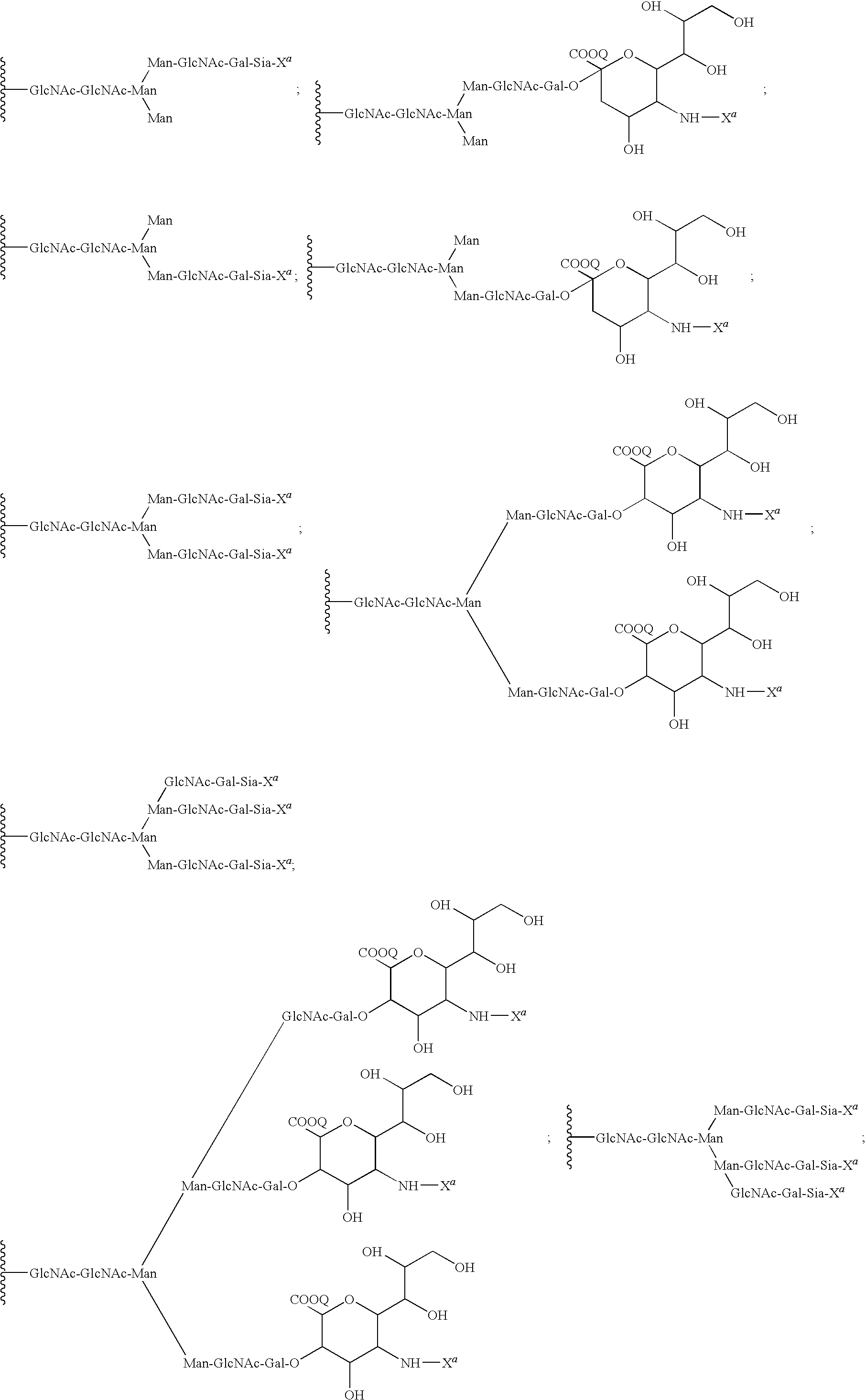

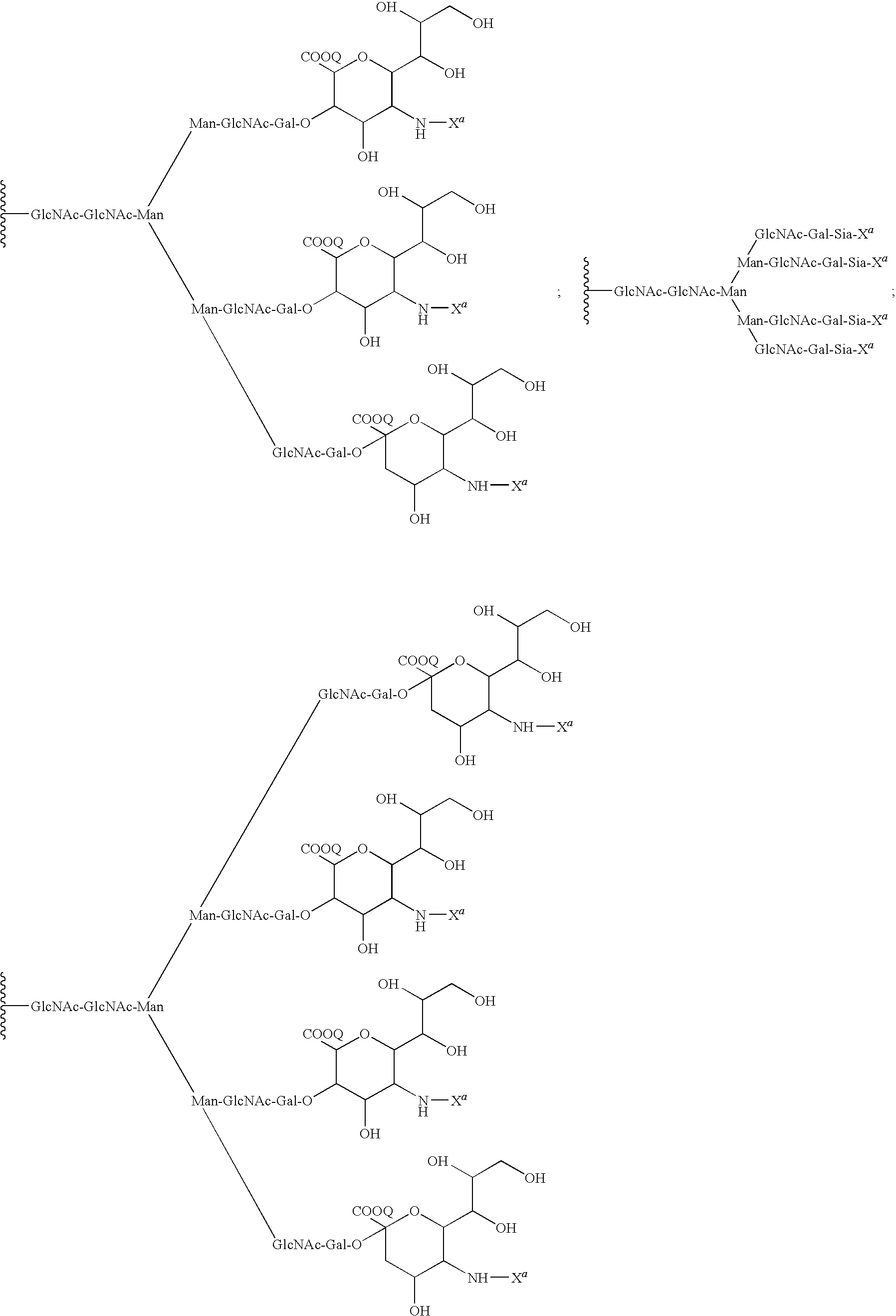

wherein each Q is a member independently selected from H, a single negative charge and a cation (e.g., Na⁺); and each X^{a} is a member independently selected from H, an alkyl group, an acyl group (e.g., acetyl) and a modifying group (X*). In an exemplary embodiment, the N-linked glycan of the invention includes at least one modifying group (at least one X^{a} is X*). Additional N-linked glycans are disclosed in WO03/31464 filed Oct. 9, 2002 and WO04/99231 filed Apr. 9, 2004, the disclosures of which are incorporated herein by reference for all purposes.

In an exemplary embodiment, Z* in Formula (III) includes a GlcNAc moiety. In another exemplary embodiment, Z* includes a GlcNH moiety. In yet another embodiment, Z* includes a GlcNAc- or GlcNH-mimetic moiety. In a further embodiment, Z* includes a bacillosamine (i.e., 2,4-diacetamido-2,4,6-trideoxyglucose) moiety or a derivative thereof. In another embodiment, Z* is selected from GlcNAc, GlcNH, Gal, Man, Glc, GalNAc, GalNH, Sia, Fuc, Xyl and a combination of these moieties. In yet another embodiment, Z* is a combination of GlcNAc, Man and Glc moieties. In a further embodiment, Z* is a combination of GlcNAc, Man, Gal and Sia moieties. In a further embodiment, Z* is a combination of bacillosamine, GalNAc and Glc moieties. In one embodiment, Z* is a GlcNAc moiety. In another embodiment Z* is a GlcNH moiety. In another embodiment, Z* is a Man moiety. In yet another embodiment, Z* is a Sia moiety. In another embodiment, Z* is a Glc moiety. In another embodiment, Z* is a Gal moiety. In another embodiment, Z* is a GalNAc moiety. In another embodiment, Z* is a GalNH moiety. In another embodiment, Z* is a Fuc moiety. In yet another embodiment, Z* is a GlcNAc-GlcNAc, GlcNH-GlcNAc, GlcNAc-GlcNH or GlcNH-GlcNH moiety. In one embodiment, Z* is a GlcNAc-Gal or GlcNH-Gal moiety. In another embodiment, Z* is a GlcNAc-GlcNAc-Gal, GlcNH-GlcNAc-Gal, GlcNAc-GlcNH-Gal or GlcNH-GlcNH-Gal moiety. In another embodiment, Z* is a GlcNAc-Gal-Sia moiety. In another embodiment, Z* is a GlcNAc-GlcNAc-Gal-Sia, GlcNH-GlcNAc-Gal-Sia, GlcNAc-GlcNH-Gal-Sia or GlcNH-GlcNH-Gal-Sia moiety. In another embodiment, Z* is a GlcNAc-GlcNAc-Man moiety.

In one embodiment, the polypeptide conjugate of the invention includes a polypeptide having an N-linked glycosylation sequence having an asparagine residue. In one example according to this embodiment, the polypeptide conjugate includes a moiety having a structure according to Formula (IV):

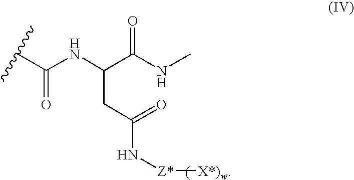

In Formula (IV), w, X* and Z* are defined as above for Formula (III).

### Glycosyl Linking Group

The saccharide component of the modified sugar, when interposed between the polypeptide and a modifying group, becomes a “glycosyl linking group.” In an exemplary embodiment, the glycosyl linking group is derived from a modified mono- or oligosaccharide donor molecule (e.g., a modified dolichol-pyrophosphate sugar) that is a substrate for an appropriate oligosaccharyl transferase. In another exemplary embodiment, the glycosyl linking group includes a glycosyl-mimetic moiety. The polypeptide conjugates of the invention can include glycosyl linking groups that are mono- or multi-valent (e.g., antennary structures). Thus, conjugates of the invention include species in which a modifying group is attached to a polypeptide via a monovalent glycosyl linking group. Also included within the invention are conjugates in which more than one modifying group is attached to a polypeptide via a multi-antennary glycosyl linking group.

In an exemplary embodiment, the moiety —Z*—(X*)_{w} in Formula (III) or (IV) includes a moiety according to Formula (V):

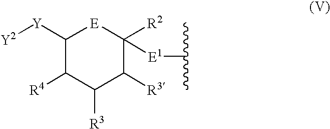

In one embodiment, in Formula (V), E is O. In another embodiment, E is S. In yet another embodiment, E is NR²⁷ or CHR²⁸, wherein R²⁷ and R²⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In one embodiment, E¹ is O. In another embodiment E¹ is S. In another embodiment E¹ is NR²⁷ (e.g., NH). In another embodiment, E¹ is a bond to an amino acid residue of the polypeptide.

In one embodiment, in Formula (V), R² is H. In another embodiment, R² is —R¹. In yet another embodiment R² is —CH₂R¹. In a further embodiment, R² is —C(X¹)R¹. In these embodiments, R¹ is selected from OR⁹, SR⁹, NR¹⁰R¹¹, substituted or unsubstituted alkyl and substituted or unsubstituted heteroalkyl, wherein R⁹ is a member selected from H, a metal ion, substituted or unsubstituted alkyl, substituted or unsubstituted heteroalkyl and acyl. R¹⁰ and R¹¹ are members independently selected from H, substituted or unsubstituted alkyl, substituted or unsubstituted heteroalkyl and acyl. In one embodiment, X¹ is O. In another embodiment, X¹ is a member selected from substituted or unsubstituted alkenyl, S and NR⁸, wherein R⁸ is a member selected from H, OH, substituted or unsubstituted alkyl and substituted or unsubstituted heteroalkyl. In a particular example, R² is COOQ, wherein Q is H, a single negative charge or a salt counterion (cation).

In one embodiment, in Formula (V), Y is CH₂. In another embodiment, Y is CH(OH)CH₂. In yet another embodiment, Y is CH(OH)CH(OH)CH₂. In a further embodiment, Y is CH. In one embodiment Y is CH(OH)CH. In another embodiment Y is CH(OH)CH(OH)CH. In yet another embodiment, Y is CH(OH). In a further embodiment, Y is CH(OH)CH(OH). In one embodiment Y is CH(OH)CH(OH)CH(OH). Y² is a member selected from H, OR⁶, R⁶, substituted or unsubstituted alkyl, substituted or unsubstituted heteroalkyl,

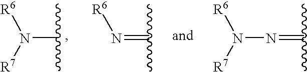

wherein R⁶ and R⁷ are members independently selected from H, substituted or unsubstituted alkyl, substituted or unsubstituted heteroalkyl and -L^{a}-R^{6b}. In one example, -L^{a}-R^{6b} includes C(O)R^{6b}, C(O)-L^{b}-R^{6b}, C(O)NH-L^{b}-R^{6b} or NHC(O)-L^{b}-R^{6b}. R^{6b} is a member selected from H, substituted or unsubstituted alkyl, substituted or unsubstituted heteroalkyl and a modifying group, such as a linear or branched polymeric modifying group of the invention.

In Formula (V), R³, R^{3′} and R⁴ are members independently selected from H, NHR^{3″}, OR^{3″}, SR^{3″}, substituted or unsubstituted alkyl, substituted or unsubstituted heteroalkyl and -L^{a}-R^{6c}. In one example, -L^{a}-R^{6c} includes —O-L^{b}-R^{6c}, —C(O)-L^{b}-R^{6c}, —C(O)NH-L^{b}-R^{6c}, —NH-L^{b}-R^{6c}, ═N-L^{b}-R^{6c}, —NHC(O)-L^{b}-R^{6c}, —NHC(O)NH-L^{b}-R^{6c} or —NHC(O)O-L^{b}-R^{6c}, wherein each R^{3″} is a member independently selected from H, substituted or unsubstituted alkyl and substituted or unsubstituted heteroalkyl. Each R^{6c} is a member independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, NR¹³R¹⁴ and a modifying group, wherein R¹³ and R¹⁴ are members independently selected from H, substituted or unsubstituted alkyl and substituted or unsubstituted heteroalkyl.

In the above embodiments, each L^{a} and each L^{b} is a member independently selected from a bond and a linker moiety selected from substituted or unsubstituted alkyl, substituted or unsubstituted heteroalkyl, substituted or unsubstituted aryl, substituted or unsubstituted heteroaryl, substituted and unsubstituted heterocycloalkyl.

In one embodiment, the moiety of Formula (V) has a structure according to Formula (VI):

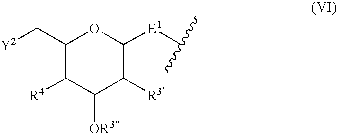

wherein E¹, R^{3′}, R^{3″} and R⁴ are defined as above. In one embodiment, in Formula (VI), E¹ is O. In another embodiment, E¹ is NH. In another embodiment, in Formula (VI), —OR^{3″} is OH. In yet another exemplary embodiment, R^{3′} is NHAc or OH.

In one embodiment, the moiety of Formula (VI) is directly bound to an amino acid residue of the polypeptide. In one example according to this embodiment, E¹ is a bond to that amino acid residue and the moiety of Formula (VI) has a structure, which is a member selected from:

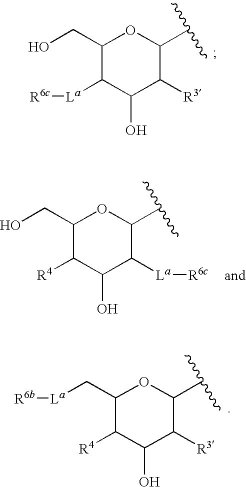

In another embodiment, the moiety of Formula (VI) is bound to the polypeptide through another sugar residue. In an exemplary embodiment, the moiety of Formula (VI) has the structure, which is selected from:

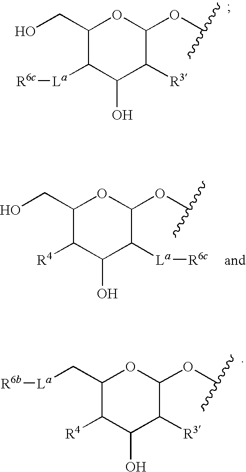

In one example, according to any of the above embodiments, R^{3′} and R⁴ are members independently selected from NHAc and OH.

In one example according to the above embodiments, the moiety of Formula (V) or (VI) is a GlcNAc moiety. In one example, the moiety has a structure selected from:

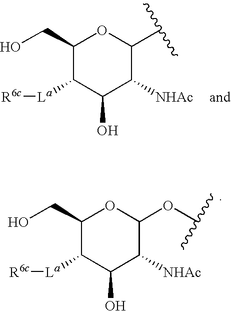

In another embodiment, the moiety of Formula (V) has a structure according to Formula (VII):

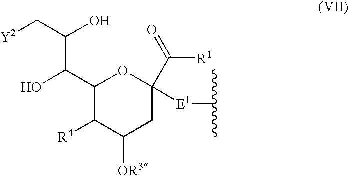

wherein Y², R¹, E¹, R^{3″} and R⁴ are defined as above. In one embodiment, in Formula (VII), E¹ is O. In another embodiment E¹ is NH. In another embodiment, E¹ is a bond to an amino acid residue of a polypeptide. In one embodiment, in Formula (VII), R¹ is OR⁹. In one example according to this embodiment, R⁹ is H, a negative charge or a salt counterion (cation). In another embodiment, in Formula (VII), R^{3″} is H.

In another embodiment, the moiety of Formula (VII) has the structure, which is a member selected from:

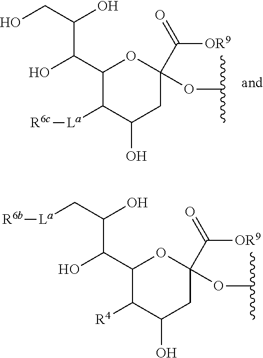

wherein R⁹ is H, a single negative charge or a salt counterion. In one example, R⁴ is a member selected from OH and NHAc.

In one example according to any of the above embodiments (e.g., in Formula V, VI or Formula VII), -L^{a}-R^{6c} includes a moiety, which is a member selected from:

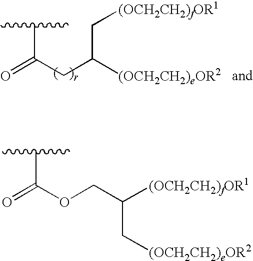

wherein r is an integer selected from 1 to 20 and f and e are integers independently selected from 1-5000. R¹ and R² are members independently selected from H and C₁-C₁₀ substituted or unsubstituted alkyl. In one example, R¹ and R² are members independently selected from H, methyl, ethyl, propyl, isopropyl, butyl and isobutyl. In one embodiment, R¹ and R² are each methyl.

In another example according to any of the above embodiments, -L^{a}-R^{6c} or -L^{a}-R^{6c} is:

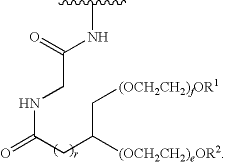

In another example according to the above embodiments (e.g., in Formula V, VI or Formula VII), -L^{a}-R^{6c} or -L^{a}-R^{6c} is:

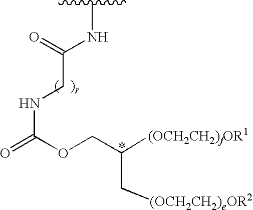

wherein r is an integer selected from 1 to 20 and f and e are integers independently selected from 1-5000. R¹ and R² are members independently selected from H, methyl, ethyl, propyl, isopropyl, butyl and isobutyl. In one embodiment, R¹ and R² are each methyl. The stereocenter indicated with “*” can be racemic or defined. In one embodiment, the stereocenter has (S) configuration. In another embodiment, the stereocenter has (R) configuration.

In yet another example according to any of the above embodiments (e.g., in Formula V, VI or Formula VII), -L^{a}-R^{6c} or -L^{a}-R^{6c} is:

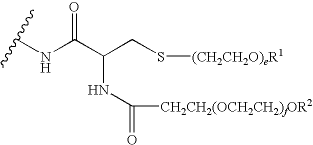

wherein e, f, R¹ and R² are defined as above.

In a further example according to any of the above embodiments (e.g., in Formula V, VI or Formula VII), -L^{a}-R^{6c} or -L^{a}-R^{6c} is:

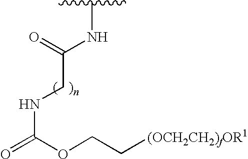

wherein e, f, R¹ and R² are defined as above.

In yet another embodiment, at least one of R^{6b} (e.g., in Formula V) o R^{6c} (e.g., in Formulae V to VII) is a member selected from:

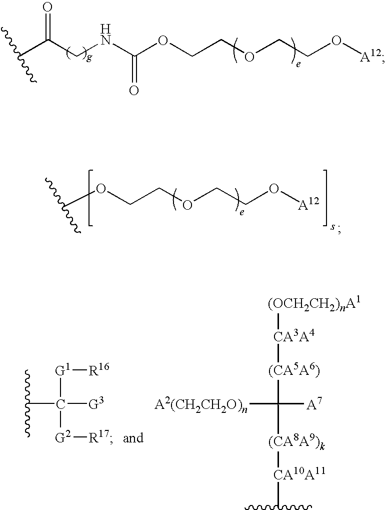

wherein g, j and k are integers independently selected from 0 to 20. Each e is an integer independently selected from 0 to 2500. The integer s is selected from 1-5. R¹⁶ and R¹⁷ are independently selected polymeric moieties. G¹ and G² are independently selected linkage fragments joining polymeric moieties R¹⁶ and R¹⁷ to C. An exemplary linkage fragment includes neither aromatic nor ester moieties. Alternatively, these linkage fragments can include one or more moiety that is designed to degrade under physiologically relevant conditions, e.g., esters, disulfides, etc.

Exemplary linkage fragments including G¹ and G² are independently selected and include S, SC(O)NH, HNC(O)S, SC(O)O, O, NH, NHC(O), (O)CNH and NHC(O)O, and OC(O)NH, CH₂S, CH₂O, CH₂CH₂O, CH₂CH₂S, (CH₂)ₒO, (CH₂)ₒS or (CH₂)ₒY′-PEG wherein, Y′ is S, NH, NHC(O), C(O)NH, NHC(O)O, OC(O)NH, or O and o is an integer from 1 to 50. In an exemplary embodiment, the linkage fragments G¹ and G² are different linkage fragments.

G³ is a member selected from H, substituted or unsubstituted alkyl, substituted or unsubstituted heteroalkyl, substituted or unsubstituted heterocycloalkyl, substituted or unsubstituted aryl, substituted or unsubstituted heteroaryl. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NA¹²A¹³, —OA¹² and —SiA¹²A¹³, wherein A¹² and A¹³ are members independently selected from H, substituted or unsubstituted alkyl, substituted or unsubstituted heteroalkyl, substituted or unsubstituted heterocycloalkyl, substituted or unsubstituted aryl, and substituted or unsubstituted heteroaryl.

### Modifying Group

The modifying group of the invention can be any chemical moiety. Exemplary modifying groups are discussed below. The modifying groups can be selected for their ability to alter the properties (e.g., biological or physicochemical properties) of a given polypeptide. Exemplary polypeptide properties that may be altered by the use of modifying groups include, but are not limited to, pharmacokinetics, pharmacodynamics, metabolic stability, biodistribution, water solubility, lipophilicity, tissue targeting capabilities and the therapeutic activity profile. Preferred modifying groups are those which improve pharmacodynamics and pharmacokinetics of a polypeptide conjugate of the invention that has been modified with such modifying group. Other modifying groups may be useful for the modification of polypeptides that find applications in in vitro biological assay systems including diagnostic products.

For example, the in vivo half-life of therapeutic glycopeptides can be enhanced with polyethylene glycol (PEG) moieties. Chemical modification of polypeptides with PEG (PEGylation) increases their molecular size and typically decreases surface- and functional group-accessibility, each of which are dependent on the number and size of the PEG moieties attached to the polypeptide. Frequently, this modification results in an improvement of plasma half-live and in proteolytic-stability, as well as a decrease in immunogenicity and hepatic uptake (Chaffee et al. J. Clin. Invest. 89: 1643-1651 (1992); Pyatak et al. Res. Commun. Chem. Pathol Pharmacol. 29: 113-127 (1980)). For example, PEGylation of interleukin-2 has been reported to increase its antitumor potency in vivo (Katre et al. Proc. Natl. Acad. Sci. USA. 84: 1487-1491 (1987)) and PEGylation of a F(ab′)2 derived from the monoclonal antibody A7 has improved its tumor localization (Kitamura et al. Biochem. Biophys. Res. Commun. 28: 1387-1394 (1990)). Thus, in another embodiment, the in vivo half-life of a polypeptide derivatized with a PEG moiety by a method of the invention is increased relative to the in vivo half-life of the non-derivatized parent polypeptide.

The increase in polypeptide in vivo half-life is best expressed as a range of percent increase relative to the parent polypeptide. The lower end of the range of percent increase is about 40%, about 60%, about 80%, about 100%, about 150% or about 200%. The upper end of the range is about 60%, about 80%, about 100%, about 150%, or more than about 250%.

### Water-soluble Polymeric Modifying Groups

In one embodiment, the modifying group is a polymeric modifying group selected from linear and branched. In one example, the modifying group includes one or more polymeric moiety, wherein each polymeric moiety is independently selected.

Many water-soluble polymers are known to those of skill in the art and are useful in practicing the present invention. The term water-soluble polymer encompasses species such as saccharides (e.g., dextran, amylose, hyalouronic acid, poly(sialic acid), heparans, heparins and the like); poly(amino acids), e.g., poly(aspartic acid) and poly(glutamic acid); nucleic acids; synthetic polymers (e.g., poly(acrylic acid), poly(ethers), such as poly(ethylene glycol); peptides, proteins, and the like. The present invention may be practiced with any water-soluble polymer with the sole limitation that the polymer must include a point at which the remainder of the conjugate can be attached.

The use of reactive derivatives of the modifying group (e.g., a reactive PEG analogs) to attach the modifying group to one or more polypeptide moiety is within the scope of the present invention. The invention is not limited by the identity of the reactive analog.

In a preferred embodiment, the modifying group is PEG or a PEG analog. Many activated derivatives of poly(ethylene glycol) are available commercially and are described in the literature. It is well within the abilities of one of skill to choose or, if necessary, synthesize an appropriate activated PEG derivative, with which to prepare a substrate useful in the present invention. See, Abuchowski et al. Cancer Biochem. Biophys., 7: 175-186 (1984); Abuchowski et al., J. Biol. Chem., 252: 3582-3586 (1977); Jackson et al., Anal. Biochem., 165: 114-127 (1987); Koide et al., Biochem Biophys. Res. Commun., 111: 659-667 (1983)), tresylate (Nilsson et al., Methods Enzymol., 104: 56-69 (1984); Delgado et al., Biotechnol. Appl. Biochem., 12: 119-128 (1990)); N-hydroxysuccinimide derived active esters (Buckmann et al., Makromol. Chem., 182: 1379-1384 (1981); Joppich et al., Makromol. Chem., 180: 1381-1384 (1979); Abuchowski et al., Cancer Biochem. Biophys., 7: 175-186 (1984); Katre et al. Proc. Natl. Acad. Sci. U.S.A., 84: 1487-1491 (1987); Kitamura et al., Cancer Res., 51: 4310-4315 (1991); Boccu et al., Z. Naturforsch., 38C: 94-99 (1983), carbonates (Zalipsky et al., POLY(ETHYLENE GLYCOL) CHEMISTRY: BIOTECHNICAL AND BIOMEDICAL APPLICATIONS, Harris, Ed., Plenum Press, New York, 1992, pp. 347-370; Zalipsky et al., Biotechnol. Appl. Biochem., 15: 100-114 (1992); Veronese et al., Appl. Biochem. Biotech., 11: 141-152 (1985)), imidazolyl formates (Beauchamp et al., Anal. Biochem., 131: 25-33 (1983); Berger et al., Blood, 71: 1641-1647 (1988)), 4-dithiopyridines (Woghiren et al., Bioconjugate Chem., 4: 314-318 (1993)), isocyanates (Byun et al., ASAIO Journal, M649-M-653 (1992)) and epoxides (U.S. Pat. No. 4,806,595, issued to Noishiki et al., (1989). Other linking groups include the urethane linkage between amino groups and activated PEG. See, Veronese, et al., Appl. Biochem. Biotechnol., 11: 141-152 (1985).

Methods for activation of polymers can be found in WO 94/17039, U.S. Pat. No. 5,324,844, WO 94/18247, WO 94/04193, U.S. Pat. No. 5,219,564, U.S. Pat. No. 5,122,614, WO 90/13540, U.S. Pat. No. 5,281,698, and more WO 93/15189, and for conjugation between activated polymers and peptides, e.g. Coagulation Factor VIII (WO 94/15625), hemoglobin (WO 94/09027), oxygen carrying molecule (U.S. Pat. No. 4,412,989), ribonuclease and superoxide dismutase (Veronese at al., App. Biochem. Biotech. 11:141-45 (1985)).

Activated PEG molecules useful in the present invention and methods of making those reagents are known in the art and are described, for example, in WO04/083259.

Activating, or leaving groups, appropriate for activating linear PEGs of use in preparing the compounds set forth herein include, but are not limited to the species:

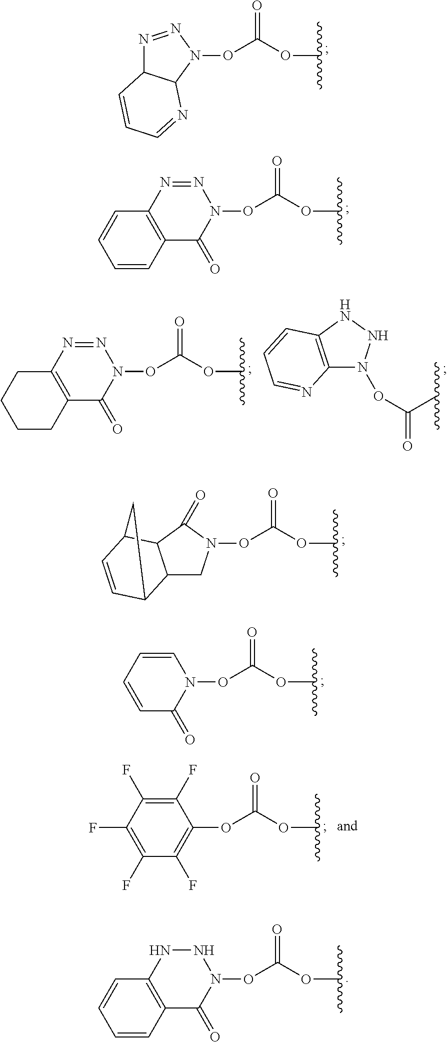

Exemplary water-soluble polymers are those in which a substantial proportion of the polymer molecules in a sample of the polymer are of approximately the same molecular weight; such polymers are “homodisperse.”

The present invention is further illustrated by reference to a poly(ethylene glycol) conjugate. Several reviews and monographs on the functionalization and conjugation of PEG are available. See, for example, Harris, Macronol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); Zalipsky, Bioconjugate Chem. 6: 150-165 (1995); and Bhadra, et al., Pharmazie, 57:5-29 (2002). Routes for preparing reactive PEG molecules and forming conjugates using the reactive molecules are known in the art. For example, U.S. Pat. No. 5,672,662 discloses a water soluble and isolatable conjugate of an active ester of a polymer acid selected from linear or branched poly(alkylene oxides), poly(oxyethylated polyols), poly(olefinic alcohols), and poly(acrylomorpholine).

U.S. Pat. No. 6,376,604 sets forth a method for preparing a water-soluble 1-benzotriazolylcarbonate ester of a water-soluble and non-peptidic polymer by reacting a terminal hydroxyl of the polymer with di(1-benzotriazoyl)carbonate in an organic solvent. The active ester is used to form conjugates with a biologically active agent such as a polypeptide.

WO 99/45964 describes a conjugate comprising a biologically active agent and an activated water soluble polymer comprising a polymer backbone having at least one terminus linked to the polymer backbone through a stable linkage, wherein at least one terminus comprises a branching moiety having proximal reactive groups linked to the branching moiety, in which the biologically active agent is linked to at least one of the proximal reactive groups. Other branched poly(ethylene glycols) are described in WO 96/21469, U.S. Pat. No. 5,932,462 describes a conjugate formed with a branched PEG molecule that includes a branched terminus that includes reactive functional groups. The free reactive groups are available to react with a biologically active species, such as a polypeptide, forming conjugates between the poly(ethylene glycol) and the biologically active species. U.S. Pat. No. 5,446,090 describes a bifunctional PEG linker and its use in forming conjugates having a peptide at each of the PEG linker termini.

Conjugates that include degradable PEG linkages are described in WO 99/34833; and WO 99/14259, as well as in U.S. Pat. No. 6,348,558. Such degradable linkages are applicable in the present invention.

The art-recognized methods of polymer activation set forth above are of use in the context of the present invention in the formation of the branched polymers set forth herein and also for the conjugation of these branched polymers to other species, e.g., sugars, sugar nucleotides and the like.

An exemplary water-soluble polymer is a poly(ethylene glycol), such as PEG or methoxy-PEG (m-PEG). The poly(ethylene glycol) used in the present invention is not restricted to any particular form or molecular weight range. For each independently selected poly(ethylene glycol) moiety, the molecular weight is preferably between about 500 Da and about 100 kDa. In one embodiment, the molecular weight of the PEG moiety is between about 2 and about 80 kDa. In another embodiment, the molecular weight of the PEG moiety is between about 2 and about 60 kDa, preferably from about 5 to about 40 kDa. In an exemplary embodiment, the PEG moiety has a molecular weight of about 1 kDa, about 2 kDa, about 5 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa or about 80 kDa.

Exemplary poly(ethylene glycol) molecules of use in the invention include, but are not limited to, those having the formula:

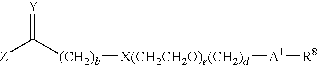

in which R⁸ is H, OH, NH₂, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—(CH₂)_{q}—, HS—(CH₂)_{q}, or —(CH₂)_{q}C(Y)Z¹. The index “e” represents an integer from 1 to 2500. The indices b, d, and q independently represent integers from 0 to 20. The symbols Z and Z¹ independently represent OH, NH₂, leaving groups, e.g., imidazole, p-nitrophenyl, HOBT, tetrazole, halide, S—R⁹, the alcohol portion of activated esters; —(CH₂)ₚC(Y¹)V, or —(CH₂)ₚU(CH₂)ₛC(Y¹)ᵥ. The symbol Y represents H(2), ═O, ═S, ═N—R¹⁰. The symbols X, Y, Y¹, A¹, and U independently represent the moieties O, S, N—R¹¹. The symbol V represents OH, NH₂, halogen, S—R¹², the alcohol component of activated esters, the amine component of activated amides, sugar-nucleotides, and proteins. The indices p, q, s and v are members independently selected from the integers from 0 to 20. The symbols R⁹, R¹⁰, R¹¹ and R¹² independently represent H, substituted or unsubstituted alkyl, substituted or unsubstituted heteroalkyl, substituted or unsubstituted aryl, substituted or unsubstituted heterocycloalkyl and substituted or unsubstituted heteroaryl.

The poly(ethylene glycol) useful in forming the conjugate of the invention is either linear or branched. Branched poly(ethylene glycol) molecules suitable for use in the invention include, but are not limited to, those described by the following formula:

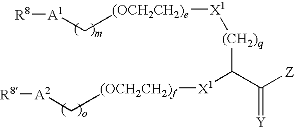

in which R⁸ and R^{8′} are members independently selected from the groups defined for R⁸, above. A¹ and A² are members independently selected from the groups defined for A¹, above. The indices e, f, o, and q are as described above. Z and Y are as described above. X¹ and X^{1′} are members independently selected from S, SC(O)NH, HNC(O)S, SC(O)O, O, NH, NHC(O), (O)CNH and NHC(O)O, OC(O)NH.

In other exemplary embodiments, the branched PEG is based upon a cysteine, serine or di-lysine core. In another exemplary embodiments, the poly(ethylene glycol) molecule is selected from the following structures:

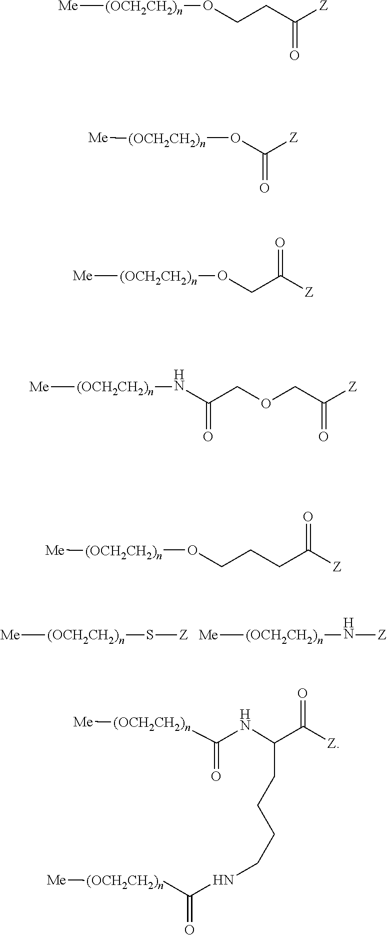

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660; WO 02/09766; Kodera Y., Bioconjugate Chemistry 5: 283-288 (1994); and Yamasaki et al., Agric. Biol. Chem., 52: 2125-2127, 1998. In a preferred embodiment the molecular weight of each poly(ethylene glycol) of the branched PEG is less than or equal to 40,000 daltons.

Representative polymeric modifying moieties include structures that are based on side chain-containing amino acids, e.g., serine, cysteine, lysine, and small peptides, e.g., lys-lys. Exemplary structures include:

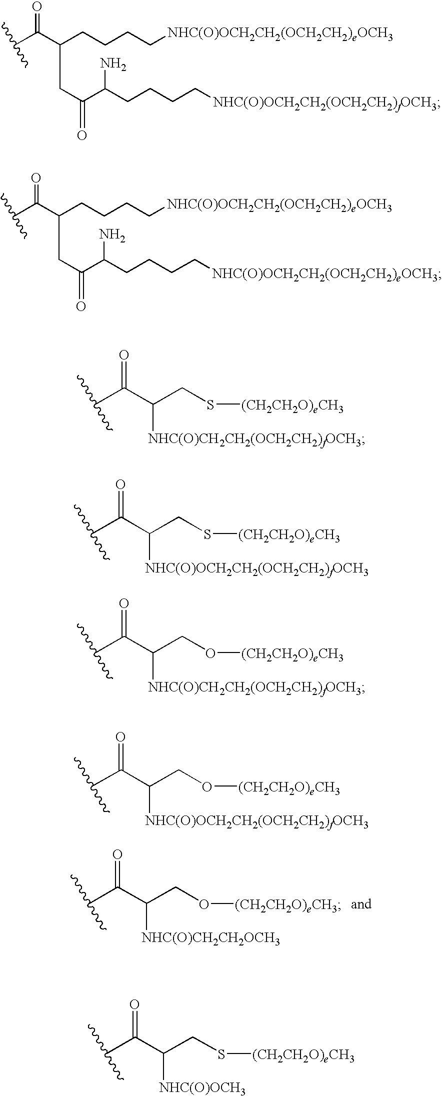

Those of skill will appreciate that the free amine in the di-lysine structures can also be pegylated through an amide or urethane bond with a PEG moiety.

In yet another embodiment, the polymeric modifying moiety is a branched PEG moiety that is based upon a tri-lysine peptide. The tri-lysine can be mono-, di-, tri-, or tetra-PEG-ylated. Exemplary species according to this embodiment have the formulae:

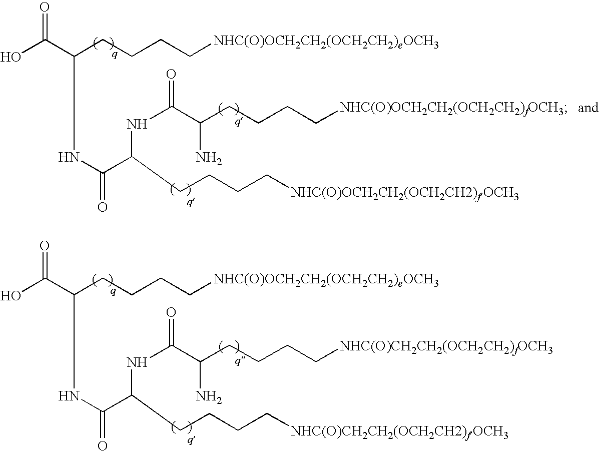

in which the indices e, f and f′ are independently selected integers from 1 to 2500; and the indices q, q′ and q″ are independently selected integers from 1 to 20.

As will be apparent to those of skill, the branched polymers of use in the invention include variations on the themes set forth above. For example the di-lysine-PEG conjugate shown above can include three polymeric subunits, the third bonded to the α-amine shown as unmodified in the structure above. Similarly, the use of a tri-lysine functionalized with three or four polymeric subunits labeled with the polymeric modifying moiety in a desired manner is within the scope of the invention.

An exemplary precursor useful to form a polypeptide conjugate with a branched modifying group that includes one or more polymeric moiety (e.g., PEG) has the formula:

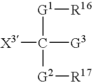

In one embodiment, the branched polymer species according to this formula are essentially pure water-soluble polymers. X^{3′} is a moiety that includes an ionizable (e.g., OH, COOH, H₂PO₄, HSO₃, NH₂, and salts thereof, etc.) or other reactive functional group, e.g., infra. C is carbon. G³ is a non-reactive group (e.g., H, CH₃, OH and the like). In one embodiment, G³ is preferably not a polymeric moiety. R¹⁶ and R¹⁷ are independently selected from non-reactive groups (e.g., H, unsubstituted alkyl, unsubstituted heteroalkyl) and polymeric arms (e.g., PEG). G¹ and G² are linkage fragments that are preferably essentially non-reactive under physiological conditions. G¹ and G² are independently selected. An exemplary linker includes neither aromatic nor ester moieties. Alternatively, these linkages can include one or more moiety that is designed to degrade under physiologically relevant conditions, e.g., esters, disulfides, etc G¹ and G² join the polymeric arms R¹⁶ and R¹⁷ to C. In one embodiment, when X^{3′} is reacted with a reactive functional group of complementary reactivity on a linker, sugar or linker-sugar cassette, X^{3′} is converted to a component of a linkage fragment.

Exemplary linkage fragments including G¹ and G² are independently selected and include S, SC(O)NH, HNC(O)S, SC(O)O, O, NH, NHC(O), (O)CNH and NHC(O)O, and OC(O)NH, CH₂S, CH₂O, CH₂CH₂O, CH₂CH₂S, (CH₂)ₒO, (CH₂)ₒS or (CH₂)ₒY′-PEG wherein, Y′ is S, NH, NHC(O), C(O)NH, NHC(O)O, OC(O)NH, or O and o is an integer from 1 to 50. In an exemplary embodiment, the linkage fragments G¹ and G² are different linkage fragments.

In an exemplary embodiment, one of the above precursors or an activated derivative thereof, is reacted with, and thereby bound to a sugar, an activated sugar or a sugar nucleotide through a reaction between X^{3′} and a group of complementary reactivity on the sugar moiety, e.g., an amine. Alternatively, X^{3′} reacts with a reactive functional group on a precursor to linker L^{a} according to Scheme 2, below.

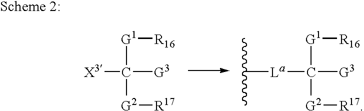

In an exemplary embodiment, the modifying group is derived from a natural or unnatural amino acid, amino acid analogue or amino acid mimetic, or a small peptide formed from one or more such species. For example, certain branched polymers found in the compounds of the invention have the formula:

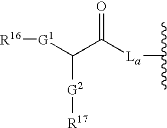

In this example, the linkage fragment C(O)L^{a} is formed by the reaction of a reactive functional group, e.g., X^{3′}, on a precursor of the branched polymeric modifying moiety and a reactive functional group on the sugar moiety, or a precursor to a linker. For example, when X^{3′} is a carboxylic acid, it can be activated and bound directly to an amine group pendent from an amino-saccharide (e.g., Sia, GalNH₂, GlcNH₂, ManNH₂, etc.), forming an amide. Additional exemplary reactive functional groups and activated precursors are described hereinbelow. The symbols have the same identity as those discussed above.

In another exemplary embodiment, L^{a} is a linking moiety having the structure:

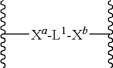

in which X^{a} and X^{b} are independently selected linkage fragments and L¹ is selected from a bond, substituted or unsubstituted alkyl or substituted or unsubstituted heteroalkyl.

Exemplary species for X^{a} and X^{b} include S, SC(O)NH, HNC(O)S, SC(O)O, O, NH, NHC(O), C(O)NH and NHC(O)O, and OC(O)NH.

In another exemplary embodiment, G² is a peptide bond to R¹⁷, which is an amino acid, di-peptide (e.g., Lys-Lys) or tri-peptide (e.g., Lys-Lys-Lys) in which the alpha-amine moiety(ies) and/or side chain heteroatom(s) are modified with a polymeric modifying moiety.

The embodiments of the invention set forth above are further exemplified by reference to species in which the polymer is a water-soluble polymer, particularly poly(ethylene glycol) (“PEG”), e.g., methoxy-poly(ethylene glycol). Those of skill will appreciate that the focus in the sections that follow is for clarity of illustration and the various motifs set forth using PEG as an exemplary polymer are equally applicable to species in which a polymer other than PEG is utilized.

In other exemplary embodiments, the polypeptide conjugate includes a moiety selected from the group:

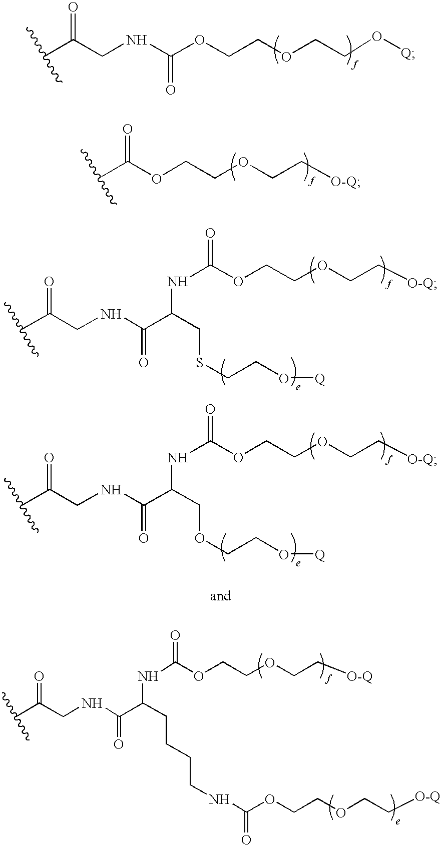

In each of the formulae above, the indices e and f are independently selected from the integers from 1 to 2500. In further exemplary embodiments, e and f are selected to provide a PEG moiety that is about 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa. The symbol Q represents substituted or unsubstituted alkyl (e.g., C₁-C₆ alkyl, e.g., methyl), substituted or unsubstituted heteroalkyl or H.

Other branched polymers have structures based on di-lysine (Lys-Lys) peptides, e.g.:

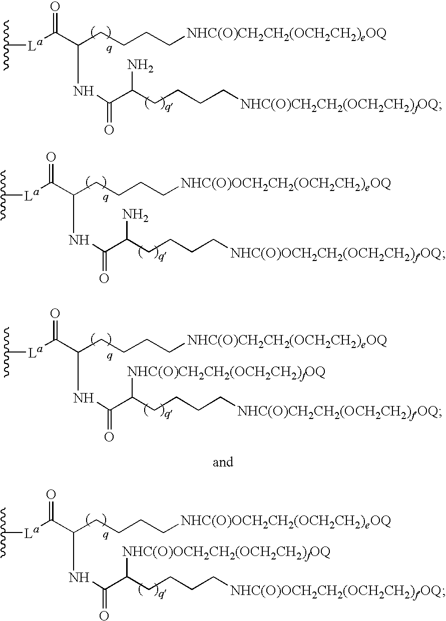

and tri-lysine peptides (Lys-Lys-Lys), e.g.:

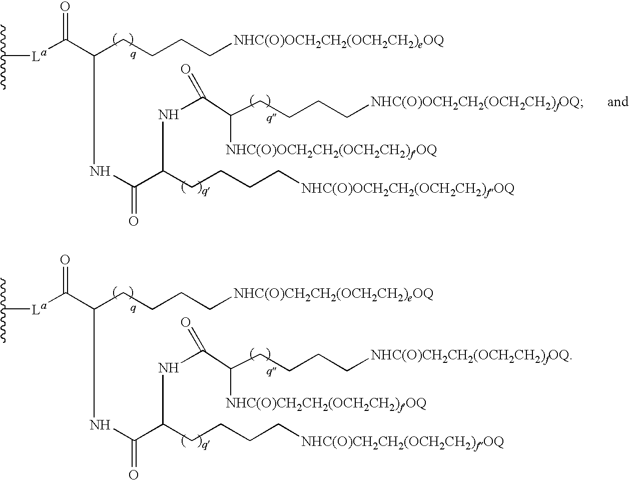

In each of the figures above, the indices e, f, f′ and f″ represent integers independently selected from 1 to 2500. The indices q, q′ and q″ represent integers independently selected from 1 to 20.

In another exemplary embodiment, the conjugates of the invention include a formula which is a member selected from:

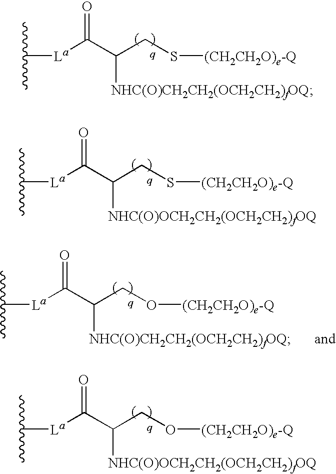

wherein Q is a member selected from H and substituted or unsubstituted C₁-C₆ alkyl. The indices e and f are integers independently selected from 1 to 2500, and the index q is an integer selected from 0 to 20.

In another exemplary embodiment, the conjugates of the invention include a formula which is a member selected from:

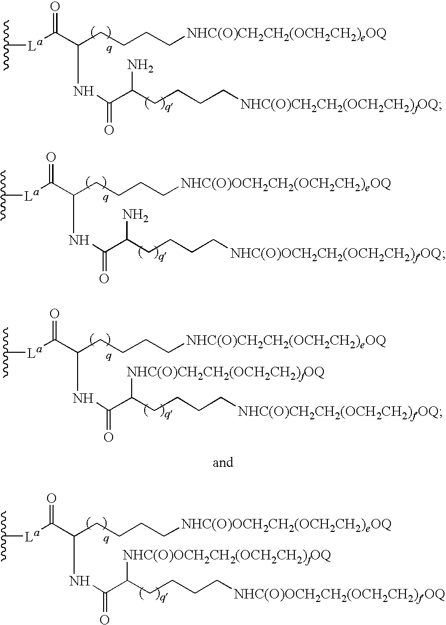

wherein Q is a member selected from H and substituted or unsubstituted C₁-C₆ alkyl, preferably Me. The indices e, f and f′ are integers independently selected from 1 to 2500, and q and q′ are integers independently selected from 1 to 20.

In another exemplary embodiment, the conjugate of the invention includes a structure according to the following formula:

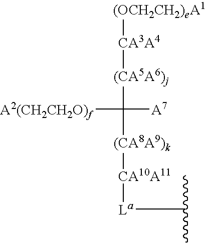

wherein the indices e and f are independently selected from 0 to 2500. The indices j and k are integers independently selected from 0 to 20. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted aryl, substituted or unsubstituted cycloalkyl, substituted or unsubstituted heterocycloalkyl, substituted or unsubstituted heteroaryl, —NA¹²A¹³, —OA¹² and —SiA¹²A¹³. A¹² and A¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In one embodiment according to the formula above, the branched polymer has a structure according to the following formula:

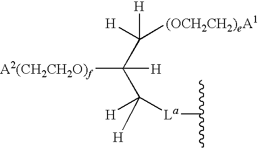

In an exemplary embodiment, A¹ and A² are members independently selected from OCH₃ and OH.

In another exemplary embodiment, the linker L^{a} is a member selected from aminoglycine derivatives. Exemplary polymeric modifying groups according to this embodiment have a structure according to the following formulae:

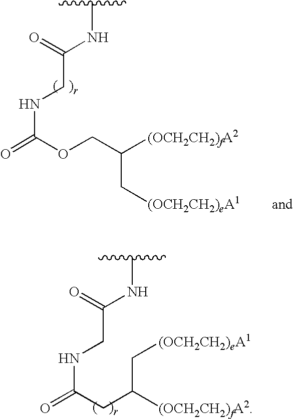

In one example, A¹ and A² are members independently selected from OCH₃ and OH. Exemplary polymeric modifying groups according to this example include:

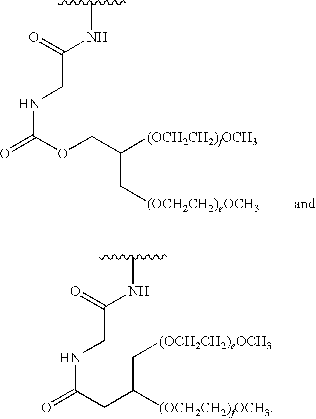

In each of the above embodiment, wherein the modifying group includes a stereocenter, for example those including an amino acid linker or a glycerol-based linker, the stereocenter can be either racemic or defined. In one embodiment, in which such stereocenter is defined, it has (S) configuration. In another embodiment, the stereocenter has (R) configuration.

Those of skill in the art will appreciate that one or more of the m-PEG arms of the branched polymer can be replaced by a PEG moiety with a different terminus, e.g., OH, COOH, NH₂, C₂-C₁₀-alkyl, etc. Moreover, the structures above are readily modified by inserting alkyl linkers (or removing carbon atoms) between the α-carbon atom and the functional group of the side chain. Thus, “homo” derivatives and higher homologues, as well as lower homologues are within the scope of cores for branched PEGs of use in the present invention.

The branched PEG species set forth herein are readily prepared by methods such as that set forth in Scheme 3, below:

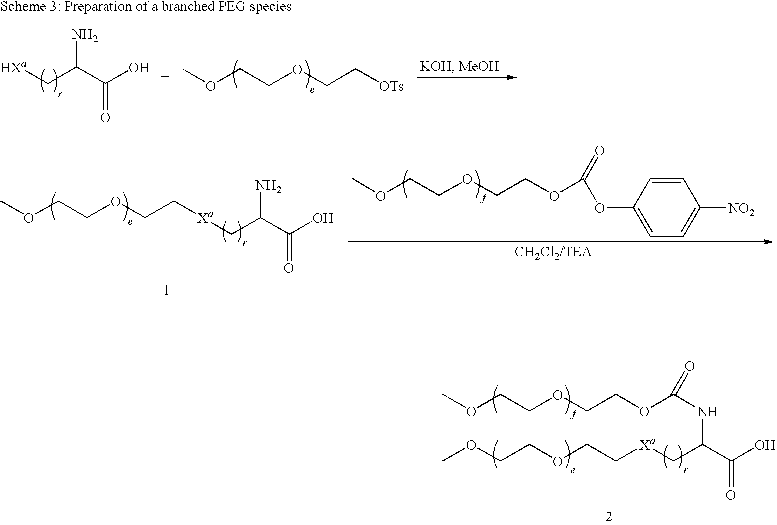

in which X^{a} is O or S and r is an integer from 1 to 5. The indices e and f are independently selected integers from 1 to 2500.

Thus, according to Scheme 3, a natural or unnatural amino acid is contacted with an activated m-PEG derivative, in this case the tosylate, forming 1 by alkylating the side-chain heteroatom X^{a}. The mono-functionalized m-PEG amino acid is submitted to N-acylation conditions with a reactive m-PEG derivative, thereby assembling branched m-PEG 2. As one of skill will appreciate, the tosylate leaving group can be replaced with any suitable leaving group, e.g., halogen, mesylate, triflate, etc. Similarly, the reactive carbonate utilized to acylate the amine can be replaced with an active ester, e.g., N-hydroxysuccinimide, etc., or the acid can be activated in situ using a dehydrating agent such as dicyclohexylcarbodiimide, carbonyldiimidazole, etc.

In an exemplary embodiment, the modifying group is a PEG moiety, however, any modifying group, e.g., water-soluble polymer, water-insoluble polymer, therapeutic moiety, etc., can be incorporated in a glycosyl moiety through an appropriate linkage. The modified sugar is formed by enzymatic means, chemical means or a combination thereof, thereby producing a modified sugar. In an exemplary embodiment, the sugars are substituted with an active amine at any position that allows for the attachment of the modifying moiety, yet still allows the sugar to function as a substrate for an enzyme capable of coupling the modified sugar to the G-CSF polypeptide. In an exemplary embodiment, when galactosamine is the modified sugar, the amine moiety is attached to the carbon atom at the 6-position.

### Water-Insoluble Polymers

In another embodiment, analogous to those discussed above, the modified sugars include a water-insoluble polymer, rather than a water-soluble polymer. The conjugates of the invention may also include one or more water-insoluble polymers. This embodiment of the invention is illustrated by the use of the conjugate as a vehicle with which to deliver a therapeutic polypeptide in a controlled manner. Polymeric drug delivery systems are known in the art. See, for example, Dunn et al., Eds. POLYMERIC DRUGS AND DRUG DELIVERY SYSTEMS, ACS Symposium Series Vol. 469, American Chemical Society, Washington, D.C. 1991. Those of skill in the art will appreciate that substantially any known drug delivery system is applicable to the conjugates of the present invention.

Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Synthetically modified natural polymers of use in conjugates of the invention include, but are not limited to, alkyl celluloses, hydroxyalkyl celluloses, cellulose ethers, cellulose esters, and nitrocelluloses. Particularly preferred members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

These and the other polymers discussed herein can be readily obtained from commercial sources such as Sigma Chemical Co. (St. Louis, Mo.), Polysciences (Warrenton, Pa.), Aldrich (Milwaukee, Wis.), Fluka (Ronkonkoma, N.Y.), and BioRad (Richmond, Calif.), or else synthesized from monomers obtained from these suppliers using standard techniques.

Representative biodegradable polymers of use in the conjugates of the invention include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

The polymers of use in the invention include “hybrid' polymers that include water-insoluble materials having within at least a portion of their structure, a bioresorbable molecule. An example of such a polymer is one that includes a water-insoluble copolymer, which has a bioresorbable region, a hydrophilic region and a plurality of crosslinkable functional groups per polymer chain.

For purposes of the present invention, “water-insoluble materials” includes materials that are substantially insoluble in water or water-containing environments. Thus, although certain regions or segments of the copolymer may be hydrophilic or even water-soluble, the polymer molecule, as a whole, does not to any substantial measure dissolve in water.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region may be either hydrophobic or hydrophilic, so long as the copolymer composition as a whole is not rendered water-soluble. Thus, the bioresorbable region is selected based on the preference that the polymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable polymers include, for example, synthetically produced resorbable block copolymers of poly(α-hydroxy-carboxylic acid)/poly(oxyalkylene, (see, Cohn et al., U.S. Pat. No. 4,826,945). These copolymers are not crosslinked and are water-soluble so that the body can excrete the degraded block copolymer compositions. See, Younes et al., J Biomed. Mater. Res. 21: 1301-1316 (1987); and Cohn et al., J Biomed. Mater. Res. 22: 993-1009 (1988).

Presently preferred bioresorbable polymers include one or more components selected from poly(esters), poly(hydroxy acids), poly(lactones), poly(amides), poly(ester-amides), poly(amino acids), poly(anhydrides), poly(orthoesters), poly(carbonates), poly(phosphazines), poly(phosphoesters), poly(thioesters), polysaccharides and mixtures thereof. More preferably still, the bioresorbable polymer includes a poly(hydroxy) acid component. Of the poly(hydroxy) acids, polylactic acid, polyglycolic acid, polycaproic acid, polybutyric acid, polyvaleric acid and copolymers and mixtures thereof are preferred.

In addition to forming fragments that are absorbed in vivo (“bioresorbed”), preferred polymeric coatings for use in the methods of the invention can also form an excretable and/or metabolizable fragment.

Higher order copolymers can also be used in the present invention. For example, Casey et al., U.S. Pat. No. 4,438,253, which issued on Mar. 20, 1984, discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other polymers based on lactic and/or glycolic acids can also be utilized. For example, Spinu, U.S. Pat. No. 5,202,413, which issued on Apr. 13, 1993, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a di-functional compound, such as, a diisocyanate, diacylchloride or dichlorosilane.

Bioresorbable regions of coatings useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene) oxide. Such poly(alkylene) oxides can include, for example, poly(ethylene) oxide, poly(propylene) oxide and mixtures and copolymers thereof.

Polymers that are components of hydrogels are also useful in the present invention. Hydrogels are polymeric materials that are capable of absorbing relatively large quantities of water. Examples of hydrogel forming compounds include, but are not limited to, polyacrylic acids, sodium carboxymethylcellulose, polyvinyl alcohol, polyvinyl pyrrolidine, gelatin, carrageenan and other polysaccharides, hydroxyethylenemethacrylic acid (HEMA), as well as derivatives thereof, and the like. Hydrogels can be produced that are stable, biodegradable and bioresorbable. Moreover, hydrogel compositions can include subunits that exhibit one or more of these properties.

Bio-compatible hydrogel compositions whose integrity can be controlled through crosslinking are known and are presently preferred for use in the methods of the invention. For example, Hubbell et al., U.S. Pat. Nos. 5,410,016, which issued on Apr. 25, 1995 and 5,529,914, which issued on Jun. 25, 1996, disclose water-soluble systems, which are crosslinked block copolymers having a water-soluble central block segment sandwiched between two hydrolytically labile extensions. Such copolymers are further end-capped with photopolymerizable acrylate functionalities. When crosslinked, these systems become hydrogels. The water soluble central block of such copolymers can include poly(ethylene glycol); whereas, the hydrolytically labile extensions can be a poly(α-hydroxy acid), such as polyglycolic acid or polylactic acid. See, Sawhney et al., Macromolecules 26: 581-587 (1993).

In another embodiment, the gel is a thermoreversible gel. Thermoreversible gels including components, such as pluronics, collagen, gelatin, hyalouronic acid, polysaccharides, polyurethane hydrogel, polyurethane-urea hydrogel and combinations thereof are presently preferred.

In yet another exemplary embodiment, the conjugate of the invention includes a component of a liposome. Liposomes can be prepared according to methods known to those skilled in the art, for example, as described in Eppstein et al., U.S. Pat. No. 4,522,811, which issued on Jun. 11, 1985. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its pharmaceutically acceptable salt is then introduced into the container. The container is then swirled by hand to free lipid material from the sides of the container and to disperse lipid aggregates, thereby forming the liposomal suspension.

The above-recited microparticles and methods of preparing the microparticles are offered by way of example and they are not intended to define the scope of microparticles of use in the present invention. It will be apparent to those of skill in the art that an array of microparticles, fabricated by different methods, are of use in the present invention.

The structural formats discussed above in the context of the water-soluble polymers, both straight-chain and branched are generally applicable with respect to the water-insoluble polymers as well. Thus, for example, the cysteine, serine, dilysine, and trilysine branching cores can be functionalized with two water-insoluble polymer moieties. The methods used to produce these species are generally closely analogous to those used to produce the water-soluble polymers.

### Other Modifying Groups

The present invention also provides conjugates analogous to those described above in which the polypeptide is conjugated to a therapeutic moiety, diagnostic moiety, targeting moiety, toxin moiety or the like via a glycosyl linking group. Each of the above-recited moieties can be a small molecule, natural polymer (e.g., polypeptide) or a synthetic polymer.

In a still further embodiment, the invention provides conjugates that localize selectively in a particular tissue due to the presence of a targeting agent as a component of the conjugate. In an exemplary embodiment, the targeting agent is a protein. Exemplary proteins include transferrin (brain, blood pool), HS-glycoprotein (bone, brain, blood pool), antibodies (brain, tissue with antibody-specific antigen, blood pool), coagulation factors V-XII (damaged tissue, clots, cancer, blood pool), serum proteins, e.g., α-acid glycoprotein, fetuin, α-fetal protein (brain, blood pool), β2-glycoprotein (liver, atherosclerosis plaques, brain, blood pool), G-CSF, GM-CSF, M-CSF, and EPO (immune stimulation, cancers, blood pool, red blood cell overproduction, neuroprotection), albumin (increase in half-life), IL-2 and IFN-α.

In an exemplary targeted conjugate, interferon alpha 2β (IFN-α2β) is conjugated to transferrin via a bifunctional linker that includes a glycosyl linking group at each terminus of the PEG moiety (Scheme 1). For example, one terminus of the PEG linker is functionalized with an intact sialic acid linker that is attached to transferrin and the other is functionalized with an intact C-linked Man linker that is attached to IFN-α2β.

### Biomolecules

In another embodiment, the modified sugar bears a biomolecule. In still further embodiments, the biomolecule is a functional protein, enzyme, antigen, antibody, peptide, nucleic acid (e.g., single nucleotides or nucleosides, oligonucleotides, polynucleotides and single- and higher-stranded nucleic acids), lectin, receptor or a combination thereof.

Preferred biomolecules are essentially non-fluorescent, or emit such a minimal amount of fluorescence that they are inappropriate for use as a fluorescent marker in an assay. Moreover, it is generally preferred to use biomolecules that are not sugars. An exception to this preference is the use of an otherwise naturally occurring sugar that is modified by covalent attachment of another entity (e.g., PEG, biomolecule, therapeutic moiety, diagnostic moiety, etc.). In an exemplary embodiment, a sugar moiety, which is a biomolecule, is conjugated to a linker arm and the sugar-linker arm cassette is subsequently conjugated to a polypeptide via a method of the invention.

Biomolecules useful in practicing the present invention can be derived from any source. The biomolecules can be isolated from natural sources or they can be produced by synthetic methods. Polypeptides can be natural polypeptides or mutated polypeptides. Mutations can be effected by chemical mutagenesis, site-directed mutagenesis or other means of inducing mutations known to those of skill in the art. polypeptides useful in practicing the instant invention include, for example, enzymes, antigens, antibodies and receptors. Antibodies can be either polyclonal or monoclonal; either intact or fragments. The polypeptides are optionally the products of a program of directed evolution

Both naturally derived and synthetic polypeptides and nucleic acids are of use in conjunction with the present invention; these molecules can be attached to a sugar residue component or a crosslinking agent by any available reactive group. For example, polypeptides can be attached through a reactive amine, carboxyl, sulfhydryl, or hydroxyl group. The reactive group can reside at a polypeptide terminus or at a site internal to the polypeptide chain. Nucleic acids can be attached through a reactive group on a base (e.g., exocyclic amine) or an available hydroxyl group on a sugar moiety (e.g., 3′- or 5′-hydroxyl). The peptide and nucleic acid chains can be further derivatized at one or more sites to allow for the attachment of appropriate reactive groups onto the chain. See, Chrisey et al. Nucleic Acids Res. 24: 3031-3039 (1996).

In a further embodiment, the biomolecule is selected to direct the polypeptide modified by the methods of the invention to a specific tissue, thereby enhancing the delivery of the polypeptide to that tissue relative to the amount of underivatized polypeptide that is delivered to the tissue. In a still further embodiment, the amount of derivatized polypeptide delivered to a specific tissue within a selected time period is enhanced by derivatization by at least about 20%, more preferably, at least about 40%, and more preferably still, at least about 100%. Presently, preferred biomolecules for targeting applications include antibodies, hormones and ligands for cell-surface receptors.

In still a further exemplary embodiment, there is provided as conjugate with biotin. Thus, for example, a selectively biotinylated polypeptide is elaborated by the attachment of an avidin or streptavidin moiety bearing one or more modifying groups.

### Therapeutic Moieties

In another embodiment, the modified sugar includes a therapeutic moiety. Those of skill in the art will appreciate that there is overlap between the category of therapeutic moieties and biomolecules; many biomolecules have therapeutic properties or potential.

The therapeutic moieties can be agents already accepted for clinical use or they can be drugs whose use is experimental, or whose activity or mechanism of action is under investigation. The therapeutic moieties can have a proven action in a given disease state or can be only hypothesized to show desirable action in a given disease state. In another embodiment, the therapeutic moieties are compounds, which are being screened for their ability to interact with a tissue of choice. Therapeutic moieties, which are useful in practicing the instant invention include drugs from a broad range of drug classes having a variety of pharmacological activities. Preferred therapeutic moieties are essentially non-fluorescent, or emit such a minimal amount of fluorescence that they are inappropriate for use as a fluorescent marker in an assay. Moreover, it is generally preferred to use therapeutic moieties that are not sugars. An exception to this preference is the use of a sugar that is modified by covalent attachment of another entity, such as a PEG, biomolecule, therapeutic moiety, diagnostic moiety and the like. In another exemplary embodiment, a therapeutic sugar moiety is conjugated to a linker arm and the sugar-linker arm cassette is subsequently conjugated to a polypeptide via a method of the invention.

Methods of conjugating therapeutic and diagnostic agents to various other species are well known to those of skill in the art. See, for example Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991.

In an exemplary embodiment, the therapeutic moiety is attached to the modified sugar via a linkage that is cleaved under selected conditions. Exemplary conditions include, but are not limited to, a selected pH (e.g., stomach, intestine, endocytotic vacuole), the presence of an active enzyme (e.g, esterase, reductase, oxidase), light, heat and the like. Many cleavable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989).

Classes of useful therapeutic moieties include, for example, non-steroidal anti-inflammatory drugs (NSAIDS). The NSAIDS can, for example, be selected from the following categories: (e.g., propionic acid derivatives, acetic acid derivatives, fenamic acid derivatives, biphenylcarboxylic acid derivatives and oxicams); steroidal anti-inflammatory drugs including hydrocortisone and the like; antihistaminic drugs (e.g., chlorpheniramine, triprolidine); antitussive drugs (e.g., dextromethorphan, codeine, caramiphen and carbetapentane); antipruritic drugs (e.g., methdilazine and trimeprazine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyramide, quinidine, encamide); β-adrenergic blocker drugs (e.g., metoprolol, acebutolol, betaxolol, labetalol and timolol); cardiotonic drugs (e.g., milrinone, amrinone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltiazem, amiodarone, isoxsuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chloroprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazepam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs).

Antimicrobial drugs which are preferred for incorporation into the present composition include, for example, pharmaceutically acceptable salts of β-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isothionate, metronidazole, pentamidine, gentamycin, kanamycin, lineomycin, methacycline, methenamine, minocycline, neomycin, netilmycin, paromomycin, streptomycin, tobramycin, miconazole and amantadine.

Other drug moieties of use in practicing the present invention include antineoplastic drugs (e.g., antiandrogens (e.g., leuprolide or flutamide), cytocidal agents (e.g., adriamycin, doxorubicin, taxol, cyclophosphamide, busulfan, cisplatin, β-2-interferon) anti-estrogens (e.g., tamoxifen), antimetabolites (e.g., fluorouracil, methotrexate, mercaptopurine, thioguanine). Also included within this class are radioisotope-based agents for both diagnosis and therapy, and conjugated toxins, such as ricin, geldanamycin, mytansin, CC-1065, the duocarmycins, Chlicheamycin and related structures and analogues thereof.

The therapeutic moiety can also be a hormone (e.g., medroxyprogesterone, estradiol, leuprolide, megestrol, octreotide or somatostatin); muscle relaxant drugs (e.g., cinnamedrine, cyclobenzaprine, flavoxate, orphenadrine, papaverine, mebeverine, idaverine, ritodrine, diphenoxylate, dantrolene and azumolen); antispasmodic drugs; bone-active drugs (e.g., diphosphonate and phosphonoalkylphosphinate drug compounds); endocrine modulating drugs (e.g., contraceptives (e.g., ethinodiol, ethinyl estradiol, norethindrone, mestranol, desogestrel, medroxyprogesterone), modulators of diabetes (e.g., glyburide or chlorpropamide), anabolics, such as testolactone or stanozolol, androgens (e.g., methyltestosterone, testosterone or fluoxymesterone), antidiuretics (e.g., desmopressin) and calcitonins).

Also of use in the present invention are estrogens (e.g., diethylstilbesterol), glucocorticoids (e.g., triamcinolone, betamethasone, etc.) and progestogens, such as norethindrone, ethynodiol, norethindrone, levonorgestrel; thyroid agents (e.g., liothyronine or levothyroxine) or anti-thyroid agents (e.g., methimazole); antihyperprolactinemic drugs (e.g., cabergoline); hormone suppressors (e.g., danazol or goserelin), oxytocics (e.g., methylergonovine or oxytocin) and prostaglandins, such as mioprostol, alprostadil or dinoprostone, can also be employed.

Other useful modifying groups include immunomodulating drugs (e.g., antihistamines, mast cell stabilizers, such as lodoxamide and/or cromolyn, steroids (e.g., triamcinolone, beclomethazone, cortisone, dexamethasone, prednisolone, methylprednisolone, beclomethasone, or clobetasol), histamine H2 antagonists (e.g., famotidine, cimetidine, ranitidine), immunosuppressants (e.g., azathioprine, cyclosporin), etc. Groups with anti-inflammatory activity, such as sulindac, etodolac, ketoprofen and ketorolac, are also of use. Other drugs of use in conjunction with the present invention will be apparent to those of skill in the art.

### Glycosyl Donor Species

In one embodiment, the polyeptide conjugates of the invention are prepared by contacting the polypeptide with a glycosyl donor species in the presence of an enzyme, for which the glycosyl donor species is a substrate. In one example, the glycosyl donor species has a structure according to Formula (X):

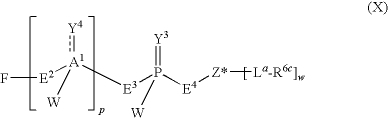

In Formula (X), p is an integer selected from 0 and 1; and w is an integer selected from 0 to 20. In one example, w is selected from 1-8. In another example, w is selected from 1 to 6. In another example, w is selected from 1 to 4. In yet another example, in which w is 0, -L^{a}-R^{6c} is replaced with H. F is a lipid moiety. Exemplary lipid moieties are described herein, below. In one example, the lipid moiety is a dolichol or an undecaprenyl moiety.

In Formula (X), Z* represents a glycosyl moiety of the invention. Glycosyl moieties are defined herein, e.g., in the context of polypetide conjugates (e.g., for Formula III) and equally apply to the glycosyl donor species of the invention. In a representative embodiment, the glycosyl moiety is selected from mono- and oligosaccharides. In another representative embodiment, Z* is selected from mono-antennary, di-antennary, tri-antennary and tetra-antennary saccharides. In another embodiment, Z* includes a C-2-N-acetamido group as in GlcNAc, GalNAc or bacillosamine.

In Formula (X), each L^{a} is a linker moiety independently selected from a single bond, a functional group, substituted or unsubstituted alkyl, substituted or unsubstituted heteroalkyl, substituted or unsubstituted aryl, substituted or unsubstituted heteroaryl and substituted or unsubstituted heterocycloalkyl. Each R^{6c} is an independently selected modifying group of the invention. A¹ is a member selected from P (phosphorus) and C (carbon). Y³ is a member selected from oxygen (O) and sulfur (S). Y⁴ is a member selected from O, S, SR¹, OR¹, OQ, CR¹R² and NR³R⁴. E², E³ and E⁴ are members independently selected from CR¹R², O, S and NR³. In one example, E² is O. In another example, E³ is O. In yet another example, E⁴ is O. In a particular example, each of E², E³ and E⁴ is O. Each W is a member independently selected from SR¹, OR¹, OQ, NR³R⁴, substituted or unsubstituted alkyl, substituted or unsubstituted heteroalkyl, substituted or unsubstituted aryl, substituted or unsubstituted heteroaryl and substituted or unsubstituted heterocycloalkyl. In Formula (X), each Q is a member independently selected from H, a negative charge and a salt counterion (cation) and each R¹, each R², each R³ and each R⁴ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In one example, the enzyme is an oligosaccharyltransferase and the glycosyl donor species is a lipid-pyrophosphate-linked glycosyl moiety.

### Lipid Moiety of the Glycosyl Donor

In one embodiment, the lipid moiety of Formula (X) includes from 1 to about 200 carbon atoms, preferably from about 5 to about 100 carbon atoms, arranged in a straight or branched chain. The carbon-carbon bonds in this chain are independently selected from saturated and unsaturated. Double-bonds can have cis- or trans-configuration. In one embodiment, the carbon chain includes at least one aromatic or non-aromatic ring structure. In one example, the lipid moiety includes at least 5, preferably at least 6, at least 7, at least 8, at least 9 or at least 10 carbon atoms. In another embodiment, the carbon chain is interrupted by at least one functional group. Exemplary functional groups include ether, thioether, amine, carboxamide, sulfonamide, hydrazine, carbonyl, carbamate, urea, thiourea, ester and carbonate.

In one embodiment, the lipid moiety is substituted or unsubstituted alkyl. In another embodiment, the lipid moiety includes at least one isoprenyl or reduced isoprenyl moiety. In yet another embodiment, the lipid moiety is selected from poly-isoprenyl, reduced poly-isoprenyl and partially reduced poly-isoprenyl. Exemplary lipid moieties include one of the following structures:

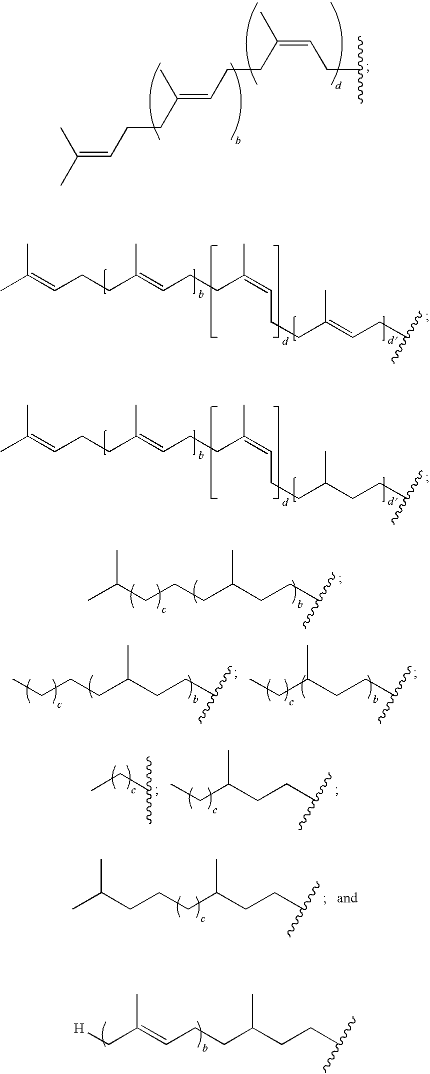

wherein b, c, d and d′ are integers independently selected from 0 to 100. In one embodiment, the lipid moiety includes a total of about 2 to about 40 isoprenyl and/or reduced isoprenyl units. In another embodiment, the lipid moiety includes a total of about 5 to about 22 isoprenyl and/or reduced isoprenyl units.

In one example according to this embodiment, the lipid moiety is undecaprenyl, a C₅₅ isoprenoid. In another example, the lipid moiety is reduced or partially reduced undecaprenyl. Exemplary lipid moieties include:

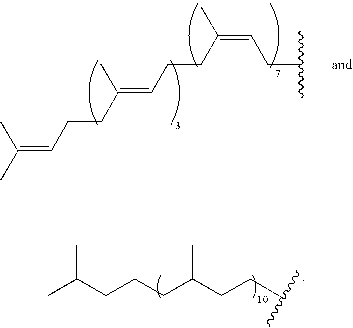

In another embodiment, the lipid moiety is derived from a fatty acid alcohol, such as those that are naturally occurring. In yet another embodiment, the lipid moiety is derived from a dolichol or a polyprenol. Dolichol-derived moieties are especially useful when using an eukaryotic oligosaccharyl transferase in the formation of a polypeptide conjugate of the invention. In one example, the lipid moiety has the general structure:

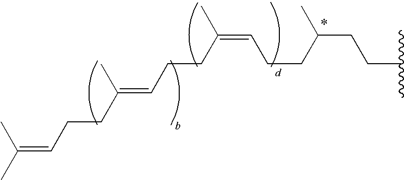

wherein b and d are integers independently selected from 0 to 100. In one example, d is selected from 1 to about 50, preferably from 1 to about 40, more preferably from 1 to about 30 and even more preferably from 1 to about 20 or 1 to about 10. In another example, d is selected from 7 to 20, preferably from 7-19, 7-18, 7-17, 7-16, 7-15, 7-14, 7-13, 7-12, 7-11, 7-10, 7-9 or 7-8. In another example, d is selected from 13-20, preferably from 14-19 and more preferably from 14-17. In another example, b is selected from 0 to 6. In yet another example, b is selected from 0 to 2. In a further example, the dolichol moiety has between about 15 and about 22 isoprenoid units. The stereocenter marked with an asterix can have (S) or (R) configuration.

Exemplary dolichol and polyprenol moieties are described, for example, in T. Chojnacki et al., Cell. Biol. Mol. Lett. 2001, 6(2), 192; T. Chojnacki and G. Dallner, Biochem. J. 1988, 251, 1-9; E. Swiezewska et al., Acta Biochim. Polon. 1994, 221-260; and G. Van Duij et al., Chem. Scripta 1987, 27, 95-100, the disclosures of which are incorporated herein in their entirety for all purposes. In a particular example, the dolichol moiety has the structure:

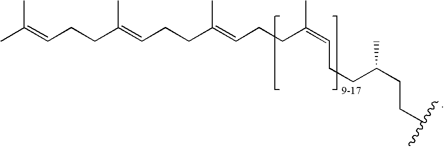

### Modifying Group of the Glycosyl Donor

In Formula (X), R^{6c} represents a modifying group of the invention. Modifying groups are described herein, e.g., in the context of polypetide conjugates and equally apply to the compounds (i.e., glycosyl donor species) of the invention. In a representative embodiment, the glycosyl donor species of Formula (X) includes a modifying group R^{6c} having a structure, which is a member selected from:

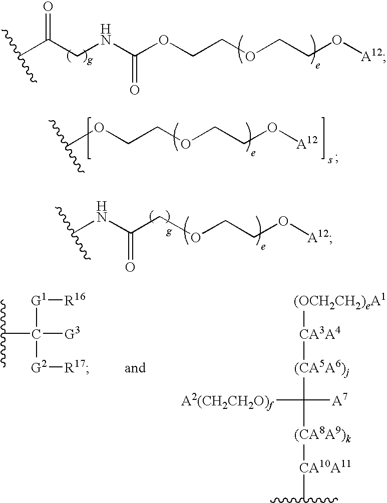

wherein g, j, k, e, f, s, R¹⁶, R¹⁷, G¹, G², G³, A¹, A², A³, A⁴, A⁵, A⁶, A⁷, A⁸, A⁹, A¹⁰, A¹¹ and A¹² are defined as above.

### Exemplary Glycosyl Donor Species

In an exemplary embodiment, the glycosyl donor species includes a phosphate or a pyrophosphate moiety and has the structure:

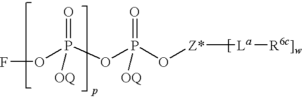

wherein w, p, L^{a}, R^{6c}, F, Q and Z* are defined herein above for Formula (X). Exemplary compounds according to this embodiment include those of the following formulae:

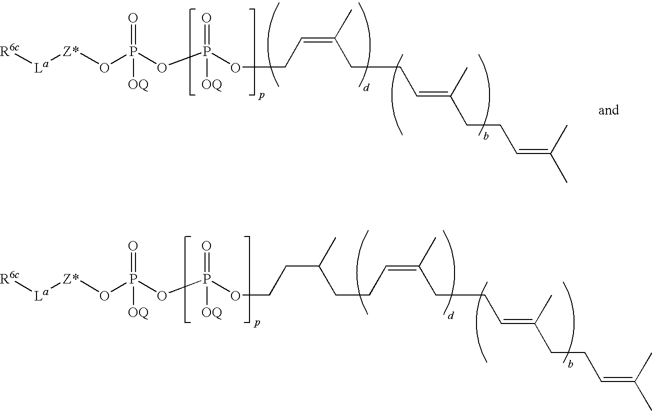

wherein b and d are independently selected from 0 to 100. In one embodiment, b is 3. In another embodiment, d is 7. In yet another embodiment, d is 7 and b is 3.

In an exemplary embodiment, the glycosyl moiety Z* in Formula (X) is a member selected from GlcNAc-GlcNAc, GlcNH-GlcNAc, GlcNAc-GlcNH or GlcNH-GlcNH moiety. In one embodiment, Z* is a GlcNAc-Gal or GlcNH-Gal moiety. In another embodiment, Z* is a GlcNAc-GlcNAc-Gal, GlcNH-GlcNAc-Gal, GlcNAc-GlcNH-Gal or GlcNH-GlcNH-Gal moiety. In another embodiment, Z* is a GlcNAc-Gal-Sia moiety. In another embodiment, Z* is a GlcNAc-GlcNAc-Gal-Sia, GlcNH-GlcNAc-Gal-Sia, GlcNAc-GlcNH-Gal-Sia or GlcNH-GlcNH-Gal-Sia moiety. Exemplary glycosyl donor species include:

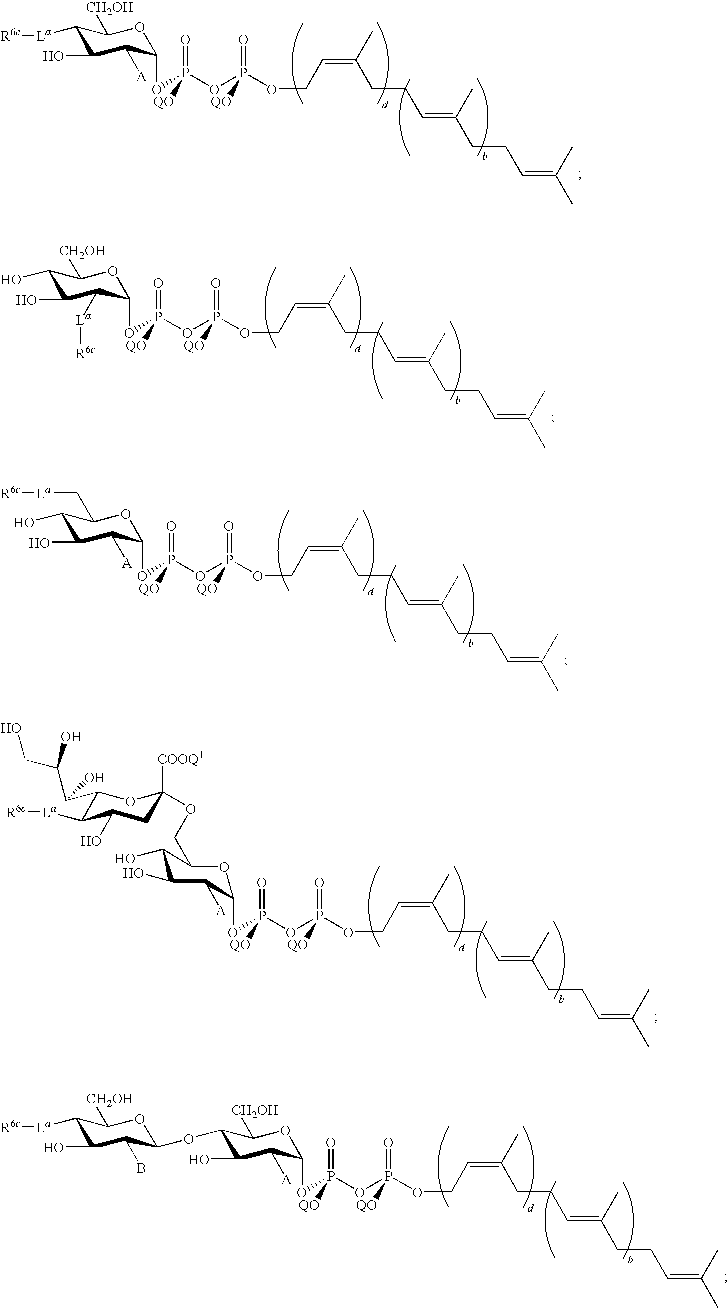

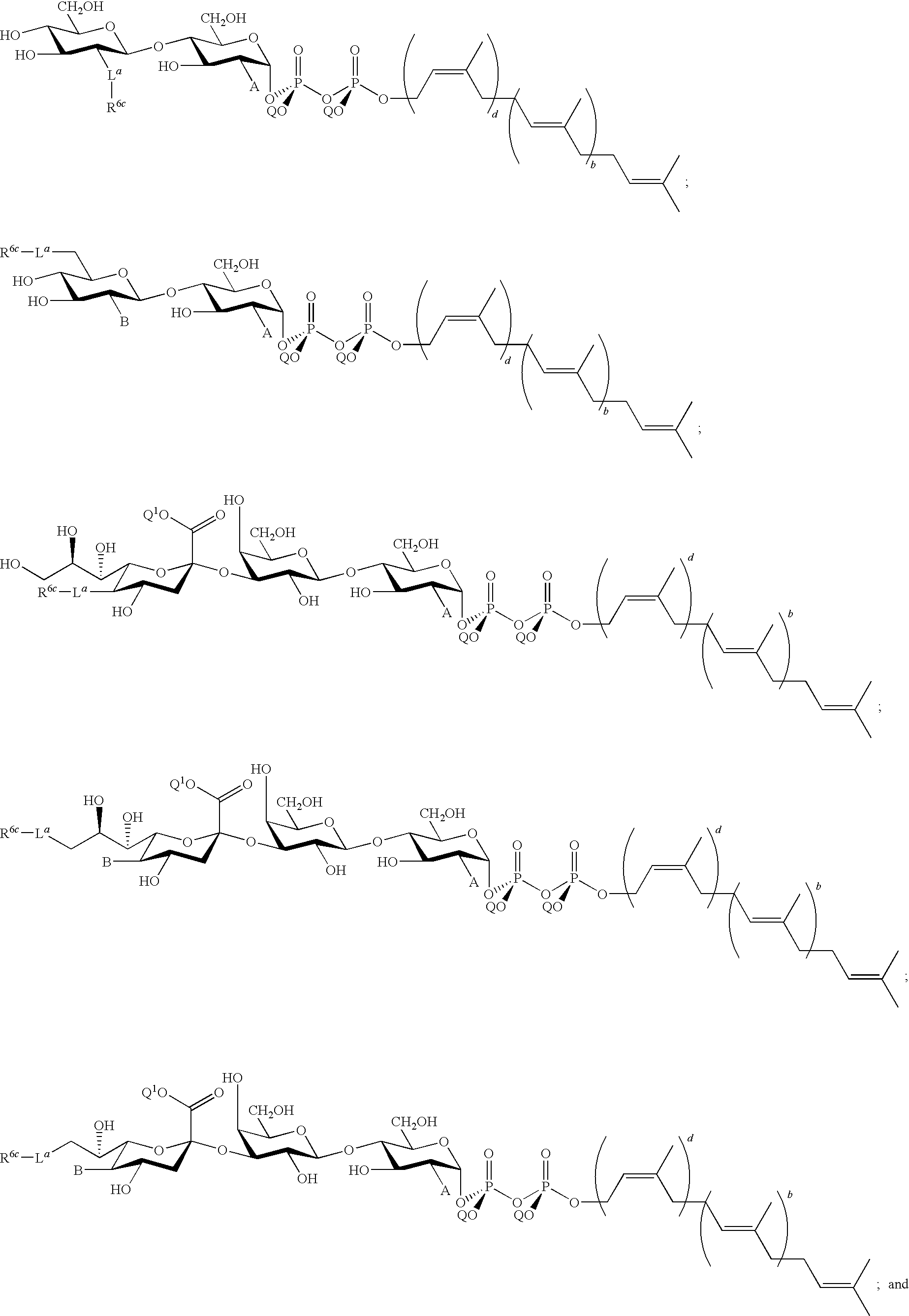

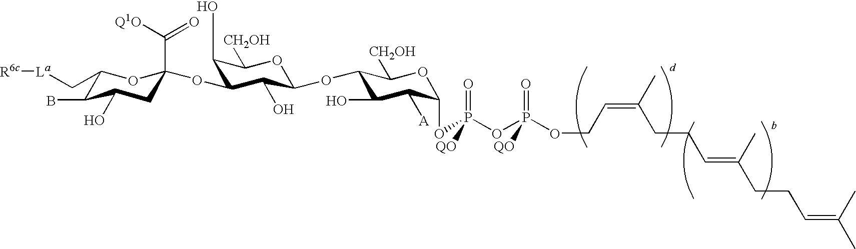

wherein b, d, Q, L^{a} and R^{6c} are as defined herein above. Q¹ is H, a single negative charge or a cation (e.g., Na⁺ or K⁺). A and B are members independently selected from OR (e.g., OH) and NHCOR (e.g., NHAc). The above shown pyrophosphates can optionally be phosphates.

Exemplary glycosyl donor species include:

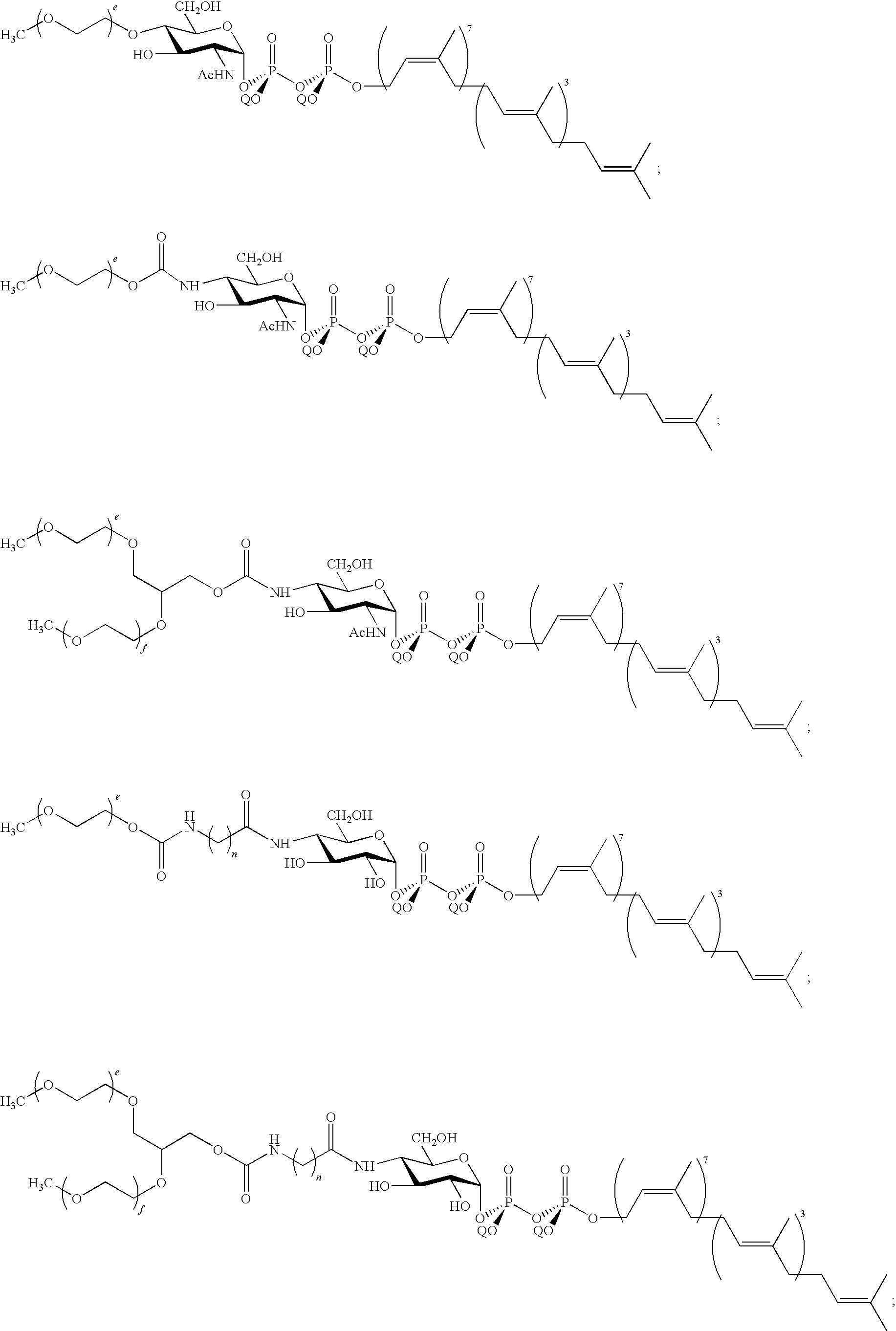

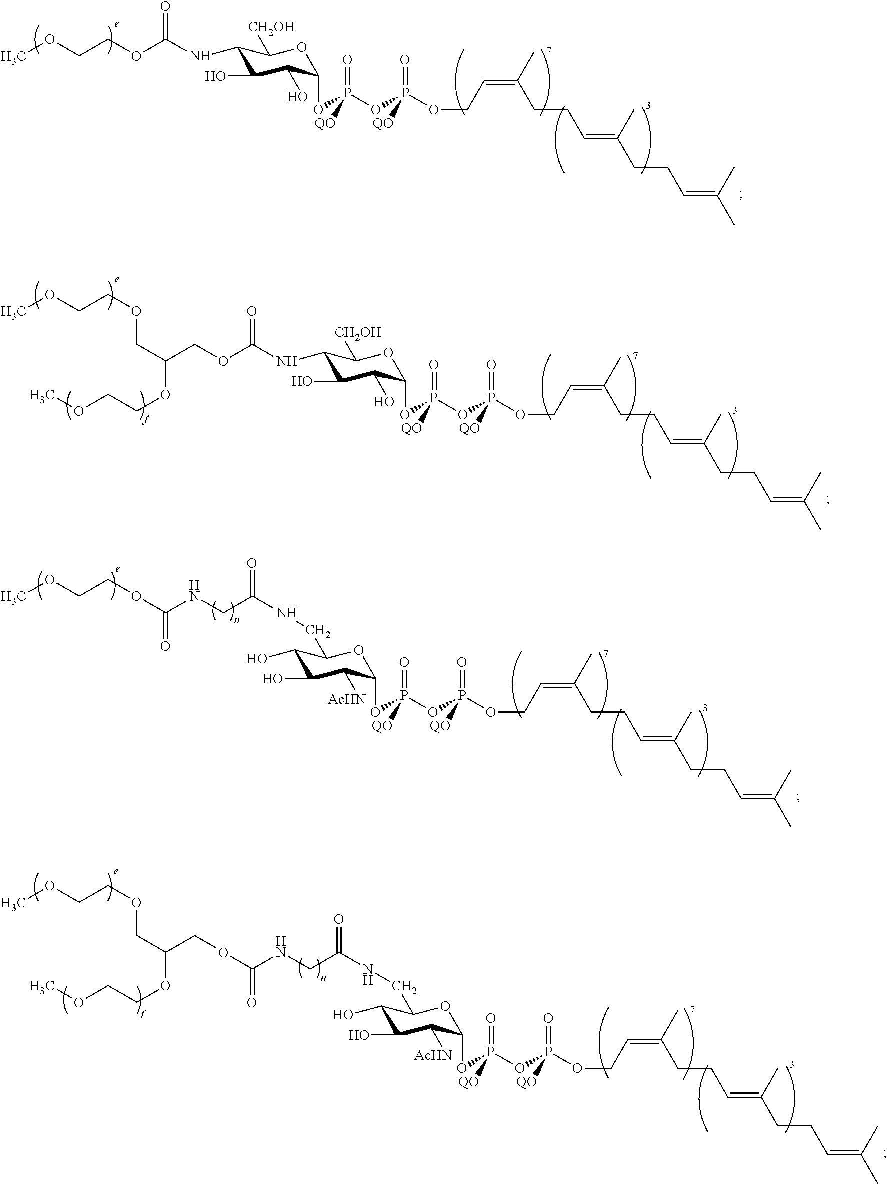

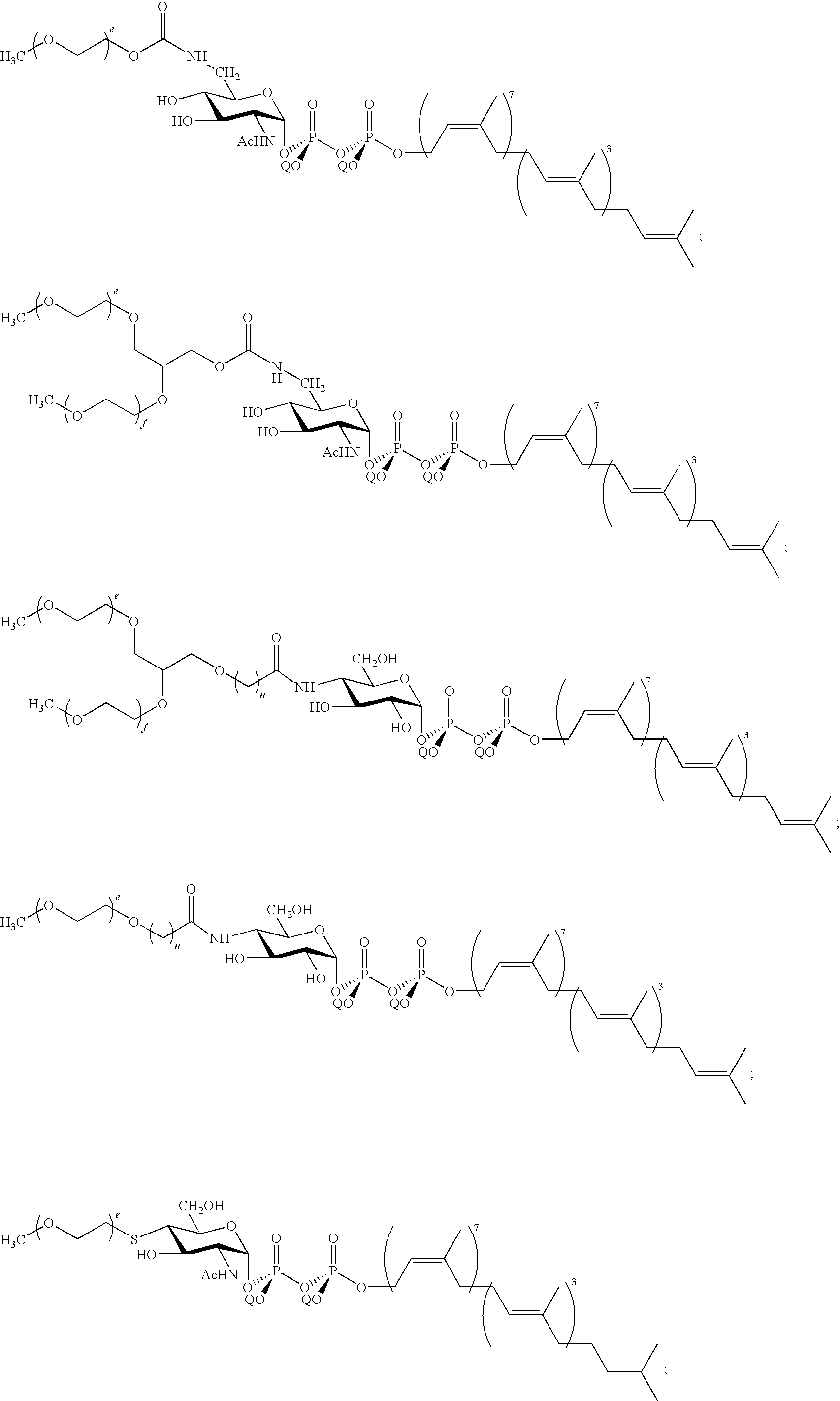

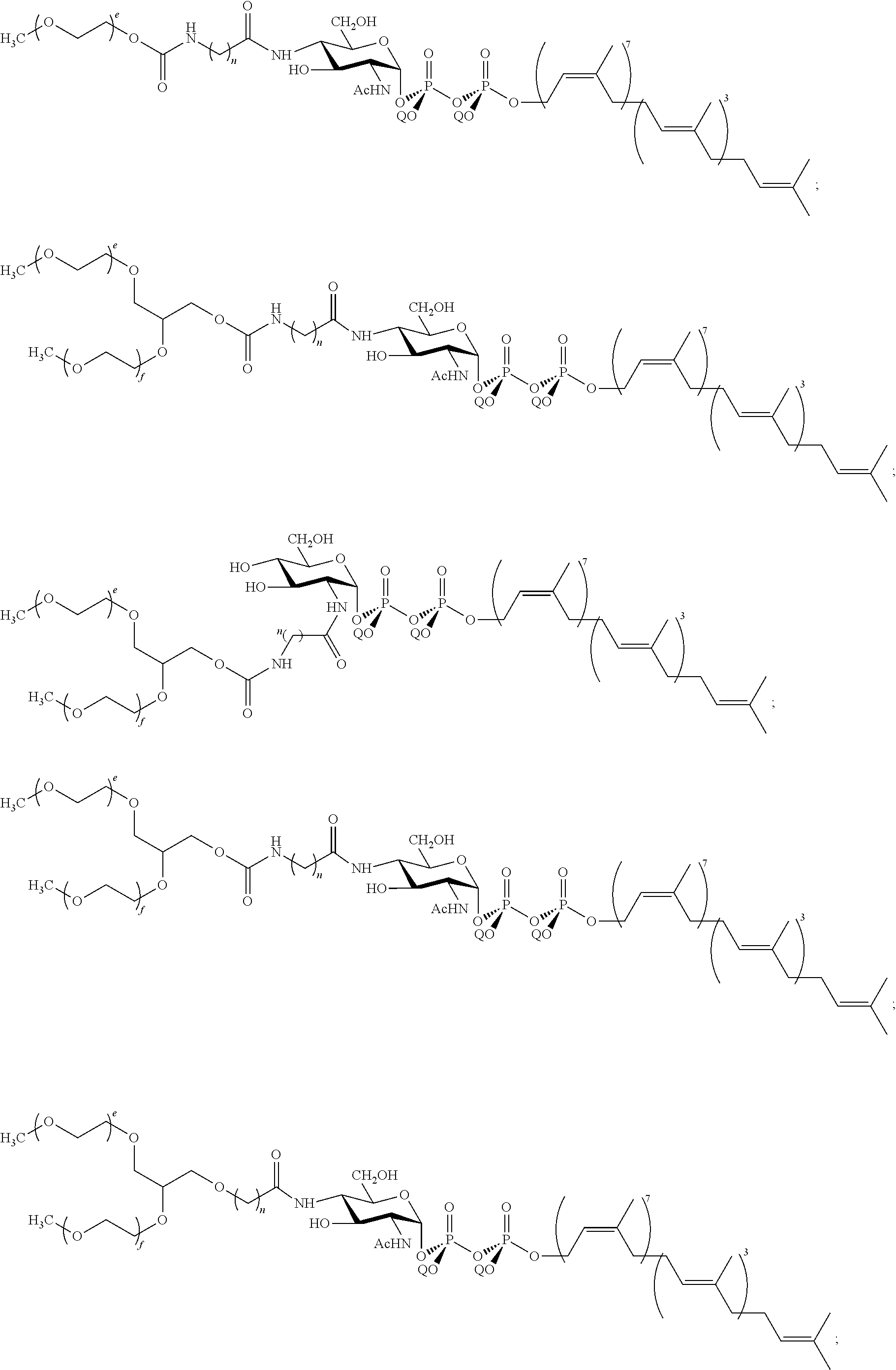

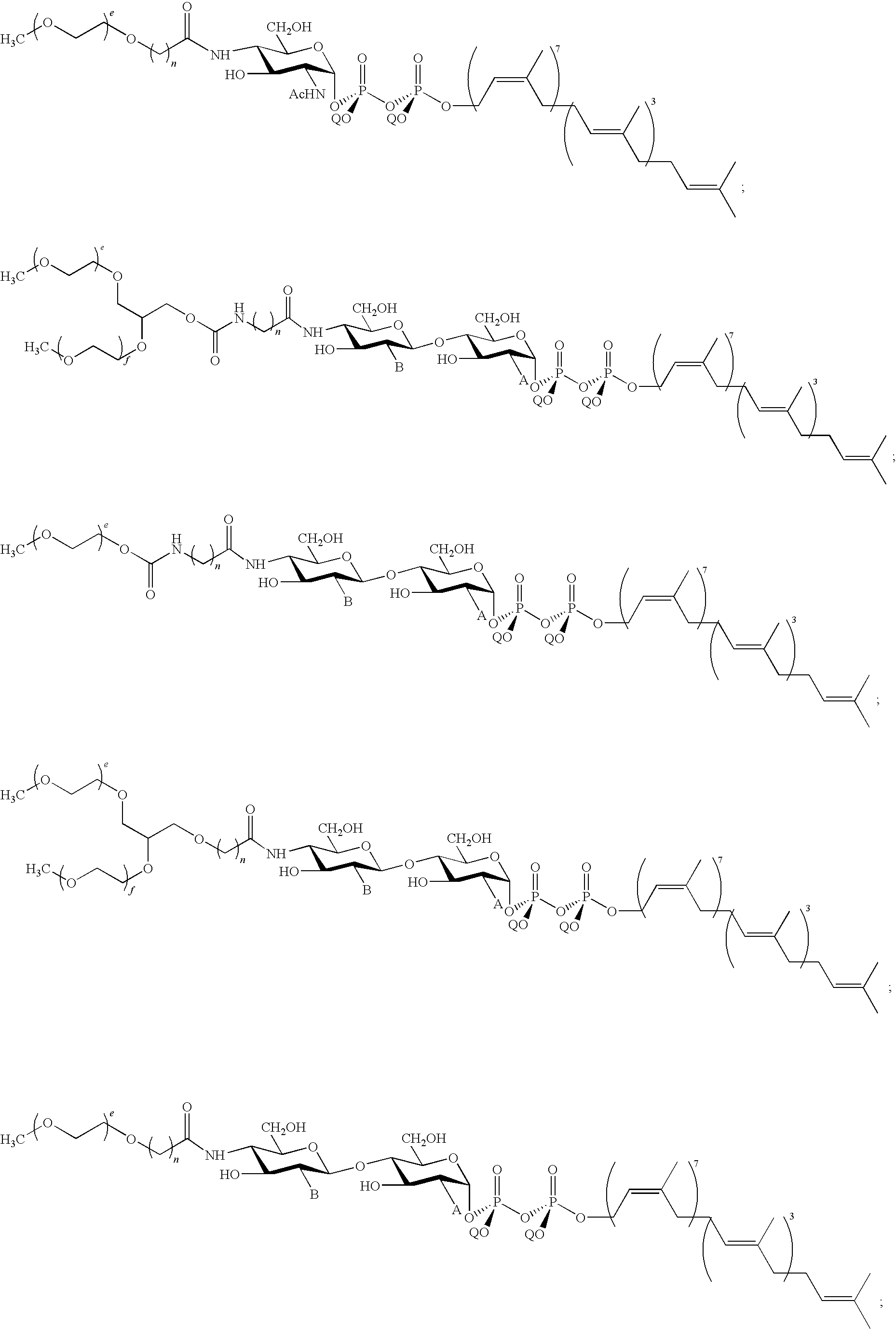

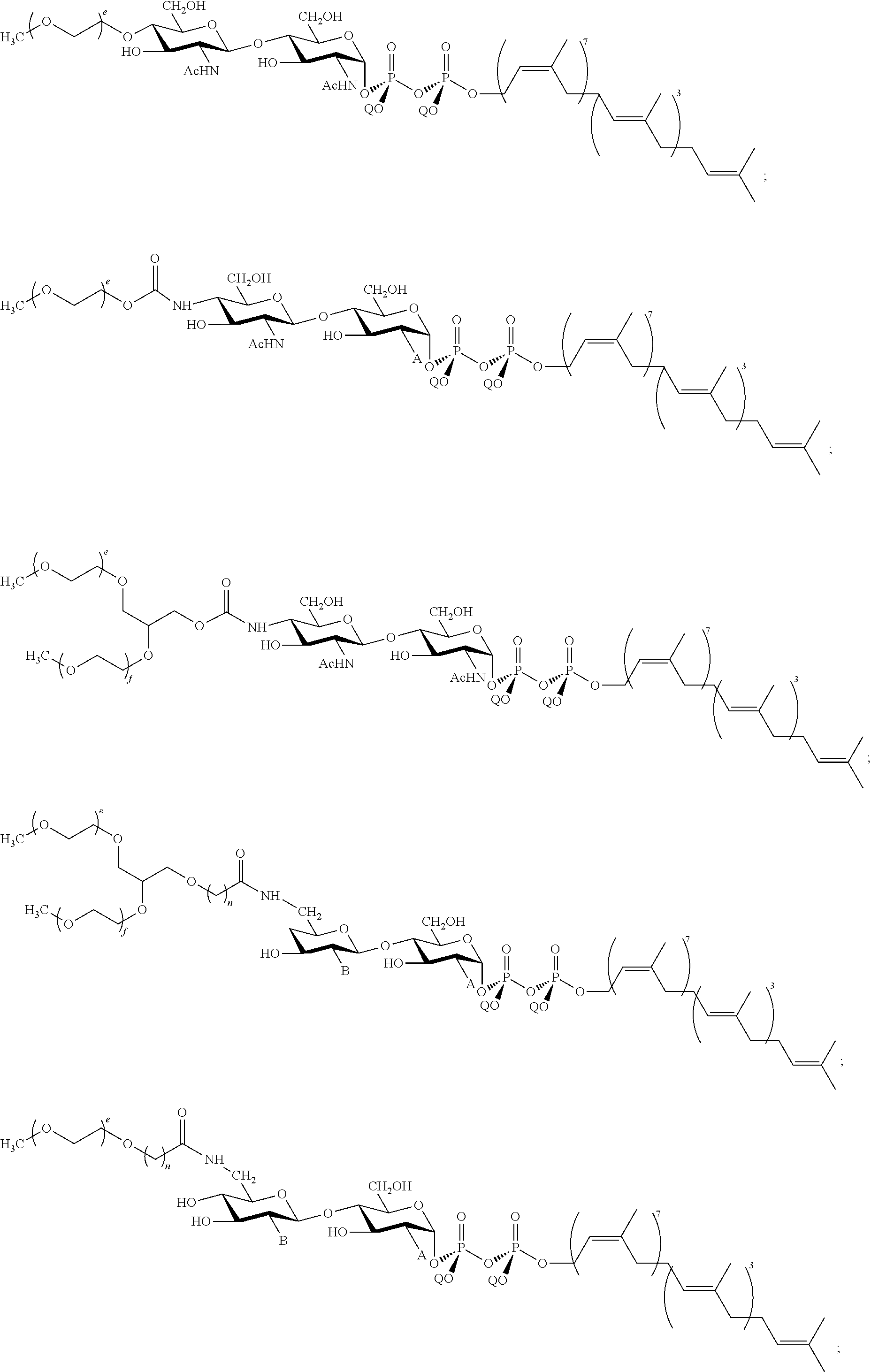

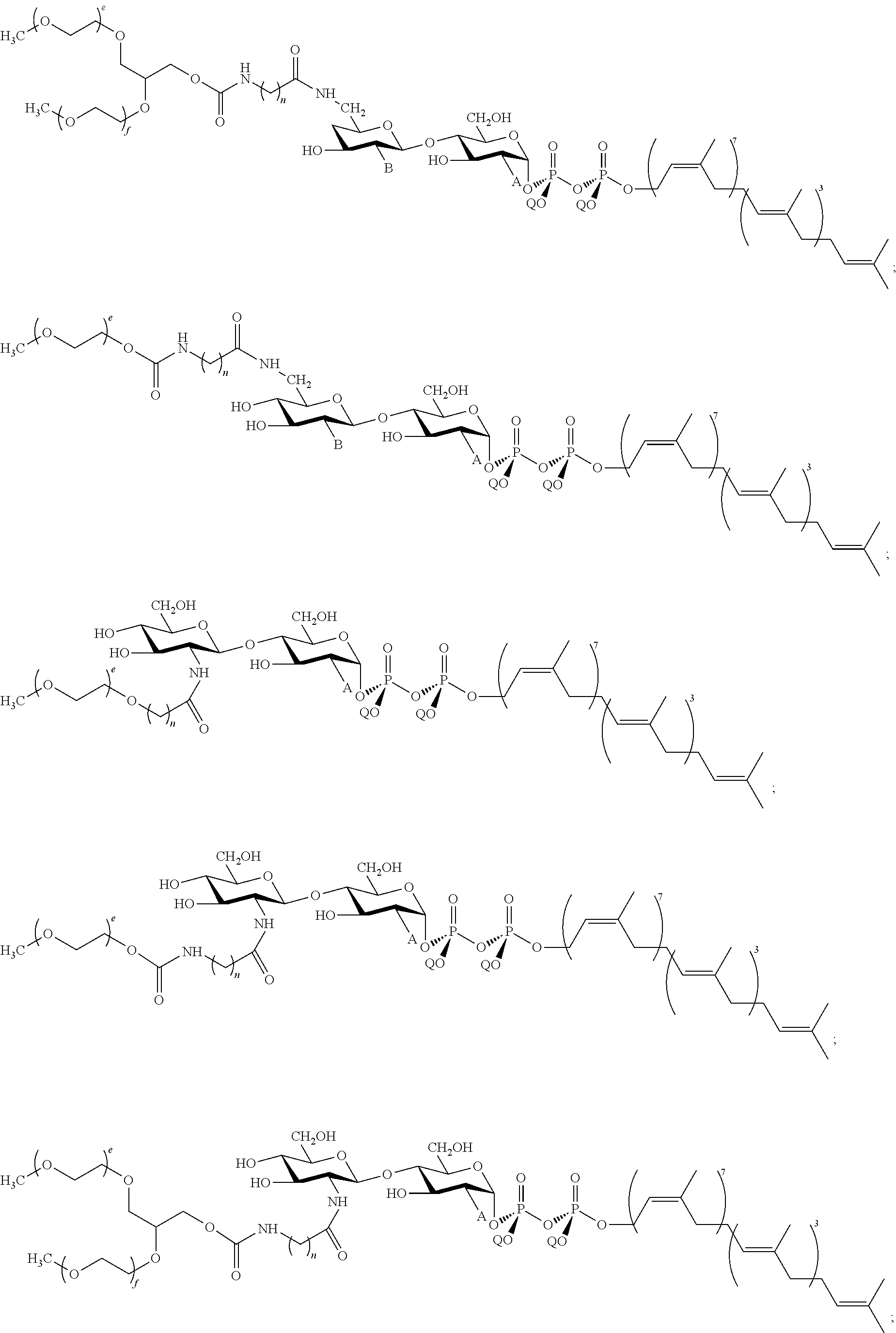

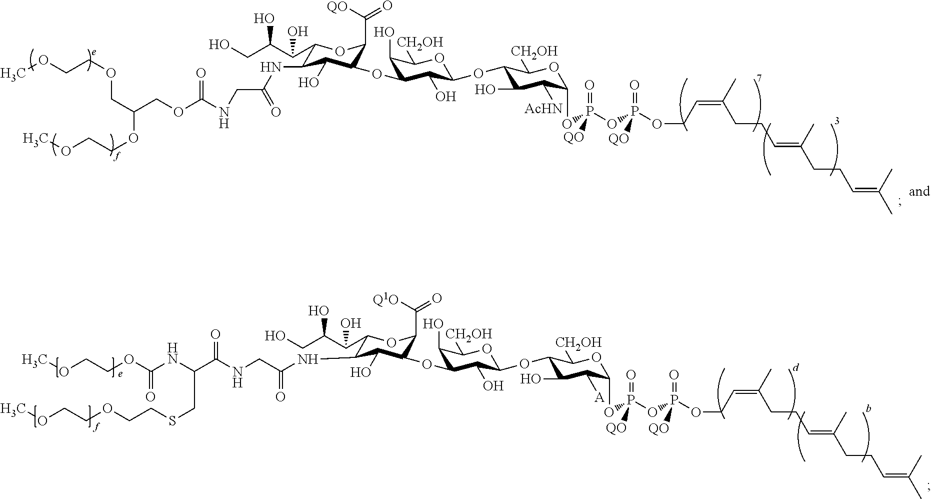

### Synthesis of Glycosyl Donor Species

The glycosyl donor species of the invention may be synthesized using a combination of art-recognized methods. For example, the synthesis of undecaprenyl-pyrophosphate-linked bacillosamine has been reported by E. Weerapana et al., J. Am. Chem. Soc. 2005, 127:13766-13767, the disclosure of which is incorporated herein by reference in its entirety. This synthetic procedure can be adopted to synthesize a variety of polyprenyl saccharides. Exemplary synthetic routes for the synthesis of lipid-pyrophosphate-linked GlcNAc moieties are shown in Scheme 3, below.

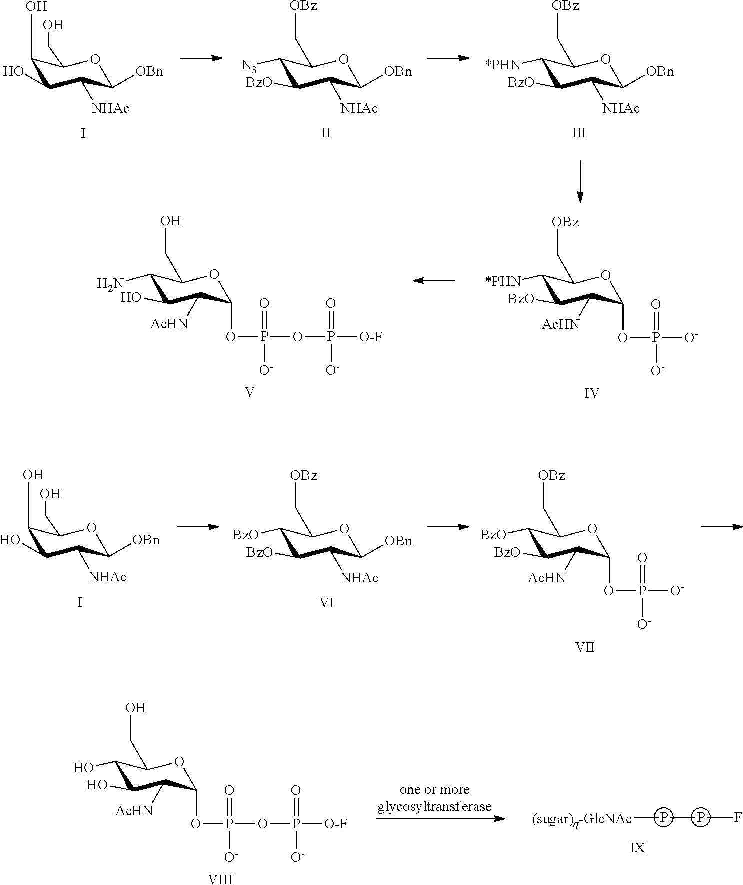

In Scheme 3a, F is a lipid moiety, such as undecaprenyl; P* is a protective group, suitable for the protection of an amino group; and the integer q is selected from 1-40.

In Scheme 3a, compound II can be prepared from known benzyl 2-acetamido-2-deoxy-β-D-galactopyranoside I through protection of the 3- and 6-hydroxyl groups, e.g., with benzoyl chloride, conversion of the 5-hydroxyl group into a leaving group (e.g., triflate) and subsequent nucleophilic substitution (e.g., using sodium azide). Compound III may then be synthesized by reduction of the azide group and protection of the resulting amino group with a suitable protective group, such as Fmoc. Selective removal of the Bn-protective group and treatment of the product with a base (e.g, LiHMDS) and a protected phosphate donor, such as a protected phospho anhydride, e.g., [(BnO)₂P(O)]₂O. Subsequent deprotection of the phosphate group gives compound IV, which may be converted to V using a lipid phosphate (undecaprenyl phosphate) and a suitable coupling reagent, such as carbonyl diimidazole, followed by deprotection of the amino group. The resulting primary amino group can be used to couple the pyrophosphate sugar to a modifying group by reaction with an activated modifying group precursor, such as those described herein. In one example, the modifying group includes a poly(etylene glycol) moiety. Activated PEG reagents are commercially available. Alternatively, the amino group can be converted to an NHAc group.

Alternatively, in Scheme 3a, compound VI may be prepared from known benzyl 2-acetamido-2-deoxy-β-D-galactopyranoside I through protection of the 3- and 6-hydroxyl groups, e.g., with benzoyl chloride and inversion of the stereocenter at C-5, e.g., through Mitsunobu chemistry. Subsequent phosphorylation and coupling of a lipid moiety as described above, gives compound VIII. Compound can be further converted to IX using one or more glycosyltransferasese and respective sugar donors, such as nucleotide sugars. In one example, the sugar donor is a modified nucleotide sugar that includes a modifying group of the invention (e.g., a modified sialic acid moiety). The modified sugar donor is used in combination with a glycosyltransferase for which the modified sugar donor is a substrate (e.g., a sialyltransferase). The reactions in Scheme 3 are exemplary and not meant to limit the scope of this invention. A person of skill in the art will appreciate that, instead of compound I, any other sugar moiety can be used as a starting material in order to create a variety of sugar phosphates through similar synthetic routes.

Another approach for the synthesis of lipid-phosphate- or lipid-pyrophosphate sugars is illustrated in Scheme 3b. In this approach, the lipid phosphate X is reacted with a sugar nucleotide containing a first sugar moiety, such as an UDP-sugar (e.g., UDP-GlcNAc, UDP-GlcNH, UDP-GalNAc, UDP-GalNH, UDP-bacillosamine, UDP-Glc, and the like) in the presence of an enzyme, which can transfer the first sugar moiety of the nucleotide sugar onto the lipid phosphate resulting in compound XI, which may include a phosphate or pyrophosphate group. In one embodiment, the enzyme is a phospho-dolichol-GlcNAc-1-phosphate transferase (GPT). Exemplary phospho-dolichol-GlcNAc-1-phosphate transferases are described herein below. Additional sugar moieties may then be added to the first sugar moiety using one or more glycosyltransferases and appropriate sugar nucleotides to give compound XII. Exemplary glycosyltransferases are also described herein, below.

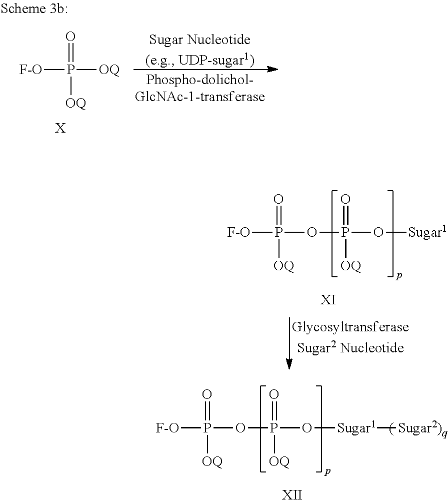

In Scheme 3b, each Q is a member independently selected from H, a single negative charge and a cation (e.g., K⁺ or Na⁺). The integer p is selected from 0 and 1; and the integer q is selected from 1 to 40.

In one embodiment, the first sugar moiety in Scheme 3b is GlcNAc and the first sugar nucleotide is UDP-GlcNAc. In one example, the first GlcNAc moiety is linked to a modified GlcNAc- or GlcNH-moiety. In another example, another GlcNAc moiety is added to the first GlcNAc moiety. The resulting GlcNAc-GlcNAc moiety may then be linked to a modified Gal moiety. Alternatively, the GlcNAc-GlcNAc moiety is first linked to a Gal moiety and a modified Sia moiety is added to the resulting GlcNAc-GlcNAc-Gal moiety. Exemplary synthetic routes according to these embodiments are illustrated in Scheme 3c, below.

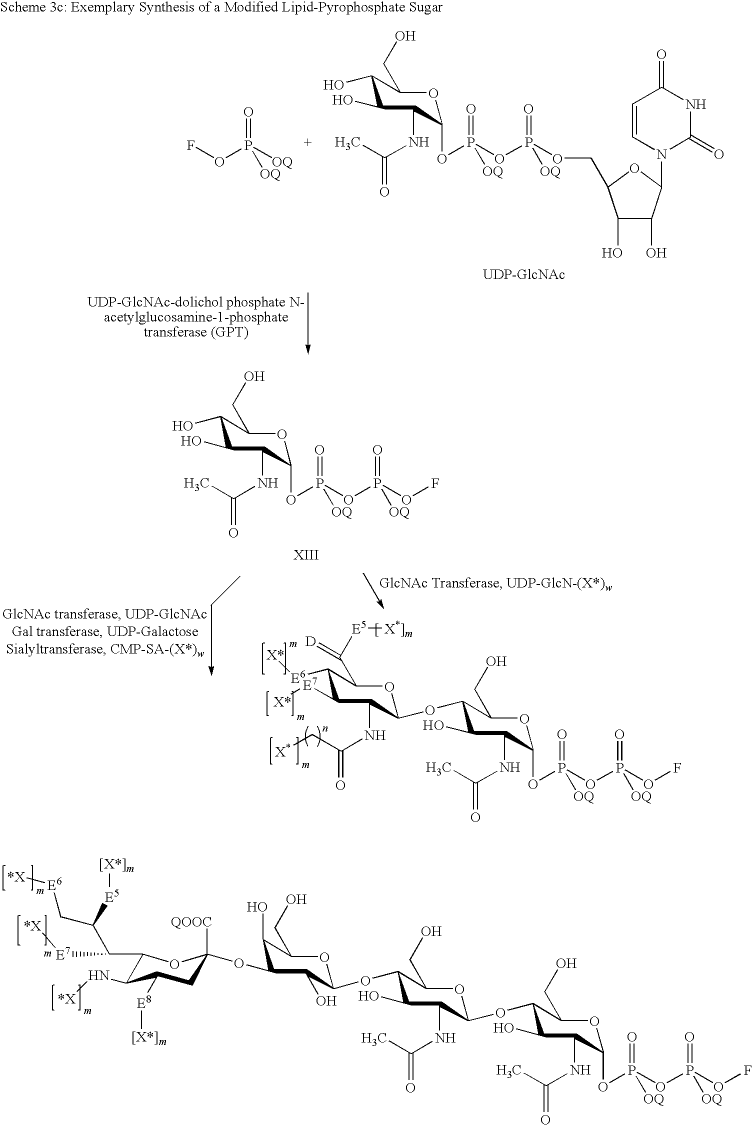

In yet another embodiment, the first GlcNAc moiety of compound XIII in Scheme 3c is linked to a modified Gal moiety. In a further embodiment, the first GlcNAc moiety of compound VIII is first linked to a Gal moiety. The Gal moiety is then linked to a modified Sia or neurominic acid moiety. These embodiments are illustrated in Scheme 3d, below:

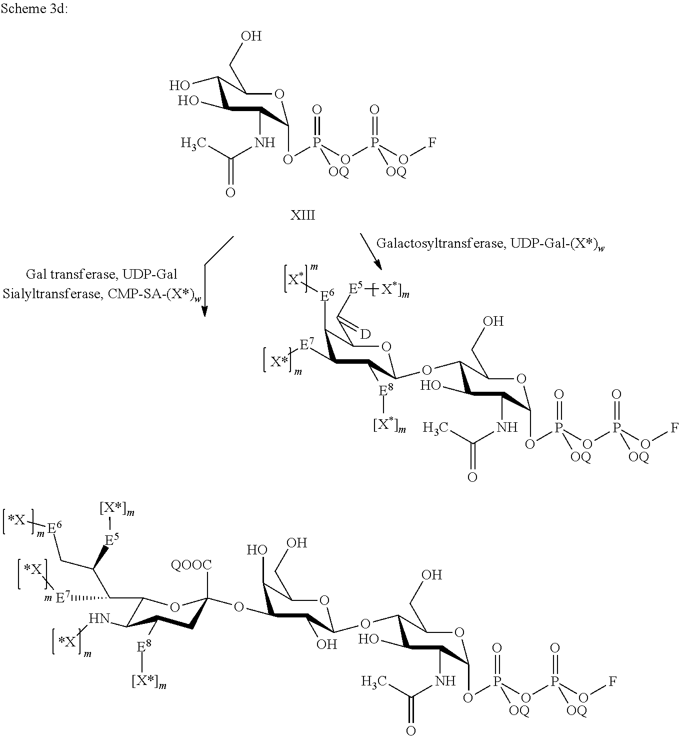

In another embodiment, the phospholipid X is reacted with a modified sugar nucleotide (e.g., modified UDP-GlcNAc) in the presence of an appropriate dolichol phosphate N-acetylglucosamine-1-phosphate transferase to yield a modified lipid-phosphate- or lipid pyrophosphate sugar. An exemplary synthetic approach according to this embodiment is illustrated in Scheme 3e, below.

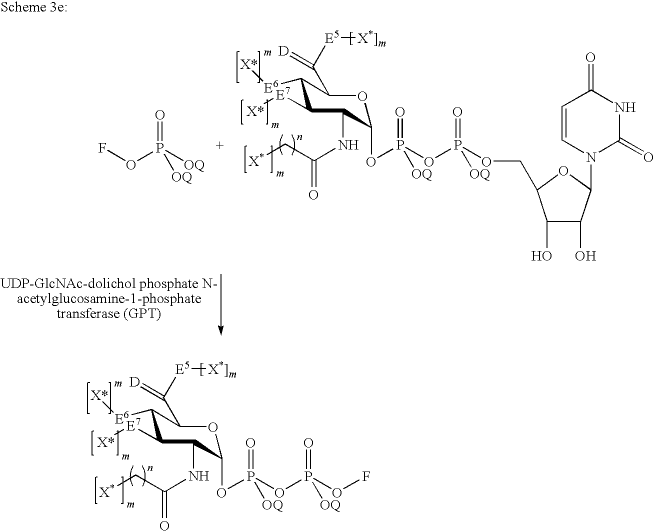

In a further embodiment, the lipid-phosphate or lipid-pyrophosphate sugar is synthesized according to the synthetic route outlined in Scheme 3f, below. In this example, a mono- or polysaccharide (e.g., a disaccharide that includes a Gal moiety) is first linked to a modified glycosyl moiety (e.g., modified Sia). In one example, the modified glycosyl moiety is linked to the starting material using a glycosyl transferase, such as a sialyltransferase, and an appropriate modified sugar nucleotide (e.g., modified CMP-Sia). Any glycosidic OH groups may then be protected, for example, as their corresponding methyl ethers, and the resulting protected modified saccharide can then be linked to a phospholipid or pyrophospholipid.

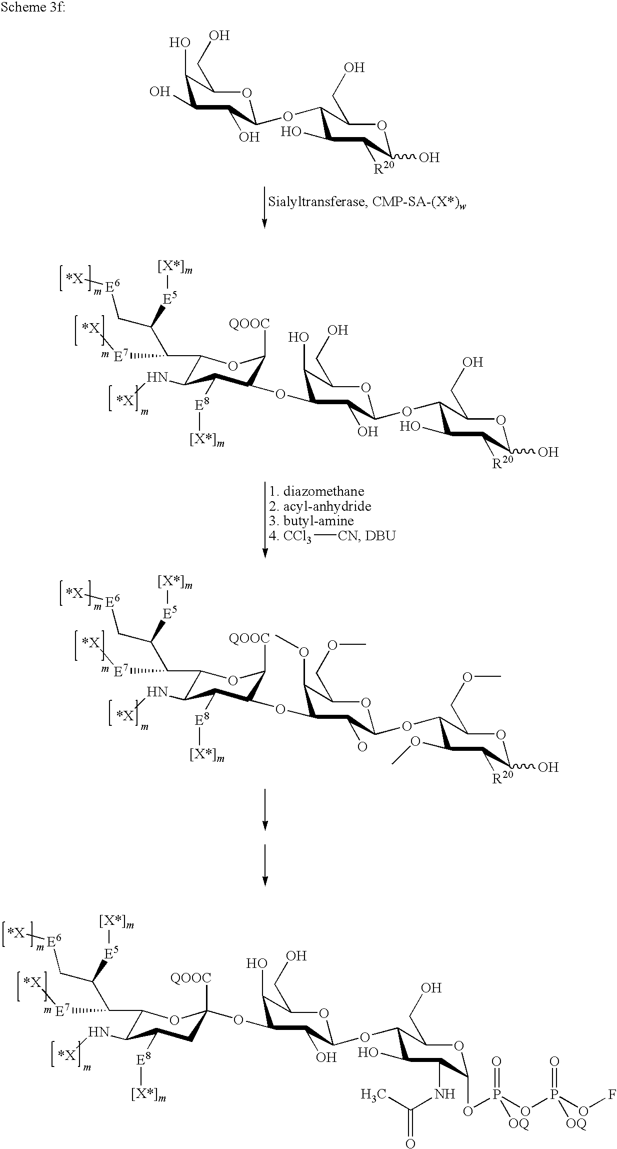

wherein R²⁰ is a member selected from OH, NH₂, NHAc, NHCOaryl and NHCOalkyl.

In Schemes 3c to 3f, F is a lipid moiety described herein; each Q is a member independently selected from H, a single negative charge and a cation (e.g., K⁺ or Na⁺). The integer w is selected from 1 to 8, preferably from 1-4 (e.g., for Glc or Gal moieties) or 1-5 (e.g., for Sia moieties); the integer n is selected from 0 to 40; and each integer m is a member independently selected from 0 and 1. When m is 0, then (X*)ₘ is replaced with H. In one example, each X* is a member independently selected from linear and branched polymeric modifying groups described herein. In another example, X* includes at least one polymeric moiety, such as a PEG moiety (e.g., mPEG). In yet another example, X* includes a linker moiety linking the polymeric modifying group to the remainder of the molecule. In a further example, each X* is L^{b}-R^{6c} described herein for Formula (V). E⁵, E⁶, E⁷ and E⁸ are members independently selected from CR¹R² (e.g., CH₂) and a functional group, such as O, S, NR³ (e.g., NH), C(O), C(O)NR³ (e.g., CONH), NHC(O), NHC(O)NH, NHC(O)O and the like; and D is a member selected from H₂ (in which case the double bond is replaced with two single bonds), O, S, NR³ (e.g., NH), wherein each R¹, each R², each R³ and each R⁴ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

Various glycosyl transferases and appropriate modified or non-modified sugar nucleotides may be used to elaborate the first sugar moiety of the phosphate or pyrophosphate sugar (e.g., compound VIII in Schemes 3). For example, a second GlcNAc moiety can be added onto a first GlcNAc moiety. The second GlcNAc moiety may optionally be modified with a modifying group of the invention (compare Scheme 3c for an example). In another example, a modified sialic acid moiety may be transferred enzymatically to a GlcNAc, GlcNAc-GlcNAc- or GlcNAc-GlcNAc-Gal-moiety of the phosphate- or pyrophosphate sugar (compare Scheme 3c for an example). Alternatively any other glycosyl moiety (e.g., Gal, GalNAc etc.) can be added to the first sugar moiety using appropriate glycosyl transferases described herein.

Modified sugar residues may be added to an existing sugar residue enzymatically using a modified sugar nucleotide or a modified activated sugar in combination with a suitable glycosyltransferase, for which the modified sugar species is a substrate. Hence, modified sugars are preferably selected from modified sugar nucleotides, activated modified sugars and modified sugars that are simple saccharides (neither nucleotides nor activated). Typically, the structure will be a monosaccharide, but the present invention is not limited to the use of modified monosaccharide sugars. Oligosaccharides, polysaccharides and glycosyl-mimetic moieties are useful as well.

In another embodiment, the glycosyl donor species are synthesized from lipid-phosphate precursors (e.g., undecaprenyl-phosphate) using purified enzymes (e.g., from the bacterial or yeast N-glycosylation pathways). Such reactions using recombinant enzymes have been described by K J Glover et al. (PNAS 2005, 102(40): 14255-14259), the disclosure of which is incorporated herein by reference in its entirety. For example, PglC may be used to add a modified or non-modified bacillosamine moiety from UDP-bacillosamine onto undecaprenyl-phosphate to give undecaprenyl-pyrophosphate-linked bacillosamine, which may be further converted to undecaprenyl-pyrophosphate-linked bacillosamine-GalNAc using PglA and UDP GalNAc, wherein the GalNAc moiety can optionally be modified. Additional sugar moieties may be added using other enzymes such as PglHJ or PglI. Two or more of these reactions may be performed in a single reaction vessel. The reagents (i.e., enzymes and nucleotide sugars) for two or more steps may be added sequentially or simultaneously. Exemplary enzymes from the yeast pathway, which may be used to make a glycosyl donor species of the invention include Alg 1-14 (e.g., Alg1, Alg2, Alg 7 and Alg13/14).

The modifying group is attached to a sugar moiety by enzymatic means, chemical means or a combination thereof, thereby producing a modified sugar. The sugars are substituted at any position that allows for the attachment of the modifying group, yet which still allows the sugar to function as a substrate for the enzyme used to ligate the modified sugar to the receiving structure. In an exemplary embodiment, when sialic acid is the sugar, the sialic acid is substituted with the modifying group at either the pyruvyl side chain or at the 5-position amine that is normally acetylated in sialic acid.

### Modified Sugar Nucleotides

In certain embodiments of the present invention, a modified sugar nucleotide is utilized to add a modified sugar moiety to the precursor of the glycosyl donor species. Exemplary sugar nucleotides that are used in the present invention in their modified form include nucleotide mono-, di- or triphosphates or analogs thereof. In a preferred embodiment, the modified sugar nucleotide is selected from a UDP-glycoside, CMP-glycoside, and a GDP-glycoside. Even more preferably, the modified sugar nucleotide is selected from an UDP-galactose, UDP-galactosamine, UDP-glucose, UDP-glucosamine, UDP-bacillosamine, UDP-6-hydroxybacillosamine, GDP-mannose, GDP-fucose, CMP-sialic acid, and CMP-NeuAc. N-acetylamine derivatives of the sugar nucleotides are also of use in the methods of the invention.

In one example, the nucleotide sugar species is modified with a water-soluble polymer. An exemplary modified sugar nucleotide bears a sugar group that is modified through an amine moiety on the sugar. Modified sugar nucleotides, e.g., saccharyl-amine derivatives of a sugar nucleotide, are also of use in the methods of the invention. For example, a saccharyl amine (without the modifying group) can be enzymatically conjugated to a polypeptide (or other species) and the free saccharyl amine moiety subsequently be conjugated to a desired modifying group. Alternatively, the modified sugar nucleotide can function as a substrate for an enzyme that transfers the modified sugar to a saccharyl acceptor on the polypeptide. Exemplary modified sugar nucleotides include modified sialic acid nucleotides such as:

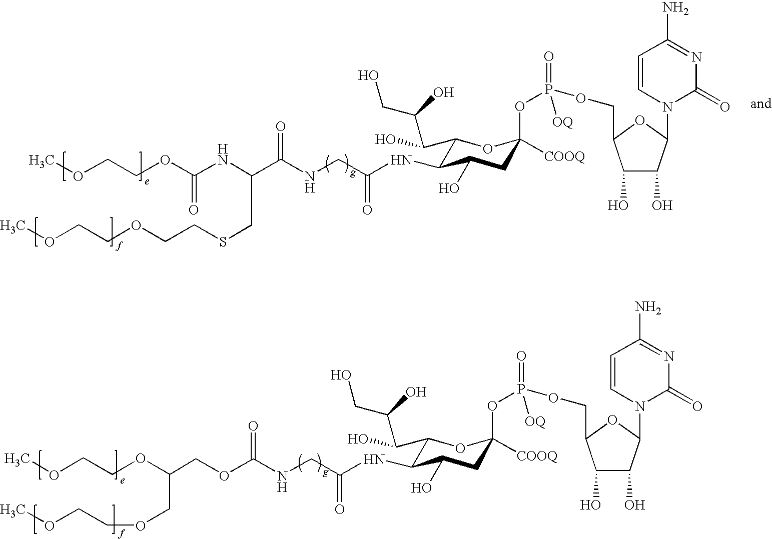

wherein e, f and Q are defined herein above and g is an integer selected from 1-20.

In an exemplary embodiment, the modified sugar is based upon a 6-amino-N-acetyl-glycosyl moiety. As shown in Scheme 4, below for N-acetylgalactosamine, the modified sugar nucleotide can be readily prepared using standard methods.

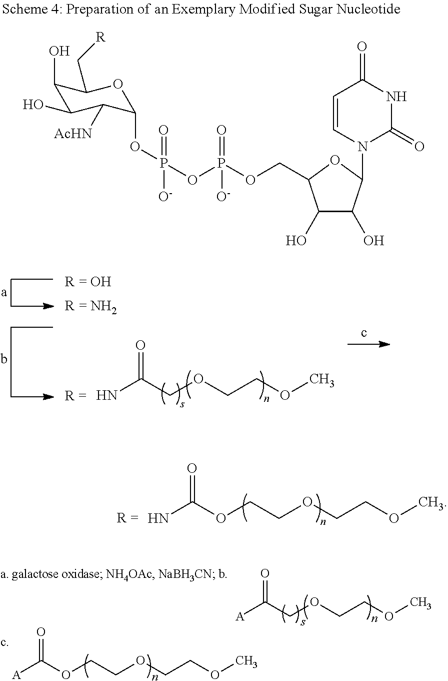

In Scheme 4, above, the index n represents an integer from 0 to 2500, preferably from 10 to 1500, and more preferably from 10 to 1200. The symbol “A” represents an activating group, e.g., a halo, a component of an activated ester (e.g., a N-hydroxysuccinimide ester), a component of a carbonate (e.g., p-nitrophenyl carbonate) and the like. Those of skill in the art will appreciate that other PEG-amide nucleotide sugars are readily prepared by this and analogous methods.

In other exemplary embodiments, the amide moiety is replaced by a group such as a urethane or a urea.

In still further embodiments, R¹ is a branched PEG, for example, one of those species set forth above. Illustrative compounds according to this embodiment include:

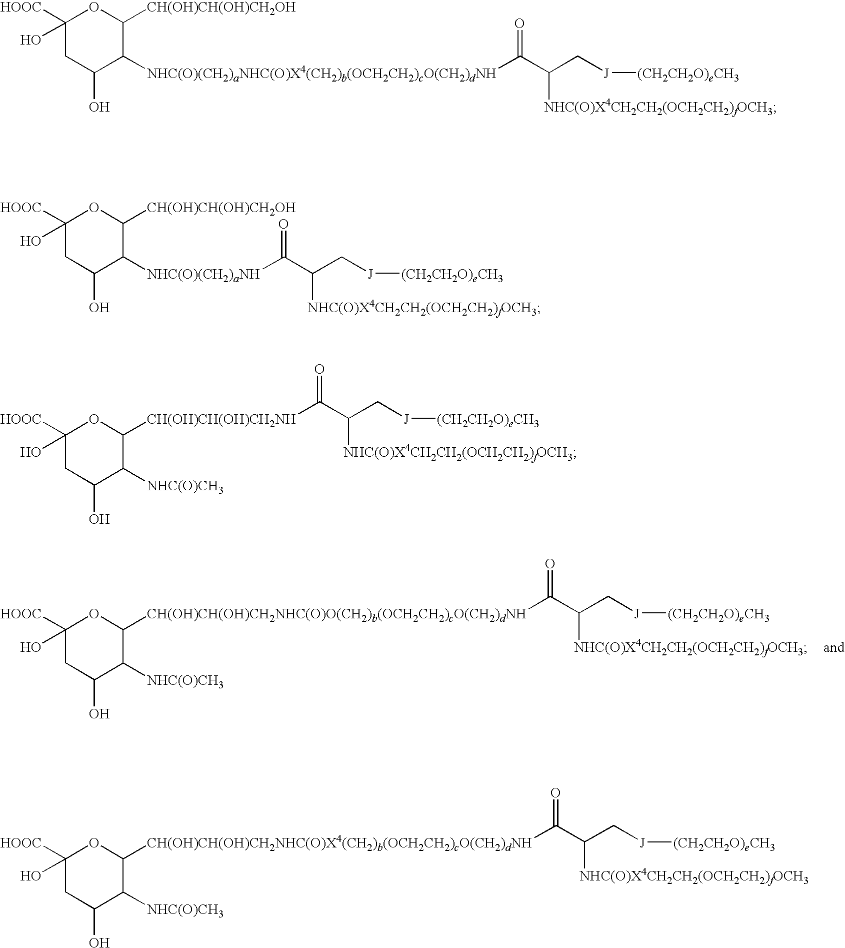

in which X⁴ is a bond or O, and J is S or O.

Moreover, as discussed above, the present invention provides nucleotide sugars that are modified with a water-soluble polymer, which is either straight-chain or branched. For example, compounds having the formula shown below are within the scope of the present invention:

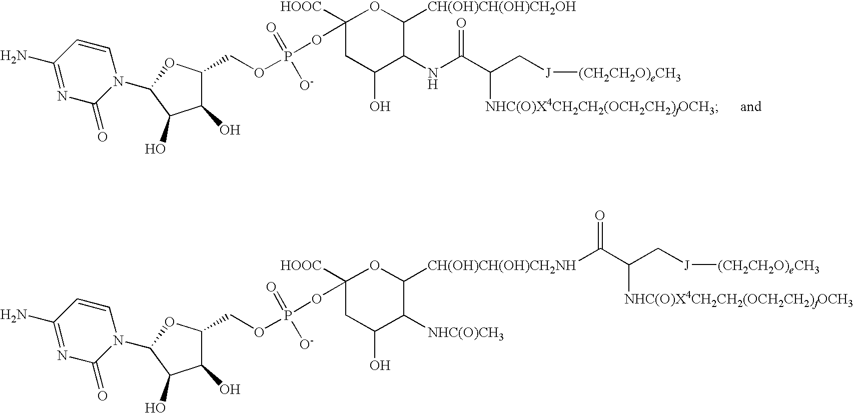

in which X⁴ is O or a bond, and J is S or O.

Similarly, the invention provides polypeptide conjugates that are formed using nucleotide sugars of those modified sugar species in which the carbon at the 6-position is modified:

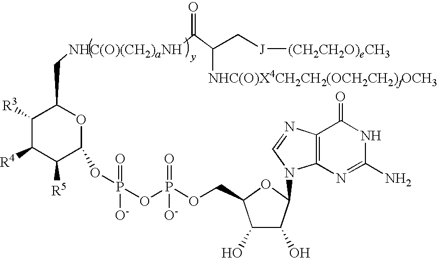

in which X⁴ is a bond or O, J is S or O, and y is 0 or 1.

Also provided are polypeptide and glycopeptide conjugates having the following formulae:

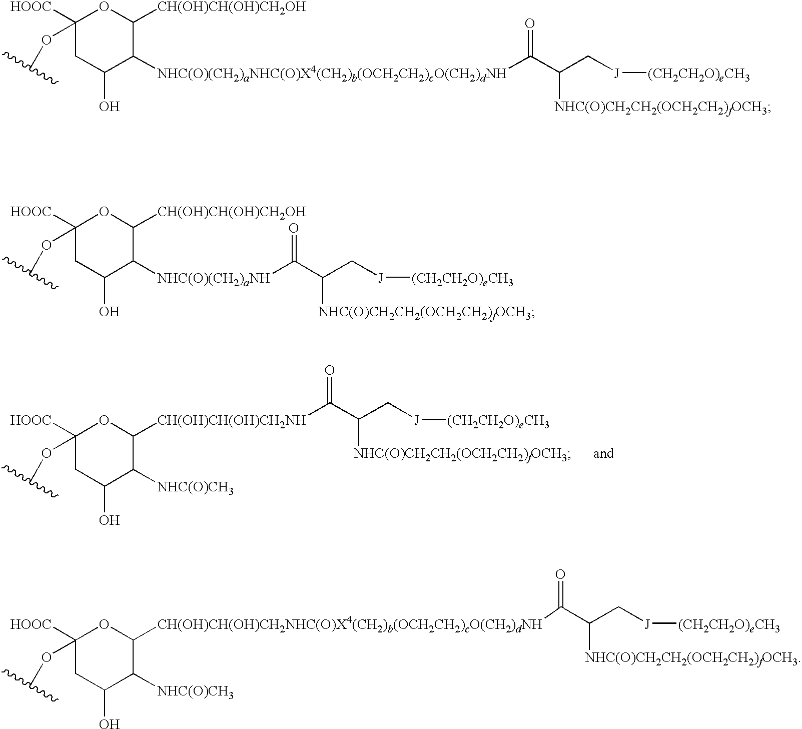

wherein J is S or O.

### Activated Sugars

In other embodiments, the modified sugar is an activated sugar. Activated, modified sugars, which are useful in the present invention, are typically glycosides which have been synthetically altered to include a leaving group. In one example, the activated sugar is used in an enzymatic reaction to transfer the activated sugar onto an acceptor on the polypeptide or glycopeptide. In another example, the activated sugar is added to the polypeptide or glycopeptide by chemical means. “Leaving group” (or activating group) refers to those moieties, which are easily displaced in enzyme-regulated nucleophilic substitution reactions or alternatively, are replaced in a chemical reaction utilizing a nucleophilic reaction partner (e.g., a glycosyl moiety carrying a sufhydryl group). It is within the abilities of a skilled person to select a suitable leaving group for each type of reaction. Many activated sugars are known in the art. See, for example, Vocadlo et al., In CARBOHYDRATE CHEMISTRY AND BIOLOGY, Vol. 2, Ernst et al. Ed., Wiley-VCH Verlag: Weinheim, Germany, 2000; Kodama et al., Tetrahedron Lett. 34: 6419 (1993); Lougheed, et al., J. Biol. Chem. 274: 37717 (1999)).

Examples of leaving groups include halogen (e.g, fluoro, chloro, bromo), tosylate ester, mesylate ester, triflate ester and the like. Preferred leaving groups, for use in enzyme mediated reactions, are those that do not significantly sterically encumber the enzymatic transfer of the glycoside to the acceptor. Accordingly, preferred embodiments of activated glycoside derivatives include glycosyl fluorides and glycosyl mesylates, with glycosyl fluorides being particularly preferred. Among the glycosyl fluorides, α-galactosyl fluoride, α-mannosyl fluoride, α-glucosyl fluoride, α-fucosyl fluoride, α-xylosyl fluoride, α-sialyl fluoride, α-N-acetylglucosaminyl fluoride, α-N-acetylgalactosaminyl fluoride, β-galactosyl fluoride, β-mannosyl fluoride, β-glucosyl fluoride, β-fucosyl fluoride, β-xylosyl fluoride, β-sialyl fluoride, β-N-acetylglucosaminyl fluoride and β-N-acetylgalactosaminyl fluoride are most preferred. For non-enzymatic, nucleophilic substitutions, these and other leaving groups may be useful. For instance, the activated donor glycoside can be a dinitrophenyl (DNP), or bromo-glycoside.

By way of illustration, glycosyl fluorides can be prepared from the free sugar by first acetylating and then treating the sugar moiety with HF/pyridine. This generates the thermodynamically most stable anomer of the protected (acetylated) glycosyl fluoride (i.e., the α-glycosyl fluoride). If the less stable anomer (i.e., the β-glycosyl fluoride) is desired, it can be prepared by converting the peracetylated sugar with HBr/HOAc or with HCl to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g. NaOMe/MeOH). In addition, many glycosyl fluorides are commercially available.

Other activated glycosyl derivatives can be prepared using conventional methods known to those of skill in the art. For example, glycosyl mesylates can be prepared by treatment of the fully benzylated hemiacetal form of the sugar with mesyl chloride, followed by catalytic hydrogenation to remove the benzyl groups.

In a further exemplary embodiment, the modified sugar is an oligosaccharide having an antennary structure. In another embodiment, one or more of the termini of the antennae bear the modifying moiety. When more than one modifying moiety is attached to an oligosaccharide having an antennary structure, the oligosaccharide is useful to “amplify” the modifying moiety; each oligosaccharide unit conjugated to the polypeptide attaches multiple copies of the modifying group to the polypeptide. The general structure of a typical conjugate of the invention as set forth in the drawing above encompasses multivalent species resulting from preparing a conjugate of the invention utilizing an antennary structure. Many antennary saccharide structures are known in the art, and the present method can be practiced with them without limitation.

### Preparation of Modified Sugars

In general, a covalent bond between a sugar moiety (including those of a lipid-pyrophosphate sugar) and the modifying group is formed through the use of reactive functional groups, which are typically transformed by the linking process into a new organic functional group or unreactive species. In order to form the bond, the modifying group and the sugar moiety carry complimentary reactive functional groups. The reactive functional group(s) can be located at any position on the sugar moiety.

Reactive groups and classes of reactions useful in practicing the present invention are generally those that are well known in the art of bioconjugate chemistry. Currently favored classes of reactions available with reactive sugar moieties are those, which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

### Reactive Functional Groups

Useful reactive functional groups pendent from a sugar nucleus or modifying group include, but are not limited to:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc; and
(j) epoxides, which can react with, for example, amines and hydroxyl compounds.

The reactive functional groups can be chosen such that they do not participate in, or interfere with, the reactions necessary to assemble the reactive sugar nucleus or modifying group. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### Cross-Linking Groups

Preparation of the Modified Sugar for Use in the Methods of the Present Invention includes attachment of a modifying group to a sugar residue and forming a stable adduct, which is a substrate for a glycosyltransferase. The sugar and modifying group can be coupled by a zero- or higher-order cross-linking agent. Exemplary bifunctional compounds which can be used for attaching modifying groups to carbohydrate moieties include, but are not limited to, bifunctional poly(ethyleneglycols), polyamides, polyethers, polyesters and the like. General approaches for linking carbohydrates to other molecules are known in the literature. See, for example, Lee et al., Biochemistry 28: 1856 (1989); Bhatia et al., Anal. Biochem. 178: 408 (1989); Janda et al., J. Am. Chem. Soc. 112: 8886 (1990) and Bednarski et al., WO 92/18135. In the discussion that follows, the reactive groups are treated as benign on the sugar moiety of the nascent modified sugar. The focus of the discussion is for clarity of illustration. Those of skill in the art will appreciate that the discussion is relevant to reactive groups on the modifying group as well.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

In addition to the use of site-specific reactive moieties, the present invention contemplates the use of non-specific reactive groups to link the sugar to the modifying group.

Exemplary non-specific cross-linkers include photoactivatable groups, completely inert in the dark, which are converted to reactive species upon absorption of a photon of appropriate energy. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In yet a further embodiment, the linker group is provided with a group that can be cleaved to release the modifying group from the sugar residue. Many cleaveable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups is commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

In the discussion that follows, a number of specific examples of modified sugars that are useful in practicing the present invention are set forth. In the exemplary embodiments, a sialic acid derivative is utilized as the sugar nucleus to which the modifying group is attached. The focus of the discussion on sialic acid derivatives is for clarity of illustration only and should not be construed to limit the scope of the invention. Those of skill in the art will appreciate that a variety of other sugar moieties can be activated and derivatized in a manner analogous to that set forth using sialic acid as an example. For example, numerous methods are available for modifying galactose, glucose, N-acetylgalactosamine and fucose to name a few sugar substrates, which are readily modified by art recognized methods. See, for example, Elhalabi et al., Curr. Med. Chem. 6: 93 (1999) and Schafer et al., J. Org. Chem. 65: 24 (2000).

In an exemplary embodiment, the polypeptide that is modified by a method of the invention is a glycopeptide that is produced in prokaryotic cells (e.g., E. coli), eukaryotic cells including yeast and mammalian cells (e.g., CHO cells), or in a transgenic animal and thus contains N- and/or O-linked oligosaccharide chains, which are incompletely sialylated. The oligosaccharide chains of the glycopeptide lacking a sialic acid and containing a terminal galactose residue can be glyco-PEG-ylated, glyco-PPG-ylated or otherwise modified with a modified sialic acid.

In Scheme 5, the amino glycoside 1, is treated with the active ester of a protected amino acid (e.g., glycine) derivative, converting the sugar amine residue into the corresponding protected amino acid amide adduct. The adduct is treated with an aldolase to form αhydroxy carboxylate 2. Compound 2 is converted to the corresponding CMP derivative by the action of CMP-SA synthetase, followed by catalytic hydrogenation of the CMP derivative to produce compound 3. The amine introduced via formation of the glycine adduct is utilized as a locus of PEG or PPG attachment by reacting compound 3 with an activated (m-) PEG or (m-) PPG derivative (e.g., PEG-C(O)NHS, PPG-C(O)NHS), producing 4 or 5, respectively.

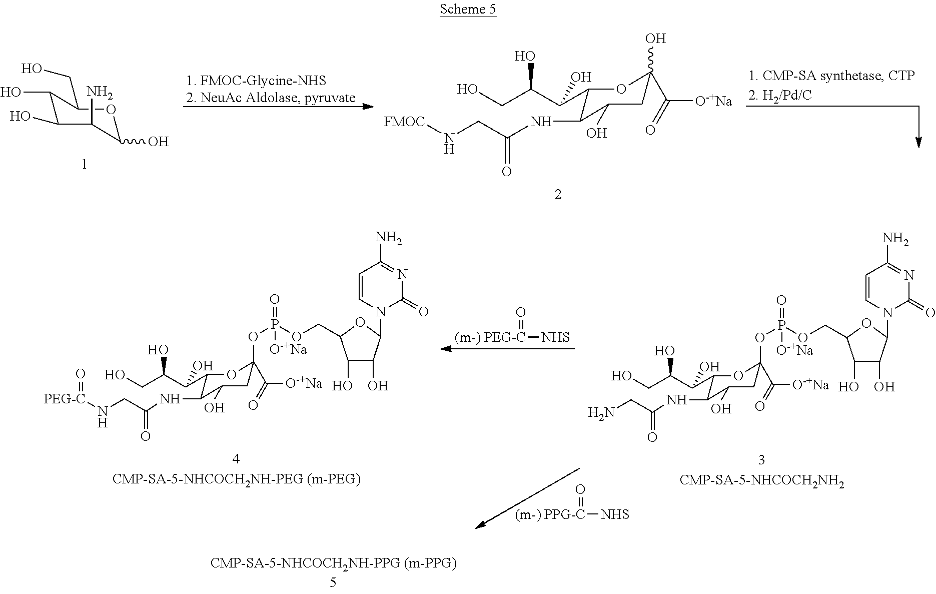

Table 11, below sets forth representative examples of sugar monophosphates that are derivatized with a PEG or PPG moiety. Certain of the compounds of Table 2 are prepared by the method of Scheme 4. Other derivatives are prepared by art-recognized methods. See, for example, Keppler et al., Glycobiology 11: 11R (2001); and Charter et al., Glycobiology 10: 1049 (2000)). Other amine reactive PEG and PPG analogues are commercially available, or they can be prepared by methods readily accessible to those of skill in the art.

TABLE 11
Examples of sugar monophosphates
derivatized with PEG or PPG

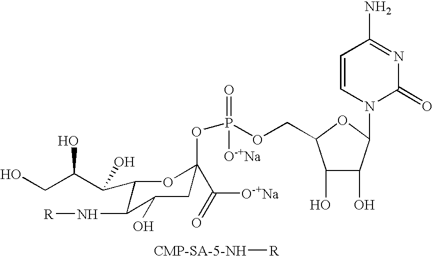

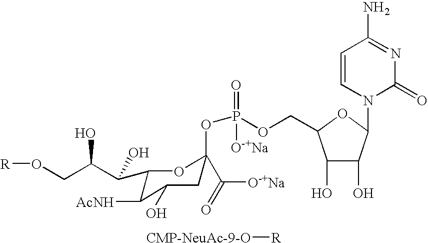

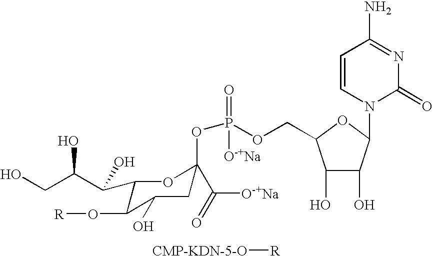

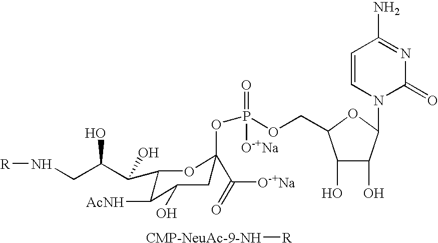

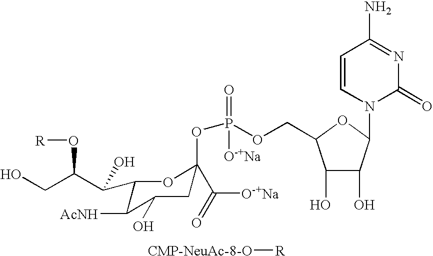

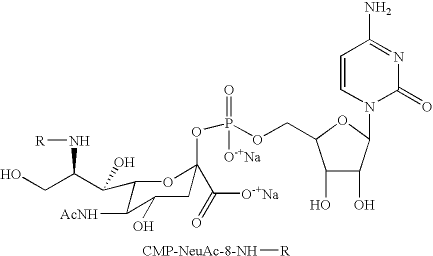

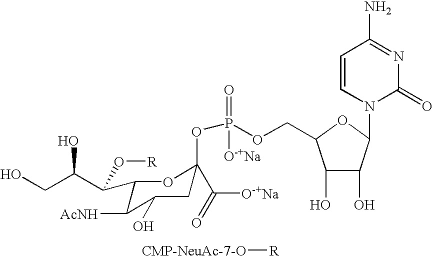

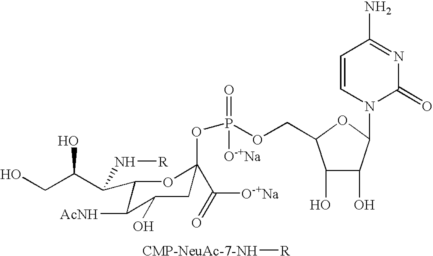

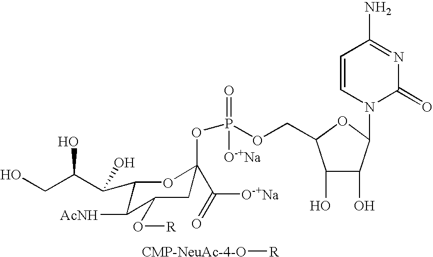

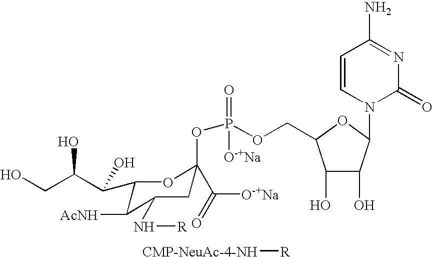

The modified sugar phosphates of use in practicing the present invention can be substituted in other positions as well as those set forth above. Presently preferred substitutions of sialic acid are set forth in Formula (VIII):

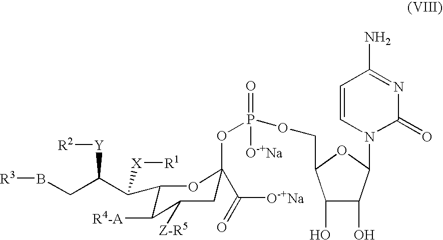

in which X is a linking group, which is preferably selected from —O—, —N(H)—, —S, CH₂—, and —N(R)₂, in which each R is a member independently selected from R¹-R⁵. The symbols Y, Z, A and B each represent a group that is selected from the group set forth above for the identity of X. X, Y, Z, A and B are each independently selected and, therefore, they can be the same or different. The symbols R¹, R², R³, R⁴ and R⁵ represent H, a water-soluble polymer, therapeutic moiety, biomolecule or other moiety. Alternatively, these symbols represent a linker that is bound to a water-soluble polymer, therapeutic moiety, biomolecule or other moiety.

Exemplary moieties attached to the conjugates disclosed herein include, but are not limited to, PEG derivatives (e.g., alkyl-PEG, acyl-PEG, acyl-alkyl-PEG, alkyl-acyl-PEG carbamoyl-PEG, aryl-PEG), PPG derivatives (e.g., alkyl-PPG, acyl-PPG, acyl-alkyl-PPG, alkyl-acyl-PPG carbamoyl-PPG, aryl-PPG), therapeutic moieties, diagnostic moieties, mannose-6-phosphate, heparin, heparan, SLeₓ, mannose, mannose-6-phosphate, Sialyl Lewis X, FGF, VFGF, proteins, chondroitin, keratan, dermatan, albumin, integrins, antennary oligosaccharides, peptides and the like. Methods of conjugating the various modifying groups to a saccharide moiety are readily accessible to those of skill in the art (POLY (ETHYLENE GLYCOL CHEMISTRY: BIOTECHNICAL AND BIOMEDICAL APPLICATIONS, J. Milton Harris, Ed., Plenum Pub. Corp., 1992; POLY (ETHYLENE GLYCOL) CHEMICAL AND BIOLOGICAL APPLICATIONS, J. Milton Harris, Ed., ACS Symposium Series No. 680, American Chemical Society, 1997; Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991).

An exemplary strategy involves incorporation of a protected sulfhydryl onto the sugar using the heterobifunctional crosslinker SPDP (n-succinimidyl-3-(2-pyridyldithio)propionate and then deprotecting the sulfhydryl for formation of a disulfide bond with another sulfhydryl on the modifying group.

If SPDP detrimentally affects the ability of the modified sugar to act as a glycosyltransferase substrate, one of an array of other crosslinkers such as 2-iminothiolane or N-succinimidyl S-acetylthioacetate (SATA) is used to form a disulfide bond. 2-iminothiolane reacts with primary amines, instantly incorporating an unprotected sulfhydryl onto the amine-containing molecule. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategy is exemplary, and not limiting, of linkers of use in the invention. Other crosslinkers are available that can be used in different strategies for crosslinking the modifying group to the polypeptide. For example, TPCH(S-(2-thiopyridyl)-L-cysteine hydrazide and TPMPH ((S-(2-thiopyridyl) mercapto-propionohydrazide) react with carbohydrate moieties that have been previously oxidized by mild periodate treatment, thus forming a hydrazone bond between the hydrazide portion of the crosslinker and the periodate generated aldehydes. TPCH and TPMPH introduce a 2-pyridylthione protected sulfhydryl group onto the sugar, which can be deprotected with DTT and then subsequently used for conjugation, such as forming disulfide bonds between components.

If disulfide bonding is found unsuitable for producing stable modified sugars, other crosslinkers may be used that incorporate more stable bonds between components. The heterobifunctional crosslinkers GMBS (N-gama-malimidobutyryloxy)succinimide) and SMCC (succinimidyl 4-(N-maleimido-methyl)cyclohexane) react with primary amines, thus introducing a maleimide group onto the component. The maleimide group can subsequently react with sulfhydryls on the other component, which can be introduced by previously mentioned crosslinkers, thus forming a stable thioether bond between the components. If steric hindrance between components interferes with either component's activity or the ability of the modified sugar to act as a glycosyltransferase substrate, crosslinkers can be used which introduce long spacer arms between components and include derivatives of some of the previously mentioned crosslinkers (i.e., SPDP). Thus, there is an abundance of suitable crosslinkers, which are useful; each of which is selected depending on the effects it has on optimal polypeptide conjugate and modified sugar production.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

### Preferred Specific Sites in Crosslinking Reagents

### 1. Amino-Reactive Groups

In one embodiment, the sites on the cross-linker are amino-reactive groups. Useful non-limiting examples of amino-reactive groups include N-hydroxysuccinimide (NHS) esters, imidoesters, isocyanates, acylhalides, arylazides, p-nitrophenyl esters, aldehydes, and sulfonyl chlorides.

NHS esters react preferentially with the primary (including aromatic) amino groups of a modified sugar component. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide. Thus, the positive charge of the original amino group is lost.

Imidoesters are the most specific acylating reagents for reaction with the amine groups of the modified sugar components. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained.

Isocyanates (and isothiocyanates) react with the primary amines of the modified sugar components to form stable bonds. Their reactions with sulfhydryl, imidazole, and tyrosyl groups give relatively unstable products.

Acylazides are also used as amino-specific reagents in which nucleophilic amines of the affinity component attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of modified sugar components, but also with sulfhydryl and imidazole groups.

p-Nitrophenyl esters of mono- and dicarboxylic acids are also useful amino-reactive groups. Although the reagent specificity is not very high, α- and ε-amino groups appear to react most rapidly.

Aldehydes such as glutaraldehyde react with primary amines of modified sugar. Although unstable Schiff bases are formed upon reaction of the amino groups with the aldehydes of the aldehydes, glutaraldehyde is capable of modifying the modified sugar with stable crosslinks. At pH 6-8, the pH of typical crosslinking conditions, the cyclic polymers undergo a dehydration to form α-β unsaturated aldehyde polymers. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product.

Aromatic sulfonyl chlorides react with a variety of sites of the modified sugar components, but reaction with the amino groups is the most important, resulting in a stable sulfonamide linkage.

### 2. Sulfhydryl-Reactive Groups

In another embodiment, the sites are sulfhydryl-reactive groups. Useful, non-limiting examples of sulfhydryl-reactive groups include maleimides, alkyl halides, pyridyl disulfides, and thiophthalimides.

Maleimides react preferentially with the sulfhydryl group of the modified sugar components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryls via disulfide exchange to give mixed disulfides. As a result, pyridyl disulfides are the most specific sulfhydryl-reactive groups.

Thiophthalimides react with free sulfhydryl groups to form disulfides.

### 3. Carboxyl-Reactive Residue

In another embodiment, carbodiimides soluble in both water and organic solvent, are used as carboxyl-reactive reagents. These compounds react with free carboxyl groups forming a pseudourea that can then couple to available amines yielding an amide linkage teach how to modify a carboxyl group with carbodiimde (Yamada et al., Biochemistry 20: 4836-4842, 1981).

### Preferred Nonspecific Sites in Crosslinking Reagents

In addition to the use of site-specific reactive moieties, the present invention contemplates the use of non-specific reactive groups to link the sugar to the modifying group.

Exemplary non-specific cross-linkers include photoactivatable groups, completely inert in the dark, which are converted to reactive species upon absorption of a photon of appropriate energy. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming crosslinks.

### Homobifunctional Reagents

1. Homobifunctional Crosslinkers Reactive with Primary Amines

Synthesis, properties, and applications of amine-reactive cross-linkers are commercially described in the literature (for reviews of crosslinking procedures and reagents, see above). Many reagents are available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxy-carbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidyl-propionate (DSP), and dithiobis(sulfosuccinimidylpropionate (sulfo-DSP). Preferred, non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-3′-(dimethylenedioxy)dipropionimidate (DDDP), dimethyl-3,3′-(tetramethylenedioxy)-dipropionimidate (DTDP), and dimethyl-3,3′-dithiobispropionimidate (DTBP).

Preferred, non-limiting examples of homobifunctional isothiocyanates include: p-phenylenediisothiocyanate (DITC), and 4,4′-diisothiocyano-2,2′-disulfonic acid stilbene (DIDS).

Preferred, non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate.

Preferred, non-limiting examples of homobifunctional arylhalides include 1,5-difluoro-2,4-dinitrobenzene (DFDNB), and 4,4′-difluoro-3,3′-dinitrophenyl-sulfone.

Preferred, non-limiting examples of homobifunctional aliphatic aldehyde reagents include glyoxal, malondialdehyde, and glutaraldehyde.

Preferred, non-limiting examples of homobifunctional acylating reagents include nitrophenyl esters of dicarboxylic acids.

Preferred, non-limiting examples of homobifunctional aromatic sulfonyl chlorides include phenol-2,4-disulfonyl chloride, and α-naphthol-2,4-disulfonyl chloride.

Preferred, non-limiting examples of additional amino-reactive homobifunctional reagents include erythritolbiscarbonate which reacts with amines to give biscarbamates.

2. Homobifunctional Crosslinkers Reactive with Free Sulfhydryl Groups

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional maleimides include bismaleimidohexane (BMH), N,N′-(1,3-phenylene) bismaleimide, N,N′-(1,2-phenylene)bismaleimide, azophenyldimaleimide, and bis(N-maleimidomethyl)ether.

Preferred, non-limiting examples of homobifunctional pyridyl disulfides include 1,4-di-3′-(2′-pyridyldithio)propionamidobutane (DPDPB).

Preferred, non-limiting examples of homobifunctional alkyl halides include 2,2′-dicarboxy-4,4′-diiodoacetamidoazobenzene, α,α′-diiodo-p-xylenesulfonic acid, α,α′-dibromo-p-xylenesulfonic acid, N,N′-bis(-bromoethyl)benzylamine, N,N′-di(bromoacetyl)phenylthydrazine, and 1,2-di(bromoacetyl)amino-3-phenylpropane.

### 3. Homobifunctional Photoactivatable Crosslinkers

Synthesis, properties, and applications of such reagents are described in the literature (for reviews of crosslinking procedures and reagents, see above). Some of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional photoactivatable crosslinker include bis-β-(4-azidosalicylamido)ethyldisulfide (BASED), di-N-(2-nitro-4-azidophenyl)-cystamine-S,S-dioxide (DNCO), and 4,4′-dithiobisphenylazide.

### HeteroBifunctional Reagents

1. Amino-Reactive HeteroBifunctional Reagents with a Pyridyl Disulfide Moiety

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-α-methyl-α-(2-pyridyldithio)toluene (SMPT), and sulfosuccinimidyl 6-α-methyl-α-(2-pyridyldithio)toluamidohexanoate (sulfo-LC-SMPT).

2. Amino-Reactive HeteroBifunctional Reagents with a Maleimide Moiety

Synthesis, properties, and applications of such reagents are described in the literature. Preferred, non-limiting examples of hetero-bifunctional reagents with a maleimide moiety and an amino-reactive NHS ester include succinimidyl maleimidylacetate (AMAS), succinimidyl 3-maleimidylpropionate (BMPS), N-γ-maleimidobutyryloxysuccinimide ester (GMBS)N-γ-maleimidobutyryloxysulfo succinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)-cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

3. Amino-Reactive HeteroBifunctional Reagents with an Alkyl Halide Moiety

Synthesis, properties, and applications of such reagents are described in the literature Preferred, non-limiting examples of hetero-bifunctional reagents with an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)-methyl)-cyclohexane-1-carbonyl)aminohexanoate (SIACX), and succinimidyl-4-((iodoacetyl)-amino)methylcyclohexane-1-carboxylate (SIAC).

An example of a hetero-bifunctional reagent with an amino-reactive NHS ester and an alkyl dihalide moiety is N-hydroxysuccinimidyl 2,3-dibromopropionate (SDBP). SDBP introduces intramolecular crosslinks to the affinity component by conjugating its amino groups. The reactivity of the dibromopropionyl moiety towards primary amine groups is controlled by the reaction temperature (McKenzie et al., Protein Chem. 7: 581-592 (1988)).

Preferred, non-limiting examples of hetero-bifunctional reagents with an alkyl halide moiety and an amino-reactive p-nitrophenyl ester moiety include p-nitrophenyl iodoacetate (NPIA).

Other cross-linking agents are known to those of skill in the art. See, for example, Pomato et al., U.S. Pat. No. 5,965,106. It is within the abilities of one of skill in the art to choose an appropriate cross-linking agent for a particular application.

### Cleavable Linker Groups

In yet a further embodiment, the linker group is provided with a group that can be cleaved to release the modifying group from the sugar residue. Many cleaveable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups is commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

Specific embodiments of reactive PEG reagents according to the invention include:

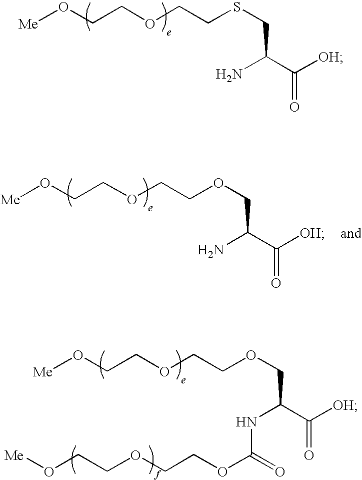

and carbonates and active esters of these species, such as:

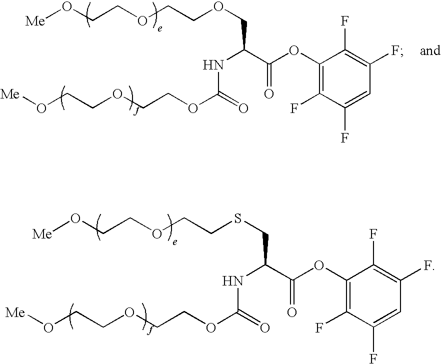

### Nucleic Acids

In another aspect, the invention provides an isolated nucleic acid encoding a polypeptide of the invention. The polypeptide includes within its amino acid sequence one or more exogenous N-linked glycosylation sequence of the invention. In one embodiment, the nucleic acid of the invention is part of an expression vector. In another related embodiment, the present invention provides a cell including the nucleic acid of the present invention. Exemplary cells include host cells such as various strains of E. coli, insect cells, yeast cells and mammalian cells, such as CHO cells.

### Pharmaceutical Compositions

Polypeptides conjugates of the invention have a broad range of pharmaceutical applications. For example, glycoconjugated erythropoietin (EPO) may be used for treating general anemia, aplastic anemia, chemo-induced injury (such as injury to bone marrow), chronic renal failure, nephritis, and thalassemia. Modified EPO may be further used for treating neurological disorders such as brain/spine injury, multiple sclerosis, and Alzheimer's disease.

A second example is interferon-α (IFN-α), which may be used for treating AIDS and hepatitis B or C, viral infections caused by a variety of viruses such as human papilloma virus (HBV), coronavirus, human immunodeficiency virus (HIV), herpes simplex virus (HSV), and varicella-zoster virus (VZV), cancers such as hairy cell leukemia, AIDS-related Kaposi's sarcoma, malignant melanoma, follicular non-Hodgkins lymphoma, Philladephia chromosome (Ph)-positive, chronic phase myelogenous leukemia (CML), renal cancer, myeloma, chronic myelogenous leukemia, cancers of the head and neck, bone cancers, as well as cervical dysplasia and disorders of the central nervous system (CNS) such as multiple sclerosis. In addition, IFN-α modified according to the methods of the present invention is useful for treating an assortment of other diseases and conditions such as Sjogren's syndrome (an autoimmune disease), Behcet's disease (an autoimmune inflammatory disease), fibromyalgia (a musculoskeletal pain/fatigue disorder), aphthous ulcer (canker sores), chronic fatigue syndrome, and pulmonary fibrosis.

Another example is interferon-β, which is useful for treating CNS disorders such as multiple sclerosis (either relapsing/remitting or chronic progressive), AIDS and hepatitis B or C, viral infections caused by a variety of viruses such as human papilloma virus (HBV), human immunodeficiency virus (HIV), herpes simplex virus (HSV), and varicella-zoster virus (VZV), otological infections, musculoskeletal infections, as well as cancers including breast cancer, brain cancer, colorectal cancer, non-small cell lung cancer, head and neck cancer, basal cell cancer, cervical dysplasia, melanoma, skin cancer, and liver cancer. IFN-β modified according to the methods of the present invention is also used in treating other diseases and conditions such as transplant rejection (e.g., bone marrow transplant), Huntington's chorea, colitis, brain inflammation, pulmonary fibrosis, macular degeneration, hepatic cirrhosis, and keratoconjunctivitis.

Granulocyte colony stimulating factor (G-CSF) is a further example. G-CSF modified according to the methods of the present invention may be used as an adjunct in chemotherapy for treating cancers, and to prevent or alleviate conditions or complications associated with certain medical procedures, e.g., chemo-induced bone marrow injury; leucopenia (general); chemo-induced febrile neutropenia; neutropenia associated with bone marrow transplants; and severe, chronic neutropenia. Modified G-CSF may also be used for transplantation; peripheral blood cell mobilization; mobilization of peripheral blood progenitor cells for collection in patients who will receive myeloablative or myelosuppressive chemotherapy; and reduction in duration of neutropenia, fever, antibiotic use, hospitalization following induction/consolidation treatment for acute myeloid leukemia (AML). Other conditions or disorders may be treated with modified G-CSF include asthma and allergic rhinitis.

As one additional example, human growth hormone (hGH) modified according to the methods of the present invention may be used to treat growth-related conditions such as dwarfism, short-stature in children and adults, cachexia/muscle wasting, general muscular atrophy, and sex chromosome abnormality (e.g., Turner's Syndrome). Other conditions may be treated using modified hGH include: short-bowel syndrome, lipodystrophy, osteoporosis, uraemaia, burns, female infertility, bone regeneration, general diabetes, type II diabetes, osteo-arthritis, chronic obstructive pulmonary disease (COPD), and insomia. Moreover, modified hGH may also be used to promote various processes, e.g., general tissue regeneration, bone regeneration, and wound healing, or as a vaccine adjunct.

Thus, in another aspect, the invention provides a pharmaceutical composition including at least one polypeptide or polypeptide conjugate of the invention and a pharmaceutically acceptable carrier. Pharmaceutically acceptable carrier includes diluents, vehicles, additives and combinations thereof. In an exemplary embodiment, the pharmaceutical composition includes a covalent conjugate between a water-soluble polymer (e.g., a non-naturally-occurring water-soluble polymer), and a glycosylated or non-glycosylated polypeptide of the invention as well as a pharmaceutically acceptable diluent.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable matrices, such as microspheres (e.g., polylactate polyglycolate), may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered subcutaneously or parenterally, e.g., intravenously. Thus, the invention provides compositions for parenteral administration, which include the compound dissolved or suspended in an acceptable carrier, preferably an aqueous carrier, e.g., water, buffered water, saline, PBS and the like. The compositions may also contain detergents such as Tween 20 and Tween 80; stabilizers such as mannitol, sorbitol, sucrose, and trehalose; and preservatives such as EDTA and meta-cresol. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the glycopeptides of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized glycopeptides of the invention.

Targeting mechanisms generally require that the targeting agents be positioned on the surface of the liposome in such a manner that the target moieties are available for interaction with the target, for example, a cell surface receptor. The carbohydrates of the invention may be attached to a lipid molecule before the liposome is formed using methods known to those of skill in the art (e.g., alkylation or acylation of a hydroxyl group present on the carbohydrate with a long chain alkyl halide or with a fatty acid, respectively). Alternatively, the liposome may be fashioned in such a way that a connector portion is first incorporated into the membrane at the time of forming the membrane. The connector portion must have a lipophilic portion, which is firmly embedded and anchored in the membrane. It must also have a reactive portion, which is chemically available on the aqueous surface of the liposome. The reactive portion is selected so that it will be chemically suitable to form a stable chemical bond with the targeting agent or carbohydrate, which is added later. In some cases it is possible to attach the target agent to the connector molecule directly, but in most instances it is more suitable to use a third molecule to act as a chemical bridge, thus linking the connector molecule which is in the membrane with the target agent or carbohydrate which is extended, three dimensionally, off of the vesicle surface.

The compounds prepared by the methods of the invention may also find use as diagnostic reagents. For example, labeled compounds can be used to locate areas of inflammation or tumor metastasis in a patient suspected of having an inflammation. For this use, the compounds can be labeled with a detectable isotope, such as ¹²⁵I, ¹⁴C, or tritium. In another example, the compound is labeled with a luminescent moiety, such as a lanthanide complex.

### Exemplary Conjugates of the Invention

In one embodiment, the conjugates of the invention include a moiety selected from:

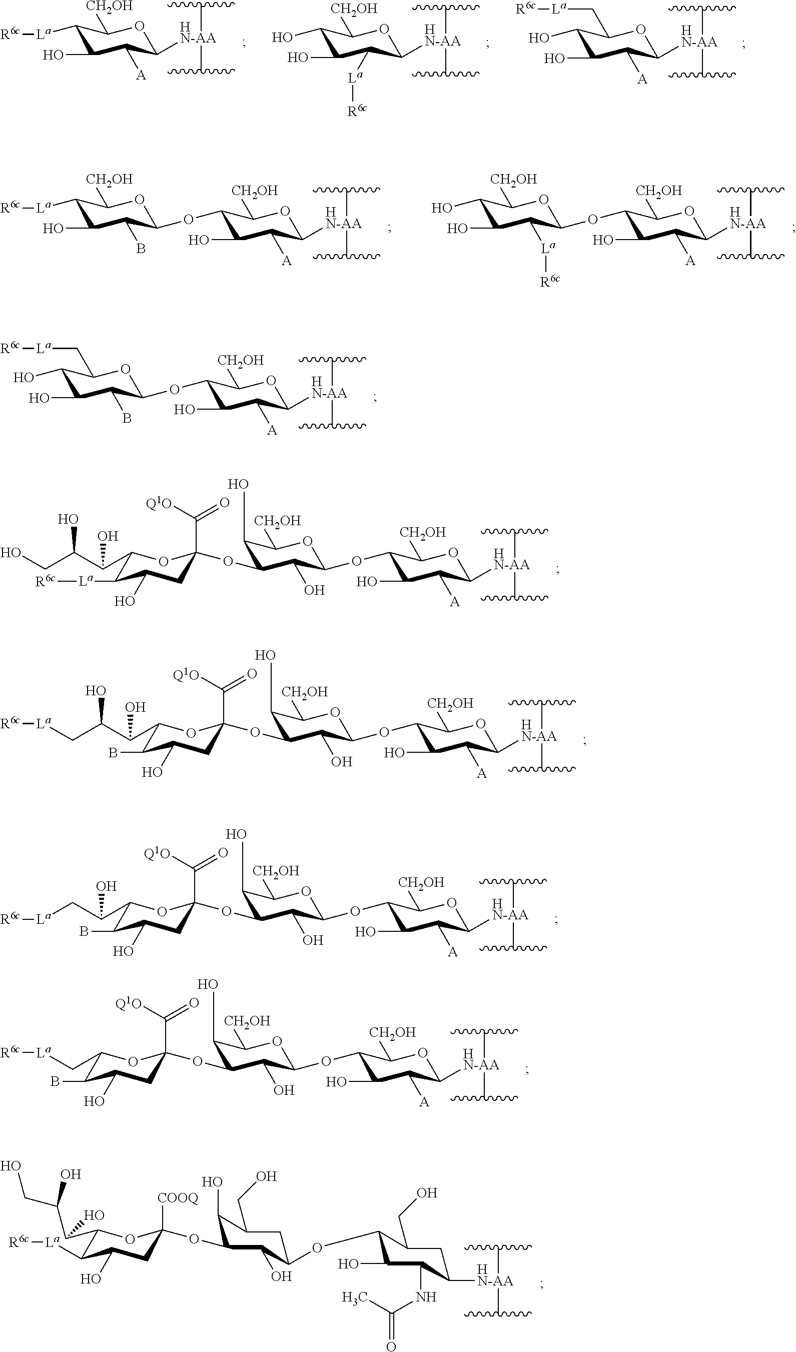

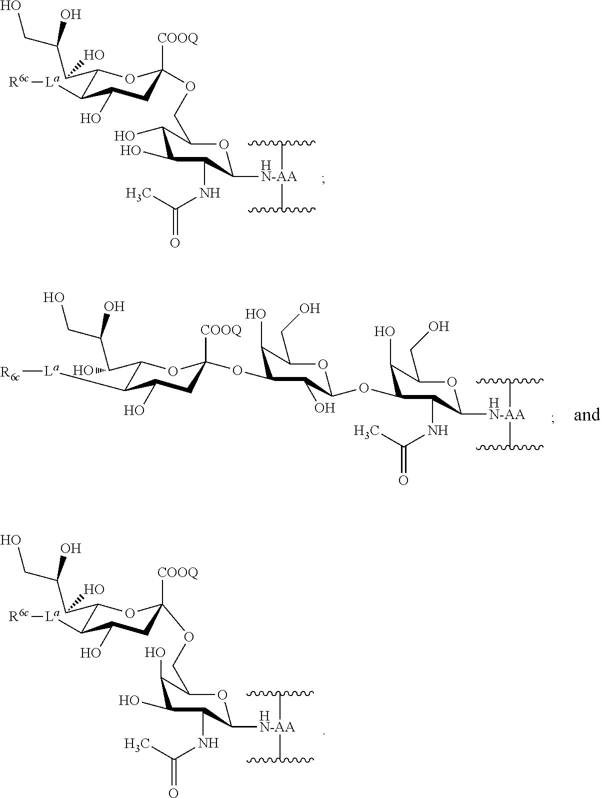

wherein AA is derived from an amino acid residue that includes an amino group. This amino acid residue is part of a polypeptide. In one example, AA is derived from an asparagine residue. Q, L^{a} and R^{6c} are as defined herein above. Q¹ is H, a single negative charge or a cation (e.g., Na⁺ or K⁺). A and B are members independently selected from OR (e.g., OH) and NHCOR (e.g., NHAc).

In one example according to any of the above embodiments, the conjugates include a moiety selected from:

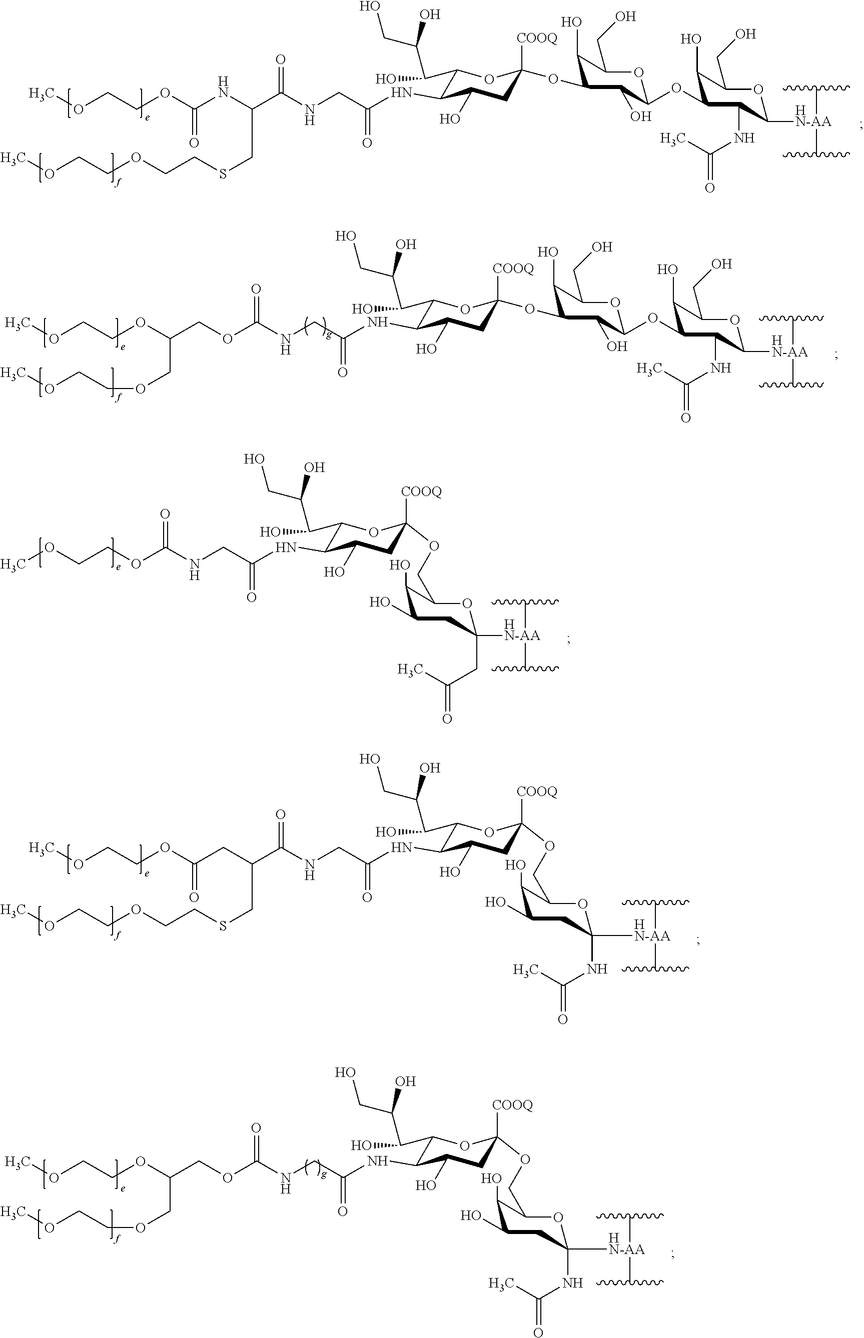

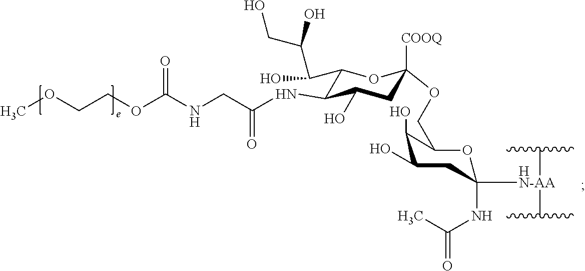

### V. Methods

### Generation of Polypeptides

Methods of generating polypeptides (e.g., through recombinant technology) are known in the art. Exemplary methods are described herein. An exemplary method includes: (i) generating an expression vector including a nucleic acid sequence encoding a polypeptide having an exogenous N-linked glycosylation sequence. The method may further include: (ii) transfecting a host cell with the expression vector. The method can further include: (iii) expressing the polypeptide in a host cell. The method may further include: (iv) isolating the polypeptide. The method may further include: (v) enzymatically glycosylating the polypeptide at the N-linked glycosylation sequence, for example using an endogenous or recombinant oligosaccharyl transferase. Exemplary glycosyl transferases, such as the bacterial PglB are described herein.

### Formation of Polypeptide Conjugates

In another aspect, the invention provides methods of forming a covalent conjugate between a modifying group and a polypeptide. The polypeptide conjugates of the invention are formed between glycosylated or non-glycosylated polypeptides and diverse species such as water-soluble polymers, therapeutic moieties, biomolecules, diagnostic moieties, targeting moieties and the like. The polymer, therapeutic moiety or biomolecule is conjugated to the polypeptide via a glycosyl linking group, which is interposed between, and covalently linked to both the polypeptide and the modifying group (e.g. water-soluble polymer).

### Cell-Free In Vitro Glycosylation of Polypeptides

In one embodiment, glycosylation and/or glycoPEGylation of the polypeptide is performed in vitro. For example, the polypeptide is synthesized or expressed in a host cell and optionally purified. The polypeptide is then subjected to glycosylation or glycoPEGylation involving a glycosyl donor species of the invention (e.g., an undecaprenyl-pyrophosphate-linked glycosyl moiety) as well as a suitable oligosaccharyl transferase.

In one embodiment, the polypeptide is covalently linked to a modifying group by contacting the polypeptide with a glycosyl donor species, wherein at least one glycosyl donor moiety of the glycosyl donor species is covalently linked to a modifying group, in the presence of an oligosaccharyl transferase for which the glycosyl donor species is a substrate. Hence, in an exemplary embodiment, the invention provides a cell-free in vitro method of forming a covalent conjugate between a polypeptide and a modifying group (e.g., a polymeric modifying group). In this method, the polypeptide includes an N-linked glycosylation sequence of the invention that includes an asparagine residue. The modifying group is covalently linked to the polypeptide at the asparagine residue via a glycosyl linking group that is interposed between and covalently linked to both the polypeptide and the modifying group. The method includes: contacting the polypeptide and a glycosyl donor species of the invention, in the presence of an oligosaccharyltransferase under conditions sufficient for the oligosaccharyltransferase to transfer the glycosyl moiety from the glycosyl donor species onto the asparagine residue of the N-linked glycosylation sequence. The method may further include: generating the polypeptide, e.g., through recombinant technology or chemical synthesis. Methods for generating polypeptides are described herein. The method may further include: isolating the covalent conjugate. In one embodiment, the polypeptide corresponds to a parent polypeptide that is a therapeutic polyeptide. Exemplary parent polypeptides are described herein.

Glycosylation within a Host Cell

Glycosylation of a polypeptide that includes a N-linked glycosylation sequence of the invention can also occur within a host cell, in which the polypeptide is expressed. In one embodiment, the host cell is contacted with and internalizes a suitable glycosyl donor species of the invention. For example, the glycosyl donor species is added to the cell culture medium, which is used to culture the host cell. An oligosaccharyl transferase within the host cell uses the internalized glycosyl donor species as a substrate and transfers a glycosyl moiety onto the expressed polypeptide. In one embodiment, this intracellular glycosylation is used to covalently link a modifying group to a polypeptide by contacting the host cell with a glycosyl donor species that includes a glycosyl moiety derivatized with a modifying group. Accordingly, the current invention provides a method of forming a covalent conjugate between a polypeptide and a modifying group (e.g., a polymeric modifying group), wherein the polypeptide includes a N-linked glycosylation sequence that includes an asparagine residue. The modifying group is covalently linked to the polypeptide at the asparagine residue via a glycosyl linking group that is interposed between and covalently linked to both the polypeptide and the modifying group. The method includes: (i) contacting the polypeptide and a glycosyl donor species of the invention in the presence of an oligosaccharyl transferase under conditions sufficient for the oligosaccharyl transferase to transfer a glycosyl moiety that is covalently linked to the modifying group from the glycosyl donor species onto the asparagine residue of the N-linked glycosylation sequence, wherein the contacting occurs within a host cell, in which the polypeptide is expressed. The method may further include (ii) contacting the host cell with a glycosyl donor species of the invention. The method may further include (iii) incubating the host cell under conditions sufficient for the host cell to internalize the glycosyl donor species. The method may further include (iii) generating the polypeptide, e.g., through recombinant technology or chemical synthesis. Methods for generating polypeptides are described herein. The method may further include (iv) isolating the covalent conjugate. In one embodiment, the polypeptide corresponds to a parent polypeptide that is a therapeutic polyeptide. Exemplary parent polypeptides are described herein.

In one example, the host cell includes an endogenous oligosaccharyl transferase which is capable of using the internalized glycosyl donor species as a substrate and can intracellularly transfers the glycosyl moiety of the glycosyl donor species onto a polypeptide.

In another exemplary embodiment, the oligosaccharyl transferase is a recombinant enzyme and is co-expressed in the host cell together with the polypeptide. Intracellular glycosylation is then accomplished by co-expressing an oligosaccharyl transferase that can use the expressed polypeptide as a substrate. The enzyme is capable of glycosylating the polypeptide at the glycosylation sequence intracellularly using the internalized glycosyl donor species as the glycosyl substrate.

The host cell can be any cell suitable for expression of the polypeptide. In one embodiment, the host cell is a bacterial cell. In another embodiment, the host cell is a eukaryotic cell, such as a yeast cell, an insect cell or a mammalian cell.

Methods are available to determine whether or not a polypeptide is efficiently glycosylated. For example, the cell lysate (after one or more sample preparation step) is analyzed by mass spectroscopy to measure the ratio between glycosylated and non-glycosylated polypeptides. In another example, the cell lysate is analyzed by gel electrophoresis separating glycosylated from non-glycosylated polypeptides.

In another exemplary embodiment, the microorganism in which the polypeptide is expressed has an intracelluar oxidizing environment. The microorganism may be genetically modified to have the intracellular oxidizing environment. Intracellular glycosylation is not limited to the transfer of a single glycosyl residue. Several glycosyl residues can be added sequentially by co-expression of required enzymes and the presence of respective glycosyl donors. This approach can also be used to produce polypeptides on a commercial scale. An exemplary technology is described in U.S. Provisional Patent Application No. 60/842,926 filed on Sep. 6, 2006, which is incorporated herein by reference in its entirety. The host cell may be a prokaryotic microorganism, such as E. coli or Pseudomonas strains). In an exemplary embodiment, the host cell is a trxB gor supp mutant E. coli cell.

### Identification of Sequon Polypeptides as Substrates for Oligosaccharyl Transferases

One strategy for the identification of sequon polypeptides that can be glycosylated with a satisfactory yield when subjected to a glycosylation reaction using an enzyme and a glycosyl donor species, is to prepare a library of sequon polypeptides, wherein each sequon polypeptide includes at least one exogenous N-linked glycosylation sequence of the invention, and to test each sequon polypeptide for its ability to function as an efficient substrate for an oligosaccharyl transferase. A library of sequon polypeptides can be generated by including a selected N-linked glycosylation sequence of the invention at different positions within the amino acid sequence of a parent polypeptide.

### Library of Sequon Polypeptides

In one aspect, the invention provides methods of generating one or more library of sequon polypeptides, wherein the sequon polypeptides corresponds to a parent polypeptide (e.g., wild-type polypeptide). In one embodiment, the parent polypeptide has an amino acid sequence including m amino acids. An exemplary method of generating a library of sequon polypeptides includes the steps of: (i) producing a first sequon polypeptide (e.g., recombinantly, chemically or by other means) by introducing an N-linked glycosylation sequence of the invention at a first amino acid position (AA)ₙ within the parent polypeptide, wherein n is a member selected from 1 to m; (ii) producing at least one additional sequon polypeptide by introducing an N-linked glycosylation sequence at an additional amino acid position. In one embodiment, the additional amino acid position is (AA)ₙ₊ₓ, for example(AA)ₙ₊₁. In another embodiment, the additional amino acid position is (AA)ₙ₋ₓ, for example (AA)ₙ₋₁. In these embodiments, x is a member selected from 1 to (m-n). In one embodiment the additional sequon polypeptide includes the same N-linked glycosylation sequence as the first sequon polypeptide. In another embodiment, the additional sequon polypeptide includes a different N-linked glycosylation sequence than the first sequon polypeptide. In an exemplary embodiment, the library of sequon polypeptides is generated by “sequon scanning” described herein above. Exemplary parent polypeptides and N-linked glycosylation sequences useful in the libraries of the invention are also described herein.

### Identification of Lead Polypeptides

It may be desirable to select among the members of the library those polypeptides that are effectively glycosylated and/or glycoPEGylated when subjected to an enzymatic glycosylation and/or glycoPEGylation reaction. Sequon polypeptides, which are found to be effectively glycosylated and/or glycoPEGylated are termed “lead polypeptides”. In an exemplary embodiment, the yield of the enzymatic glycosylation or glycoPEGylation reaction is used to select one or more lead polypeptides. In another exemplary embodiment, the yield of the enzymatic glycosylation or glycoPEGylation for a lead polypeptide is between about 10% and about 100%, preferably between about 30% and about 100%, more preferably between about 50% and about 100% and most preferably between about 70% and about 100%. When the polypeptide includes more than one N-linked glycosylation sequence, then the yield is determined separately for each N-linked glycosylation sequence. Lead polypeptides that can be efficiently glycosylated are optionally further evaluated, e.g., by subjecting the glycosylated lead polypeptide to another enzymatic glycosylation or glycoPEGylation reaction.

Thus, the invention provides methods for identifying a lead polypeptide. An exemplary method includes the steps of: (i) generating a library of sequon polypeptides of the invention; (ii) subjecting at least one member of the library to an enzymatic glycosylation reaction (or optionally an enzymatic glycoPEGylation reaction). In one embodiment, during this reaction, a glycosyl moiety is transferred from a glycosyl donor molecule onto at least one N-linked glycosylation sequence, wherein the glycosyl moiety is optionally derivatized with a modifying group. The method may further include: (iii) measuring the yield for the enzymatic glycosylation or glycoPEGylation reaction for at least one member of the library. The measuring can be accomplished using any method known in the art and those described herein below. The method may further include prior to step (ii): (iv) purifying at least one member of the library.

The transferred glycosyl moiety of step (ii) can be any glycosyl moiety including mono- and oligosaccharides as well as glycosyl-mimetic groups, which are optionally derivatized with a modifying group, such as a water-soluble polymeric modifying group. In an exemplary embodiment, the glycosyl moiety, which is added to the sequon polypeptide in an initial glycosylation reaction, is a GlcNAc moiety, a GalNAc moiety, a GlcNAc-GlcNAc moiety or a 6-hydroxy-bacilloseamine moiety. Subsequent glycosylation reactions can optionally be employed to add at least one additional glycosyl residues (e.g, a modified Sia moiety) to the resulting glycosylated polypeptide. The modifying group can be any modifying group of the invention, including water soluble polymers such as mPEG. In one embodiment, the enzymatic glycosylation reaction of step (ii) occurs in a host cell, in which the polypeptide is expressed. The method may further include (v): subjecting the product of step (ii) to a PEGylation reaction. In one embodiment, step (ii) and step (v) are performed in the same reaction vessel. In one embodiment, the PEGylation reaction is an enzymatic glycoPEGylation reaction. In another embodiment, the PEGylation reaction is a chemical PEGylation reaction. The method may further include: (vi) measuring the yield for the PEGylation reaction. Methods useful for measuring the yield of the PEGylation reaction are described below. The method may further include: (vii) generating an expression vector including a nucleic acid sequence encoding the sequon polypeptide. The method may further include: (viii): transfecting a host cell with the expression vector.

In an exemplary embodiment, each member of a library of sequon polypeptides is subjected to an enzymatic glycosylation reaction. For example, each sequon polypeptide is separately subjected to a glycosylation reaction and the yield of the glycosylation reaction is determined for one or more selected reaction condition.

In an exemplary embodiment, one or more sequon polypeptide of the library is purified prior to further processing, such as glycosylation and/or glycoPEGylation.

In another example, groups of sequon polypeptides can be combined and the resulting mixture of sequon polypeptides can be subjected to a glycosylation or glycoPEGylation reaction. In one exemplary embodiment, a mixture containing all members of the library is subjected to a glycosylation reaction. In one example, according to this embodiment, the glycosyl donor reagent can be added to the glycosylation reaction mixture in a less than stoichiometric amount (with respect to glycosylation sites present) creating an environment in which the sequon polypeptides compete as substrates for the enzyme. Those sequon polypeptides, which are substrates for the enzyme, can then be identified, for instance by virtue of mass spectral analysis with or without prior separation or purification of the glycosylated mixture. This same approach may be used for a group of sequon polypeptides which each contain a different O-linked glycosylation sequences of the invention.

The yield for the enzymatic glycosylation reaction, enzymatic glycoPEGylation reaction or chemical glycoPEGylation reaction can be determined using any suitable method known in the art. In an exemplary embodiment, the method used to distinguish between a glycosylated or glycoPEGylated polypeptide and an unreacted (e.g., non-glycosylated or glycoPEGylated) polypeptide is determined using a technique involving mass spectroscopy (e.g., LC-MS, MALDI-TOF). In another exemplary embodiment, the yield is determined using a technique involving gel electrophoresis. In yet another exemplary embodiment, the yield is determined using a technique involving nuclear magnetic resonace (NMR). In a further exemplary embodiment, the yield is determined using a technique involving chromatography, such as HPLC or GC. In one embodiment a multi-well plate (e.g., a 96-well plate) is used to carry out a number of glycosylation reactions in parallel. The plate may optionally be equipped with a separation or filtration medium (e.g., gel-filtration membrane) in the bottom of each well. Spinning may be used to pre-condition each sample prior to analysis by mass spectroscopy or other means.

A sequon polypeptide of interest (e.g., a selected lead polypeptide) can be expressed on an industrial scale (e.g., leading to the isolation of more than 250 mg, preferably more than 500 mg of protein).

### Further Evaluation of Lead Polypeptides

In one embodiment, in which the initial screening procedure involves enzymatic glycosylation using an unmodified glycosyl moiety (e.g., transfer of a GlcNAc moiety), selected lead polypeptides may be further evaluated for their capability of being an efficient substrate for further modification, e.g., through another enzymatic reaction or a chemical modification. In an exemplary embodiment, subsequent “screening” involves subjecting a glycosylated lead polypeptide to another glycosylation- and/or PEGylation reaction.

A PEGylation reaction can, for instance, be a chemical PEGylation reaction or an enzymatic glycoPEGylation reaction. In order to identify a lead polypeptide, which is efficiently glycoPEGylated, at least one lead polypeptide (optionally previously glycosylated) is subjected to a PEGylation reaction and the yield for this reaction is determined. In one example, PEGylation yields for each lead polypeptide are determined. In an exemplary embodiment, the yield for the PEGylation reaction is between about 10% and about 100%, preferably between about 30% and about 100%, more preferably between about 50% and about 100% and most preferably between about 70% and about 100%. The PEGylation yield can be determined using any analyical method known in the art, which is suitable for polypeptide analysis, such as mass spectroscopy (e.g., MALDI-TOF, Q-TOF), gel electrophoresis (e.g., in combination with means for quantification, such as densitometry), NMR techniques as well as chromatographic methods, such as HPLC using appropriate column materials useful for the separation of PEGylated and non-PEGylated species of the analyzed polypeptide. As described above for glycosylation, a multi-well plate (e.g., a 96-well plate) can be used to carry out a number of PEGylation reactions in parallel. The plate may optionally be equipped with a separation or filtration medium (e.g., gel-filtration membrane) in the bottom of each well. Spinning and reconstitution may be used to pre-condition each sample prior to analysis by mass spectroscopy or other means.

In another exemplary embodiment, glycosylation and glycoPEGylation of a sequon polypeptide occur in a “one pot reaction” as described below. In one example, the sequon polypeptide is contacted with a first enzyme (e.g., GalNAc-T2) and an appropriate donor molecule (e.g., UDP-GalNAc). The mixture is incubated for a suitable amount of time before a second enzyme (e.g., Core-1-GalT1) and a second glycosyl donor (e.g., UDP-Gal) are added. Any number of additional glycosylation/glycoPEGylation reactions can be performed in this manner. Alternatively, more than one enzyme and more than one glycosyl donor can be contacted with the mutant polypeptide to add more than one glycosyl residue in one reaction step. For example, the mutant polypeptide is contacted with 3 different enzymes (e.g., GalNAc-T2, Core-1-GalT1 and ST3Gal1) and three different glycosyl donor moieties (e.g, UDP-GalNAc, UDP-Gal and CMP-SA-PEG) in a suitable buffer system to generate a glycoPEGylated mutant polypeptide, such as polypeptide-GalNAc-Gal-SA-PEG (see, Example 4.6). Overall yields can be determined using the methods described above.

### Removal Glycosyl Moieties

The present invention also provides means of adding (or removing) one or more selected glycosyl residues to a polypeptide, after which a modified sugar is conjugated to at least one of the selected glycosyl residues of the polypeptide. The present embodiment is useful, for example, when it is desired to conjugate the modified sugar to a selected glycosyl residue that is either not present on a polypeptide or is not present in a desired amount. Thus, prior to coupling a modified sugar to a polypeptide, the selected glycosyl residue is conjugated to the polypeptide by enzymatic or chemical coupling. In another embodiment, the glycosylation pattern of a glycopeptide is altered prior to the conjugation of the modified sugar by the removal of a carbohydrate residue from the glycopeptide. See, for example WO 98/31826.

Addition or removal of any carbohydrate moieties present on the glycopeptide is accomplished either chemically or enzymatically. Chemical deglycosylation is preferably brought about by exposure of the polypeptide to trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., Arch. Biochem. Biophys. 259: 52 (1987) and by Edge et al., Anal. Biochem. 118: 131 (1981). Enzymatic cleavage of carbohydrate moieties on polypeptide variants can be achieved by the use of a variety of endo- and exo-glycosidases as described by Thotakura et al., Meth. Enzymol. 138: 350 (1987).

Chemical addition of glycosyl moieties is carried out by any art-recognized method. Enzymatic addition of sugar moieties is preferably achieved using a modification of the methods set forth herein, substituting native glycosyl units for the modified sugars used in the invention. Other methods of adding sugar moieties are disclosed in U.S. Pat. Nos. 5,876,980; 6,030,815; 5,728,554 and 5,922,577. Exemplary methods of use in the present invention are described in WO 87/05330 published Sep. 11, 1987, and in Aplin and Wriston, CRC CRIT. REV. BIOCHEM., pp. 259-306 (1981).

### Polypeptide Conjugates Including Two or More Polypeptides

Also provided are conjugates that include two or more polypeptides linked together through a linker arm, i.e., multifunctional conjugates; at least one polypeptide being N-glycosylated or including an exogenous N-linked glycosylation sequence. The multi-functional conjugates of the invention can include two or more copies of the same polypeptide or a collection of diverse polypeptides with different structures, and/or properties. In exemplary conjugates according to this embodiment, the linker between the two polypeptides is attached to at least one of the polypeptides through an N-linked glycosyl residue, such as an N-linked glycosyl intact glycosyl linking group.

In one embodiment, the invention provides a method for linking two or more polypeptides through a linking group. The linking group is of any useful structure and may be selected from straight- and branched-chain structures. Preferably, each terminus of the linker, which is attached to a polypeptide, includes a modified sugar (i.e., a nascent intact glycosyl linking group). In one embodiment, linkage of two polypeptides is accomplished by using a glycosyl donor species that is modified with a polypeptide.

### Enzymatic Conjugation of Modified Sugars to Polypeptides

The modified sugars are conjugated to a glycosylated polypeptide using an appropriate enzyme to mediate the conjugation. Preferably, the concentrations of the modified donor sugar(s), enzyme(s) and acceptor polypeptide(s) are selected such that glycosylation proceeds until the acceptor is consumed. The considerations discussed below, while set forth in the context of a sialyltransferase, are generally applicable to other glycosyltransferase reactions.

A number of methods of using glycosyltransferases to synthesize desired oligosaccharide structures are known and are generally applicable to the instant invention. Exemplary methods are described, for instance, in WO 96/32491 and Ito et al., Pure Appl. Chem. 65: 753 (1993), as well as U.S. Pat. Nos. 5,352,670; 5,374,541 and 5,545,553.

The present invention is practiced using a single glycosyltransferase or a combination of glycosyltransferases. For example, one can use a combination of a sialyltransferase and a galactosyltransferase. In those embodiments using more than one enzyme, the enzymes and substrates are preferably combined in an initial reaction mixture, or the enzymes and reagents for a second enzymatic reaction are added to the reaction medium once the first enzymatic reaction is complete or nearly complete. By conducting two enzymatic reactions in sequence in a single vessel, overall yields are improved over procedures in which an intermediate species is isolated. Moreover, cleanup and disposal of extra solvents and by-products is reduced.

The O-linked glycosyl moieties of the conjugates of the invention are generally originate with a GalNAc moiety that is attached to the polypeptide. Any member of the family of GalNAc transferases (e.g., those described herein in Table 13) can be used to bind a GalNAc moiety to the polypeptide (see e.g., Hassan H, Bennett E P, Mandel U, Hollingsworth M A, and Clausen H (2000); and Control of Mucin-Type O-Glycosylation: O-Glycan Occupancy is Directed by Substrate Specificities of Polypeptide GalNAc-Transferases; Eds. Ernst, Hart, and Sinay; Wiley-VCH chapter “Carbohydrates in Chemistry and Biology—a Comprehension Handbook”, 273-292). The GalNAc moiety itself can be the glycosyl linking group and derivatized with a modifying group. Alternatively, the saccharyl residue is built out using one or more enzyme and one or more appropriate glycosyl donor substrate. The modified sugar may then be added to the extended glycosyl moiety.

The enzyme catalyzes the reaction, usually by a synthesis step that is analogous to the reverse reaction of the endoglycanase hydrolysis step. In these embodiments, the glycosyl donor molecule (e.g., a desired oligo- or mono-saccharide structure) contains a leaving group and the reaction proceeds with the addition of the donor molecule to a GlcNAc residue on the protein. For example, the leaving group can be a halogen, such as fluoride. In other embodiments, the leaving group is a Asn, or a Asn-peptide moiety. In yet further embodiments, the GlcNAc residue on the glycosyl donor molecule is modified. For example, the GlcNAc residue may comprise a 1,2 oxazoline moiety.

In another embodiment, each of the enzymes utilized to produce a conjugate of the invention are present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

The temperature at which an above process is carried out can range from just above freezing to the temperature at which the most sensitive enzyme denatures. Preferred temperature ranges are about 0° C. to about 55° C., and more preferably about 20° C. to about 32° C. In another exemplary embodiment, one or more components of the present method are conducted at an elevated temperature using a thermophilic enzyme.

The reaction mixture is maintained for a period of time sufficient for the acceptor to be glycosylated, thereby forming the desired conjugate. Some of the conjugate can often be detected after a few hours, with recoverable amounts usually being obtained within 24 hours or less. Those of skill in the art understand that the rate of reaction is dependent on a number of variable factors (e.g, enzyme concentration, donor concentration, acceptor concentration, temperature, solvent volume), which are optimized for a selected system.

The present invention also provides for the industrial-scale production of modified polypeptides. As used herein, an industrial scale generally produces at least about 250 mg, preferably at least about 500 mg, and more preferably at least about 1 gram of finished, purified conjugate, preferably after a single reaction cycle, i.e., the conjugate is not a combination the reaction products from identical, consecutively iterated synthesis cycles.

In the discussion that follows, the invention is exemplified by the conjugation of modified sialic acid moieties to a glycosylated polypeptide. The exemplary modified sialic acid is labeled with (m-) PEG. The focus of the following discussion on the use of PEG-modified sialic acid and glycosylated polypeptides is for clarity of illustration and is not intended to imply that the invention is limited to the conjugation of these two partners. One of skill understands that the discussion is generally applicable to the additions of modified glycosyl moieties other than sialic acid. Moreover, the discussion is equally applicable to the modification of a glycosyl unit with agents other than PEG including other water-soluble polymers, therapeutic moieties, and biomolecules.

An enzymatic approach can be used for the selective introduction of a modifying group (e.g., mPEG or mPPG) onto a polypeptide or glycopeptide. In one embodiment, the method utilizes modified sugars, which include the modifying group in combination with an appropriate glycosyltransferase or glycosynthase. By selecting the glycosyltransferase that will make the desired carbohydrate linkage and utilizing the modified sugar as the donor substrate, the modifying group can be introduced directly onto the polypeptide backbone, onto existing sugar residues of a glycopeptide or onto sugar residues that have been added to a polypeptide. In another embodiment, the method utilizes modified sugars, which carry a masked reactive functional group, which can be used for attachment of the modifying group after transfer of the modified sugar onto the polypeptide or glycopeptide.

In one example, the glycosyltransferase is a sialyltransferase, used to append a modified sialyl residue to a glycopeptide. The glycosidic acceptor for the sialyl residue can be added to an O-linked glycosylation sequence, e.g., during expression of the polypeptide or can be added chemically or enzymatically after expression of the polypeptide, using the appropriate glycosidase(s), glycosyltransferase(s) or combinations thereof. Suitable acceptor moieties, include, for example, galactosyl acceptors such as GalNAc, Galβ1,4-GlcNAc, Galβ1,4GalNAc, Galβ1,3GalNAc, lacto-N-tetraose, Galβ1,3GlcNAc, Galβ1,3Ara, Galβ1,6GlcNAc, Galβ1,4-Glc (lactose), and other acceptors known to those of skill in the art (see, e.g., Paulson et al., J. Biol. Chem. 253: 5617-5624 (1978)).

In an exemplary embodiment, a GalNAc residue is added to an O-linked glycosylation sequence by the action of a GalNAc transferase. Hassan H, Bennett E P, Mandel U, Hollingsworth M A, and Clausen H (2000), Control of Mucin-Type O-Glycosylation: O-Glycan Occupancy is Directed by Substrate Specificities of Polypeptide GalNAc-Transferases (Eds. Ernst, Hart, and Sinay), Wiley-VCH chapter “Carbohydrates in Chemistry and Biology—a Comprehension Handbook”, pages 273-292. The method includes incubating the polypeptide to be modified with a reaction mixture that contains a suitable amount of a galactosyltransferase and a suitable galactosyl donor. The reaction is allowed to proceed substantially to completion or, alternatively, the reaction is terminated when a preselected amount of the galactose residue is added. Other methods of assembling a selected saccharide acceptor will be apparent to those of skill in the art.

In the discussion that follows, the method of the invention is exemplified by the use of modified sugars having a water-soluble polymer attached thereto. The focus of the discussion is for clarity of illustration. Those of skill will appreciate that the discussion is equally relevant to those embodiments in which the modified sugar bears a therapeutic moiety, a biomolecule or the like.

In another exemplary embodiment, a water-soluble polymer is added to a GalNAc residue via a modified galactosyl (Gal) residue. Alternatively, an unmodified Gal can be added to the terminal GalNAc residue.

In yet a further example, a water-soluble polymer (e.g., PEG) is added onto a terminal Gal residue using a modified sialic acid moiety and an appropriate sialyltransferase. This embodiment is illustrated in Scheme 9, below.

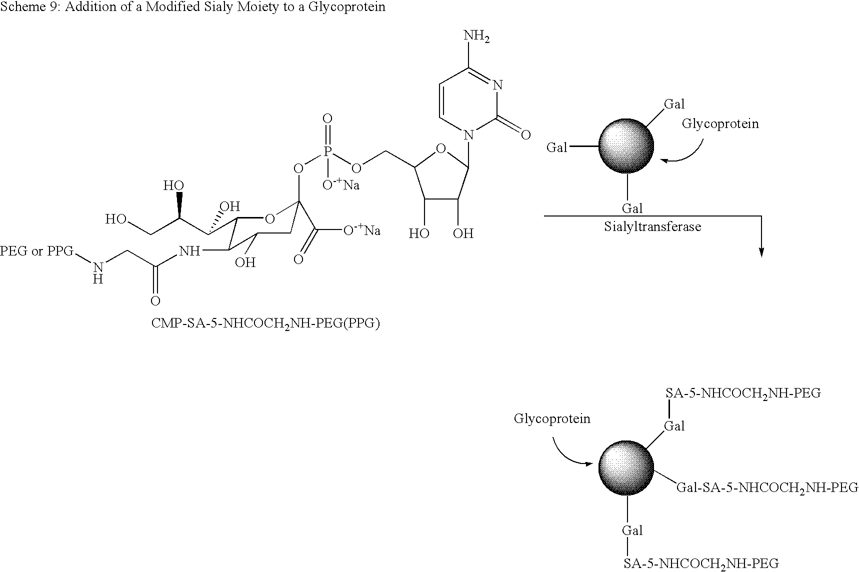

In yet a further approach, a masked reactive functionality is present on the sialic acid. The masked reactive group is preferably unaffected by the conditions used to attach the modified sialic acid to the polypeptide. After the covalent attachment of the modified sialic acid to the polypeptide, the mask is removed and the polypeptide is conjugated to the modifying group, such as a water soluble polymer (e.g., PEG or PPG) by reaction of the unmasked reactive group on the modified sugar residue with a reactive modifying group. This strategy is illustrated in Scheme 10, below.

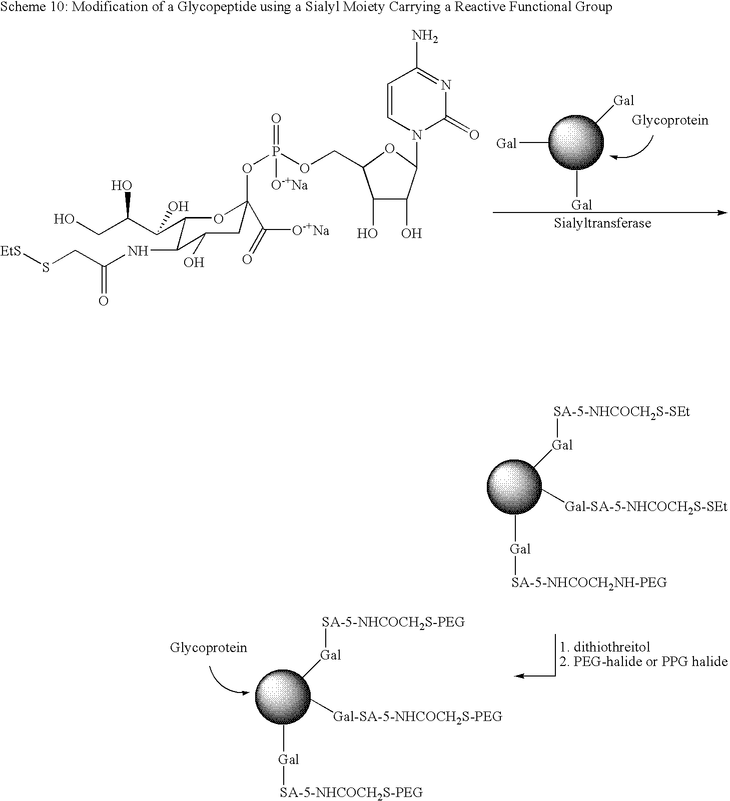

Any modified sugar can be used in combination with an appropriate glycosyltransferase, depending on the terminal sugars of the oligosaccharide side chains of the glycopeptide (Table 12).

TABLE 12
Exemplary Modified Sugars

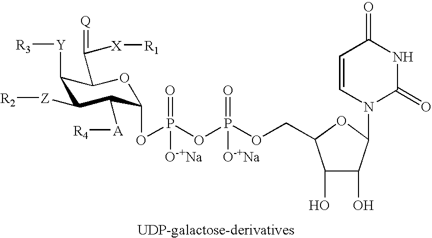

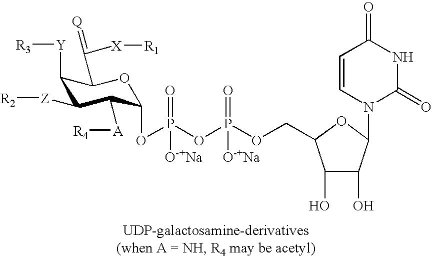

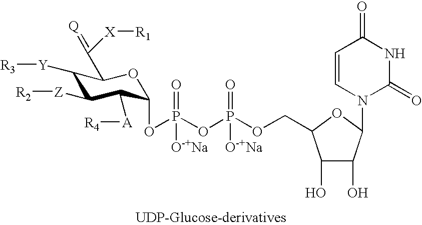

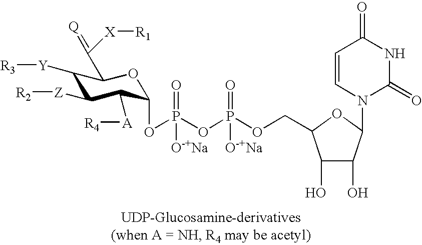

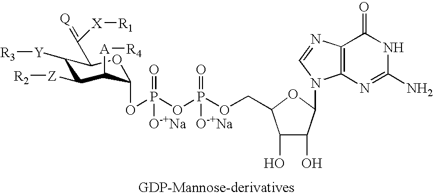

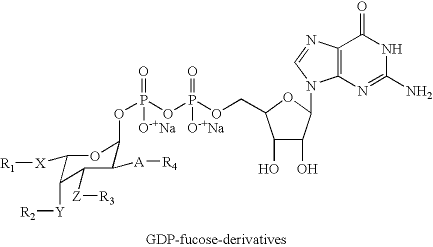

X = O, NH, S, CH₂, N—(R₁₋₅)₂.
Y= X; Z = X; A = X; B = X.
Q = H₂, O, S, NH, N—R.
R, R₁₋₄ = H, Linker-M, M.
M = Ligand of interest
Ligand of interest = acyl-PEG, acyl-PPG, alkyl-PEG, acyl-alkyl-PEG, acyl-alkyl-PEG, carbamoyl-PEG, carbamoyl-PPG, PEG, PPG, acyl-aryl-PEG, acyl-aryl-PPG, aryl-PEG, aryl-PPG, Mannose-₆-phosphate, heparine, heparan, SLex, Mannose, FGF, VFGF, protein, chondroitin, keratan, dermatan, albumin, integrins, peptides, etc.

In an alternative embodiment, the modified sugar is added directly to the peptide backbone using a glycosyltransferase known to transfer sugar residues to the O-linked glycosylation sequence on the polypeptide backbone. This exemplary embodiment is set forth in Scheme 11, below. Exemplary glycosyltransferases useful in practicing the present invention include, but are not limited to, GalNAc transferases (GalNAc T1 to GalNAc T20), GlcNAc transferases, fucosyltransferases, glucosyltransferases, xylosyltransferases, mannosyltransferases and the like. Use of this approach allows for the direct addition of modified sugars onto polypeptides that lack any carbohydrates or, alternatively, onto existing glycopeptides.

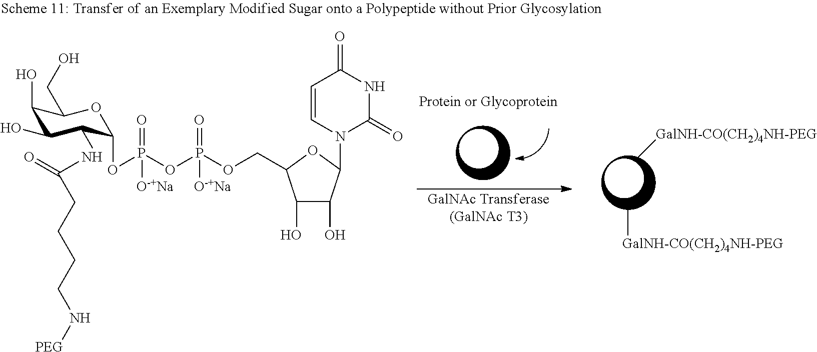

In each of the exemplary embodiments set forth above, one or more additional chemical or enzymatic modification steps can be utilized following the conjugation of the modified sugar to the polypeptide. In an exemplary embodiment, an enzyme (e.g., fucosyltransferase) is used to append a glycosyl unit (e.g., fucose) onto the terminal modified sugar attached to the polypeptide. In another example, an enzymatic reaction is utilized to “cap” (e.g., sialylate) sites to which the modified sugar failed to conjugate. Alternatively, a chemical reaction is utilized to alter the structure of the conjugated modified sugar. For example, the conjugated modified sugar is reacted with agents that stabilize or destabilize its linkage with the polypeptide component to which the modified sugar is attached. In another example, a component of the modified sugar is deprotected following its conjugation to the polypeptide. One of skill will appreciate that there is an array of enzymatic and chemical procedures that are useful in the methods of the invention at a stage after the modified sugar is conjugated to the polypeptide. Further elaboration of the modified sugar-peptide conjugate is within the scope of the invention.

In another exemplary embodiment, the glycopeptide is conjugated to a targeting agent, e.g., transferrin (to deliver the polypeptide across the blood-brain barrier, and to endosomes), carnitine (to deliver the polypeptide to muscle cells; see, for example, LeBorgne et al., Biochem. Pharmacol. 59: 1357-63 (2000), and phosphonates, e.g., bisphosphonate (to target the polypeptide to bone and other calciferous tissues; see, for example, Modern Drug Discovery, August 2002, page 10). Other agents useful for targeting are apparent to those of skill in the art. For example, glucose, glutamine and IGF are also useful to target muscle.

The targeting moiety and therapeutic polypeptide are conjugated by any method discussed herein or otherwise known in the art. Those of skill will appreciate that polypeptides in addition to those set forth above can also be derivatized as set forth herein. Exemplary polypeptides are set forth in the Appendix attached to copending, commonly owned U.S. Provisional Patent Application No. 60/328,523 filed Oct. 10, 2001.

In an exemplary embodiment, the targeting agent and the therapeutic polypeptide are coupled via a linker moiety. In this embodiment, at least one of the therapeutic polypeptide or the targeting agent is coupled to the linker moiety via an intact glycosyl linking group according to a method of the invention. In an exemplary embodiment, the linker moiety includes a poly(ether) such as poly(ethylene glycol). In another exemplary embodiment, the linker moiety includes at least one bond that is degraded in vivo, releasing the therapeutic polypeptide from the targeting agent, following delivery of the conjugate to the targeted tissue or region of the body.

In yet another exemplary embodiment, the in vivo distribution of the therapeutic moiety is altered via altering a glycoform on the therapeutic moiety without conjugating the therapeutic polypeptide to a targeting moiety. For example, the therapeutic polypeptide can be shunted away from uptake by the reticuloendothelial system by capping a terminal galactose moiety of a glycosyl group with sialic acid (or a derivative thereof).

### Enzymes

### Oligosaccharyl Transferases

The oligosaccharyl transferase useful in the methods of the invention can be an eukaryotic or prokaryotic enzyme. In one embodiment, the oligosaccharyl transferase is endogenous to a particular host cell, in which the polypeptide is expressed. For example, when the polypeptide is expressed in a bacterial host cell, the endogenous enzyme may be PglB or another enzyme having significant sequence identity with PglB. In one example, the endogenous enzyme has at least about 50%, at least about 60% at least about 70%, at least about 80%, at least about 90%, at least about 92%, at least about 94%, at least about 96%, at least about 98% or greater than 98% sequence identity with PglB or the corresponding part of PglB. In one example, the enzyme is smaller than PglB but has an amino acid sequence that corresponds to at least part of the PglB sequence. In another example, the polypeptide is expressed in a eukaryotic host cell, such as a yeast cell. In this example, the endogenous oligosaccharyl transferase may include a Stt3p enzyme or another enzyme exhibiting significant sequence identity with Stt3p.

The oligosaccharyl transferase can be a single enzyme or part of a protein complex, optionally membrane-bound. For example, a membrane preparation including membrane-bound enzymes having oligosaccharyl transferase activity may be used as a reagent for a glycosylation reaction. In one particular example, the bacterial enzyme PglB is over-expressed in a host cell (e.g., bacterial cell) and a membrane-preparation of such host cell is used for a cell-free in vitro glycosylation reaction.

In one embodiment, the oligosaccharyl transferase is a recombinant enzyme. In one example according to this embodiment, the recombinant oligosaccharyl transferase is co-expressed in the host cell, in which the polypeptide is expressed. Hence, in one example, the host cell includes a vector that includes the nucleic acid sequence encoding the oligosaccharyl transferase (e.g., PglB) and another vector including the nucleic acid sequence encoding the substrate polyeptide. Alternatively, the nucleic acid sequences of both the oligosaccharyl transferase and the polyeptide are part of the same transfection vector.

In another embodiment, the oligosaccharyl transferase is a soluble protein that is devoid of a functional membrane anchoring domain. For example, the enzyme may be PglB, wherein at least part of the N-terminal hydrophobic portion is removed. Such truncation can involve any number of amino acid residues as long the remaining sequence represents an enzyme that has at least some oligosaccharyl transferase activity. In one example, the soluble enzyme is expressed in a host cell and is then isolated. The isolated enzyme may be used in an in vitro glycosylation protocol.

The oligosaccharyl transferase can be derived from any species. Representative examples of oligosaccharyl transferases include eukaryotic (e.g., yeast, mammalian) proteins, such as Stt3p, bacterial (e.g., E. coli, Campylobacter jejuni) proteins, such as PglB, insect proteins and the like. In one example, the current invention uses recombinant PglB, or an enzyme having high sequence identity to a PglB enzyme. An exemplary oligosaccharyltransferase of the invention comprises an amino acid sequence according to SEQ ID NO: 102. Exemplary oligosaccharyltransferases have an amino acid sequence that is at least about 50%, at least about 60% at least about 70%, at least about 80%, at least about 90%, at least about 92%, at least about 94%, at least about 96%, at least about 98% or greater than 98% sequence identity with the amino acid sequence of SEQ ID NO: 102 or its STT3 subunit (amino acid residues 9-626).

PgIB (Campylobacter jejuni, Accession CAL35243)
(SEQ ID NO: 102)

MLKKEYLKNPYLVLFAMIVLAYVFSVFCRFYWVWWASEFNEYFFNNQLMIISNDGYAFAEG
ARDMIAGFHQPNDLSYYGSSLSTLTYWLYKITPFSFESIILYMSTFLSSLVVIPIILLANEYKRPL
MGFVAALLASVANSYYNRTMSGYYDTDMLVIVLPMFILFFMVRMILKKDFFSLIALPLFIGIY
LWWYPSSYTLNVALIGLFLIYTLIFHRKEKIFYIAVILSSLTLSNIAWFYQSAIIVILFALFALEQ
KRLNFMIIGILGSATLIFLILSGGVDPILYQLKFYIFRSDESANLTQGFMYFNVNQTIQEVENVD
FSEFMRRISGSEIVFLFSLFGFVWLLRKHKSMIMALPILVLGFLALKGGLRFTIYSVPVMALGF
GFLLSEFKAILVKKYSQLTSNVCIVFATILTLAPVFIHIYNYKAPTVFSQNEASLLNQLKNIANR
EDYVVTWWDYGYPVRYYSDVKTLVDGGKHLGKDNFFPSFSLSKDEQAAANMARLSVEYTE
KSFYAPQNDILKSDILQAMMKDYNQSNVDLFLASLSKPDFKIDTPKTRDIYLYMPARMSLIFS
TVASFSFINLDTGVLDKPFTFSTAYPLDVKNGEIYLSNGVVLSDDFRSFKIGDNVVSVNSIVEI
NSIKQGEYKITPIDDKAQFYIFYLKDSAIPYAQFILMDKTMFNSAYVQMFFLGNYDKNLFDLV
INSRDAKVFKLKI
PgIB (Neisseria gonorrhoeae, Accession YP_207258)

(SEQ ID NO: 103)
MSKAVKRLFDIIASASGLIVLSPVFLVLIYLIRKNLGSPVFFIRERPGKDGKPFKMVKFRSMRD
ALDSDGIPLPDSERLTDFGKKLRATSLDELPELWNVLKGEMSLVGPRPLLMQYLPLYNKFQN
RRHEMKPGITGWAQVNGRNALSWDEKFSCDVWYTDNFSFWLDMKILFLTVKKVLIKEGISA
QGEATMPPFAGNRKLAVIGAGGHGKVVAELAAALGTYGEIVFLDDRTQGSVNGFPVIGTTLL
LENSLSPEQFDITVAVGNNRIRRQITENAAALGFKLPVLIHPDATVSPSAIIGQGSVVMAKAVV
QAGSVLKDGVIVNTAATVDHDCLLDAFVHISPGAHLSGNTRIGEESRIGTGACSRQQTTVGSG
VTAGAGAVIVCDIPDGMTVAGNPAKPLTGKNPKTGTA
Oligosaccharyltransferase (Saccharomyces cerevisiae,
Accession EDN64373)
(SEQ ID NO: 104)
MKWCSTYIIIWLAIIFHKFQKSTATASHNIDDILQLKGDTGVITVTADNYPLLSRGVPGY
FNILYITMRGTNSNGMSCQLCHDFEKTYQAVADVIRSQAPQSLNLFFTVDVNEVPQLVKD
LKLQNVPHLVVYPPAESNKQSQFEWKTSPFYQYSLVPENAENTLQFGDFLAKILNISITV
PQAFNVQEFVYYFVACMVVFIFIKKVILPKVTNKWKLFSMILSLGILLPSITGYKFVEMN
AIPFIARDAKNRIMYFSGGSGWQFGIEIFSVSLMYIVMSALSVLLIYVPKISCVSEKMRG
LLSSFLACVLFYFFSYFISCYLIKNPGYPIVF
Oligosaccharyltransferase (Homo sapiens, Accession BAA23670)
(SEQ ID NO: 105)
MGYFRCAGAGSFGRRRKMEPSTAARAWALFWLLLPLLGAVCASGPRTLVLLDNLNVRETHS
LFFRSLKDRGFELTFKTADDPSLSLIKYGEFLYDNLIIFSPSVEDFGGNINVETISAFIDGGGSVL
VAASSDIGDPLRELGSECGIEFDEEKTAVIDHHNYDISDLGQHTLIVADTENLLKAPTIVGKSS
LNPILFRGVGMVADPDNPLVLDILTGSSTSYSFFPDKPITQYPHAVGKNTLLIAGLQARNNAR
VIFSGSLDFFSDSFFNSAVQKAAPGSQRYSQTGNYELAVALSRWVFKEEGVLRVGPVSHHRV
GETAPPNAYTVTDLVEYSIVIQQLSNAKWVPFDGDDIQLEFVRIDPFVRTFLKKKGGKYSVQF
KLPDVYGVFQFKVDYNRLGYTHLYSSTQVSVRPLQHTQYERFIPSAYPYYASAFPMMLGLFI
FSIVFLHMKEKEKSD
Oligosaccharyltransferase (Mus musculus, Accession BAA23671)
(SEQ ID NO: 106)
MKMDPRLAVRAWPLCGLLLAVLGCVCASGPRTLVLLDNLNVRDTHSLFFRSLKDRGFELTF
KTADDPSLSLIKYGEFLYDNLIIFSPSVEDFGGNINVETISAFIDGGGSVLVAASSDIGDPLRELG
SECGIEFDEEKTAVIDHHNYDVSDLGQHTLIVADTENLLKAPTIVGKSSLNPILFRGVGMVAD
PDNPLVLDILTGSSTSYSFFPDKPITQYPHAVGRNTLLIAGLQARNNARVIFSGSLDFFSDAFFN
SAVQKATPGAQRYSQTGNYELAVALSRWVFKEEGVLRVGPVSHHRVGEMAPPNAYTVTDL
VEYSIVIEQLSNGKWVPFDGDDIQLEFVRIDPFVRTFLKRKGGKYSVQFKLPDVYGVFQFKVD
YNRLGYTHLYSSTQVSVRPLQHTQYERFIPSAYPYYASAFSMMAGLFIFSIVFLHMKEKEKSD
Oligosaccharyltransferase (Candida albicans,
Accession XP_714366 or XP_440145)
(SEQ ID NO: 107)
MAKSASNKKSIPTTSSSSTTTSAASSSVVLKEVKSTLTTTINNYFDTISAQPRLKLIDLFLIFLVL
LGILQFIYVLIIGNFPFNSFLGGFISCVGQFVLLVSLRLQINDSTTTTTNKESDDQLELDEDKIEN
GTTGGGNGRLFKEITPERSFGDFIFASLILHFIVIHFIN
Phospho-dolichol-GlcNAc-1-phosphate transferases
UDP-N-Acetylglucosamine-dolichyl-phosphate-N-acetylglucosamine-
phosphotransferase Isoform b (Homo sapiens,
Accession NP_976061)
(SEQ ID NO: 108)
MIFLGFADDVLNLRWRHKLLLPTAASLPLLMVYFTNFGNTTIVVPKPFRPILGLHLDLGILYY
VYMGLLAVFCTNAINILAGINGLEAGQSLVISASIIVFNLVELEGDCRDDHVFSLYFMIPFFFTT
LGLLYHNWYPSRVFVGDTFCYFAGMTFAVVGILGHFSKTMLLFFMPQVFNFLYSLPQLLHIIP
CPRHRIPRLNIKTGKLEMSYSKFKTKSLSFLGTFILKVAESLQLVTVHQSETEDGEFTECNNMT
LINLLLKVLGPIHERNLTLLLLLLQILGSAITFSIRYQLVRLFYDV
UDP-N-Acetylglucosamine-dolichyl-phosphate-N-acetylglucosamine-
phosphotransferase Isoform a (Homo sapiens,
Accession NP_001373)
(SEQ ID NO: 109)
MWAFSELPMPLLINLIVSLLGFVATVTLIPAFRGHFIAARLCGQDLNKTSRQQIPESQGVISGA
VFLIILFCFIPFPFLNCFVKEQCKAFPHHEFVALIGALLAICCMIFLGFADDVLNLRWRHKLLLP
TAASLPLLMVYFTNFGNTTIVVPKPFRPILGLHLDLGILYYVYMGLLAVFCTNAINILAGINGL
EAGQSLVISASIIVFNLVELEGDCRDDHVFSLYFMIPFFFTTLGLLYHNWYPSRVFVGDTFCYF
AGMTFAVVGILGHFSKTMLLFFMPQVFNFLYSLPQLLHIIPCPRHRIPRLNIKTGKLEMSYSKF
KTKSLSFLGTFILKVAESLQLVTVHQSETEDGEFTECNNMTLINLLLKVLGPIHERNLTLLLLL
LQILGSAITFSIRYQLVRLFYDV
UDP-N-Acetylglucosamine-dolichyl-phosphate-N-acetylglucosamine-
phosphotransferase (GPT, G1PT, GlcNAc-1-P-transferase)
Mus musculus, Accession P42867)
(SEQ ID NO: 110)
MWAFPELPLPLPLLVNLIGSLLGFVATVTLIPAFRSHFIAARLCGQDLNKLSQQQIPESQGVISG
AVFLIILFCFIPFPFLNCFVEEQCKAFPHHEFVALIGALLAICCMIFLGFADDVLNLRWRHKLLL
PTAASLPLLMVYFTNFGNTTIVVPKPFRWILGLHLDLGILYYVYMGLLAVFCTNAINILAGIN
GLEAGQSLVISASIIVFNLVELEGDYRDDHIFSLYFMIPFFFTTLGLLYHNWYPSRVFVGDTFC
YFAGMTFAVVGILGHFSKTMLLFFMPQVFNFLYSLPQLFHIIPCPRHRMPRLNAKTGKLEMSY
SKFKTKNLSFLGTFILKVAENLRLVTVHQGESEDGAFTECNNMTLINLLLKVFGPIHERNLTLL
LLLLQVLSSAATFSIRYQLVRLFYDV
UDP-N-Acetylglucosamine-dolichyl-phosphate-N-acetylglucosamine-
phosphotransferase (GPT, G1PT, GlcNAc-1-P-transferase)
(Saccharomyces cerevisiae, Accession P07286)
(SEQ ID NO: 111)
MLRLFSLALITCLIYYSKNQGPSALVAAVGFGIAGYLATDMLIPRVGKSFIKIGLFGKDLSKPG
RPVLPETIGAIPAAVYLFVMFIYIPFIFYKYMVITTSGGGHRDVSVVEDNGMNSNIFPHDKLSE
YLSAILCLESTVLLGIADDLFDLRWRHKFFLPAIAAIPLLMVYYVDFGVTHVLIPGFMERWLK
KTSVDLGLWYYVYMASMAIFCPNSINILAGVNGLEVGQCIVLAILALLNDLLYFSMGPLATR
DSHRFSAVLIIPFLGVSLALWKWNRWPATVFVGDTYCYFAGMVFAVVGILGHFSKTMLLLFI
PQIVNFIYSCPQLFKLVPCPRHRLPKFNEKDGLMYPSRANLKEEPPKSIFKPILKLLYCLHLIDL
EFDENNEIISTSNMTLINLTLVWFGPMREDKLCNTILKLQFCIGILALLGRHAIGAIIFGHDNLW
TVR
Other Enzymes Useful for the Synthesis of Glycosyl Donor Molecules
PglC (Campylobacter jejuni, Accession AAD51385)
(SEQ ID NO: 112)
MYEKVFKRIFDFILALVLLVLFSPVILITALLLKITQGSVIFTQNRPGLDEKIFKIYKFKTMSDER
DEKGELLSDELRLKAFGKIVRSLSLDELLQLFNVLKGDMSFVGPRPLLVEYLSLYNEEQKLRH
KVRPGITGWAQVNGRNAISWQKKFELDVYYVKNISFLLDLKIMFLTALKVLKRSGVSKEGH
VTTEKFNGKN
PglC (Neisseria gonorrhoea, Accession YP_207257)
(SEQ ID NO: 113)
MLNTALSPWPSFTREEADAVSKVLLSNKVNYWTGSECREFEKEFAAFAGTRYAVALSNGTL
ALDAALKAIGIGAGDDVIVTSRTFLASASCIVNAGANPVFADVDLNSQNISAETVKAVLTPNT
KAVIVVHLAGMPAEMDGIMALAKEHDLWVIEDCAQAHGATYKGKSVGSIGHVGAWSFCQD
KIITTGGEGGMVTTNDKTLWEKMWAYKDHGKSYDAVYHREHAPGFRWLHESFGTNWRM
MEMQAVIGRIQLKHLPEWTARRQENAAKLAESLRKFKSIRLIEVAGYIGHAQYKFYAFVKPE
HLKDDWTRDRIVSELNARNVPCYQGGCSEVYLEKAFDNTPWRPKERLKNAVELGGTALTFL
VHPTLTDDEIAFCKKHIEAVLTEAAR
Glycosyltransferase (Methanobrevibacter smithii, Accession
YP_001273863)
(SEQ ID NO: 114)
MKTAVLIPCYNEELTIKKVILDFKKALPKADIYVYDNNSTDNSYEIAKDTGAIVKREYRQGKG
NVVRSMFRDIDADCYILVDGDDTYPAEASKEIEELILSKKADMVIGDRLSSTYFEENKRRFHN
SGNKLVRKLINTIFNSDISDIMTGMRGFSYEFVKSFPISSKEFEIETEMTIFALNHNFLIKELPIEY
RDRMDGSESKLNTFSDGYKVISLLFGLFRDIRPLFFFSLVTLVLLIIAGLYFFPILIDFYRTGFVE
KVPTLITVGVVAIVAVIIFFTGVVLHVIRKQHDENFEHHLNLIAQNKKR

### Glycosyltransferases

In one embodiment, glycosyltransferases are used in the synthesis of a glycosyl donor species of the invention. In another embodiment, glycosyltransferases may be used in a method for making a polypeptide conjugate of the invention. Glycosyltransferases catalyze the addition of activated sugars (donor NDP-sugars), in a step-wise fashion, to a protein, glycopeptide, lipid or glycolipid or to the non-reducing end of a growing oligosaccharide. For example, in a first step a polypptide may be glycosylated using a glycosyl donor species of the invention (e.g., a lipid-pyrophosphate-linked glycosyl moiety) and a suitable oligosaccharyl transferase. This glycosylation reaction may optionally occur in the host cell, in which the polypeptide is expressed. In a second step, the glycosylated polypeptide is subjected to a glycosylation or glycoPEGylation reaction involving a modified or non-modified sugar nucleotide and a suitable glycosyl transferase.

A large number of glycosyltransferases are known in the art. Examples of such enzymes include Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like.

For enzymatic saccharide syntheses that involve glycosyltransferase reactions, glycosyltransferase can be cloned, or isolated from any source. Many cloned glycosyltransferases are known, as are their polynucleotide sequences. Glycosyltransferase amino acid sequences and nucleotide sequences encoding glycosyltransferases from which the amino acid sequences can be deduced are found in various publicly available databases, including GenBank, Swiss-Prot, EMBL, and others.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligosaccharyltransferases. Suitable glycosyltransferases include those obtained from eukaryotes, as well as from prokaryotes.

DNA encoding glycosyltransferases may be obtained by chemical synthesis, by screening reverse transcripts of mRNA from appropriate cells or cell line cultures, by screening genomic libraries from appropriate cells, or by combinations of these procedures. Screening of mRNA or genomic DNA may be carried out with oligonucleotide probes generated from the glycosyltransferases gene sequence. Probes may be labeled with a detectable group such as a fluorescent group, a radioactive atom or a chemiluminescent group in accordance with known procedures and used in conventional hybridization assays. In the alternative, glycosyltransferases gene sequences may be obtained by use of the polymerase chain reaction (PCR) procedure, with the PCR oligonucleotide primers being produced from the glycosyltransferases gene sequence (See, for example, U.S. Pat. No. 4,683,195 to Mullis et al. and U.S. Pat. No. 4,683,202 to Mullis).

The glycosyltransferase may be synthesized in host cells transformed with vectors containing DNA encoding the glycosyltransferases enzyme. Vectors are used either to amplify DNA encoding the glycosyltransferases enzyme and/or to express DNA which encodes the glycosyltransferases enzyme. An expression vector is a replicable DNA construct in which a DNA sequence encoding the glycosyltransferases enzyme is operably linked to suitable control sequences capable of effecting the expression of the glycosyltransferases enzyme in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences which control the termination of transcription and translation. Amplification vectors do not require expression control domains. All that is needed is the ability to replicate in a host, usually conferred by an origin of replication, and a selection gene to facilitate recognition of transformants.

In an exemplary embodiment, the invention utilizes a prokaryotic enzyme. Such glycosyltransferases include enzymes involved in synthesis of lipooligosaccharides (LOS), which are produced by many gram negative bacteria (Preston et al., Critical Reviews in Microbiology 23(3): 139-180 (1996)). Such enzymes include, but are not limited to, the proteins of the rfa operons of species such as E. coli and Salmonella typhimurium, which include a β1,6 galactosyltransferase and a β1,3 galactosyltransferase (see, e.g., EMBL Accession Nos. M80599 and M86935 (E. coli); EMBL Accession No. S56361 (S. typhimurium)), a glucosyltransferase (Swiss-Prot Accession No. P25740 (E. coli), an β1,2-glucosyltransferase (rfaJ)(Swiss-Prot Accession No. P27129 (E. coli) and Swiss-Prot Accession No. P19817 (S. typhimurium)), and an β1,2-N-acetylglucosaminyltransferase (rfaK)(EMBL Accession No. U00039 (E. coli). Other glycosyltransferases for which amino acid sequences are known include those that are encoded by operons such as rfaB, which have been characterized in organisms such as Klebsiella pneumoniae, E. coli, Salmonella typhimurium, Salmonella enterica, Yersinia enterocolitica, Mycobacterium leprosum, and the rhl operon of Pseudomonas aeruginosa.

Also suitable for use in the present invention are glycosyltransferases that are involved in producing structures containing lacto-N-neotetraose, D-galactosyl-β-1,4-N-acetyl-D-glucosaminyl-β-1,3-D-galactosyl-β-1,4-D-glucose, and the P^{k} blood group trisaccharide sequence, D-galactosyl-α-1,4-D-galactosyl-β-1,4-D-glucose, which have been identified in the LOS of the mucosal pathogens Neisseria gonnorhoeae and N. meningitidis (Scholten et al., J. Med. Microbiol. 41: 236-243 (1994)). The genes from N. meningitidis and N. gonorrhoeae that encode the glycosyltransferases involved in the biosynthesis of these structures have been identified from N. meningitidis immunotypes L3 and L1 (Jennings et al., Mol. Microbiol. 18: 729-740 (1995)) and the N. gonorrhoeae mutant F62 (Gotshlich, J. Exp. Med. 180: 2181-2190 (1994)). In N. meningitidis, a locus consisting of three genes, lgtA, lgtB and lg E, encodes the glycosyltransferase enzymes required for addition of the last three of the sugars in the lacto-N-neotetraose chain (Wakarchuk et al., J. Biol. Chem. 271: 19166-73 (1996)). Recently the enzymatic activity of the lgtB and lgtA gene product was demonstrated, providing the first direct evidence for their proposed glycosyltransferase function (Wakarchuk et al., J. Biol. Chem. 271(45): 28271-276 (1996)). In N. gonorrhoeae, there are two additional genes, lgtD which adds β-D-GalNAc to the 3 position of the terminal galactose of the lacto-N-neotetraose structure and lgtC which adds a terminal α-D-Gal to the lactose element of a truncated LOS, thus creating the P^{k} blood group antigen structure (Gotshlich (1994), supra.). In N. meningitidis, a separate immunotype L1 also expresses the P^{k} blood group antigen and has been shown to carry an lgtC gene (Jennings et al., (1995), supra.). Neisseria glycosyltransferases and associated genes are also described in U.S. Pat. No. 5,545,553 (Gotschlich). Genes for α1,2-fucosyltransferase and α1,3-fucosyltransferase from Helicobacter pylori has also been characterized (Martin et al., J. Biol. Chem. 272: 21349-21356 (1997)). Also of use in the present invention are the glycosyltransferases of Campylobacter jejuni (see, for example, http://afmb.cnrs-mrs.fr/˜pedro/CAZY/gtf_{—}42.html).

### (a) GalNAc Transferases

In one embodiment, the glycosyl transferase is a member of a large family of UDP-GalNAc: polypeptide N-acetylgalactosaminyltransferases (GalNAc-transferases), which normally transfer GalNAc to serine and threonine acceptor sites (Hassan et al., J. Biol. Chem. 275: 38197-38205 (2000)). To date twelve members of the mammalian GalNAc-transferase family have been identified and characterized (Schwientek et al., J. Biol. Chem. 277: 22623-22638 (2002)), and several additional putative members of this gene family have been predicted from analysis of genome databases. The GalNAc-transferase isoforms have different kinetic properties and show differential expression patterns temporally and spatially, suggesting that they have distinct biological functions (Hassan et al., J. Biol. Chem. 275: 38197-38205 (2000)). Sequence analysis of GalNAc-transferases have led to the hypothesis that these enzymes contain two distinct subunits: a central catalytic unit, and a C-terminal unit with sequence similarity to the plant lectin ricin, designated the “lectin domain” (Hagen et al., J. Biol. Chem. 274: 6797-6803 (1999); Hazes, Protein Eng. 10: 1353-1356 (1997); Breton et al., Curr. Opin. Struct. Biol. 9: 563-571 (1999)). Previous experiments involving site-specific mutagenesis of selected conserved residues confirmed that mutations in the catalytic domain eliminated catalytic activity. In contrast, mutations in the “lectin domain” had no significant effects on catalytic activity of the GalNAc-transferase isoform, GalNAc-T1 (Tenno et al., J. Biol. Chem. 277(49): 47088-96 (2002)). Thus, the C-terminal “lectin domain” was believed not to be functional and not to play roles for the enzymatic functions of GalNAc-transferases (Hagen et al., J. Biol. Chem. 274: 6797-6803 (1999)).

Polypeptide GalNAc-transferases, which have not displayed apparent GalNAc-glycopeptide specificities, also appear to be modulated by their putative lectin domains (PCT WO 01/85215 A2). Recently, it was found that mutations in the GalNAc-T1 putative lectin domain, similarly to those previously analysed in GalNAc-T4 (Hassan et al., J. Biol. Chem. 275: 38197-38205 (2000)), modified the activity of the enzyme in a similar fashion as GalNAc-T4. Thus, while wild type GalNAc-T1 added multiple consecutive GalNAc residues to a polypeptide substrate with multiple acceptor sites, mutated GalNAc-T1 failed to add more than one GalNAc residue to the same substrate (Tenno et al., J. Biol. Chem. 277(49): 47088-96 (2002)). More recently, the x-ray crystal structures of murine GalNAc-T1 (Fritz et al., PNAS 2004, 101(43): 15307-15312) as well as human GalNAc-T2 (Fritz et al., J. Biol. Chem. 2006, 281(13):8613-8619) have been determined. The human GalNAc-T2 structure revealed an unexpected flexibility between the catalytic and lectin domains and suggested a new mechanism used by GalNAc-T2 to capture glycosylated substrates. Kinetic analysis of GalNAc-T2 lacking the lectin domain confirmed the importance of this domain in acting on glycopeptide substrates. However, the enzymes activity with respect to non-glycosylated substrates was not significantly affected by the removal of the lectin domain. Thus, truncated human GalNAc-T2 enzymes lacking the lectin domain or those enzymes having a truncated lectin domain can be useful for the glycosylation of polypeptide substrates where further glycosylation of the resulting mono-glycosylated polypeptide is not desired.

Production of proteins such as the enzyme GalNAc T_{I-XX} from cloned genes by genetic engineering is well known. See, eg., U.S. Pat. No. 4,761,371. One method involves collection of sufficient samples, then the amino acid sequence of the enzyme is determined by N-terminal sequencing. This information is then used to isolate a cDNA clone encoding a full-length (membrane bound) transferase which upon expression in the insect cell line Sf9 resulted in the synthesis of a fully active enzyme. The acceptor specificity of the enzyme is then determined using a semiquantitative analysis of the amino acids surrounding known glycosylation sequences in 16 different proteins followed by in vitro glycosylation studies of synthetic peptides. This work has demonstrated that certain amino acid residues are overrepresented in glycosylated peptide segments and that residues in specific positions surrounding glycosylated serine and threonine residues may have a more marked influence on acceptor efficiency than other amino acid moieties.

Since it has been demonstrated that mutations of GalNAc transferases can be utilized to produce glycosylation patterns that are distinct from those produced by the wild-type enzymes, it is within the scope of the present invention to utilize one or more mutant or truncated GalNAc transferase in the invention. Catalytic domains and truncation mutants of GalNAc-T2 proteins are described, for example, in U.S. Provisional Patent Application 60/576,530 filed Jun. 3, 2004; and U.S. Provisional Patent Application 60/598,584, filed Aug. 3, 2004; both of which are herein incorporated by reference for all purposes. Catalytic domains can also be identified by alignment with known glycosyltransferases. Truncated GalNAc-T2 enzymes, such as human GalNAc-T2 (Δ51), human GalNAc-T2 (Δ51 Δ445) and methods of obtaining those enzymes are also described in WO 06/102652 (PCT/US06/011065, filed Mar. 24, 2006) and PCT/US05/00302, filed Jan. 6, 2005, which are herein incorporated by reference for all purposes.

### (b) Fucosyltransferases

In some embodiments, a glycosyltransferase used in the method of the invention is a fucosyltransferase. Fucosyltransferases are known to those of skill in the art. Exemplary fucosyltransferases include enzymes, which transfer L-fucose from GDP-fucose to a hydroxy position of an acceptor sugar. Fucosyltransferases that transfer non-nucleotide sugars to an acceptor are also of use in the present invention.

In some embodiments, the acceptor sugar is, for example, the GlcNAc in a Galβ(1→3,4)GlcNAcβ-group in an oligosaccharide glycoside. Suitable fucosyltransferases for this reaction include the Galβ(1→3,4)GlcNAcβ1-α(1→3,4)fucosyltransferase (FTIII E.C. No. 2.4.1.65), which was first characterized from human milk (see, Palcic, et al., Carbohydrate Res. 190:1-11 (1989); Prieels, et al., J. Biol. Chem. 256: 10456-10463 (1981); and Nunez, et al., Can. J. Chem. 59: 2086-2095 (1981)) and the Galβ(1→4)GlcNAcβ-αfucosyltransferases (FTIV, FTV, FTVI) which are found in human serum. FTVII (E.C. No. 2.4.1.65), a sialyl α(2→3)Galβ(1→3)GlcNAcβ fucosyltransferase, has also been characterized. A recombinant form of the Galβ(1→3,4) GlcNAcβ-α(1→3,4)fucosyltransferase has also been characterized (see, Dumas, et al., Bioorg. Med. Letters 1: 425-428 (1991) and Kukowska-Latallo, et al., Genes and Development 4: 1288-1303 (1990)). Other exemplary fucosyltransferases include, for example, α1,2 fucosyltransferase (E.C. No. 2.4.1.69). Enzymatic fucosylation can be carried out by the methods described in Mollicone, et al., Eur. J. Biochem. 191: 169-176 (1990) or U.S. Pat. No. 5,374,655. Cells that are used to produce a fucosyltransferase will also include an enzymatic system for synthesizing GDP-fucose.

### (c) Galactosyltransferases

In another group of embodiments, the glycosyltransferase is a galactosyltransferase. Exemplary galactosyltransferases include α(1,3) galactosyltransferases (E.C. No. 2.4.1.151, see, e.g., Dabkowski et al., Transplant Proc. 25:2921 (1993) and Yamamoto et al. Nature 345: 229-233 (1990), bovine (GenBank j04989, Joziasse et al., J. Biol. Chem. 264: 14290-14297 (1989)), murine (GenBank m26925; Larsen et al., Proc. Nat'l. Acad. Sci. USA 86: 8227-8231 (1989)), porcine (GenBank L36152; Strahan et al., Immunogenetics 41: 101-105 (1995)). Another suitable α1,3 galactosyltransferase is that which is involved in synthesis of the blood group B antigen (EC 2.4.1.37, Yamamoto et al., J. Biol. Chem. 265: 1146-1151 (1990) (human)). Also suitable in the practice of the invention are soluble forms of α1,3-galactosyltransferase such as that reported by Cho, S. K. and Cummings, R. D. (1997) J. Biol. Chem., 272, 13622-13628.

In another embodiment, the galactosyltransferase is a β(1,3)-galactosyltransferases, such as Core-1-GalT1. Human Core-1-β1,3-galactosyltransferase has been described (see, e.g., Ju et al., J. Biol. Chem. 2002, 277(1): 178-186). Drosophila melanogaster enzymes are described in Correia et al., PNAS 2003, 100(11): 6404-6409 and Muller et al., FEBS J. 2005, 272(17): 4295-4305. Additional Core-1-β3 galactosyltransferases, including truncated versions thereof, are disclosed in WO/0144478 and U.S. Provisional Patent Application No. 60/842,926 filed Sep. 6, 2006. In an exemplary embodiment, the β(1,3)-galactosyltransferase is a member selected from enzymes described by PubMed Accession Number AAF52724 (transcript of CG9520-PC) and modified versions thereof, such as those variations, which are codon optimized for expression in bacteria. The sequence of an exemplary, soluble Core-1-GalT1 (Core-1-GalT1 Δ31) enzyme is shown below:

Also suitable for use in the methods of the invention are β(1,4) galactosyltransferases, which include, for example, EC 2.4.1.90 (LacNAc synthetase) and EC 2.4.1.22 (lactose synthetase) (bovine (D'Agostaro et al., Eur. J. Biochem. 183: 211-217 (1989)), human (Masri et al., Biochem. Biophys. Res. Commun. 157: 657-663 (1988)), murine (Nakazawa et al., J. Biochem. 104: 165-168 (1988)), as well as E.C. 2.4.1.38 and the ceramide galactosyltransferase (EC 2.4.1.45, Stahl et al., J. Neurosci. Res. 38: 234-242 (1994)). Other suitable galactosyltransferases include, for example, α1,2 galactosyltransferases (from e.g., Schizosaccharomyces pombe, Chapell et al., Mol. Biol. Cell 5: 519-528 (1994)).

### (d) Sialyltransferases

Sialyltransferases are another type of glycosyltransferase that is useful in the recombinant cells and reaction mixtures of the invention. Cells that produce recombinant sialyltransferases will also produce CMP-sialic acid, which is a sialic acid donor for sialyltransferases. Examples of sialyltransferases that are suitable for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al., Glycobiology 6: v-xiv (1996)). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem. 256: 3159 (1981), Weinstein et al., J. Biol. Chem. 257: 13845 (1982) and Wen et al., J. Biol. Chem. 267: 21011 (1992). Another exemplary α2,3-sialyltransferase (EC 2.4.99.4) transfers sialic acid to the non-reducing terminal Gal of the disaccharide or glycoside. see, Rearick et al., J. Biol. Chem. 254: 4444 (1979) and Gillespie et al., J. Biol. Chem. 267: 21004 (1992). Further exemplary enzymes include Gal-β-1,4-GlcNAc α-2,6 sialyltransferase (See, Kurosawa et al. Eur. J. Biochem. 219: 375-381 (1994)).

Preferably, for glycosylation of carbohydrates of glycopeptides the sialyltransferase will be able to transfer sialic acid to the sequence Galβ1,4-GlcNAc-, the most common penultimate sequence underlying the terminal sialic acid on fully sialylated carbohydrate structures (see, Table 14, below).

TABLE 14
Sialyltransferases which use the Galβ1,4GlcNAc sequence as an acceptor
substrate

| Sialyltransferase | Source | Sequence(s) formed | Ref. |
|---|---|---|---|
| ST6Gal I | Mammalian | NeuAcα2,6Galβ1,4GlcNAc- | 1 |
| ST3Gal III | Mammalian | NeuAcα2,3Galβ1,4GlcNAc- | 1 |
| | | NeuAcα2,3Galβ1,3GlcNAc- | |
| ST3Gal IV | Mammalian | NeuAcα2,3Galβ1,4GlcNAc- | 1 |
| | | NeuAcα2,3Galβ1,3GlcNAc- | |
| ST6Gal II | Mammalian | NeuAcα2,6Galβ1,4GlcNAc | |
| ST6Gal II | photo- | NeuAcα2,6Galβ1,4GlcNAc- | 2 |
| | bacterium | | |
| ST3Gal V | N. meningitides | NeuAcα2,3Galβ1,4GlcNAc- | 3 |
| | N. | | |
| | gonorrhoeae | | |

1) Goochee et al., Bio/Technology 9: 1347-1355 (1991)
2) Yamamoto et al., J. Biochem. 120: 104-110 (1996)
3) Gilbert et al., J. Biol. Chem. 271: 28271-28276 (1996)

An example of a sialyltransferase that is useful in the claimed methods is ST3Gal III, which is also referred to as α(2,3)sialyltransferase (EC 2.4.99.6). This enzyme catalyzes the transfer of sialic acid to the Gal of a Galβ1,3GlcNAc or Galβ1,4GlcNAc glycoside (see, e.g., Wen et al., J. Biol. Chem. 267: 21011 (1992); Van den Eijnden et al., J. Biol. Chem. 256: 3159 (1991)) and is responsible for sialylation of asparagine-linked oligosaccharides in glycopeptides. The sialic acid is linked to a Gal with the formation of an α-linkage between the two saccharides. Bonding (linkage) between the saccharides is between the 2-position of NeuAc and the 3-position of Gal. This particular enzyme can be isolated from rat liver (Weinstein et al., J. Biol. Chem. 257: 13845 (1982)); the human cDNA (Sasaki et al. (1993) J. Biol. Chem. 268: 22782-22787; Kitagawa & Paulson (1994) J. Biol. Chem. 269: 1394-1401) and genomic (Kitagawa et al. (1996) J. Biol. Chem. 271: 931-938) DNA sequences are known, facilitating production of this enzyme by recombinant expression. In another embodiment, the claimed sialylation methods use a rat ST3Gal III.

Other exemplary sialyltransferases of use in the present invention include those isolated from Campylobacter jejuni, including the α(2,3). See, e.g, WO99/49051.

Sialyltransferases other those listed in Table 5, are also useful in an economic and efficient large-scale process for sialylation of commercially important glycopeptides. As a simple test to find out the utility of these other enzymes, various amounts of each enzyme (1-100 mU/mg protein) are reacted with asialo-α₁ AGP (at 1-10 mg/ml) to compare the ability of the sialyltransferase of interest to sialylate glycopeptides relative to either bovine ST6Gal I, ST3Gal III or both sialyltransferases. Alternatively, other glycopeptides or glycopeptides, or N-linked oligosaccharides enzymatically released from the polypeptide backbone can be used in place of asialo-α₁ AGP for this evaluation. Sialyltransferases with the ability to sialylate N-linked oligosaccharides of glycopeptides more efficiently than ST6Gal I are useful in a practical large-scale process for polypeptide sialylation (as illustrated for ST3Gal III in this disclosure). Other exemplary sialyltransferases are shown in FIG. 10.

In the conjugates of the invention, the Sia-modifying group cassette can be linked to the Gal in an α-2,6, or α-2,3 linkage.

### Fusion Proteins

In other exemplary embodiments, the methods of the invention utilize fusion proteins that have more than one enzymatic activity that is involved in synthesis of a desired glycopeptide conjugate. The fusion polypeptides can be composed of, for example, a catalytically active domain of a glycosyltransferase that is joined to a catalytically active domain of an accessory enzyme. The accessory enzyme catalytic domain can, for example, catalyze a step in the formation of a nucleotide sugar that is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressible nucleotide sequence. In other embodiments the fusion protein includes the catalytically active domains of two or more glycosyltransferases. See, for example, U.S. Pat. No. 5,641,668. The modified glycopeptides of the present invention can be readily designed and manufactured utilizing various suitable fusion proteins (see, for example, PCT Patent Application PCT/CA98/01180, which was published as WO 99/31224 on Jun. 24, 1999.)

### Immobilized Enzymes

In addition to cell-bound enzymes, the present invention also provides for the use of enzymes that are immobilized on a solid and/or soluble support. In an exemplary embodiment, there is provided a glycosyltransferase that is conjugated to a PEG via an intact glycosyl linker according to the methods of the invention. The PEG-linker-enzyme conjugate is optionally attached to solid support. The use of solid supported enzymes in the methods of the invention simplifies the work up of the reaction mixture and purification of the reaction product, and also enables the facile recovery of the enzyme. The glycosyltransferase conjugate is utilized in the methods of the invention. Other combinations of enzymes and supports will be apparent to those of skill in the art.

### Purification of Polypeptide Conjugates

The polypeptide conjugates produced by the processes described herein above can be used without purification. However, it is usually preferred to recover such products. Standard, well-known techniques for the purification of glycosylated saccharides, such as thin or thick layer chromatography, column chromatography, ion exchange chromatography, or membrane filtration. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have a molecular weight cutoff of about 3000 to about 10,000 can be used to remove proteins such as glycosyl transferases. Nanofiltration or reverse osmosis can then be used to remove salts and/or purify the product saccharides (see, e.g., WO 98/15581). Nanofilter membranes are a class of reverse osmosis membranes that pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

If the modified glycoprotein is produced intracellularly, as a first step, the particulate debris, including cells and cell debris, is removed, for example, by centrifugation or ultrafiltration. Optionally, the protein may be concentrated with a commercially available protein concentration filter, followed by separating the polypeptide variant from other impurities by one or more chromatographic steps, such as immunoaffinity chromatography, ion-exchange chromatography (e.g., on diethylaminoethyl (DEAE) or matrices containing carboxymethyl or sulfopropyl groups), hydroxy apatite chromatography and hydrophobic interaction chromatography (HIC). Exemplary stationary phases include Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, SP-Sepharose, or protein A Sepharose.

Other chromatographic techniques include SDS-PAGE chromatography, silica chromatography, chromatofocusing, reverse phase HPLC (e.g., silica gel with appended aliphatic groups), gel filtration using, e.g., Sephadex molecular sieve or size-exclusion chromatography, chromatography on columns that selectively bind the polypeptide, and ethanol or ammonium sulfate precipitation.

Modified glycopeptides produced in culture are usually isolated by initial extraction from cells, enzymes, etc., followed by one or more concentration, salting-out, aqueous ion-exchange, or size-exclusion chromatography steps, e.g., SP Sepharose. Additionally, the modified glycoprotein may be purified by affinity chromatography. HPLC may also be employed for one or more purification steps.

A protease inhibitor, e.g., methylsulfonylfluoride (PMSF) may be included in any of the foregoing steps to inhibit proteolysis and antibiotics may be included to prevent the growth of adventitious contaminants.

Within another embodiment, supernatants from systems which produce the modified glycopeptide of the invention are first concentrated using a commercially available protein concentration filter, for example, an Amicon or Millipore Pellicon ultrafiltration unit. Following the concentration step, the concentrate may be applied to a suitable purification matrix. For example, a suitable affinity matrix may comprise a ligand for the polypeptide, a lectin or antibody molecule bound to a suitable support. Alternatively, an anion-exchange resin may be employed, for example, a matrix or substrate having pendant DEAE groups. Suitable matrices include acrylamide, agarose, dextran, cellulose, or other types commonly employed in protein purification. Alternatively, a cation-exchange step may be employed. Suitable cation exchangers include various insoluble matrices comprising sulfopropyl or carboxymethyl groups. Sulfopropyl groups are particularly preferred.

Finally, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

The modified glycopeptide of the invention resulting from a large-scale fermentation may be purified by methods analogous to those disclosed by Urdal et al., J. Chromatog. 296:171 (1984). This reference describes two sequential, RP-HPLC steps for purification of recombinant human IL-2 on a preparative HPLC column. Alternatively, techniques such as affinity chromatography may be utilized to purify the modified glycoprotein.

### Acquisition of Polypeptide Coding Sequences

### General Recombinant Technology

The creation of mutant polypeptides, which incorporate an O-linked glycosylation sequence of the invention can be accomplished by altering the amino acid sequence of a corresponding parent polypeptide, by either mutation or by full chemical synthesis of the polypeptide. The polypeptide amino acid sequence is preferably altered through changes at the DNA level, particularly by mutating the DNA sequence encoding the polypeptide at preselected bases to generate codons that will translate into the desired amino acids. The DNA mutation(s) are preferably made using methods known in the art.

This invention relies on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

Nucleic acid sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Entire genes can also be chemically synthesized. Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of the cloned wild-type polypeptide genes, polynucleotide encoding mutant polypeptides, and synthetic oligonucleotides can be verified after cloning using, e.g., the chain termination method for sequencing double-stranded templates of Wallace et al., Gene 16: 21-26 (1981).

In an exemplary embodiment, the glycosylation sequence is added by shuffling polynucleotides. Polynucleotides encoding a candidate polypeptide can be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer, Proc. Natl. Acad. Sci. USA 91:10747-10751 (1994); Stemmer, Nature 370:389-391 (1994); and U.S. Pat. Nos. 5,605,793, 5,837,458, 5,830,721 and 5,811,238.

### Cloning and Subcloning of a Wild-Type Peptide Coding Sequence

Numerous polynucleotide sequences encoding wild-type polypeptides have been determined and are available from a commercial supplier, e.g., human growth hormone, e.g., GenBank Accession Nos. NM 000515, NM 002059, NM 022556, NM 022557, NM 022558, NM 022559, NM 022560, NM 022561, and NM 022562.

The rapid progress in the studies of human genome has made possible a cloning approach where a human DNA sequence database can be searched for any gene segment that has a certain percentage of sequence homology to a known nucleotide sequence, such as one encoding a previously identified polypeptide. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding a polypeptide can be isolated from a human cDNA or genomic DNA library using standard cloning techniques such as polymerase chain reaction (PCR), where homology-based primers can often be derived from a known nucleic acid sequence encoding a polypeptide. Most commonly used techniques for this purpose are described in standard texts, e.g., Sambrook and Russell, supra.

cDNA libraries suitable for obtaining a coding sequence for a wild-type polypeptide may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full-length polynucleotide sequence encoding the wild-type polypeptide from the cDNA library. A general description of appropriate procedures can be found in Sambrook and Russell, supra.

A similar procedure can be followed to obtain a full length sequence encoding a wild-type polypeptide, e.g., any one of the GenBank Accession Nos mentioned above, from a human genomic library. Human genomic libraries are commercially available or can be constructed according to various art-recognized methods. In general, to construct a genomic library, the DNA is first extracted from an tissue where a polypeptide is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a wild-type polypeptide is obtained.

Upon acquiring a nucleic acid sequence encoding a wild-type polypeptide, the coding sequence can be subcloned into a vector, for instance, an expression vector, so that a recombinant wild-type polypeptide can be produced from the resulting construct. Further modifications to the wild-type polypeptide coding sequence, e.g., nucleotide substitutions, may be subsequently made to alter the characteristics of the molecule.

Introducing Mutations into a Polypeptide Sequence

From an encoding polynucleotide sequence, the amino acid sequence of a wild-type polypeptide can be determined. Subsequently, this amino acid sequence may be modified to alter the protein's glycosylation pattern, by introducing additional glycosylation sequence(s) at various locations in the amino acid sequence.

Several types of protein glycosylation sequences are well known in the art. For instance, in eukaryotes, N-linked glycosylation occurs on the asparagine of the consensus sequence Asn-Xₐₐ-Ser/Thr, in which Xₐₐ is any amino acid except proline (Kornfeld et al., Ann Rev Biochem 54:631-664 (1985); Kukuruzinska et al., Proc. Natl. Acad. Sci. USA 84:2145-2149 (1987); Herscovics et al., FASEB J 7:540-550 (1993); and Orlean, Saccharomyces Vol. 3 (1996)). O-linked glycosylation takes place at serine or threonine residues (Tanner et al., Biochim. Biophys. Acta. 906:81-91 (1987); and Hounsell et al., Glycoconj. J. 13:19-26 (1996)). Other glycosylation patterns are formed by linking glycosylphosphatidylinositol to the carboxyl-terminal carboxyl group of the protein (Takeda et al., Trends Biochem. Sci. 20:367-371 (1995); and Udenfriend et al., Ann. Rev. Biochem. 64:593-591 (1995). Based on this knowledge, suitable mutations can thus be introduced into a wild-type polypeptide sequence to form new glycosylation sequences.

Although direct modification of an amino acid residue within a polypeptide sequence may be suitable to introduce a new N-linked or O-linked glycosylation sequence, more frequently, introduction of a new glycosylation sequence is accomplished by mutating the polynucleotide sequence encoding a polypeptide. This can be achieved by using any of known mutagenesis methods, some of which are discussed below.

A variety of mutation-generating protocols are established and described in the art. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

### Modification of Nucleic Acids for Preferred Codon Usage in a Host Organism

The polynucleotide sequence encoding a polypeptide variant can be further altered to coincide with the preferred codon usage of a particular host. For example, the preferred codon usage of one strain of bacterial cells can be used to derive a polynucleotide that encodes a polypeptide variant of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell. U.S. Pat. No. 5,824,864, for example, provides the frequency of codon usage by highly expressed genes exhibited by dicotyledonous plants and monocotyledonous plants.

At the completion of modification, the polpeptide variant coding sequences are verified by sequencing and are then subcloned into an appropriate expression vector for recombinant production in the same manner as the wild-type polypeptides.

### Expression of Mutant Polypeptides

Following sequence verification, the polypeptide variant of the present invention can be produced using routine techniques in the field of recombinant genetics, relying on the polynucleotide sequences encoding the polypeptide disclosed herein.

### Expression Systems

To obtain high-level expression of a nucleic acid encoding a mutant polypeptide of the present invention, one typically subclones a polynucleotide encoding the mutant polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the wild-type or mutant polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the mutant polypeptide in host cells. A typical expression cassette thus contains a promoter operably linked to the nucleic acid sequence encoding the mutant polypeptide and signals required for efficient polyadenylation of the transcript, ribosome binding sites, and translation termination. The nucleic acid sequence encoding the polypeptide is typically linked to a cleavable signal peptide sequence to promote secretion of the polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322-based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

In some exemplary embodiments the expression vector is chosen from pCWin1, pCWin2, pCWin2/MBP, pCWin2-MBP-SBD (pMS₃₉), and pCWin2-MBP-MCS-SBD (pMXS₃₉) as disclosed in co-owned U.S. patent application filed Apr. 9, 2004 which is incorporated herein by reference.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the mutant polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

When periplasmic expression of a recombinant protein (e.g., a hgh mutant of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that various conservative substitutions can be made to any wild-type or mutant polypeptide or its coding sequence while still retaining the biological activity of the polypeptide. Moreover, modifications of a polynucleotide coding sequence may also be made to accommodate preferred codon usage in a particular expression host without altering the resulting amino acid sequence.

### Transfection Methods

Standard transfection methods are used to produce bacterial, mammalian, yeast or insect cell lines that express large quantities of the mutant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the mutant polypeptide.

### Detection of Expression of MutantPolypeptides in Host Cells

After the expression vector is introduced into appropriate host cells, the transfected cells are cultured under conditions favoring expression of the mutant polypeptide. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a mutant polypeptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a mutant polypeptide of the present invention (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the mutant polypeptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6: 511-519 (1976). More detailed descriptions of preparing antibody against the mutant polypeptide of the present invention and conducting immunological assays detecting the mutant polypeptide are provided in a later section.

### Purification of Recombinantly Produced Mutant Polypeptides

Once the expression of a recombinant mutant polypeptide in transfected host cells is confirmed, the host cells are then cultured in an appropriate scale for the purpose of purifying the recombinant polypeptide.

1. Purification from Bacteria

When the mutant polypeptides of the present invention are produced recombinantly by transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a mutant polypeptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 2. Standard Protein Separation Techniques for Purification

When a recombinant polypeptide, e.g., the mutant polypeptide of the present invention, is expressed in host cells in a soluble form, its purification can follow standard protein purification procedures, for instance those described herein, below or purification can be accomplished using methods disclosed elsewhere, e.g., in PCT Publication No. WO2006/105426, which is incorporated by reference herein.

### Solubility Fractionation

Often as an initial step, and if the protein mixture is complex, an initial salt fractionation can separate many of the unwanted host cell proteins (or proteins derived from the cell culture media) from the recombinant protein of interest, e.g., a mutant polypeptide of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

### Ultrafiltration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a mutant polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

### Column Chromatography

The proteins of interest (such as the mutant polypeptide of the present invention) can also be separated from other proteins on the basis of their size, net surface charge, hydrophobicity, or affinity for ligands. In addition, antibodies raised against polypeptide can be conjugated to column matrices and the polypeptide be immunopurified. All of these methods are well known in the art.

It will be apparent to one of skill that chromatographic techniques can be performed at any scale and using equipment from many different manufacturers (e.g., Pharmacia Biotech).

### Immunoassays for Detection of Mutant Polypeptide Expression

To confirm the production of a recombinant mutant polypeptide, immunological assays may be useful to detect in a sample the expression of the polypeptide. Immunological assays are also useful for quantifying the expression level of the recombinant hormone. Antibodies against a mutant polypeptide are necessary for carrying out these immunological assays.

### Production of Antibodies Against Mutant Polypeptides

Methods for producing polyclonal and monoclonal antibodies that react specifically with an immunogen of interest are known to those of skill in the art (see, e.g., Coligan, Current Protocols in Immunology Wiley/Greene, NY, 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256: 495-497, 1975). Such techniques include antibody preparation by selection of antibodies from libraries of recombinant antibodies in phage or similar vectors (see, Huse et al., Science 246: 1275-1281, 1989; and Ward et al., Nature 341: 544-546, 1989).

In order to produce antisera containing antibodies with desired specificity, the polypeptide of interest (e.g., a mutant polypeptide of the present invention) or an antigenic fragment thereof can be used to immunize suitable animals, e.g., mice, rabbits, or primates. A standard adjuvant, such as Freund's adjuvant, can be used in accordance with a standard immunization protocol. Alternatively, a synthetic antigenic peptide derived from that particular polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be performed subsequently, see, Harlow and Lane, supra, and the general descriptions of protein purification provided above.

Monoclonal antibodies are obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. The general principles and methods of recombinant polypeptide production discussed above are applicable for antibody production by recombinant methods.

When desired, antibodies capable of specifically recognizing a mutant polypeptide of the present invention can be tested for their cross-reactivity against the wild-type polypeptide and thus distinguished from the antibodies against the wild-type protein. For instance, antisera obtained from an animal immunized with a mutant polypeptide can be run through a column on which a wild-type polypeptide is immobilized. The portion of the antisera that passes through the column recognizes only the mutant polypeptide and not the wild-type polypeptide. Similarly, monoclonal antibodies against a mutant polypeptide can also be screened for their exclusivity in recognizing only the mutant but not the wild-type polypeptide.

Polyclonal or monoclonal antibodies that specifically recognize only the mutant polypeptide of the present invention but not the wild-type polypeptide are useful for isolating the mutant protein from the wild-type protein, for example, by incubating a sample with a mutant peptide-specific polyclonal or monoclonal antibody immobilized on a solid support.

### Immunoassays for Detecting Recombinant Poypeptide Expression

Once antibodies specific for a mutant polypeptide of the present invention are available, the amount of the polypeptide in a sample, e.g., a cell lysate, can be measured by a variety of immunoassay methods providing qualitative and quantitative results to a skilled artisan. For a review of immunological and immunoassay procedures in general see, e.g., Stites, supra; U.S. Pat. Nos. 4,366,241; 4,376,110; 4,517,288; and 4,837,168.

### Labeling in Immunoassays

Immunoassays often utilize a labeling agent to specifically bind to and label the binding complex formed by the antibody and the target protein. The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In some cases, the labeling agent is a second antibody bearing a detectable label. Alternatively, the second antibody may lack a label, but it may, in turn, be bound by a labeled third antibody specific to antibodies of the species to which the second antibody corresponds. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al., J. Immunol., 135: 2589-2542 (1985)).

### Immunoassay Formats

Immunoassays for detecting a target protein of interest (e.g., a mutant human growth hormone) from samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second or third antibody bearing a label, as described above.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized.

In some cases, western blot (immunoblot) analysis is used to detect and quantify the presence of a mutant polypeptide in the samples. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against a mutant polypeptide.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5: 34-41 (1986)).

### Methods of Treatment

In addition to the conjugates discussed above, the present invention provides methods of preventing, curing or ameliorating a disease state by administering a polypeptide conjugate of the invention to a subject at risk of developing the disease or a subject that has the disease. Additionally, the invention provides methods for targeting conjugates of the invention to a particular tissue or region of the body.

The following examples are provided to illustrate the compositions and methods of the present invention, but not to limit the claimed invention.

What is claimed is:

1. A covalent conjugate between a glycosylated or non-glycosylated polypeptide and a polymeric modifying group, said polypeptide comprising an exogenous N-linked glycosylation sequence selected from SEQ ID NO: 1 and SEQ ID NO: 2:

| | |
|---|---|
| X¹ N X² X³ X⁴; | (SEQ ID NO: 1) |
| and | |
| X¹ D X^{2′} N X² X³X⁴, | (SEQ ID NO: 2) |

wherein
N is asparagine;
D is aspartic acid;
X³ is a member selected from threonine (T) and serine (S);
X¹ is either present or absent and when present is an amino acid;
X⁴ is either present or absent and when present is an amino acid; and
X² and X^{2′} are members independently selected amino acids, with the proviso that X² and X^{2′} are not proline (P),
wherein said polymeric modifying group is covalently conjugated to said polypeptide at said asparagine of said N-linked glycosylation sequence via a glycosyl linking group interposed between and covalently linked to both said asparagine and said polymeric modifying group, wherein said glycosyl linking group is a member selected from monosaccharides and oligosaccharides.

2. The covalent conjugate according to claim 1, wherein said exogenous N-linked glycosylation sequence is a member selected from N X² T and N X² S.

3. The covalent conjugate according to claim 1, wherein said polymeric modifying group is a member selected from linear and branched polymeric moieties.

4. The covalent conjugate according to claim 3, wherein said polymeric modifying group is a water-soluble polymer.

5. The covalent conjugate according to claim 4, wherein said water-soluble polymer is a member selected from poly(alkylene oxide), dextran and polysialic acid.

6. The covalent conjugate according to claim 5, wherein said poly(alkylene oxide) is a member selected from poly(ethylene glycol) (PEG), polypropylene glycol) (PPG) and derivatives thereof.

7. The covalent conjugate according to claim 1, said polypeptide corresponding to a parent-polypeptide, said parent polypeptide being a therapeutic polypeptide.

8. The covalent conjugate according to claim 1, wherein said polypeptide corresponds to a parent-polypeptide, which is a member selected from hepatocyte growth factor (HGF), nerve growth factors (NGF), epidermal growth factors (EGF), fibroblast growth factor-1 (FGF-1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, keratinocyte growth factor (KGF), megakaryocyte growth and development factor (MGDF), platelet-derived growth factor (PDGF), transforming growth factor-alpha (TGF-alpha), TGF-beta, TGF-beta2, TGF-beta3, vascular endothelial growth factors (VEGF), VEGF inhibitors, bone growth factor (BGF), glial growth factor, heparin-binding neurite-promoting factor (HBNF), C1 esterase inhibitor, human growth hormone (hGH), follicle stimulating hormone (FSH), thyroid stimulating hormone (TSH), parathyroid hormone, follitropin-alpha, follitropin-beta, follistatin, luteinizing hormone (LH), interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, interferon-alpha (INF-alpha), INF-beta, INF-gamma, INF-omega, INF-tau, insulin, glucocerebrosidase, alpha-galactosidase, acid-alpha-glucosidase (acid maltase), iduronidases, thyroid peroxidase (TPO), beta-glucosidase, arylsulfatase, asparaginase, alpha-glucoceramidase, sphingomyelinase, butyrylcholinesterase, urokinase, alpha-galactosidase A, bone morphogenetic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, NT-3, NT-4, NT-5, erythropoietins (EPO), novel erythropoiesis stimulating protein (NESP), growth differentiation factors (GDF), glial cell line-derived neurotrophic factor (GDNF), brain derived neurotrophic factor (BDNF), myostatin, nerve growth factor (NGF), von Willebrand factor (vWF), vWF-cleaving protease (vWF-protease, vWF-degrading protease), granulocyte colony stimulating factor (G-CSF), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), osteopontin, osteoprotegrin, protein C, somatomedin-1, somatotropin, somatropin, chimeric diphtheria toxin-IL-2, glucagon-like peptides (GLP), thrombin, thrombopoietin, thrombospondin-2, anti-thrombin III (AT-III), prokinetisin, CD4, α-CD20, tumor necrosis factors (TNF), TNF-alpha inhibitor, TNF receptor (TNF-R), P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, extendin-4, BDNF, beta-2-microglobulin, ciliary neurotrophic factor (CNTF), lymphotoxin-beta receptor (LT-beta receptor), fibrinogen, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, GLP-1, insulin-like growth factors, insulin-like growth factor binding proteins (IGB), IGF/IBP-2, IGF/IBP-3, IGF/IBP-4, IGF/IBP-5, IGF/IBP-6, IGF/IBP-7, IGF/IBP-8, IGF/IBP-9, IGF/IBP-10, IGF/IBP-11, IGF/IBP-12, IGF/IBP-13, Factor V, Factor VII, Factor VIII, Factor IX, Factor X, complex between von Willebrandt Factor (vWF) and Factor VIII, antibodies to endothelial growth factor (EGF), antibodies to vascular endothelial growth factors (VEGF), antibodies to fibroblast growth factors (FGF), anti-TNF antibodies, TNF receptor-IgG Fc region fusion protein, anti-HER2 antibodies, antibodies to protein F of respiratory syncytial virus, antibodies to TNF-α, antibodies to glycoprotein IIb/IIIa, antibodies to CD20, antibodies to CD4, antibodies to alpha-CD3, antibodies to CD40L, antibodies to CD154, antibodies to PSGL-1 and antibodies to carcinoembryonic antigen (CEA).

9. The covalent conjugate according to claim 1, wherein said exogenous N-linked glycosylation sequence is a substrate for an oligosaccharyltransferase.

10. The covalent conjugate according to claim 9, wherein said oligosaccharyltransferase is a recombinant enzyme.

11. The covalent conjugate according to claim 9, wherein said oligosaccharyltransferase is a member selected from PglB and Stt3p and soluble variants thereof.

12. The covalent conjugate according to claim 1, wherein said glycosyl linking group is an intact glycosyl linking group.

13. The covalent conjugate according to claim 1, wherein said glycosyl linking group is a residue which is a member selected from GlcNAc, GlcNH, bacillosamine, 6-hydroxybacillosamine, GalNAc, GalNH, GlcNAc-GlcNAc, GlcNAc-GlcNH, 6-hydroxybacillosamine-GalNAc, GalNAc-Gal-Sia, GlcNAc-GlcNAc-Gal-Sia, GlcNAc-Gal, GlcNAc-Gal-Sia, GlcNAc-GlcNAc-Man, GlcNAc-GlcNAc-Man(Man)₂ and combinations thereof.

14. A composition comprising a covalent conjugate according to claim 1 and a cell, in which said polypeptide is expressed.

15. A pharmaceutical composition comprising a covalent conjugate according to claim 1 and a pharmaceutically acceptable carrier.

16. A compound having a structure according to Formula (X): 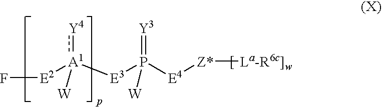
wherein
w is an integer selected from 1 to 8;
F is a lipid moiety;
Z* is a glycosyl moiety selected from monosaccharides and oligosaccharides;
each L^{a} is a linker moiety independently selected from a single bond, a functional group, substituted or unsubstituted alkyl, substituted or unsubstituted heteroalkyl, substituted or unsubstituted aryl, substituted or unsubstituted heteroaryl and substituted or unsubstituted heterocycloalkyl;
each R^{6c} is a member independently selected from a polymeric modifying group, a cytotoxin and a targeting moiety;
A is a member selected from P (phosphorus) and C (carbon);
Y³ is a member selected from oxygen (O) and sulfur (S);
Y⁴ is a member selected from O, S, SR¹, OR¹, OQ, CR¹R² and NR³R⁴;
E², E³ and E⁴ are members independently selected from CR¹R², O, S and NR³; and
each W is a member independently selected from SR¹, OR¹, OQ, NR³R⁴, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
each Q is a member independently selected from H, a negative charge and a cation; and
each R¹, each R², each R³ and each R⁴ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

17. The compound according to claim 16, wherein said polymeric modifying group is a member selected from linear and branched polymeric moieties.

18. The compound according to claim 17, wherein said polymeric modifying group is a water-soluble polymer.

19. The compound according to claim 18, wherein said water-soluble polymer is a member selected from poly(alkylene oxide), dextran and polysialic acid.

20. The compound according to claim 19, wherein said poly(alkylene oxide) is a member selected from poly(ethylene glycol) (PEG), poly(propylene glycol) (PPG) and derivatives thereof.

21. The compound according to claim 16, wherein Z* is a member selected from a mono-antennary, a di-antennary, a tri-antennary and a tetra-antennary glycan.

22. The compound according to claim 16, wherein Z* is a member selected from GlcNAc, GlcNH, bacillosamine, 6-hydroxybacillosamine, GalNAc, GalNH, GlcNAc-GlcNAc, GlcNAc-GlcNH, 6-hydroxybacillosamine-GalNAc, GalNAc-Gal-Sia, GlcNAc-GlcNAc-Gal-Sia, GlcNAc-Gal, GlcNAc-Gal-Sia, GlcNAc-GlcNAc-Man, GlcNAc-GlcNAc-Man(Man)₂ and combinations thereof.

23. The compound according to claim 16, wherein said lipid moiety comprises from 1 to about 100 carbon atoms, arranged in a straight or branched chain, said chain comprising carbon-carbon bonds, which are independently selected from saturated and unsaturated, said chain optionally including one or more aromatic or non-aromatic ring structures and optionally including at least one functional group.

24. The compound according to claim 23, wherein said functional group is a member selected from ether, thioether, amine, carboxamide, sulfonamide, hydrazine, carbonyl, carbamate, urea, thiourea, ester and carbonate.

25. The compound according to claim 16, wherein said lipid moiety is substituted alkyl.

26. The compound according to claim 25, wherein said lipid moiety is a member selected from dolichols, reduced or partially reduced dolichols, isoprenyl moieties, reduced isoprenyl moieties, poly-isoprenyl moieties and reduced or partially reduced poly-isoprenyl moieties.

27. The compound according to claim 26, wherein said poly-isoprenyl moiety is undecaprenyl.

28. The compound according to claim 25, wherein said lipid moiety has a structure, which is a member selected from: 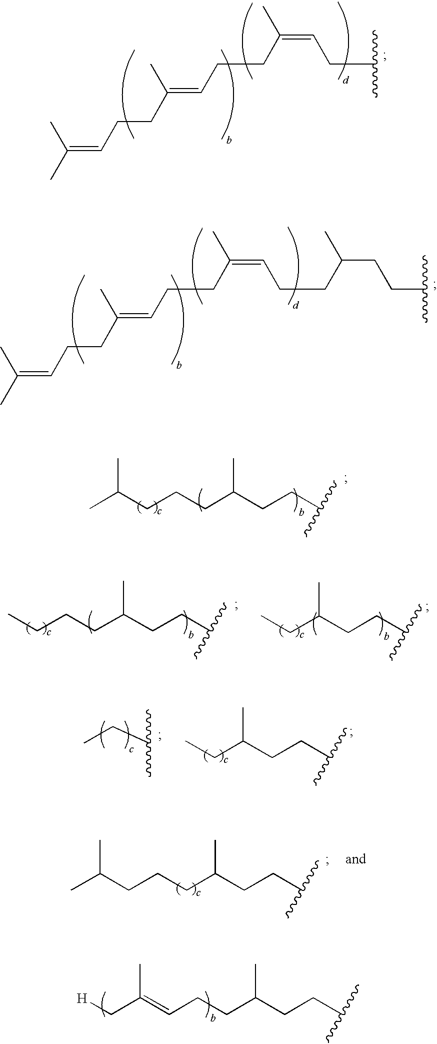
wherein b, c and d are integers independently selected from 0 to 100.

29. The compound according to claim 16, wherein R^{6c} has a structure, which is a member selected from: 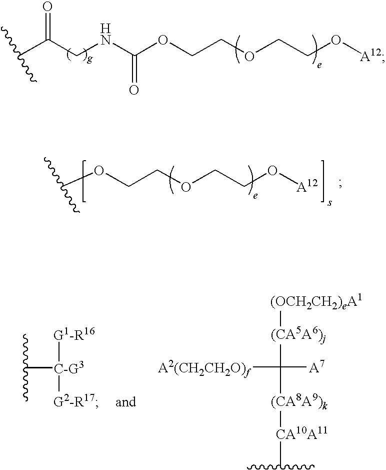
wherein
g, j and k are integers independently selected from 0 to 20;
each e and each f are integers independently selected from 0 to 2500;
s is an integer from 1-5;
R¹⁶ and R¹⁷ are independently selected polymeric moieties;
G¹ and G² are linkage fragments independently selected from O, S, SC(O)NH, HNC(O)S, SC(O)O, O, NH, NHC(O), (O)CNH and NHC(O)O, and OC(O)NH, CH₂S, CH₂O, CH₂CH₂O, CH₂CH₂S, (CH₂)ₒO, (CH₂)ₒS or (CH₂)ₒY′-PEG,
wherein
o is an integer from 1 to 50; and
Y′ is S, NH, NHC(O), C(O)NH, NHC(O)O, OC(O)NH or O;
G³ is a member selected from H, substituted or unsubstituted alkyl, substituted or unsubstituted heteroalkyl, substituted or unsubstituted heterocycloalkyl, substituted or unsubstituted aryl, and substituted or unsubstituted heteroaryl; and
A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NA¹²A¹³, —OA¹² and —SiA¹²A¹³
wherein
A¹² and A¹³ are members independently selected from H, substituted or unsubstituted alkyl, substituted or unsubstituted heteroalkyl, substituted or unsubstituted heterocycloalkyl, substituted or unsubstituted aryl, and substituted or unsubstituted heteroaryl.

30. The compound according to claim 16, having the structure:

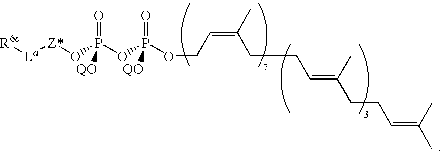

31. The compound according to claim 30, having a structure, which is a member selected from:

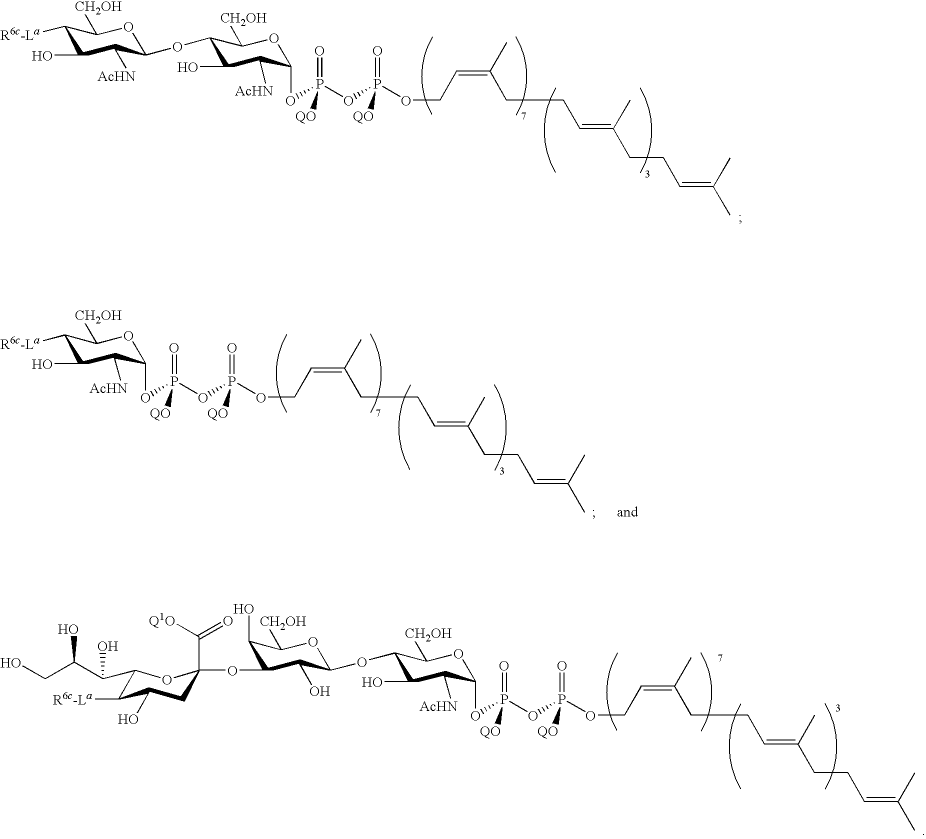

32. The compound according to claim 31, having a structure, which is a member selected from: 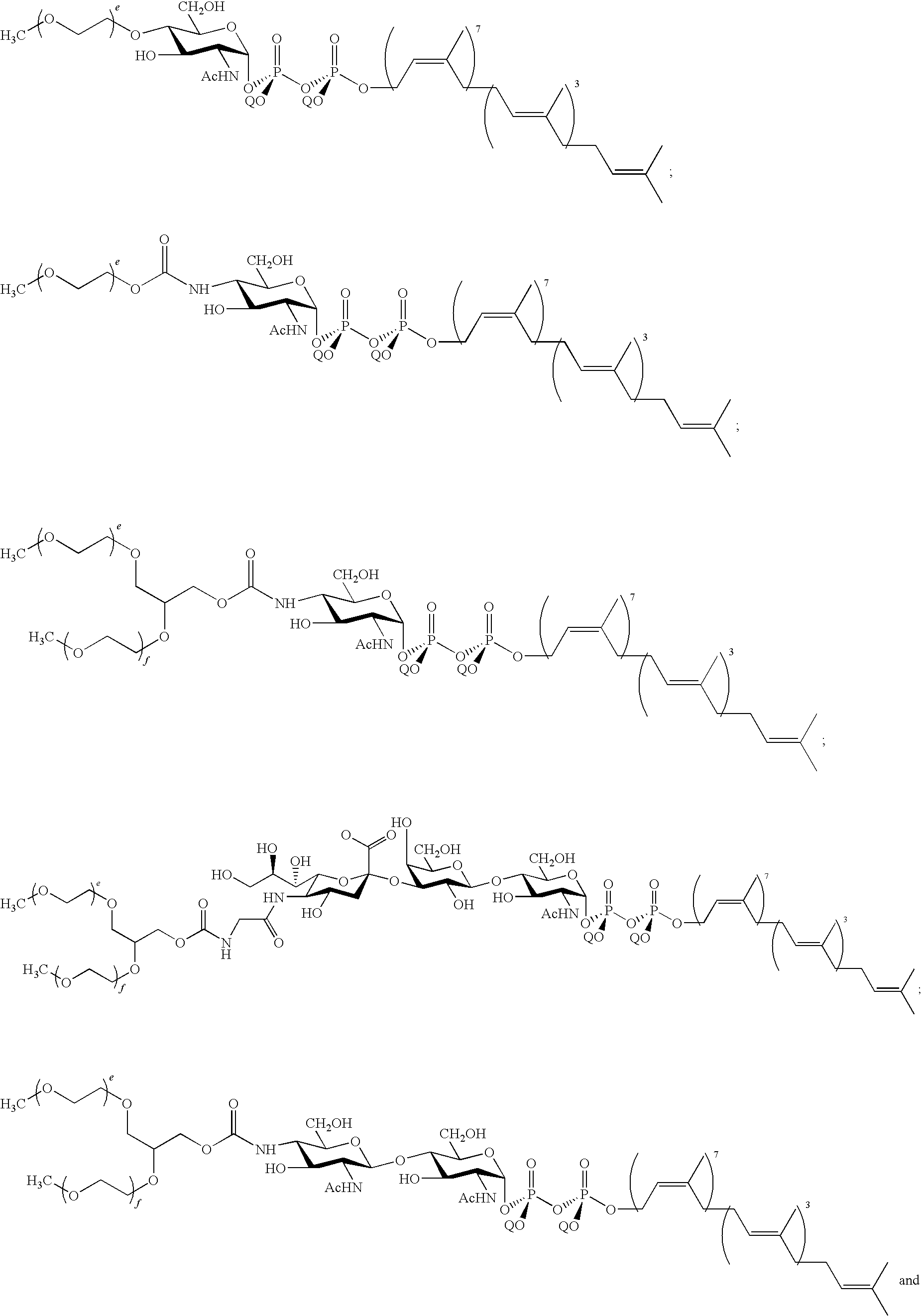
wherein
e and f are integers independently selected from 1 to 2500; and
Q¹ is a member selected from H, a negative charge and a counter-ion.

33. A composition comprising a cell and a compound according to claim 16.

34. A polypeptide comprising an exogenous N-linked glycosylation sequence selected from SEQ ID NO: 1 and SEQ ID NO: 2:

| | |
|---|---|
| X¹ N X² X³ X⁴; | (SEQ ID NO: 1) |
| and | |
| X¹ D X^{2′} N X² X³ X⁴, | (SEQ ID NO: 2) |

wherein
N is asparagine;
D is aspartic acid;
X³ is a member selected from threonine (T) and serine (S);
X¹ is either present or absent and when present is an amino acid;
X⁴ is either present or absent and when present is an amino acid; and
X² and X^{2′} are independently selected amino acids, with the proviso that X² and X^{2′} are not proline (P).

35. An isolated nucleic acid encoding said polypeptide of claim 34.

36. An expression vector comprising said nucleic acid of claim 35.

37. A cell comprising said nucleic acid of claim 35.

38. A library of polypeptides comprising a plurality of different members, wherein each member of said library corresponds to a common parent polypeptide and wherein each member of said library comprises an exogenous N-linked glycosylation sequence, wherein each of said N-linked glycosylation sequence is a member independently selected from SEQ ID NO: 1 and SEQ ID NO: 2:

| | |
|---|---|
| X¹ N X² X³ X⁴; | (SEQ ID NO: 1) |
| and | |
| X¹ D X^{2′} N X² X³ X⁴ | (SEQ ID NO: 2) |

wherein
N is asparagine;
D is aspartic acid;
X³ is a member selected from threonine (T) and serine (S);
X¹ is either present or absent and when present is an amino acid;
X⁴ is either present or absent and when present is an amino acid; and
X² and X^{2′} are independently selected amino acids, with the proviso that X² and X^{2′} are not proline (P).

39. The library according to claim 38, wherein said exogenous N-linked glycosylation sequence is a member selected from N X² T and N X² S.

40. The library according to claim 38, wherein each member of said library comprises the same N-linked glycosylation sequence at a different amino acid position within said parent polypeptide.

41. The library according to claim 38, wherein each member of said library comprises a different N-linked glycosylation sequence at the same amino acid position within said parent polypeptide.

42. The library according to claim 38, wherein said N-linked glycosylation sequence is a substrate for an oligosaccharyltransferase.

43. The library according to claim 42, wherein said oligosaccharyltransferase is a recombinant enzyme.

44. The library according to claim 42, wherein said oligosaccharyltransferase is a member selected from PglB and Stt3 and soluble variants thereof.

45. The library according to claim 38, wherein said parent-polypeptide is a member selected from hepatocyte growth factor (HGF), nerve growth factors (NGF), epidermal growth factors (EGF), fibroblast growth factor-1 (FGF-1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, keratinocyte growth factor (KGF), megakaryocyte growth and development factor (MGDF), platelet-derived growth factor (PDGF), transforming growth factor-alpha (TGF-alpha), TGF-beta, TGF-beta2, TGF-beta3, vascular endothelial growth factors (VEGF), VEGF inhibitors, bone growth factor (BGF), glial growth factor, heparin-binding neurite-promoting factor (HBNF), C1 esterase inhibitor, human growth hormone (hGH), follicle stimulating hormone (FSH), thyroid stimulating hormone (TSH), parathyroid hormone, follitropin-alpha, follitropin-beta, follistatin, luteinizing hormone (LH), interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, interferon-alpha (INF-alpha), INF-beta, INF-gamma, INF-omega, INF-tau, insulin, glucocerebrosidase, alpha-galactosidase, acid-alpha-glucosidase (acid maltase), iduronidases, thyroid peroxidase (TPO), beta-glucosidase, arylsulfatase, asparaginase, alpha-glucoceramidase, sphingomyelinase, butyrylcholinesterase, urokinase, alpha-galactosidase A, bone morphogenetic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, NT-3, NT-4, NT-5, erythropoietins (EPO), novel erythropoiesis stimulating protein (NESP), growth differentiation factors (GDF), glial cell line-derived neurotrophic factor (GDNF), brain derived neurotrophic factor (BDNF), myostatin, nerve growth factor (NGF), von Willebrand factor (vWF), vWF-cleaving protease (vWF-protease, vWF-degrading protease), granulocyte colony stimulating factor (G-CSF), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), osteopontin, osteoprotegrin, protein C, somatomedin-1, somatotropin, somatropin, chimeric diphtheria toxin-IL-2, glucagon-like peptides (GLP), thrombin, thrombopoietin, thrombospondin-2, anti-thrombin III (AT-III), prokinetisin, CD4, α-CD20, tumor necrosis factors (TNF), TNF-alpha inhibitor, TNF receptor (TNF-R), P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, extendin-4, BDNF, beta-2-microglobulin, ciliary neurotrophic factor (CNTF), lymphotoxin-beta receptor (LT-beta receptor), fibrinogen, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, GLP-1, insulin-like growth factors, insulin-like growth factor binding proteins (IGB), IGF/IBP-2, IGF/IBP-3, IGF/IBP-4, IGF/IBP-5, IGF/IBP-6, IGF/IBP-7, IGF/IBP-8, IGF/IBP-9, IGF/IBP-10, IGF/IBP-11, IGF/IBP-12, IGF/IBP-13, Factor V, Factor VII, Factor VIII, Factor IX, Factor X, complex between von Willebrandt Factor (vWF) and Factor VIII, antibodies to endothelial growth factor (EGF), antibodies to vascular endothelial growth factors (VEGF), antibodies to fibroblast growth factors (FGF), anti-TNF antibodies, TNF receptor-IgG Fc region fusion protein, anti-HER2 antibodies, antibodies to protein F of respiratory syncytial virus, antibodies to TNF-α, antibodies to glycoprotein IIb/IIIa, antibodies to CD20, antibodies to CD4, antibodies to alpha-CD3, antibodies to CD40L, antibodies to CD154, antibodies to PSGL-1 and antibodies to carcinoembryonic antigen (CEA).

46. A cell-free in vitro method of forming a covalent conjugate between a polypeptide and a polymeric modifying group, wherein said polypeptide comprises an N-linked glycosylation sequence including an asparagine residue, said modifying group covalently linked to said polypeptide at said asparagine residue via a glycosyl linking group interposed between and covalently linked to both said asparagine and said modifying group, said method comprising: contacting said polypeptide and a compound according to claim 16, in the presence of an oligosaccharyltransferase under conditions sufficient for said oligosaccharyltransferase to transfer a glycosyl moiety from said compound onto said asparagine residue of said N-linked glycosylation sequence, thereby forming said covalent conjugate.

47. The method according to claim 46, further comprising: expressing said polypeptide in a host-cell.

48. The method according to claim 47, further comprising: generating an expression vector comprising a nucleic acid sequence encoding said polypeptide.

49. The method according to claim 48, further comprising: transfecting said host cell with said expression vector.

50. The method according to claim 46, further comprising: isolating said covalent conjugate.

51. The method according to claim 46, wherein said polymeric modifying group is a member selected from linear and branched polymeric moieties.

52. The method according to claim 51, wherein said polymeric modifying group is a water-soluble polymer.

53. The method according to claim 52, wherein said water-soluble polymer is a member selected from poly(alkylene oxide), dextran and polysialic acid.

54. The method according to claim 53, wherein said poly(alkylene oxide) is a member selected from poly(ethylene glycol) (PEG), polypropylene glycol) (PPG) and derivatives thereof.

55. The method according to claim 46, wherein said polypeptide corresponds to a parent-polypeptide that is a therapeutic polypeptide.

56. The method according to claim 46, wherein said polypeptide corresponds to a parent-polypeptide, which is a member selected from hepatocyte growth factor (HGF), nerve growth factors (NGF), epidermal growth factors (EGF), fibroblast growth factor-1 (FGF-1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, keratinocyte growth factor (KGF), megakaryocyte growth and development factor (MGDF), platelet-derived growth factor (PDGF), transforming growth factor-alpha (TGF-alpha), TGF-beta, TGF-beta2, TGF-beta3, vascular endothelial growth factors (VEGF), VEGF inhibitors, bone growth factor (BGF), glial growth factor, heparin-binding neurite-promoting factor (HBNF), C1 esterase inhibitor, human growth hormone (hGH), follicle stimulating hormone (FSH), thyroid stimulating hormone (TSH), parathyroid hormone, follitropin-alpha, follitropin-beta, follistatin, luteinizing hormone (LH), interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, interferon-alpha (INF-alpha), INF-beta, INF-gamma, INF-omega, INF-tau, insulin, glucocerebrosidase, alpha-galactosidase, acid-alpha-glucosidase (acid maltase), iduronidases, thyroid peroxidase (TPO), beta-glucosidase, arylsulfatase, asparaginase, alpha-glucoceramidase, sphingomyelinase, butyrylcholinesterase, urokinase, alpha-galactosidase A, bone morphogenetic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, NT-3, NT-4, NT-5, erythropoietins (EPO), novel erythropoiesis stimulating protein (NESP), growth differentiation factors (GDF), glial cell line-derived neurotrophic factor (GDNF), brain derived neurotrophic factor (BDNF), myostatin, nerve growth factor (NGF), von Willebrand factor (vWF), vWF-cleaving protease (vWF-protease, vWF-degrading protease), granulocyte colony stimulating factor (G-CSF), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), osteopontin, osteoprotegrin, protein C, somatomedin-1, somatotropin, somatropin, chimeric diphtheria toxin-IL-2, glucagon-like peptides (GLP), thrombin, thrombopoietin, thrombospondin-2, anti-thrombin III (AT-III), prokinetisin, CD4, α-CD20, tumor necrosis factors (TNF), TNF-alpha inhibitor, TNF receptor (TNF-R), P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, extendin-4, BDNF, beta-2-microglobulin, ciliary neurotrophic factor (CNTF), lymphotoxin-beta receptor (LT-beta receptor), fibrinogen, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, GLP-1, insulin-like growth factors, insulin-like growth factor binding proteins (IGB), IGF/IBP-2, IGF/IBP-3, IGF/IBP-4, IGF/IBP-5, IGF/IBP-6, IGF/IBP-7, IGF/IBP-8, IGF/IBP-9, IGF/IBP-10, IGF/IBP-11, IGF/IBP-12, IGF/IBP-13, Factor V, Factor VII, Factor VIII, Factor IX, Factor X, complex between von Willebrandt Factor (vWF) and Factor VIII, antibodies to endothelial growth factor (EGF), antibodies to vascular endothelial growth factors (VEGF), antibodies to fibroblast growth factors (FGF), anti-TNF antibodies, TNF receptor-IgG Fc region fusion protein, anti-HER2 antibodies, antibodies to protein F of respiratory syncytial virus, antibodies to TNF-α, antibodies to glycoprotein IIb/IIIa, antibodies to CD20, antibodies to CD4, antibodies to alpha-CD3, antibodies to CD40L, antibodies to CD154, antibodies to PSGL-1 and antibodies to carcinoembryonic antigen (CEA).

57. The method according to claim 46, wherein said oligosaccharyltransferase is a recombinant enzyme.

58. The method according to claim 46, wherein said oligosaccharyltransferase is a member selected from PglB and Stt3 and soluble variants thereof.

59. The method according to claim 46, wherein said glycosyl linking group is an intact glycosyl linking group.

60. The method according to claim 46, wherein said glycosyl linking group is a residue which is a member selected from GlcNAc, GlcNH, bacillosamine, 6-hydroxybacillosamine, GalNAc, GalNH, GlcNAc-GlcNAc, GlcNAc-GlcNH, 6-hydroxybacillosamine-GalNAc, GalNAc-Gal-Sia, GlcNAc-GlcNAc-Gal-Sia, GlcNAc-Gal, GlcNAc-Gal-Sia, GlcNAc-GlcNAc-Man, GlcNAc-GlcNAc-Man(Man)₂ and combinations thereof.

61. A method of forming a covalent conjugate between a polypeptide and a polymeric modifying group, said polypeptide comprising a N-linked glycosylation sequence including an asparagine residue, said modifying group covalently linked to said polypeptide at said asparagine residue via a glycosyl linking group interposed between and covalently linked to both said asparagine and said modifying group, said method comprising:
(i) contacting said polypeptide and a compound according to claim 16, in the presence of an oligosaccharyltransferase under conditions sufficient for said oligosaccharyltransferase to transfer a glycosyl moiety covalently linked to said modifying group from said compound onto said asparagine residue of said N-linked glycosylation sequence, wherein said contacting occurs within a host cell, in which said polypeptide is expressed,
thereby forming said covalent conjugate.

62. The method according to claim 61, further comprising:
(ii) contacting said host cell with said compound; and
(iii) incubating said host cell under conditions sufficient for said host cell to internalize said compound.

63. The method according to claim 61, wherein said cell is present in a cell-culture media, said cell-culture media supplemented with said compound.

64. The method according to claim 61, further comprising: isolating said covalent conjugate.

65. The method according to claim 61, further comprising: generating an expression vector comprising a nucleic acid sequence encoding said polypeptide.

66. The method according to claim 65, further comprising: transfecting said host cell with said expression vector.

67. The method according to claim 61, wherein said polymeric modifying group is a member selected from linear and branched polymeric moieties.

68. The method according to claim 61, wherein said polymeric modifying group is a water-soluble polymer.

69. The method according to claim 68, wherein said water-soluble polymer is a member selected from poly(alkylene oxide), dextran and polysialic acid.

70. The method according to claim 69, wherein said poly(alkylene oxide) is a member selected from poly(ethylene glycol) (PEG), polypropylene glycol) (PPG) and derivatives thereof.

71. The method according to claim 61, wherein said polypeptide corresponds to a parent-polypeptide, said parent-polypeptide being a therapeutic polypeptide.

72. The method according to claim 61, wherein said polypeptide corresponds to a parent-polypeptide, which is a member selected from hepatocyte growth factor (HGF), nerve growth factors (NGF), epidermal growth factors (EGF), fibroblast growth factor-1 (FGF-1), FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, keratinocyte growth factor (KGF), megakaryocyte growth and development factor (MGDF), platelet-derived growth factor (PDGF), transforming growth factor-alpha (TGF-alpha), TGF-beta, TGF-beta2, TGF-beta3, vascular endothelial growth factors (VEGF), VEGF inhibitors, bone growth factor (BGF), glial growth factor, heparin-binding neurite-promoting factor (HBNF), C1 esterase inhibitor, human growth hormone (hGH), follicle stimulating hormone (FSH), thyroid stimulating hormone (TSH), parathyroid hormone, follitropin-alpha, follitropin-beta, follistatin, luteinizing hormone (LH), interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, interferon-alpha (INF-alpha), INF-beta, INF-gamma, INF-omega, INF-tau, insulin, glucocerebrosidase, alpha-galactosidase, acid-alpha-glucosidase (acid maltase), iduronidases, thyroid peroxidase (TPO), beta-glucosidase, arylsulfatase, asparaginase, alpha-glucoceramidase, sphingomyelinase, butyrylcholinesterase, urokinase, alpha-galactosidase A, bone morphogenetic protein-1 (BMP-1), BMP-2, BMP-3, BMP-4, BMP-5, BMP-6, BMP-7, BMP-8, BMP-9, BMP-10, BMP-11, BMP-12, BMP-13, BMP-14, BMP-15, NT-3, NT-4, NT-5, erythropoietins (EPO), novel erythropoiesis stimulating protein (NESP), growth differentiation factors (GDF), glial cell line-derived neurotrophic factor (GDNF), brain derived neurotrophic factor (BDNF), myostatin, nerve growth factor (NGF), von Willebrand factor (vWF), vWF-cleaving protease (vWF-protease, vWF-degrading protease), granulocyte colony stimulating factor (G-CSF), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), osteopontin, osteoprotegrin, protein C, somatomedin-1, somatotropin, somatropin, chimeric diphtheria toxin-IL-2, glucagon-like peptides (GLP), thrombin, thrombopoietin, thrombospondin-2, anti-thrombin III (AT-III), prokinetisin, CD4, α-CD20, tumor necrosis factors (TNF), TNF-alpha inhibitor, TNF receptor (TNF-R), P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, extendin-4, BDNF, beta-2-microglobulin, ciliary neurotrophic factor (CNTF), lymphotoxin-beta receptor (LT-beta receptor), fibrinogen, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, GLP-1, insulin-like growth factors, insulin-like growth factor binding proteins (IGB), IGF/IBP-2, IGF/IBP-3, IGF/IBP-4, IGF/IBP-5, IGF/IBP-6, IGF/IBP-7, IGF/IBP-8, IGF/IBP-9, IGF/IBP-10, IGF/IBP-11, IGF/IBP-12, IGF/IBP-13, Factor V, Factor VII, Factor VIII, Factor IX, Factor X, complex between von Willebrandt Factor (vWF) and Factor VIII, antibodies to endothelial growth factor (EGF), antibodies to vascular endothelial growth factors (VEGF), antibodies to fibroblast growth factors (FGF), anti-TNF antibodies, TNF receptor-IgG Fc region fusion protein, anti-HER2 antibodies, antibodies to protein F of respiratory syncytial virus, antibodies to TNF-α, antibodies to glycoprotein IIb/IIIa, antibodies to CD20, antibodies to CD4, antibodies to alpha-CD3, antibodies to CD40L, antibodies to CD154, antibodies to PSGL-1 and antibodies to carcinoembryonic antigen (CEA).

73. The method according to claim 61, wherein said oligosaccharyltransferase is a recombinant enzyme co-expressed in said host cell.

74. The method according to claim 61, wherein said oligosaccharyltransferase is endogenous to said host cell.

75. The method according to claim 61, wherein said oligosaccharyltransferase is a member selected from PglB and Stt3 and soluble variants thereof.

76. The method according to claim 61, wherein said glycosyl linking group is an intact glycosyl linking group.

77. The method according to claim 61, wherein said glycosyl linking group is a residue which is a member selected from GlcNAc, GlcNH, bacillosamine, 6-hydroxybacillosamine, GalNAc, GalNH, GlcNAc-GlcNAc, GlcNAc-GlcNH, 6-hydroxybacillosamine-GalNAc, GalNAc-Gal-Sia, GlcNAc-GlcNAc-Gal-Sia, GlcNAc-Gal, GlcNAc-Gal-Sia, GlcNAc-GlcNAc-Man, GlcNAc-GlcNAc-Man(Man)₂ and combinations thereof.
